(19) United States
(12) Patent Application Publication
(10) Pub. No.: US 2026/0308552 A1
(43) Pub. Date: Oct. 8, 2026

(54) NOVEL BASE EDITORS AND USES THEREOF

(71) Applicant: HuidaGene Therapeutics (Singapore) Pte. Ltd., Singapore (SG)

(72) Inventor: Huawei Tong, Shanghai (CN)

(21) Appl. No.: 19/477,356

(22) PCT Filed: Apr. 25, 2024
(86) PCT No.: PCT/CN2024/089874

§ 371 Date: Oct. 21, 2025

(30) Foreign Application Priority Data
Apr. 25, 2023 (WO) PCT/CN2023/090660
Apr. 28, 2023 (WO) PCT/CN2023/091734
May 16, 2023 (WO) PCT/CN2023/094565
Jan. 2, 2024 (WO) PCT/CN2024/070217
Mar. 28, 2024 (WO) PCT/CN2024/084498

(51) Int. Cl. C12N 9/22; C12N 9/24; C12N 15/11

(52) U.S. Cl. CPC C12N 9/226; C12N 9/2497; C12N 15/111; C07K 2319/80; C12N 2310/20; C12Y 302/02021; C12Y 302/02027

(57) ABSTRACT

Provided in the disclosure are novel base editors and uses thereof.

### REFERENCE TO RELATED APPLICATIONS

The instant application claims the priority to and the benefit of the filing date of PCT/CN2023/090660, filed on Apr. 25, 2023; PCT/CN2023/091734, filed on Apr. 28, 2023; PCT/CN2023/094565, filed on May 16, 2023; PCT/CN2024/070217, filed on Jan. 2, 2024; and PCT/CN2024/084498, filed on Mar. 28, 2024, the entire contents of which, including any drawings and sequence listing, are incorporated herein by reference.

### REFERENCE TO AN ELECTRONIC SEQUENCE LISTING

The disclosure contains a Sequence Listing XML file which has been submitted electronically in XML format and is hereby incorporated by reference in its entirety. Said XML copy, created on Apr. 25, 2024, by software “WIPO Sequence” according to WIPO Standard ST. 26, is named HGP032PCT. xml, and is 2,772,603 bytes in size.

According to WIPO Standard ST. 26, symbol “t” is used to denote both T in DNA and U in RNA. Thus, in the instant sequence listing prepared according to ST. 26, wherever a sequence is an RNA, the T in the sequence shall be deemed as U.

### BACKGROUND

Base editing is a powerful technology for basic research and therapeutic applications [1, 2]. Current base editors mainly contain a nucleic acid programmable DNA binding protein, such as a catalytically impaired CRISPR-associated (Cas) nuclease, that was fused with a single-stranded DNA deaminase enzyme and sometimes an additional protein that could modulate DNA repair machinery [3, 4]. In the past several years, two main classes of DNA base editors, adenine base editors (ABEs) [5] and cytosine base editors (CBEs) [4] have been developed and widely used for A-to-G and C-to-T conversions, respectively (FIG. 1a). Recently, C-to-G base editors (CGBEs) [6-10] and adenine transversion base editor (AYBE) [11] were constructed by fusing existing CBE or ABE with a DNA glycosylase variant to generate new tools for achieving more versatile base editing outcomes, including C-to-G, A-to-C and A-to-T editing (FIG. 9). CRISPR-free CBEs (DdCBEs) were reported for performing C-to-T base editing in mitochondria DNA, by fusing two halves of a double-strand DNA cytidine deaminase (DddA) variants with two separate TALE (transcription activator-like effector) proteins [12-14]. So far, these base editing methods all begin with deamination of C or A as an essential step to produce uridine (U) or inosine (I) intermediate, respectively, which in turn is transformed into another base by endogenous DNA repair or replication mechanisms [4-11]. Although G and T in the non-edited strand could be modified along with the editing of C and A in the edited strand, respectively, no existing base editor is capable of directly editing G or T.

It would be desired to develop novel base editors and base editing methods for base editing of a deoxyribonucleotide, such as, dG or dT, beyond the current base editors.

Citation or identification of any document in the disclosure is not an admission that such a document is available as prior art to the disclosure. Each of the references mentioned or cited in the disclosure is incorporated by reference in its entirety.

### SUMMARY

Provided in the disclosure includes at least in part base editors and base editing methods capable of direct base editing of a target deoxyribonucleotide (e.g., dG, dT) in a target dsDNA. Provided in the disclosure includes at least in part base editors and base editing methods capable of base editing of a target deoxyribonucleotide (e.g., dC) in a target dsDNA in the absence of deamination.

In an aspect, the disclosure provides a fusion protein comprising:
(1) a nucleic acid programmable DNA binding domain (napDNAbd) capable of binding a target dsDNA comprising:
(a) a first deoxyribonucleotide (e.g., dG (deoxyguanosine), dT (thymidine), dC (deoxycytidine)) in a protospacer sequence on a nontarget strand (edited strand) of the target dsDNA, and
(b) a second deoxyribonucleotide (e.g., dC (deoxycytidine), dA (deoxyadenosine), dG (deoxyguanosine)) base pairing with the first deoxyribonucleotide (e.g., dG, dT, dC) and in a target sequence on the target strand (non-edited strand) of the target dsDNA, wherein the protospacer sequence is fully reverse complementary to the target sequence; and
(2) a base excising domain capable of excising the base (e.g., guanine, thymine, cytosine) of the first deoxyribonucleotide.

In some embodiments, the fusion protein does not comprise a deaminase domain, e.g., an adenine or cytosine deaminase domain, e.g., TadA and variants thereof.

In some embodiments, the first deoxyribonucleotide is deoxyguanosine (dG), thymidine (dT), or deoxycytidine (dC).

In some embodiments, the conversion of the first deoxyribonucleotide to the fourth deoxyribonucleotide is dG-to-dA, dG-to-dT, dG-to-dC, dT-to-dA, dT-to-dC, dT-to-dG, dC-to-dA, dC-to-dT, or dC-to-dG.

In some embodiments, the base excising domain comprises a glycosylase.

In some embodiments, the glycosylase is selected from the group consisting of N-methylpurine DNA glycosylase (MPG), 8-oxoguanine DNA glycosylase (OGG1), methyl-CpG binding domain 4, DNA glycosylase (MBD4), thymine DNA glycosylase (TDG), uracil DNA glycosylase (UNG), single-strand-selective monofunctional uracil-DNA glycosylase 1 (SMUG1), mutY DNA glycosylase (MUTYH), nth like DNA glycosylase 1 (NTHL1), nei like DNA glycosylase 1 (NEIL1), nei like DNA glycosylase 2 (NEIL2), nei like DNA glycosylase 3 (NEIL3), and mutants thereof capable of recognizing and excising a base from a nucleotide of a nucleic acid.

In some embodiments, the base excising domain comprises an N-methylpurine DNA glycosylase (MPG).

In some embodiments, the MPG comprises an amino acid substitution relative to a reference MPG of SEQ ID NO: 7 at a position that is corresponding to a position or that is a position selected from the group consisting of N169, D175, C178, and/or Q294 of the reference MPG, wherein the position is numbered according to SEQ ID NO: 1.

In some embodiments, the amino acid substitution is a substitution with R, A, N, or G.

In some embodiments, the MPG comprises an amino acid substitution relative to a reference MPG of SEQ ID NO: 7 that is corresponding to a substitution or that is a substitution selected from the group consisting of N169G, D175R, C178N, Q294R, and a combination of any two or more substitutions thereof, wherein the position is numbered according to SEQ ID NO: 1.

In some embodiments, the MPG comprises a combination substitution relative to a reference MPG of SEQ ID NO: 7 that is corresponding to a combination substitution of N169G, D175R, C178N, and Q294R, wherein the position is numbered according to SEQ ID NO: 1.

In some embodiments, the MPG comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 8, 16, 18, 20, 22, 24, 26, 28, 30, 32, 34, 36, or 38.

In some embodiments, the MPG is (substantially) capable of excising guanine of dG.

In some embodiments, the base excising domain comprises an uracil-DNA glycosylase (UNG).

In some embodiments, the UNG comprises an amino acid substitution relative to a reference UNG of SEQ ID NO: 135 or 137 at a position that is corresponding to a position or that is a position selected from the group consisting of K184, A214, Q259, and/or Y284 of the reference UNG, wherein the position is numbered according to SEQ ID NO: 133.

In some embodiments, the amino acid substitution is a substitution with A, D, V, or T.

In some embodiments, the UNG comprises an amino acid substitution relative to a reference UNG of SEQ ID NO: 135 or 137 that is corresponding to a substitution or that is a substitution selected from the group consisting of K184A, A214V, A214T, Q259A, Y284D, and a combination of any two or more substitutions thereof, wherein the position is numbered according to SEQ ID NO: 133.

In some embodiments, the UNG comprises a deletion of amino acids at positions that are corresponding to positions or that are positions 1-65, 1-66, 1-67, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of the reference UNG of SEQ ID NO: 135 or 137, wherein the position is numbered according to SEQ ID NO: 133.

In some embodiments, the UNG comprises an amino acid substitution relative to a reference UNG of SEQ ID NO: 135 that is corresponding to a substitution or that is a substitution selected from the group consisting of A214T, Q259A, Y284D, and a combination of any two or more substitutions thereof, and comprises a deletion of amino acids at positions that are corresponding to positions or that are positions 1-88 of the reference UNG, wherein the position is numbered according to SEQ ID NO: 133.

In some embodiments, the UNG comprises an amino acid substitution relative to a reference UNG of SEQ ID NO: 137 that is corresponding to a substitution or that is a substitution selected from the group consisting of K184A, A214V, and a combination of the two substitutions, and comprises a deletion of amino acids at positions that are corresponding to positions or that are positions 1-88 of the reference UNG, wherein the position is numbered according to SEQ ID NO: 133.

In some embodiments, the UNG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 56, 58, 60, 62, 135, 137, 139, 141, 144, 146, 148, 150, 152, 155, 157, and 159 or an N-terminal truncation thereof lacking the most N-terminal Methionine (M) (coded by start codon ATG).

In some embodiments, the UNG is (substantially) capable of excising thymine of dT.

In some embodiments, the UNG is (substantially) capable of excising cytosine of dC.

In some embodiments, the napDNAbd is RNA programmable DNA binding protein.

In some embodiments, the napDNAbd is selected from the group consisting of CRISPR-associated (Cas) protein, IscB, IsrB, Argonaute, and TnpB.

In some embodiments, the napDNAbd is a nickase, e.g., a Cas9 nickase, an IscB nickase.

In some embodiments, the napDNAbd is nuclease-inactive, e.g., dead Cas9, dead Cas12i.

In some embodiments, the napDNAbd comprise an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 2, 48, 50, 52, or 163.

In some embodiments, the fusion protein comprises, from N-terminal to C-terminal, (1) the napDNAbp and the base excising domain; or (2) the base excising domain and the napDNAbp.

In some embodiments, the napDNAbd (e.g., Cas9) is a two-part napDNAbd, for example, a two-part split Cas9, comprising a N-terminal portion and a C-terminal portion, and wherein the fusion protein comprises, from N-terminal to C-terminal, (1) the N-terminal portion of the napDNAbd, the base excising domain, and the C-terminal portion of the napDNAbd; (2) the C-terminal portion of the napDNAbd, the base excising domain, and the N-terminal portion of the napDNAbd; or (3) the base excising domain, the C-terminal portion of the napDNAbd (e.g., amino acids at positions 1249-1368), and the N-temrinal portion (e.g., amino acids at positions 1-1248) of the napDNAbd.

In some embodiments, the napDNAbd is SpCas9 (e.g., a SpCas9 nickase) or a mutant thereof (e.g., a SpG Cas9 nickase).

In some embodiments, the N-terminal portion of the napDNAbd is the amino acids of the napDNAbp at positions 1 or 2 to 1012, 1028, 1041, 1046, 1047, 1248, 1249, or 1300.

In some embodiments, the C-terminal portion of the napDNAbd is the amino acids of the napDNAbp at positions 1013, 1029, 1042, 1047, 1048, 1249, 1063, 1064, 1230, 1249, or 1301 to 1368.

In some embodiments, the fusion protein comprises the base excising domain embedded between positions 2-1248 of nCas9 (SEQ ID NO: 2) and positions 1249-1368 of nCas9 (SEQ ID NO: 2), wherein the first amino acid residue D of nCas9 (SEQ ID NO: 2) was designated as position 2; or embedded between positions 2-1047 of nCas9 (SEQ ID NO: 2) and positions 1064-1368 of nCas9 (SEQ ID NO: 2), wherein the first amino acid residue D of nCas9 (SEQ ID NO: 2) was designated as position 2.

In some embodiments, the fusion protein comprises an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 12, 14, 15, 17, 19, 21, 23, 25, 27, 29, 31, 33, 35, 37, 39, 55, 57, 59, 61, 63, 136, 138, 140, 142, 143, 145, 147, 149, 151, 153, 154, 156, 158, 160, 161, 162, and 164.

In another aspect, the disclosure provides a system comprising:
(i) the fusion protein of the disclosure or a polynucleotide encoding the fusion protein; and
(ii) a guide nucleic acid or a polynucleotide encoding the guide nucleic acid, the guide nucleic acid comprising:
(1) a scaffold sequence capable of forming a complex with the napDNAbd; and
(2) a guide sequence capable of hybridizing to the target sequence on the target strand of the target dsDNA, thereby guiding the complex to the target dsDNA.

In some embodiments, the guide nucleic acid is a guide RNA (gRNA).

In some embodiments, the scaffold sequence has substantially the same secondary structure as the secondary structure of the sequence of SEQ ID NO: 40, 73, or 74.

In some embodiments, the scaffold sequence comprises (1) a sequence of SEQ ID NO: 40, 73, or 74 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 40, 73, or 74 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 40, 73, or 74.

In some embodiments, the fusion protein or system of the disclosure further comprises a translesion synthesis (TLS) polymerase or a recruiting domain or component capable of recruiting a TLS polymerase.

In some embodiments, the TLS polymerase is selected from the group consisting of Polα (alpha), Polβ (beta), Polδ (delta) (PCNA), Polγ (gamma), Polη (eta), Polι (iota), Polκ (kappa), Polλ (lamda), Polμ (mu), Polν (nu), Polθ (theta), and REV1.

In yet another aspect, the disclosure provides a polynucleotide encoding the fusion protein of the disclosure and optionally the guide nucleic acid of the disclosure.

In yet another aspect, the disclosure provides a delivery system comprising (1) the fusion protein of the disclosure, the polynucleotide of the disclosure, or the system of the disclosure; and (2) a delivery vehicle.

In yet another aspect, the disclosure provides a vector comprising the polynucleotide of the disclosure.

In yet another aspect, the disclosure provides a complex comprising the fusion protein or the polynucleotide (e.g., a mRNA) encoding the fusion protein of the disclosure and a guide nucleic acid (e.g., a gRNA) of the disclosure.

In yet another aspect, the disclosure provides a pharmaceutical composition comprising (1) the system of the disclosure, the vector of the disclosure, the ribonucleoprotein of the disclosure, the lipid nanoparticle of the disclosure, or the cell of the disclosure; and (2) a pharmaceutically acceptable excipient. In yet another aspect, the disclosure provides a cell or a progeny thereof comprising the system of the disclosure. In yet another aspect, the disclosure provides a cell or a progeny thereof modified by the system of the disclosure or the method of the disclosure.

In yet another aspect, the disclosure provides a method of modifying a target dsDNA, comprising contacting the target dsDNA with the system of the disclosure,
the target dsDNA comprising:
(a) a first deoxyribonucleotide (e.g., dG (deoxyguanosine), dT (thymidine), dC (deoxycytidine)) in a protospacer sequence on a nontarget strand (edited strand) of the target dsDNA, and
(b) a second deoxyribonucleotide (e.g., dC (deoxycytidine), dA (deoxyadenosine), dG (deoxyguanosine)) base pairing with the first deoxyribonucleotide (e.g., dG, dT, dC) and in a target sequence on the target strand (non-edited strand) of the target dsDNA, wherein the protospacer sequence is fully reverse complementary to the target sequence.

In some embodiments, the method does not include deamination of the base of the first deoxyribonucleotide before the excision of the base of the first deoxyribonucleotide.

In some embodiments, the method does not include deamination of the base of the first deoxyribonucleotide.

In yet another aspect, the disclosure provides an MPG described herein, or of the disclosure.

In yet another aspect, the disclosure provides an UNG described herein, or of the disclosure.

The details of one or more embodiments of the disclosure are set forth in the description below. Other features or advantages of the disclosure will be apparent from the following drawings and detailed description of several embodiments, and also from the appended claims. It is understood that any aspect or embodiment of the disclosure can be combined with any other one or more aspects or embodiments of the disclosure, including aspects or embodiments only described in one sub-section, only in the examples, or only in the claims, to constitute another embodiment explicitly or implicitly disclosed herein unless otherwise indicated.

### Definitions

The disclosure will be described with respect to particular embodiments, but the disclosure is not limited thereto in any respect. Unless defined otherwise, all technical and scientific terms used herein have the same meaning as is commonly understood by one of ordinary skill in the art to which this disclosure belongs. Terms as set forth hereinafter are generally to be understood in their plain and ordinary meaning or common sense unless indicated otherwise.

### Overview

Nucleic acid programmable binding protein (napBP), for example, nucleic acid programmable DNA binding protein, (napDNAbp), such as Cas9, Cas12, IscB, nucleic acid programmable RNA binding protein (napRNAbp), such as, Cas13, is capable of binding to a target nucleic acid (e.g., dsDNA, mRNA) as guided by a guide nucleic acid (e.g., a guide RNA) comprising a guide sequence targeting the target nucleic acid. In some embodiments, the target nucleic acid is eukaryotic.

Without wishing to be bound by theory, in some embodiments, the guide nucleic acid comprises a scaffold sequence responsible for forming a complex with the napBP, and a guide sequence that is intentionally designed to be responsible for hybridizing to a target sequence of the target nucleic acid, thereby guiding the complex comprising the napBP and the guide nucleic acid to the target nucleic acid.

Referring to FIG. 6, an exemplary target dsDNA is depicted to comprise a 5′ to 3′ single DNA strand and a 3′ to 5′ single DNA strand.

An exemplary guide nucleic acid (e.g., a guide RNA) is depicted to comprise a guide sequence and a scaffold sequence. The guide sequence is designed to hybridize to a part of the 3′ to 5′ single DNA strand, and so the guide sequence “targets” that part. And thus, the 3′ to 5′ single DNA strand is referred to as a “target strand (TS)” of the target dsDNA, while the opposite 5′ to 3′ single DNA strand is referred to as a “nontarget strand (NTS)” of the target dsDNA. That part of the target strand based on which the guide sequence is designed and to which the guide sequence may hybridize is referred to as a “target sequence”, while the opposite part on the nontarget strand corresponding to that part is referred to as the “protospacer sequence”, which is 100% (fully) reversely complementary to the target sequence and is said to be “corresponding to” the target sequence in the disclosure.

Referring to FIG. 6, an exemplary target dsDNA is depicted to comprise a 5′ to 3′ single DNA strand and a 3′ to 5′ single DNA strand. According to conventional transcription process, an exemplary target RNA (transcript, e.g., a pre-mRNA) may be transcribed using the 3′ to 5′ single DNA strand as a synthesis template, and thus the 3′ to 5′ single DNA strand is referred to as a “template strand” or a “antisense strand”. The transcript so transcribed has the same primary sequence as the 5′ to 3′ single DNA strand except for the replacement of T with U, and thus the 5′ to 3′ single DNA strand is referred to as a “coding strand” or a “sense strand”.

An exemplary guide nucleic acid (e.g., a guide RNA) is depicted to comprise a guide sequence and a scaffold sequence. The guide sequence is designed to hybridize to a part of the transcript (target RNA), and so the guide sequence “targets” that part. And thus, that part of the target RNA based on which the guide sequence is designed and to which the guide sequence may hybridize is referred to as a “target sequence”. In some embodiments, the guide sequence is 100% (fully) reversely complementary to the target sequence. In some other embodiments, the guide sequence is reversely complementary to the target sequence and contains a mismatch with the target sequence.

Generally, as is conventional in the art, a nucleic acid sequence (e.g., a DNA sequence, an RNA sequence) is written in 5′ to 3′ direction/orientation unless explicitly indicated otherwise.

For example, for a DNA sequence of ATGC, it is usually understood as 5′-ATGC-3′ unless otherwise indicated. Its reverse sequence is 5′-CGTA-3′. Its fully complementary sequence is 5′-TACG-3′. Its fully reverse complementary sequence is 5′-GCAT-3′. Note that the fully complementary sequence usually does not have the ability to base-pair/hybridize with the original sequence.

Generally, the double-strand sequence of a dsDNA may be represented with the sequence of its 5′ to 3′ single DNA strand conventionally written in 5′ to 3′ direction/orientation unless otherwise indicated.

For example, for a dsDNA having a 5′ to 3′ single DNA strand of 5′-ATGC-3′ and a 3′ to 5′ single DNA strand of 3′-TACG-5′, the dsDNA may be simply represented as 5′-ATGC-3′.

5′-----ATGC-----3′
3′-----TACG-----5′

It should be noted that either the 5′ to 3′ single DNA strand or the 3′ to 5′ single DNA strand of a dsDNA can be a nontarget strand from which a protospacer sequence is selected.

Generally, for a gene as a dsDNA, the 5′ to 3′ single DNA strand is the sense strand of the gene, and the 3′ to 5′ single DNA strand is the antisense strand of the gene. It should be noted that either the sense strand or the antisense strand of a gene can be a nontarget strand from which a protospacer sequence is selected.

Normally, the transcript (target RNA) transcribed from the dsDNA then has a (target) sequence of 5′-AUGC-3′.

To hybridize to a target dsDNA, in one embodiment, the guide sequence of a guide nucleic acid is designed to have a sequence of 5′-AUGC-3′ that is fully reversely complementary to the 3′ to 5′ strand of the target dsRNA, which would be set forth in ATGC in the electric sequence listing but marked as an RNA sequence; and in another embodiment, the guide sequence of a guide nucleic acid is designed to have a sequence of 5′-GCAU-3′ that is fully reversely complementary to the 5′ to 3′ strand of the target dsRNA, which would be set forth in GCAT in the electric sequence listing but marked as an RNA sequence.

In the case that the guide sequence of a guide nucleic acid is fully reversely complementary to the target sequence and the target sequence is fully reversely complementary to the protospacer sequence, the guide sequence is identical to the protospacer sequence except for the U in the guide sequence due to its RNA nature and correspondingly the T in the protospacer sequence due to its DNA nature. According to WIPO standard ST.26, symbol “t” is used to denote both T in DNA and U in RNA (See “Table 1: List of nucleotides symbols”, the definition of symbol “t” is “thymine in DNA/uracil in RNA (t/u)”). Thus, in the electronic sequence listing of the disclosure prepared according to ST. 26, such a guide sequence could be set forth in the same sequence as a corresponding protospacer sequence. For convenience, a single SEQ ID NO in the electronic sequence listing can be used to denote both such guide sequence and protospacer sequence, regardless whether such a single SEQ ID NO is marked as DNA or RNA in the electronic sequence listing. When a reference is made to such a SEQ ID NO that sets forth a protospacer/guide sequence, it refers to either a protospacer sequence that is a DNA sequence or a guide sequence that is an RNA sequence depending on the context, no matter whether it is marked as a DNA or an RNA in the electronic sequence listing.

To hybridize to the target RNA, in one embodiment, the guide sequence of a guide nucleic acid is designed to have a sequence of 5′-GCAU-3′ that is fully reversely complementary to the (target) sequence of the target RNA, which would be set forth in GCAT in the electric sequence listing but marked as an RNA sequence.

### Term

As used herein, if a DNA sequence, for example, 5′-ATGC-3′ is transcribed to an RNA sequence, with each dT (deoxythymidine, or “T” for short) in the primary sequence replaced with a U (uridine) and other dA (deoxyadenosine, or “A” for short), dG (deoxyguanosine, or “G” for short), and dC (deoxycytidine, or “C” for short) replaced with A (adenosine), G (guanosine), and C (cytidine), respectively, for example, 5′-AUGC-3′, it is said in the disclosure that the DNA sequence “encodes” the RNA sequence.

As used herein, the term “activity” refers to a biological activity. In some embodiments, the activity includes enzymatic activity, e.g., catalytic ability of an effector. For example, the activity can include nuclease activity, e.g., dsDNA endonuclease activity, RNA endonuclease activity.

As used herein, the term “nucleic acid programmable binding protein (napBP)” may be used interchangeably with “nucleic acid programmable binding domain (napBD)” to refer to a protein that can associate (e.g., bind) with a programmable nucleic acid (e.g., DNA or RNA), such as a guide nucleic acid (e.g., gRNA), that is able to be programmed to guide the protein to a specific sequence of a target nucleic acid via the interaction (e.g., hybridization) between the programmable nucleic acid (e.g., the guide sequence of the programmable nucleic acid) and the target nucleic acid (e.g., the target sequence of the target nucleic acid). The napBP may be indirectly associated with (e.g., bound to) the target nucleic acid via the interaction (e.g., binding) between the napBP and the programmable nucleic acid (e.g., scaffold sequence of the programmable nucleic acid) and the interaction (e.g., hybridization) between the programmable nucleic acid (e.g., the guide sequence of the programmable nucleic acid) and the target nucleic acid (e.g., the target sequence of the target nucleic acid). In some embodiments, the napBP is a nucleic acid programmable DNA binding protein (napDNAbp). In some embodiments, the napBP is a nucleic acid programmable RNA binding protein (napRNAbp).

As used herein, the term “complex” refers to a grouping of two or more molecules. In some embodiments, the complex comprises a polypeptide and a nucleic acid interacting with (e.g., binding to, coming into contact with, adhering to) one another. As used herein, the term “complex” can refer to a grouping of a guide nucleic acid and a polypeptide (e.g., a napBP). As used herein, the term “complex” can refer to a grouping of a guide nucleic acid, a polypeptide (e.g., a napBP), and a target nucleic acid.

As used herein, the term “protospacer adjacent motif” or “PAM” refers to a short DNA sequence (or a DNA motif) adjacent to a protospacer sequence on the nontarget strand of a dsDNA. As used herein, the term “adjacent” includes instances wherein there is no nucleotide between the protospacer sequence and the PAM and also instances wherein there are a small number (e.g., 1, 2, 3, 4, or 5) of nucleotides between the protospacer sequence and the PAM. As used herein, A “immediately adjacent (to)” B, A “immediately 5′ to” B, and A “immediately 3′ to” B mean that there is no nucleotide between A and B. In some embodiments, the PAM is immediately 5′ to a protospacer sequence. In some embodiments, the PAM is immediately 3′ to a protospacer sequence.

As used herein, the term “guide nucleic acid” refers to any nucleic acid that facilitates the targeting of a napBP to a target nucleic acid. For this purpose, the guide nucleic acid may be designed to include a guide sequence capable of hybridizing to a specific sequence of a target nucleic acid, and the guide nucleic acid may also comprise a scaffold sequence facilitating the guiding of a napBP to the target nucleic acid. In some embodiments, the guide nucleic acid is a guide RNA. In some embodiments, the guide nucleic acid is a nucleic acid encoding a guide RNA.

As used herein, the terms “nucleic acid”, “polynucleotide”, and “nucleotide sequence” are used interchangeably to refer to a polymeric form of nucleotides of any length, including deoxyribonucleotides, ribonucleotides, combinations thereof, and analogs or modifications thereof.

As used in the context of CRISPR-Cas techniques (e.g., CRISPR-Cas12 techniques), the term “guide RNA” is used interchangeably with the term “CRISPR RNA (crRNA)”, “single guide RNA (sgRNA)”, or “RNA guide”, the term “guide sequence” is used interchangeably with the term “spacer sequence”, and the term “scaffold sequence” is used interchangeably with the term “direct repeat sequence”.

As described herein, the guide sequence is so designed to be capable of hybridizing to a target sequence. As used herein, the term “hybridize”, “hybridizing”, or “hybridization” refers to a reaction in which one or more polynucleotide sequences react to form a complex that is stabilized via hydrogen bonding between the bases of the polynucleotide sequences. The hydrogen bonding may occur by Watson Crick base pairing, Hoogstein binding, or in any other sequence specific manner. A polynucleotide sequence capable of hybridizing to a given polynucleotide sequence is referred to as the “complement” of the given polynucleotide sequence. As used herein, the hybridization of a guide sequence and a target sequence is so stabilized to permit an effector polypeptide (e.g., a napBP) that is complexed with a nucleic acid comprising the guide sequence or a function domain associated (e.g., fused) with the effector polypeptide to act (e.g., cleave, deaminize) on the target sequence or its complement or nearby sequence.

For the purpose of hybridization, in some embodiments, the guide sequence is reversely complementary to a target sequence. As used herein, the term “reverse complementary” refers to the ability of nucleobases of a first polynucleotide sequence, such as a guide sequence, to base pair with nucleobases of a second polynucleotide sequence, such as a target sequence, by traditional Watson-Crick base-pairing. Two reverse complementary polynucleotide sequences are able to non-covalently bind under appropriate temperature and solution ionic strength conditions. In some embodiments, a first polynucleotide sequence (e.g., a guide sequence) comprises 100% (fully) reverse complementarity to a second nucleic acid (e.g., a target sequence). In some embodiments, a first polynucleotide sequence (e.g., a guide sequence) is reverse complementary to a second polynucleotide sequence (e.g., a target sequence) if the first polynucleotide sequence comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid (i.e., at least about 80%, 81%, 82%, 83%, 84%, 85%, 86%, 87%, 88%, 89%, 90%, 91%, 92%, 93%, 94%, 95%, 96%, 97%, 98%, or 99% of the nucleotides of the first polynucleotide sequence can base-pair with the nucleotides of the second polynucleotide sequence). As used herein, the term “substantially complementary” refers to a first polynucleotide sequence (e.g., a guide sequence) that has a certain level of complementarity to a second polynucleotide sequence (e.g., a target sequence) (e.g., at least about 80%, 81%, 82%, 83%, 84%, 85%, 86%, 87%, 88%, 89%, 90%, 91%, 92%, 93%, 94%, 95%, 96%, 97%, 98%, or 99% of the nucleotides of the first polynucleotide sequence can base-pair with the nucleotides of the second polynucleotide sequence, or at most 1, 2, 3, 4, or 5 contiguous or non-contiguous nucleotides of the first polynucleotide sequence mismatch the nucleotides of the second polynucleotide sequence). In some embodiments, the level of complementarity is such that the first polynucleotide sequence (e.g., a guide sequence) can hybridize to the second polynucleotide sequence (e.g., a target sequence) with sufficient affinity to permit an effector polypeptide (e.g., a napBP) that is complexed with a nucleic acid comprising the first polynucleotide sequence or a function domain associated (e.g., fused) with the effector polypeptide to act (e.g., cleave, deaminize) on the target sequence or its complement or nearby sequence. In some embodiments, a guide sequence that is substantially complementary to a target sequence has less than 100% complementarity to the target sequence. In some embodiments, a guide sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence, and/or has at most 1, 2, 3, 4, or 5 contiguous or non-contiguous nucleotide mismatches from the target sequence.

As used herein, the term “sequence identity” is related to sequence homology. Homology comparisons may be conducted by eye, or more usually, with the aid of readily available sequence comparison programs. These commercially available computer programs may calculate percentage sequence identity (%) between two or more sequences (polypeptide or polynucleotide sequences). Sequence homologies may be generated by any of a number of computer programs known in the art, for example, BLAST, FASTA. A suitable computer program for carrying out such an alignment is the GCG Wisconsin Bestfit package (University of Wisconsin, U.S.A; Devereux et al., 1984, Nucleic Acids Research 12: 387). Examples of other software than may perform sequence comparisons include, but are not limited to, the BLAST package (see Ausubel et al., 1999 ibid-Chapter 18), FASTA (Atschul et al., 1990, J. Mol. Biol., 403-410), and the GENEWORKS suite of comparison tools. Both BLAST and FASTA are available for offline and online searching (see Ausubel et al., 1999 ibid, pages 7-58 to 7-60). A commonly used online tool to calculate percentage sequence identity between two or more sequences (polypeptide or polynucleotide sequences) is available on the website of EMBL's European Bioinformatics Institute (www dot ebi dot ac dot uk slash jdispatcher slash), allowing fast online calculation of percentage sequence identity by global alignment or local alignment.

As used herein, the terms “polypeptide” and “peptide” are used interchangeably herein to refer to polymers of amino acids of any length. A protein may have one or more polypeptides. An amino acid polymer can also be modified, for example, by disulfide bond formation, glycosylation, lipidation, acetylation, phosphorylation, or any other manipulation, such as conjugation with a labeling component.

As used herein, a “variant” is interpreted to mean a polynucleotide or polypeptide that differs from a reference polynucleotide or polypeptide, respectively, but retains essential properties, e.g., binding property of a napBP. A typical variant of a polynucleotide differs in nucleic acid sequence from another reference polynucleotide. A change in the nucleic acid sequence of the polynucleotide variant may or may not alter the amino acid sequence of a polypeptide encoded by the reference polynucleotide. A change in the nucleic acid sequence of the polynucleotide variant may result in an amino acid substitution, addition, and/or deletion in the polypeptide encoded by the reference polynucleotide. A typical variant of a polypeptide differs in amino acid sequence from another reference polypeptide. Generally, the difference is limited so that the sequences of the reference polypeptide and the polypeptide variant are closely similar overall and, in many regions, identical. The polypeptide variant and reference polypeptide may differ in amino acid sequence by one or more substitutions, additions, and/or deletions in any combination. A variant of a polynucleotide or polypeptide may be naturally occurring, such as, an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the terms “upstream” and “downstream” refer to the relative positions of two or more elements within a nucleic acid in 5′ to 3′ direction. A first sequence is upstream of a second sequence when the 3′ end of the first sequence is present at the left side of the 5′ end of the second sequence. A first sequence is downstream of a second sequence when the 5′ end of the first sequence is present at the right side of the 3′ end of the second sequence. In some embodiments, the PAM is upstream of a napBP-induced indel, and a napBP-induced indel is downstream of the PAM. In some embodiments, the PAM is downstream of a napBP-induced indel, and a napBP-induced indel is upstream of the PAM.

As used herein, the term “wild type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the terms “non-naturally occurring” and “engineered” are used interchangeably and refer to artificial participation. When these terms are used to describe a nucleic acid or a polypeptide, it is meant that the nucleic acid or polypeptide is at least substantially freed from at least one other component of its association in nature or as found in nature.

As used herein, the term “regulatory element” is intended to include promoters, enhancers, internal ribosome entry sites (IRES), and other expression control elements (e.g., transcription termination signals, such as, polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cells and those that direct expression of a nucleotide sequence only in certain host cells (e.g., tissue-specific regulatory sequences). Regulatory elements may also direct expression in a time-dependent manner, e.g., in a cell cycle-dependent or developmental stage-dependent manner, which may or may not be tissue or cell type specific.

As used herein, the term “cell” is understood to refer not only to a particular individual cell, but to the progeny or potential progeny of the cell. Because certain modifications may occur in succeeding generations due to either mutation or environmental influences, such progeny may not, in fact, be identical to the parent cell, but are still included within the scope of the term.

As used herein, the term “in vivo” refers to inside the body of an organism, and the terms “ex vivo” or “in vitro” means outside the body of an organism.

As used herein, the term “treat”, “treatment”, or “treating” is an approach for obtaining beneficial or desired results including clinical results. For purposes of the disclosure, the beneficial or desired clinical results include, but are not limited to, one or more of the following: alleviating one or more symptoms resulting from a disease, diminishing the extent of a disease, stabilizing a disease (e.g., delaying the worsening of a disease), delaying the spread (e.g., metastasis) of a disease, delaying the recurrence of a disease, reducing recurrence rate of a disease, delay or slowing the progression of a disease, ameliorating a disease state, providing a remission (partial or total) of a disease, decreasing the dose of one or more other medications required to treat a disease, delaying the progression of a disease, increasing the quality of life, and/or prolonging survival. Also encompassed by “treatment” is a reduction of pathological consequence of a disease (such as cancer). The methods of the disclosure contemplate any one or more of these aspects of treatment.

As used herein, the term “disease” includes the terms “disorder” and “condition” and is not limited to those have been specifically medically defined.

As used herein, the term “transcript” includes any transcription product by transcription from a DNA, including subgenomic RNA, mRNA, non-coding RNA, and any variants, derivatives, or ancestors thereof, for example, pre-mRNA, and any transcripts or isoforms produced from the DNA or the pre-mRNA by, e.g., alternative promoter usage, alternative splicing, alternative initiation, and any naturally occurring variants thereof or processed products therefrom.

As used herein, reference to “not” a value or parameter generally means and describes “other than” a value or parameter. For example, the method is not used to treat cancer of type X means the method may be used to treat cancer of types other than X.

As used herein, the singular forms “a”, “an”, and “the” include plural referents unless the context clearly dictates otherwise.

As used herein, the term “and/or” in a phrase such as “A and/or B” is intended to include both A and B; A or B; A (alone); and B (alone). Likewise, the term “and/or” in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, when the term “about” is ahead of a serious of numbers (for example, about 1, 2, 3), it is understood that each of the serious of numbers is modified by the term “about” (that is, about 1, about 2, about 3). The term “about X-Y” or “about X to Y” used herein has the same meaning as “about X to about Y.”

It is understood that embodiments of the disclosure described herein include “consisting” and/or “consisting essentially of” embodiments.

It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1. Design and mechanisms of base editing by conventional base editors and glycosylase-based base editors of the disclosure. FIG. 1a: Schematic diagrams of ABE (left) and CBE (middle) and the deaminase-free glycosylase-based guanine base editor (gGBE, right) of the disclosure. A nCas9-sgRNA complex creates an R-loop at the target site in the DNA. In ABE and CBE, the evolved adenine deaminase (tRNA adenosine deaminase, TadA) and AID/APOBEC-like cytidine deaminase converts the exposed adenine (A) into deoxyinosine (I) and cytosine (C) into deoxyuridine (U), respectively. In CBE, an additional linked protein, uracil glycosylase inhibitor (UGI), protects U from uracil DNA N-glycosylase (UNG). After deamination, the resulting I is recognized as G, and U as T by DNA polymerase during DNA repair or replication. In the gGBE of the disclosure, a prototype version of glycosylase-based guanine base editor (gGBE) is designed to remove G, and the AP site generated thereby is repaired by TLS and/or DNA replication, leading to G-to-C or G-to-T conversion. PAM, protospacer adjacent motif. AP, apurinic/apyrimidinic sites. FIG. 1b: A screening reporter system for detecting G-to-T conversion by gGBE. P2A, 2A peptide from porcine teschovirus-1. FIG. 1c: Percentage of EGFP⁺ cells for G editing efficiency evaluation of gGBE with various MPG (mean±s.e.m., n=3). WT: wild-type.

FIG. 2. Mutagenesis of the MPG moiety in gGBEs. FIG. 2a: Schematic diagram of mutagenesis and screening strategy for engineered gGBE. The EGFP reporter plasmids were transiently co-transfected into cultured cells along with the gGBE expression plasmids. FIG. 2b: Genotypes of a subset of engineered gGBEs, with percentage of EGFP⁺ cells for each gGBE on the far-right column (more engineered gGBEs listed in FIG. 13). Different steps of mutagenesis are marked by different shaded colors. FIG. 2c-d: G base editing outcomes with different gGBE at the edited G7 position in site 3 (FIG. 2c) and site 10 (FIG. 2d) in transfected HEK293T cells by target deep sequencing (mean±s.e.m., n=3).

FIG. 3. Characterization of editing profiles of gGBE via target deep sequencing. FIG. 3a: Bar plots showing the on-target DNA base editing at positions with the highest G conversion frequencies at each genomic site in HEK293T cells (mean±s.e.m., n=3). G #: G position with highest on-target base editing frequencies across protospacer positions 1-20. site #: genomic site number. FIG. 3b: The ratio of G-to-C/T to G-to-A/C/T conversion frequency by gGBEv6.3 editing at the sites shown in FIG. 3a. FIG. 3c: Frequencies of G conversions by gGBEv6.3 across protospacer positions 1-20 at the edited sites in FIG. 3a (in which PAM was at positions 21-23). Single dots represent individual data point from 3 independent replicates per site. Boxes span the interquartile range (25th to 75th percentile); horizontal line in the box indicates the median (50th percentile); and small horizontal bars mark the minimal and maximal values. FIG. 3d: The guide sequence-dependent off-target analysis for gGBEv6.3 editing efficiency at site 7 (mean±s.e.m, n=3). OT: off-target. FIG. 3e: The guide sequence-independent off-target editing efficiency detected by the orthogonal R-loop assay at each R-loop site (mean±s.e.m, n=3).

FIG. 4. Gene editing applications of gGBE. FIG. 4a: Application of gGBEv6.3 for editing splicing sites, introduction of premature termination codons (PTCs), as well as editing that bypasses PTCs. FIG. 4b: Schematic diagram illustrating gGBE-indued skipping of DMD exon 45. FIG. 4c: Bar plots showing the on-target DNA base editing frequencies of G editing and the ratio of G-to-C/T to G-to-A/C/T editing frequencies by gGBEv6.3 at two DMD sites in HEK293T cells (mean±s.e.m, n=3). SpG, a PAM-flexible Cas9 variant (SEQ ID NO: 163), was used for targeting DMD site 2. FIG. 4d: Schematic diagram illustrating the introduction of PTCs in the mouse Tyr gene by gGBE. FIG. 4e: Bar plots showing the on-target G editing frequencies and G-to-Y Ratio by gGBEv6.3 at three Tyr sites in N2a cultured cells (mean±s.e.m, n=3). FIG. 4f: Percentages of G conversion-induced PTCs by gGBEv6.3 at two Tyr sites in mouse pups (mean±s.e.m., n=25 and 21 pups for site 2 and site 3, respectively). FIG. 4g: Phenotype of F0 mice generated by gGBE editing in mouse zygotes. The Image showing the presence of edited P6 mice. Red arrowhead, albino; blue arrowhead, mosaic pigmentation. FIG. 4h: Bar plots showing the on-target G editing frequencies for individual mouse pups, with gGBEv6.3 targeting Tyr site 3. FIG. 4i: Genotyping of representative F0 pups from (FIG. 4h). The frequencies of mutant alleles were determined by high-throughput sequencing. Red arrowhead, albino pups.

FIG. 5 illustrates example nucleotide conversion by base excision and translesion synthesis (TLS).

FIG. 6 illustrates an exemplify target dsDNA containing a first exemplify deoxyribonucleotide dG, an exemplify guide nucleic acid, and an exemplify napDNAbp before base editing.

FIG. 7 illustrates an exemplify target dsDNA containing a fourth exemplify deoxyribonucleotide dC, an exemplify guide nucleic acid, and an exemplify napDNAbp after base editing.

FIG. 8 illustrates an exemplify target dsDNA containing a fourth exemplify deoxyribonucleotide dT, an exemplify guide nucleic acid, and an exemplify napDNAbp after base editing.

FIG. 9. Overview of AYBE and CGBE. FIG. 9a: Schematic diagram of AYBE. N-methylpurine DNA glycosylase (MPG) excises the inosine (I) resulting from deamination of adenine (A) by the adenine deaminase (TadA), triggering base excision repair (BER) pathway in cells, thus causing more versatile base editing outcomes, including A-to-C and A-to-T editing. FIG. 9b: Schematic diagram of CGBE. Uracil DNA N-glycosylase (UNG) excises the uridine (U) resulting from deamination of cytosine (C) by the AID/APOBEC-like cytidine deaminase, triggering base excision repair (BER) pathway in cells, thus causing dominant C-to-G editing. PAM, Protospacer adjacent motif. AP, apurinic/apyrimidinic sites.

FIG. 10. Characterization of A-to-T and G-to-T editing with an intron-split EGFP reporter system. FIG. 10a: Design of the reporter for A-to-T or G-to-T editing detection. P2A, 2A peptide from the porcine teschovirus-1. FIG. 10b: Percentage of EGFP⁺ cells for evaluation of A editing efficiency by gABE with various MPG. WT: wild-type; v0.2: MPG-N169S; v1: MPG with N169S, S198A, K202A, G203A, S206A and K210A mutations; v2: MPG with mutations N169S and G163R; v3: MPG with G163R, N169S, S198A, K202A, G203A, S206A and K210A mutations (mean±s.e.m., n=3). FIG. 10c: Percentage of EGFP⁺ cells representing the efficiency of G-to-T conversion for gGBE containing various MPG and sgRNA. dMPG, inactive dead MPG; T: targeting sgRNA; NT: nontargeting sgRNA (mean±s.e.m., n=3). FIG. 10d: Representative flow cytometry scatter plots showing gating strategy and the percentages of EGFP⁺ cells for gGBEv3.

FIG. 11. View of MPG structure and the first round of mutagenesis of MPG. FIG. 11a: Structures for aa 78-298 region (left) and 163-179 region (right) of human MPG protein (shown in gray), as predicted by AlphaFold (alphafold. com/entry/P29372) aligned with the crystal structure of MPG (PDB entry 1ewn, not shown), in which εA was mutated to G in the DNA. FIG. 11b: Percentage of EGFP⁺ cells for evaluating G editing efficiency with different engineered gGBEs with various MPG mutants (mean±s.e.m., n=3). FIG. 11c: Performance of various engineered gGBEs measured by the percentage of EGFP⁺ cells in the first round of screening. Dotted line, mean value of the MPGv3 group. Fold changes were calculated relative to gGBEv3 (mean±s.e.m., n=3). FIG. 11d: Percentage of EGFP⁺ cells for each gGBE (mean±s.e.m., n=3).

FIG. 12. Performance of engineered gGBEs in the second round of screening. FIG. 12a-d: Percentage of EGFP⁺ cells of gGBEs with various MPG mutants from sequential substitutions of glutamic acid (FIG. 12a), valine (FIG. 12b), glycine (FIG. 12c), and tyrosine (FIG. 12d) (X-to-E, V, G, or Y). n=3. All values are presented as mean±s.e.m.

FIG. 13. Progressive engineering and G editing efficiency of gGBEs. FIG. 13a: Progressive mutations of gGBEs. Different rounds of mutations are marked with different color shades. FIG. 13b: Percentage of EGFP⁺ cells for each gGBE. n=3. All values are presented as mean±s.e.m.

FIG. 14. Further characterization of editing profiles for gGBEv6.3. FIG. 14a-c: Frequencies of C (FIG. 14a), T (FIG. 14b) and A (FIG. 14c) conversions by gGBEv6.3 across the protospacer positions 1-20 (where PAM is at positions 21-23) from the edited sites in FIG. 3a. FIG. 14d: Frequencies of G-to-T and G-to-C editing by gGBEv6.3. In FIG. 14a-d, single dot represents individual replicate (n=3 independent replicates per site), and boxes span the interquartile range (25th to 75th percentile); horizontal lines within the boxes indicate the median (50%); and whiskers extend to the minimal and maximal values. FIG. 14e-g: Percentage of G-to-C (FIG. 14e), G-to-T (FIG. 14f) or G-to-A (FIG. 14g) editing by gGBEv6.3 at various edited sites shown in FIG. 3a (mean±s.e.m., n=3). FIG. 14h: Indels frequencies with gGBEv6.3 at 24 on-target sites (mean±s.e.m., n=3). FIG. 14i: Bar plots showing the on-target DNA base editing at positions with G conversion frequencies>10% at each genomic site in HEK293T cells (mean±s.e.m., n=3). FIG. 14j: The statistical analysis of on-target DNA base editing for each NG motif from the edited sites in (FIG. 14i). Each dot represents the mean of three biological replicates for each edited position at various edited sites.

FIG. 15. The guide sequence-dependent and guide sequence-independent off-target analysis. FIG. 15a: The guide sequence-dependent off-target analysis for gGBEv6.3 editing at different sites (n=3). OT: off-target. FIG. 15b: The guide sequence-independent off-target editing detected by the orthogonal R-loop assay at each R-loop site for gGBE, AYBE, and ABE8e (n=3), respectively. Data for AYBE and ABE8e were adopted from Tong et al. [1]. All values are presented as mean±s.e.m.

FIG. 16. The percentage of G-to-C and G-to-T among all G-to-C/T/A conversion events at DMD or Tyr sites targeted by gGBEv6.3. FIG. 16a: Percentages of G-to-C and G-to-T editing events in HEK293T cells with gGBEv6.3 at two DMD sites (corresponding to FIG. 4C). n=3. SA, splicing acceptor site. PAM, Protospacer adjacent motif. FIG. 16b: Percentages of G-to-C and G-to-T editing events in N2a cells with gGBEv6.3 at three Tyr sites (corresponding to FIG. 4e). n=3. All values are presented as mean±s.e.m.

FIG. 17. G editing in mouse embryos with gGBEv6.3. FIG. 17a: On-target base editing efficiencies for gGBEv6.3 targeting three Tyr sites in mouse embryos (mean±s.e.m., n=20). FIG. 17b-c: Percentages of G conversion-induced PTCs by gGBEv6.3 at three Tyr sites in mouse embryos (mean±s.e.m., n=20 embryos). FIG. 17d: Indel frequencies induced by gGBEv6.3 targeting at three Tyr sites in mouse embryos shown in FIG. 17a (n=20). FIG. 17e: Bar plots showing the on-target G editing frequencies for individual mouse embryos, with gGBEv6.3 targeting Tyr site 1, Tyr site 2, and Tyr site 3.

FIG. 18. Phenotypes and genotyping of F0 mouse pups. FIG. 18a-c: Phenotypes of F0 mice generated by microinjection of gGBEv6.3 encoding mRNA and sgRNA for targeting Tyr site 1 (FIG. 18a), site 2 (FIG. 18b) and site 3 (FIG. 18c). The images were obtained for P6 mice. Arrowheads: red, albino mice; blue, mice with mosaic pigmentation. FIG. 18d: Bar plots showing the on-target G editing frequencies for individual mouse pups, with gGBEv6.3 targeting Tyr site 1 and Tyr site 2.

FIG. 19. Design and mechanisms of two orthogonal glycosylase-based base editors. FIG. 19a, Prototype versions of a deaminase-free glycosylase-based thymine base editor (gTBE) and a deaminase-free glycosylase-based cytosine base editor (gCBE). PAM, Protospacer adjacent motif. AP, apurinic/apyrimidinic sites. Star (*) in magenta indicates the nick generated by nCas9. FIG. 19b, Schematic diagram of potential pathway for T (or C) editing and outcomes. A glycosylase mutant is designed to remove normal T or C, an nCas9-sgRNA complex creates an R-loop at the target site and nicks the non-edited strand, then the AP site generated is repaired by translesion synthesis (TLS) and/or DNA replication, leading to T or C editing. DSB, double-strand break. indel, insertion and deletion. FIG. 19c, Schematic of various gTBE and gCBE candidate architectures. Note that Y156A and N213D of UNG2 are equivalent to Y147A and N204D of UNG1, respectively. FIG. 19d, Percentage of EGFP⁺ cells for T editing efficiency evaluation of different gTBE using T-to-G reporter (n=3). NT, non-target sgRNA. T: target sgRNA. FIG. 19e, Percentage of EGFP⁺ cells for C editing efficiency evaluation of different gCBE using C-to-G reporter (n=3). NT, non-target sgRNA. T: target sgRNA. FIG. 19f, the orthogonality of gTBE and gCBE for base editing evaluated using two different reporters (n=3). All values are presented as mean±s.e.m.

FIG. 20. Protein engineering and evolution of gTBEs. FIG. 20a, Schematic diagram of mutagenesis and screening strategy for engineering gTBE. The EGFP reporter plasmids were transiently co-transfected into cultured cells along with the gTBE plasmids, and the fluorescence intensity of EGFP was detected with flow cytometry. FIG. 20b, Left, the selected residues (shown as surface) for mutagenesis nearby the catalytic site pocket of human UNG-DNA complex (PDB entry 1EMH²⁴), in which dWU was mutated to T in the DNA (dT). Right, location of the effective residues in gTBEv3 shown as spheres in red on the three-dimensional structure. FIG. 20c, Gradual improvement of EGFP activation for each gTBE (n=3). WT, wild-type UNG2Δ88. dead, catalytically inactive UNG2Δ88 (carrying D154N and H277N mutations, equivalent to D145N and H268N of UNG1)⁶⁰. FIG. 20d, Frequencies of T base editing outcomes (left) and indels (right) with different gTBE at the edited T5 position in site 9 (CLYBL gene) in transfected HEK293T cells by target deep sequencing (n=3). All values are presented as mean±s.e.m.

FIG. 21. Characterization of editing profiles of gTBE via target deep sequencing. FIG. 21a, Bar plots showing the on-target DNA base editing at positions with the highest T conversion frequencies at each genomic site in HEK293T cells (mean±s.e.m., n=3). T #: T position with highest on-target base editing frequencies across protospacer positions 1-20. site #: genomic site number. FIG. 21b, The ratio of T-to-C/G to T-to-A/C/G conversion frequency by gTBEv3 editing at the sites shown in FIG. 21a. FIG. 21c, indels frequencies with gTBEv3 at 20 on-target sites (n=3). FIG. 21d-e, The guide sequence-dependent off-target analysis for gTBEv3 editing efficiency at site 1 and site 15 (n=3). OT: off-target. FIG. 21f, The guide sequence-independent off-target editing efficiency detected by the orthogonal R-loop assay at each R-loop site (n=3). All values are presented as mean±s.e.m.

FIG. 22. Enhancement of gCBE editing efficiency through protein engineering. FIG. 22a, Schematic diagram of mutagenesis and screening strategy for engineering gCBE. FIG. 22b, Gradual improvement of EGFP activation for each gCBE (n=3). WT, wild-type UNG2Δ88. dead, catalytically inactive UNG2Δ88 (carrying D154N and H277N mutations, equivalent to D145N and H268N of UNG1)⁶⁰. FIG. 22c, Bar plots showing the on-target DNA base editing at positions with the highest C conversion frequencies at each genomic site in HEK293T cells (n=3). C#: C position with highest on-target base editing frequencies across protospacer positions 1-20. site #: genomic site number. FIG. 22d, Bar plots showing the on-target DNA base editing of different positions at three loci with gCBEv2 or CGBE1 (n=3). FIG. 22e, On-target base editing frequencies for gCBEv2 at C6 of site 22 in HEK293T cells for the orthogonal R-loop assay (n=3). FIG. 22f, gRNA-independent cumulative off-target editing frequencies detected by the orthogonal R-loop assay at each R-loop site. Each R-loop was performed by co-transfection of each base editor, and an SpCas9 sgRNA targeting corresponding site with dSaCas9 and a SaCas9 sgRNA (n=3). All values are presented as mean±s.e.m.

FIG. 23. Gene editing applications of gTBE and gCBE. FIG. 23a, principle for exon skipping with base editors. FIG. 23b, Bar plots showing the numbers of sgRNA candidates targeting the splicing sites in 16 genes by different base editors. gCBE, gCBEv2; gGBE, gGBEv6.3; gTBE, gTBEv3. The 16 genes are AGT, ANGPTL3, APOC3, B2M, CD33, DMD, DNMT3A, HPD, KLKB1, PCSK9, PDCD1, PRDM1, TGFBR2, TRAC, TTR, and VEGFA. FIG. 23c, Venn diagram showing the distribution of sgRNAs for 4 base editors in FIG. 23b. FIG. 23d, Schematic diagram illustrating sgRNA candidates specifically targeting SD or SA sites in human DMD with gTBEv3 (red lines) or gCBEv2 (black lines), but not ABE or CBE. FIG. 23e, Schematic diagram illustrating the skipping of human DMD exon 45 induced by gTBE-induced disruption of the splicing donor site. FIG. 23f, On-target base editing efficiency for gTBEv3 targeting the splicing donor site of humanized DMD exon 45 in mouse embryos (mean±s.e.m., n=20). FIG. 23g, DNA sequencing chromatograms from wild-type (WT) and representative embryos co-injected with gTBEv3 mRNA and sgRNA targeting the SD site of human DMD exon 45.

FIG. 24. Comparison of different gTBEs. FIG. 24a, the strategies for protein engineering and screening used in three studies. FIG. 24b, Schematic of the basic architectures for various base editors. UNG2*, UNG2 mutant from the corresponding base editor. ΔNTD, deletion of the N-terminal domain. FIG. 24c, The frequencies of T conversions at 17 endogenous loci. The thymines with editing frequencies>25% for any base editors were showed. The highest frequencies at corresponding positions were highlighted as Heat map (n=3). FIG. 24d, Frequencies of T conversions by various base editors across the protospacer positions 1-20 (where PAM is at positions 21-23) from the edited sites in FIG. 24c. Single dot represents individual replicate (n=3 independent replicates per site), and boxes span the interquartile range (25th to 75th percentile); horizontal lines within the boxes indicate the median (50%); and whiskers extend to the minimal and maximal values.

FIG. 25. Characteristic sequences and motifs of human UNG1 and UNG2. FIG. 25a, UNG1-specific N-terminal residues (amino acid 1-35) are marked in grey. UNG2-specific N-terminal residues (amino acid 1-44) are light blue. The common RPA-binding site (yellow) and the globular catalytic domain (light green) are indicated. RPA, Replication protein A. FIG. 25b, UNGs contain five conserved motifs numbered from UNG2 as follows: the catalytic water-activating loop (152-GQDPYH-157); the proline (Pro)-rich loop compressing the DNA backbone 5′ to the lesion (174-PPPPS-178); the uracil-binding motif (210-LLLN-213); the glycine-serine (Gly-Ser) loop that compresses the DNA backbone 3′ to the lesion (255-GS-256); and the leucine (Leu)-intercalation loop penetrating the minor groove (277-HPSPLS-282).

FIG. 26. Characterizations of T-to-G and C-to-G reporter system. FIG. 26a, Schematic construct designs of the reporter for T-to-G or C-to-G editing detection. PAM, Protospacer adjacent motif. FIG. 26b, Representative flow cytometry scatter plots showing gating strategy and the percentages of gated cells for the negative control (upper panel) and gCBEv0.3 (lower panel).

FIG. 27. Editing efficiency of gTBE and gCBE candidates with various UNG-NTD truncations. FIG. 27a, Percentage of EGFP⁺ cells for evaluating T editing efficiency of gTBE candidates with various UNG mutants (n=3). FIG. 27b, Percentage of EGFP⁺ cells for evaluating C editing efficiency of gCBE candidates with various UNG mutants (n=3). WT, wild-type UNG2Δ88. dead, catalytically inactive UNG2Δ88 (carrying D154N and H277N mutations, equivalent to D145N and H268N of UNG1). All values are presented as mean±s.e.m.

FIG. 28. Performance of UNG mutants in the background of gTBEv0.3. FIG. 28a, Percentage of EGFP⁺ cells of gTBE from alanine-scanning mutagenesis of regions covering the catalytic water-activating loop, the Pro-rich loop, and the uracil-binding motif (n=3). Replacement of alanine with valine, A-to-V, is intended to cover all the residues in the interested regions. FIG. 28b, Percentage of EGFP⁺ cells of gTBE variants from site-saturation mutagenesis of the residue at position 214 (n=3). FIG. 28c, Percentage of EGFP⁺ cells of gTBE with mutations of selected spatial neighbors of residue T214 (n=3). dead, catalytically inactive UNG2Δ88 (carrying D154N and H277N mutations, equivalent to D145N and H268N of UNG1). All values are presented as mean±s.e.m.

FIG. 29. Performance of UNG mutants in the background of gTBEv2. FIG. 29a-c, Percentage of EGFP⁺ cells of gTBE with different UNG mutants from sequential substitutions of arginine (FIG. 29a), aspartic acid (FIG. 29b), and valine (FIG. 29c) (X-to-R, D, or V) (n=3). dead, catalytically inactive UNG2Δ88 (carrying D154N and H277N mutations, equivalent to D145N and H268N of UNG1). All values are presented as mean±s.e.m.

FIG. 30. Further characterization of editing profiles of gTBEv3. FIG. 30a, Bar plots showing the on-target DNA base editing at positions with T conversion frequencies>10% at each genomic site in HEK293T cells (mean±s.e.m., n=3). FIG. 30b-e, Frequencies of T (FIG. 30b), G (FIG. 30c), C (FIG. 30d) and A (FIG. 30e) conversions by gTBEv3 across the protospacer positions 1-20 (where PAM is at positions 21-23) from the edited sites in FIG. 21a. FIG. 30f, Frequencies of T-to-G and T-to-C editing by gTBEv3. In FIG. 30b-f, single dot represents individual replicate (n=3 independent replicates per site), and boxes span the interquartile range (25th to 75th percentile); horizontal lines within the boxes indicate the median (50%); and whiskers extend to the minimal and maximal values. FIG. 30g-i, Percentage of T-to-G (FIG. 30g), T-to-C (FIG. 30h) or T-to-A (FIG. 30i) editing by gTBEv3 at various edited sites shown in FIG. 3a (mean±s.e.m., n=3). T #: T position with highest on-target base editing frequencies across protospacer positions 1-20. site #: genomic site number. FIG. 30j, The ratio of T-to-Sto total T editing (base conversions and indels) by gTBEv3 editing at the sites shown in FIG. 21a. FIG. 30k. The statistical analysis of on-target DNA base editing for each NT motif from the edited sites in (FIG. 30a). Each dot represents the mean of three biological replicates for each edited position at various edited sites. n=8, 14, 6, 6 for motif AT, CT, GT, TT.

FIG. 31. Guide sequence-dependent off-target analysis for gTBEv3 at more sites. The guide sequence-dependent off-target analysis for gTBEv3 editing at site 9 (a) and site 15 (b) (n=3). OT: off-target. All values are presented as mean±s.e.m.

FIG. 32. Performance of UNG mutants in the background of gCBEv0.3. FIG. 32a, Percentage of EGFP+ cells of gCBE by introduction of the mutation A214V (n=3). FIG. 32b, Percentage of EGFP+ cells of gCBE from alanine-scanning mutagenesis of regions covering the catalytic water-activating loop and the Pro-rich loop (n=3). Replacement of alanine with valine (A-to-V) is intended to cover all the residues in the interested regions. dead, catalytically inactive UNG2Δ88 (carrying D154N and H277N mutations, equivalent to D145N and H268N of UNG1). All values are presented as mean±s.e.m.

FIG. 33. Further characterization of editing profiles of gCBEv2. FIG. 33a, Frequency of C base editing outcomes with different gCBE at the edited C2 position in site 28 in transfected HEK293T cells by target deep sequencing (mean±s.e.m., n=3). FIG. 33b, Bar plots showing the on-target DNA base editing at two or more positions with C conversion frequencies>10% at each genomic site in HEK293T cells (mean±s.e.m., n=3). FIG. 33c-d, Frequencies of C (FIG. 33c) and T (FIG. 33d) conversions by gCBEv2 across the protospacer positions 1-20 (where PAM is at positions 21-23) from the edited sites in FIG. 4c. Single dot represents individual replicate (n=3 independent replicates per site), and boxes span the interquartile range (25th to 75th percentile); horizontal lines within the boxes indicate the median (50%); and whiskers extend to the minimal and maximal values. FIG. 33e, The ratio of C-to-G/T to C-to-A/G/T conversion frequency by gCBEv2 editing at the sites shown in FIG. 22c. FIG. 33f-h, Percentage of C-to-G (FIG. 33f), C-to-T (FIG. 33g) or C-to-A (FIG. 33h) editing by gCBEv2 at various edited sites shown in FIG. 3a (mean±s.e.m., n=3). FIG. 33i, indels frequencies with gCBEv2 at 16 on-target sites (mean±s.e.m., n=3). In FIG. 33e-i, C#: C position with highest on-target base editing frequencies across protospacer positions 1-20. site #: genomic site number. j, The statistical analysis of on-target DNA base editing for each NC motif from the 16 edited sites. Each dot represents the mean of three biological replicates for each edited position at various edited sites. n=8, 9, 10, 13 for motif AC, CC, GC, TC. FIG. 33k, indels frequencies with gCBEv2 and CGBE1 at 3 on-target sites from FIG. 4d (mean±s.e.m., n=3). FIG. 33l, On-target base editing frequencies for CGBE1 at C6 of site 22 in HEK293T cells for the orthogonal R-loop assay (mean±s.e.m., n=3).

FIG. 34. Base editing at spicing sites with gTBEv3. FIG. 34a, The optimal editing windows for various base editors. FIG. 34b, Venn diagram showing the distribution of sgRNAs for CBE and gCBEv2 in FIG. 23b. FIG. 34c, Bar plots showing the frequency of T or C conversations at several splicing acceptor (SA) or splicing donor (SD) sites of interest targeted by gTBEv3 or gCBEv2 (mean±s.e.m., n=3). T # or C#: The position of targeted T or C across protospacer positions 1-20. FIG. 34d, DNA sequencing chromatograms for targeting the SD site of human DMD exon 37 and exon 12 with gTBEv3. Sanger sequencing results were quantified by EditR.

FIG. 35. PTCs editing and introduction for various base editors. FIG. 35a, principle for bypassing premature termination codons (PTCs) with various base editors. FIG. 35b, the possible codon outcomes from stop codons (TAA, TAG or TGA) editing with different base editors. FIG. 35c, principle for introduction of PTCs with various base editors. FIG. 35d, the available codons for editing into stop codons (TAA, TAG or TGA) with different base editors. FIG. 35e, The 10×10 dot plot diagram showing the percentage of possible sgRNAs for introduction of premature termination codons (PTCs) by targeting different codons (with the number of available sgRNAs presented in the right) in 15 well-studied genes (AGT, ANGPTL3, APOC3, B2M, CD33, DNMT3A, HPD, KLKB1, PCSK9, PDCD1, PRDM1, TGFBR2, TRAC, TTR, VEGFA) for gene and cell therapy research with gGBEv6.3 and CBE. In FIG. 35b and FIG. 35d, AYBE, AYBEv3; gCBE, gCBEv2; gGBE, gGBEv6.3; gTBE, gTBEv3.

FIG. 36. Additional comparison of different gTBEs. FIG. 36a-b, The frequencies of T-to-G (FIG. 36a) or T-to-G (FIG. 36b) conversions. The highest frequencies (>20%) of edited thymines at corresponding positions were highlighted as Heat map (n=3). FIG. 36c-d, Heat map showing the percentages of T-to-S to total base conversions (FIG. 36c) or T-to-S to total T editing (base conversions and indels; FIG. 36d) for various base editors (n=3). FIG. 36e, Heat map showing the indels frequencies for various base editors (n=3). FIG. 36f-h, The statistical analysis of T base editing (FIG. 36f, n=35), T-to-S percentages (FIG. 36g, n=35) and indels (FIG. 36h, n=17). All values are presented as mean±s.e.m. Each dot represents the mean of three biological replicates for each edited position at various edited sites. FIG. 36a-h, the graphs were derived from the data for various base editors shown in FIG. 24c. Dunnett's multiple comparisons test after one-way ANOVA was used to compare the gTBEv3 or gTBEv5 with other base editors in FIG. 36f-h.

FIG. 37. T editing in the dsDNA upstream from the target site. FIG. 37a, Bar plots showing the frequency of T conversation at −5T, −3T, −2T and −1T in the dsDNA upstream from the target site 38 (VEGFA) (n=3). FIG. 37b, Bar plots showing the frequency of T conversation at −4T, −3T, −2T and −1T in the dsDNA upstream from the target site 44 (EMX1-siteH1F) (n=3). All values are presented as mean±s.e.m., the graphs were derived from the data for various base editors shown in FIG. 24c.

FIG. 38. Comparison of various glycosylase-based base editors for cytosine editing. FIG. 38a, Schematic of the basic architectures for various base editors. UNG2*, UNG2 mutant from the corresponding base editor. ΔNTD, deletion of the N-terminal domain. FIG. 38b-c, The frequencies of total C editing (base conversions and indels, FIG. 38b) or C base conversions (FIG. 38c) for various base editors at 19 endogenous loci. The cytosines with editing frequencies>25% for any base editors were showed. The highest frequencies at corresponding positions were highlighted as Heat map (n=3). FIG. 38d, Heat map showing the indels frequencies for various base editors (n=3). FIG. 38e-g, The statistical analysis of total C editing (FIG. 38e, n=56), C base conversions (FIG. 38f, n=49) and indels (FIG. 38g, n=19). All values are presented as mean±s.e.m. Each dot represents the mean of three biological replicates for each edited position at various edited sites. Dunnett's multiple comparisons test after one-way ANOVA was used to compare the gCBEv2 or gCBEv3 with other base editors.

FIG. 39. Additional comparison of various glycosylase-based base editors for cytosine editing. FIG. 39a-b, Heat map showing the C-to-G frequencies (FIG. 39a) or C-to-G purity (FIG. 39b) for various base editors (n=3). The cytosines with C-to-G editing frequencies>25% for any base editors were highlighted. FIG. 39c, Frequencies of C base conversions by various base editors across the protospacer positions 1-20 (where PAM is at positions 21-23). Single dot represents individual replicate (n=3 independent replicates per site), and boxes span the interquartile range (25th to 75th percentile); horizontal lines within the boxes indicate the median (50%); and whiskers extend to the minimal and maximal values. The graphs were derived from the data for various base editors shown in FIG. 38c.

FIG. 40. Off-target analysis of various glycosylase-based base editors. FIG. 40a-b, The guide sequence-dependent off-target analysis of cumulative T editing (FIG. 40a) or C editing (FIG. 40b) frequencies for various base editors at corresponding sites (n=3). OT: off-target. FIG. 40c, The guide sequence-independent cumulative off-target T editing detected by the orthogonal R-loop assay at each R-loop site (n=3). FIG. 40d, The statistical analysis of guide sequence-independent off-target T editing (n=5). Each dot represents the mean of three biological replicates for each edited position at various edited sites. Dunnett's multiple comparisons test after one-way ANOVA was used to compare the gTBEv3 or gTBEv5 with other base editors. FIG. 40e, RNA off-target analysis for various base editors (n=3). The mCherry was used as control. D=A or G or U; V=A or C or G. For multiple comparisons, the Dunnett's multiple comparisons test after one-way ANOVA was used to compare the gCBEv3 or gTBEv5 with other groups. For comparison of RNA U-to-V SNVs for gTBEv5 and mCherry, two-tailed unpaired two-sample t test was used. All values are presented as mean±s.e.m.

FIG. 41. Comparison between gTBEs or gCBEs and PEs. FIG. 41a, Bar plots showing the on-target T-to-G editing frequency for various editors at each genomic site in HEK293T cells (n=3). OT: off-target. FIG. 41b, Bar plots showing the on-target C-to-G editing frequency for various editors at each genomic site in HEK293T cells (n=3). The PE6d was used together with epegRNA and nick sgRNA. For PE6d max, PE6d was co-expressed with the codon-optimized hMLH1dn, a dominant negative MMR protein. All values are presented as mean±s.e.m.

FIG. 42. Characterization of editing profiles for gTBEs or gCBEs in HEK293T, HuH-7, and U2OS cells. FIG. 42a, Bar plots showing the on-target T base editing frequency, T-to-G editing purity or T-to-C editing purity for gTBEv3 and gTBEv5 in different cell lines (n=3). FIG. 42b, Bar plots showing the on-target C base editing frequency, C-to-G editing purity or C-to-T editing purity for gCBEv2 and gCBEv3 in different cell lines (n=3). HuH-7, a cell line established from a human hepatocellular carcinoma; U2OS, a cell line established from a human bone osteosarcoma. All values are presented as mean±s.e.m.

The figures herein are for illustrative purposes only and are not necessarily drawn to scale.

### DETAILED DESCRIPTION

### Overview

Traditional DNA base editors such as ABE and CBE can directly edit A and C with the help of deamination. However, G and T can only be indirectly edited by directly editing C and T on the strand opposite to G and T. In contrast, provided in the disclosure includes at least in part base editors and base editing methods capable of direct base editing of a target deoxyribonucleotide (e.g., dG, dT, dC) in a target dsDNA.

Traditional DNA base editors such as ABE and CBE require a deaminase for the base editing of A and C. In contrast, provided in the disclosure includes at least in part base editors and base editing methods capable of base editing of a target deoxyribonucleotide (e.g., dG, dT, dC) in a target dsDNA in the absence of a deaminase.

The base editors and base editing methods of the disclosure rely on the base excision domain of the base editor. The base excision domain is capable of directly excising the base of a target deoxyribonucleotide in a target dsDNA to generate an abasic site in situ, trigging a base excision repair (BER) pathway. As a result of base excision and subsequent repair, the target deoxyribonucleotide may be converted to another deoxyribonucleotide, leading to base editing of the target deoxyribonucleotide.

The base editors and base editing methods of the disclosure also rely on a nucleic acid programmable DNA binding domain (napDNAbd) to specifically direct the base editor to a target dsDNA via a guide nucleic acid capable of interacting with both the napDNAbd and the target dsDNA. The napDNAbd may be associated (e.g., complex) with a guide nucleic acid (e.g., a guide RNA). The guide nucleic acid is designed to localize or target the napDNAbd to the target dsDNA, by relying on the hybridization between a target sequence of the target dsDNA and a corresponding guide sequence of the guide nucleic acid. Specifically, the guide nucleic acid comprises a guide sequence that is capable of hybridizing to a target sequence of the target dsDNA due to the substantial complementarity between the guide sequence and the target sequence. The guide nucleic acid also comprises a scaffold sequence capable of forming a complex with the napDNAbd. In this way, the guide nucleic acid “programs” the napDNAbd such that the napDNAbd can specifically localize and (indirectly) bind to the region on and around the target sequence of the target dsDNA via the guide nucleic acid. The binding of the napDNAbd to the target dsDNA enables the base excising domain associated with the napDNAbd to specifically access to and function on the base of the target deoxyribonucleotide in the target sequence of the target dsDNA in a guide sequence-specific/dependent way.

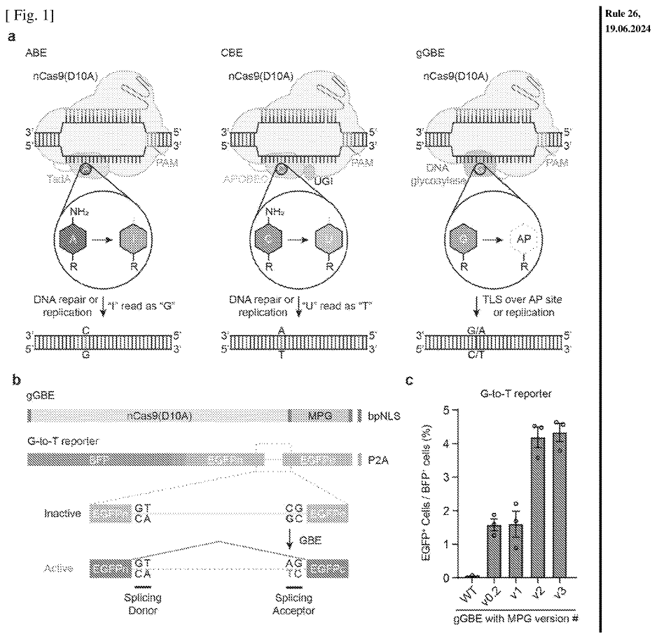

Referring to FIGS. 1a, 6-8, and 19a, the base excision domain of the base editor of the disclosure directly excises the base of a target deoxyribonucleotide (the first deoxyribonucleotide in FIG. 6), generates an abasic site where the base is removed, which is an apurinic site where a purine (e.g., guanine) is removed or an apyrimidinic site where a pyrimidine (e.g., thymine, cytosine) is removed. The abasic site may be repaired by translesion synthesis (TLS) (by, e.g., TLS polymerase) and/or DNA replication, leading to base editing, in which case nicking in the strand opposite to the abasic site may not be necessary.

Alternatively, if a nucleic acid programmable DNA nickase such as Cas9 nickase is used as the napDNAbd, it creates a nick in the strand (non-edited strand) opposite to the abasic site, and the apyrimidinic site may be removed by AP lyase to generate another nick on the edited strand, which two nicks trigger double-strand break (DSB) repair and introduction of indel mutation, also leading to highly potential change of the target deoxyribonucleotide.

As a specific example, FIG. 6 shows, before base editing, a first deoxyribonucleotide of dG as target deoxyribonucleotide to be edited on the edited strand (nontarget strand) and a second deoxyribonucleotide of dC on the opposite strand (non-edited strand/target strand) base pairing with the dG. The dG is located in a protospacer sequence on the nontarget strand of the target dsDNA, and the dC is located in a target sequence on the target strand of the target dsDNA. A guide nucleic acid is designed to comprise a guide sequence capable of hybridizing to the target sequence and comprises a scaffold sequence capable of forming a complex with a napDNAbd. The napDNAbd is capable of nicking the target stand and is fused with a base excising domain capable of excising guanine of the dG. As a specific example, FIG. 7 shows, after base editing, a fourth deoxyribonucleotide of dC as outcome deoxyribonucleotide on the edited strand (nontarget strand) and a third deoxyribonucleotide of dG on the opposite strand (non-edited strand/target strand) base pairing with the dC. FIG. 6 and FIG. 7 together show direct dG-to-dC base editing. As a specific example, FIG. 8 shows, after base editing, a fourth deoxyribonucleotide of dT as outcome deoxyribonucleotide on the edited strand (nontarget strand) and a third deoxyribonucleotide of dA on the opposite strand (non-edited strand/target strand) base pairing with the dT. FIG. 6 and FIG. 8 together show direct dG-to-dT base editing.

The base editing approach of the disclosure allows direct base editing of a target deoxyribonucleotide (e.g., dG, dT, dC) in a target dsDNA, expanding the scope of target design and screening for the direct base editing. For example, if editing of a target dG to dA is desired, the traditional base editors incapable of directly editing dG would have to be applied to edit dC on the opposite strand to dT, thereby indirectly editing dG to dA. However, the editing ability of the traditional CBE might not be able to edit the dC with a desired outcome dT; the PAM limitation of the CBE might not allow designing a target/guide sequence targeting the dC to specifically direct the CBE to the dC; and even if such a guide sequence can be designed, the base editing efficiency of the CBE might not be sufficient. With the base editing approach of the disclosure, the target dG can be directly base edited, and therefore, developers would have much more chance to design, screen, and obtain a suitable target/guide sequence targeting the dG to specifically direct the base editor of the disclosure to the dG.

The base editing approach of the disclosure may function in the absence of deamination of the base of the target deoxyribonucleotide before the excision of the base of the target deoxyribonucleotide, or in the absence of deamination at all. On the contrary, traditional ABE needs to deaminase the base (adenine) of a target dA to a hypoxanthine, thereby converting the target dA to inosine (I), which reads as dG in DNA repair replication; and traditional CBE needs to deaminase the base (cytosine) of a target dC to an uracil, thereby converting the target dC to uridine (U), which reads as dT in DNA repair or replication. Deamination is unlikely for G (due to spontaneous remediation) [18] and impossible for T (due to the absence of amine), making the development of deaminase-based G and T base editors a challenging task. The omission of a deaminase domain in the base editor of the disclosure opens the way to base editing of G and T, and may also avoid undesired effects caused by the deaminase domain and deamination of traditional deaminase-based base editors and reduce base editor size.

As a non-limiting example of the disclosure, a deaminase-free, glycosylase-based guanine base editor (gGBE) was developed with G editing ability, by fusing a nucleic acid programmable DNA nickase such as Cas9 nickase with a human N-methylpurine DNA glycosylase (MPG) mutant capable of excising guanine of dG developed by several rounds of MPG mutagenesis via unbiased and rational screening. It was demonstrated that the gGBE has high G editing efficiency. Furthermore, the gGBE exhibited high base editing efficiency (up to 81.2%) and high G-to-T or G-to-C (i.e., G-to-Y) conversion ratio (up to 0.95) in both cultured human cells and mouse embryos. The editing profile of the gGBE was characterized by targeting dozens of endogenous genomic loci in cultured mammalian cells as well as mouse embryos, demonstrating its high G-to-Y (Y=C or T) base editing efficiency.

As another non-limiting example of the disclosure, by fusing a nucleic acid programmable DNA nickase such as Cas9 nickase with a human uracil DNA glycosylase (UNG) mutant capable of excising thymine of dT or cytosine of dC separately developed by mutagenesis of UNG, two deaminase-free, glycosylase-based base editors for direct T editing (gTBE) and direct C editing (gCBE) were developed to achieve orthogonal base editing, that is gTBE for direct T editing and gCBE for direct C editing, respectively. By several rounds of structure-informed rational mutagenesis on UNG in cultured human cells, gTBE and gCBE were obtained with high activity of T-to-S (i.e., T-to-C or T-to-G) and C-to-G conversions, respectively. Furthermore, by embedding the UNG mutant into a nucleic acid programmable DNA nickase such as Cas9 nickase, more gTBE and gCBE were generated, showing enhanced average editing efficiency and alternative editing windows. The editing profile of gTBE and gCBE were characterized by targeting dozens of endogenous genomic loci in cultured mammalian cells as well as mouse embryos, demonstrating their high base editing efficiency.

### Base Editor

The base editor of the disclosure may be provided in the form of a fusion protein. In an aspect, the disclosure provides a fusion protein comprising:
(1) a nucleic acid programmable DNA binding domain (napDNAbd) capable of binding a target dsDNA comprising:
(a) a first deoxyribonucleotide (e.g., dG (deoxyguanosine), dT (thymidine), dC (deoxycytidine)) in a protospacer sequence on a nontarget strand (edited strand) of the target dsDNA, and
(b) a second deoxyribonucleotide (e.g., dC (deoxycytidine), dA (deoxyadenosine), dG (deoxyguanosine)) base pairing with the first deoxyribonucleotide (e.g., dG, dT, dC) and in a target sequence on the target strand (non-edited strand) of the target dsDNA, wherein the protospacer sequence is fully reverse complementary to the target sequence; and
(2) a base excising domain capable of excising the base (e.g., guanine, thymine, cytosine) of the first deoxyribonucleotide.

In some embodiments, the fusion protein does not comprise a deaminase domain, e.g., an adenine or cytosine deaminase domain, e.g., TadA and variants thereof.

The components of the fusion protein are described more specifically in the other sub-sections herein.

### Base Editing System

In another aspect, the disclosure provides a system comprising:
(i) a fusion protein or a polynucleotide encoding the fusion protein, the fusion protein comprising:
(1) a nucleic acid programmable DNA binding domain (napDNAbd) capable of binding a target dsDNA comprising:
(a) a first deoxyribonucleotide (e.g., dG (deoxyguanosine), dT (thymidine), dC (deoxycytidine)) in a protospacer sequence on a nontarget strand (edited strand) of the target dsDNA, and
(b) a second deoxyribonucleotide (e.g., dC (deoxycytidine), dA (deoxyadenosine), dG (deoxyguanosine)) base pairing with the first deoxyribonucleotide (e.g., dG, dT, dC) and in a target sequence on the target strand (non-edited strand) of the target dsDNA, wherein the protospacer sequence is fully reverse complementary to the target sequence; and
(2) a base excising domain capable of excising the base (e.g., guanine, thymine, cytosine) of the first deoxyribonucleotide; and
(ii) a guide nucleic acid or a polynucleotide encoding the guide nucleic acid, the guide nucleic acid comprising:
(1) a scaffold sequence capable of forming a complex with the napDNAbd; and
(2) a guide sequence capable of hybridizing to the target sequence on the target strand of the target dsDNA, thereby guiding the complex to the target dsDNA.

In some embodiments, the system is a complex comprising the fusion protein complexed with the guide nucleic acid. In some embodiments, the complex further comprises the target dsDNA hybridized with the guide sequence. In some embodiments, the system is a composition comprising the component (i) and the component (ii).

In some embodiments, the guide nucleic acid as described herein is a guide RNA (gRNA). As used herein, the term “gRNA” is used interchangeably with single guide RNA (sgRNA).

The components of the system are described more specifically in the other sub-sections herein.

### Base Editing Method

In yet another aspect, the disclosure provides a method of modifying a target dsDNA, comprising contacting the target dsDNA with a system,
the target dsDNA comprising:
(a) a first deoxyribonucleotide (e.g., dG (deoxyguanosine), dT (thymidine), dC (deoxycytidine)) in a protospacer sequence on a nontarget strand (edited strand) of the target dsDNA, and
(b) a second deoxyribonucleotide (e.g., dC (deoxycytidine), dA (deoxyadenosine), dG (deoxyguanosine)) base pairing with the first deoxyribonucleotide (e.g., dG, dT, dC) and in a target sequence on the target strand (non-edited strand) of the target dsDNA, wherein the protospacer sequence is fully reverse complementary to the target sequence; and
the system comprising:
(i) a fusion protein or a polynucleotide encoding the fusion protein, the fusion protein comprising:
(1) a nucleic acid programmable DNA binding domain (napDNAbd) capable of binding the target dsDNA; and
(2) a base excising domain capable of excising the base (e.g., guanine, thymine, cytosine) of the first deoxyribonucleotide; and
(ii) a guide nucleic acid or a polynucleotide encoding the guide nucleic acid, the guide nucleic acid comprising:
(1) a scaffold sequence capable of forming a complex with the napDNAbd; and
(2) a guide sequence capable of hybridizing to the target sequence on the target strand of the target dsDNA, thereby guiding the complex to the target dsDNA.

In some embodiments, the method does not include deamination of the base of the first deoxyribonucleotide before the excision of the base of the first deoxyribonucleotide.

In some embodiments, the method does not include deamination of the base of the first deoxyribonucleotide.

In some embodiments, the method comprises inducing strand separation of the target dsDNA.

The components of the method are described more specifically in the other sub-sections herein.

Target deoxyribonucleotide and target dsDNA

Referring to FIG. 6-8, the target deoxyribonucleotide to be edited in the target dsDNA may be termed as “first deoxyribonucleotide”, and the outcome deoxyribonucleotide converted from the target deoxyribonucleotide by base editing may be termed as “fourth deoxyribonucleotide”.

In some embodiments, the first deoxyribonucleotide is deoxyguanosine (dG), thymidine (dT), deoxyadenosine (dA), or deoxycytidine (dC). In some embodiments, the first deoxyribonucleotide is dG. In some embodiments, the first deoxyribonucleotide is dT. In some embodiments, the fourth deoxyribonucleotide is dA, dT, dC, or dG. In some embodiments, the second deoxyribonucleotide is dC, dA, dT, or dG. In some embodiments, the third deoxyribonucleotide is dA, dT, dC, or dG.

As would be desired, in some embodiments, the first deoxyribonucleotide is converted to a fourth deoxyribonucleotide that is different from the first deoxyribonucleotide. In some embodiments, the conversion of the first deoxyribonucleotide to the fourth deoxyribonucleotide is dG-to-dA, dG-to-dT, dG-to-dC, dT-to-dA, dT-to-dC, dT-to-dG, dC-to-dA, dC-to-dT, or dC-to-dG.

In some embodiments, the target dsDNA is a wild type or naturally-occuring. In some embodiments, the target dsDNA is not a wild type or naturally-occuring. In some embodiments, the target dsDNA is eukaryotic or prokaryotic. In some embodiments, the target dsDNA is from an animal (e.g., human, monkey, mouse) or plant. In some embodiments, the target dsDNA is a target gene. In some embodiments, the gene is an animal (e.g., human, monkey, mouse) or plant gene. In some embodiments, the dsDNA is in a target cell.

In some embodiments, the first deoxyribonucleotide is native or nonnative to the target dsDNA. In some embodiments, the first deoxyribonucleotide is a mutation in the target dsDNA. In some embodiments, the first deoxyribonucleotide is a pathogenic mutation in the target dsDNA. In some embodiments, the first deoxyribonucleotide is a mutation resulting in a stop codon in the target dsDNA.

In some embodiments, the conversion of the first deoxyribonucleotide to the fourth deoxyribonucleotide directly or indirectly converts a stop codon to a non-stop codon or directly or indirectly converts a non-stop codon to a stop codon, either on the target strand or the nontarget strand. In some embodiments, the stop codon is on the sense strand of the dsDNA.

In some embodiments, the the conversion of the first deoxyribonucleotide to the fourth deoxyribonucleotide occurs on the sense strand or the nonsense strand of the dsDNA. In some embodiments, the the conversion of the first deoxyribonucleotide to the fourth deoxyribonucleotide (e.g., dG-to-dT) occurs on the sense strand of the dsDNA, converting a stop codon on the sense strand to a non-stop codon or converting a non-stop codon (e.g., GAA) on the sense strand to a stop codon (e.g., TAA). In some embodiments, the the conversion of the first deoxyribonucleotide to the fourth deoxyribonucleotide (e.g., dG-to-dC) occurs on the nonsense strand of the dsDNA, converting a stop codon on the sense strand to a non-stop codon or converting a non-stop codon (e.g., TCA) on the sense strand to a stop codon (e.g., TGA).

In some embodiments, the the conversion of the first deoxyribonucleotide to the fourth deoxyribonucleotide (e.g., dG-to-dC) occurs at the splicing site (e.g., splicing donor, splicing acceptor) of the target dsDNA. In some embodiments, the the conversion of the first deoxyribonucleotide to the fourth deoxyribonucleotide (e.g., dG-to-dC) occurring at the splicing site (e.g., splicing donor, splicing acceptor) increases or decreases the translation of a transcript transcribed from the target dsDNA.

In some embodiments, the first deoxyribonucleotide is at a position of the protospacer sequence selected from the group consisting of position 1, position 2, position 3, position 4, position 5, position 6, position 7, position 8, position 9, position 10, position 11, position 12, position 13, position 14, position 15, position 16, position 17, position 18, position 19, position 20, and a combination thereof; or wherein the first deoxyribonucleotide is at a position of the protospacer sequence between position 1 and position 20, both inclusive; or wherein the first deoxyribonucleotide is at a position of the protospacer sequence between position 1 and position 14, both inclusive. In some embodiments, the first deoxyribonucleotide is at a position of the protospacer sequence selected from the group consisting of position 6, position 7, position 8, position 9, position 10, position 11, and a combination thereof; or wherein the first deoxyribonucleotide is at a position of the protospacer sequence between position 6 and position 11, both inclusive. In some embodiments, the first deoxyribonucleotide is at position 7 of the protospacer sequence.

In some embodiments, the first deoxyribonucleotide is the N1 or N2 nucleotide in a motif of N1N2, wherein N1 or N2 is A, T, G, or C. In some embodiments, the first deoxyribonucleotide is the N2 nucleotide in a motif of N1N2, wherein N1 is A or T, and N2 is C. In some embodiments, the first deoxyribonucleotide is the N1, N2, or N3 nucleotide in a motif of N1N2N3, wherein N1, N2, or N3 is A, T, G, or C.

### Base Excising Domain

As used herein, the term “base excising domain (BED)” is used interchangeably with “base excising protein (BEP)” or “base excising enzyme (BEE)” and refers to a protein capable of recognizing and excising a base (e.g., A, T, C, G, or U) of a nucleotide of a nucleic acid (e.g., DNA (ssDNA or dsDNA) or RNA). As used herein, the term “base” is used interchangeably with “nucleobase” or “nitrogenous base”. Base includes, for example, adenine (A), cytosine (C), guanine (G), thymine (T), and uracil (U), and they may be termed as primary, normal, or canonical base. As well known in the art, a deoxyribonucleotide is composed of a base, a deoxyribose, and a phosphate, and a deoxyribonucleoside is composed of a base and a deoxyribose. Excising the base of a deoxyribonucleoside releases the base from the deoxyribonucleoside. In some embodiments, excising the base of a deoxyribonucleoside comprises cleaving or hydrolyzing the glycosidic bond linking the base to the deoxyribose of the first deoxyribonucleotide, thereby releasing the base from the first deoxyribonucleotide.

In some embodiments, the base excising domain is (substantially) capable of excising the base (e.g., guanine, thymine, cytosine) of the first deoxyribonucleotide (e.g., dG, dT, dC). In some embodiments, the first deoxyribonucleotide is dG, dT, dA, or dC. In some embodiments, the base excising domain is (substantially) capable of excising guanine of dG. In some embodiments, the base excising domain is (substantially) capable of excising thymine of dT. In some embodiments, the base excising domain is (substantially) capable of excising cytosine of dC. In some embodiments, the base excising domain is (substantially) capable of excising adenine of dA.

In some cases, it would be desired to apply highly specific base editing, for example, in disease treatment. Therefore, it would be desired that the base excising domain can only excise one type of bases but not excise the other types of bases. In some embodiments, the base excising domain is (substantially) incapable of excising guanine of dG. In some embodiments, the base excising domain is (substantially) incapable of excising thymine of dT. In some embodiments, the base excising domain is (substantially) incapable of excising cytosine of dC. In some embodiments, the base excising domain is (substantially) incapable of excising adenine of dA.

In some cases, it would be desired to apply mutilplexed base editing. Therefore, it would be desired that the base excising domain can excise more than one type of bases. In some embodiments, the base excising domain is (substantially) capable of excising any two, three, or four of guanine of dG, thymine of dT, cytosine of dC, and adenine of dA. For example, in some embodiments, the base excising domain is (substantially) capable of excising both guanine of dG and thymine of dT.

In some embodiment, the base excising domain is (substantially) capable of excising uracil. In some embodiment, the base excising domain is (substantially) incapable of excising uracil. In some embodiment, the base excising domain is (substantially) capable of excising hypoxanthine. In some embodiment, the base excising domain is (substantially) incapable of excising hypoxanthine.

In some embodiments, the fusion protein of the disclosure does not comprise a base excising domain (substantially) capable of excising guanine of dG, thymine of dT, cytosine of dC, adenine of dA, uracil, and/or hypoxanthine.

In some embodiment, the base excising domain is (substantially) incapable of excising bases on both strands of a target dsDNA. In some embodiments, the base excising domain is (substantially) incapable of excising both bases of a pair of base-paired deoxyribonucleotides on both strands of a dsDNA.

In some embodiments, the base excision domain comprises an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to a naturally-occurring base excision domain, such as a naturally-occurring base excision domain provided herein.

In some embodiments, the fusion protein of the disclosure comprises one, two, three, or more base excising domains. In some embodiments, the fusion protein comprises two, three, or more base excising domains, which are the same or different.

The base excising domain could be a glycosylase having the desired base exising ability. In some embodiments, the base excising domain comprises a glycosylase. In some embodiments, the glycosylase is selected from the group consisting of N-methylpurine DNA glycosylase (MPG), 8-oxoguanine DNA glycosylase (OGG1), methyl-CpG binding domain 4, DNA glycosylase (MBD4), thymine DNA glycosylase (TDG), uracil DNA glycosylase (UNG), single-strand-selective monofunctional uracil-DNA glycosylase 1 (SMUG1), mutY DNA glycosylase (MUTYH), nth like DNA glycosylase 1 (NTHL1), nei like DNA glycosylase 1 (NEIL1), nei like DNA glycosylase 2 (NEIL2), nei like DNA glycosylase 3 (NEIL3), and mutants thereof capable of recognizing and excising a base from a nucleotide of a nucleic acid.

Exemplary glycosylases capable of excising a base include, without limitation, UDG-N204D and UDG-Y147A as described in Kavli, B. et al. Excision of cytosine and thymine from DNA by mutants of human uracil-DNA glycosylase. EMBO J 15, 3442-3447 (1996); the entire contents of which are hereby incorporated by reference.

In some embodiments, the base excision domain is not wild type or naturally-occurring.

### MPG

In some embodiments, the base excising domain comprises an N-methylpurine DNA glycosylase (MPG). In some embodiments, the MPG comprises a motif GxxYxxxxYGxxxxxN, wherein x represents any amino acid. In some embodiments, the MPG is obtained from a species selected from Table A. In some embodiments, the MPG comprises an amino acid mutation relative to (compared to; with reference to) a wild type or reference MPG. In some embodiments, the wild type or reference MPG is human MPG (SEQ ID NO: 9) or an MPG obtained from a species selected from Table A or any MPG as set forth in Table D or a homology or mutant (e.g., comprising an amino acid sequence of SEQ ID NO: 5, 6, or 7) thereof or an N-terminal truncation thereof lacking the most N-terminal Methionine (Met) (coded by start codon ATG) (e.g., SEQ ID NO: 1). In some embodiments, the wild type or reference MPG comprises the amino acid sequence of SEQ ID NO: 7.

In some embodiments, the amino acid mutation comprises an amino acid substitution at a position that is corresponding to a position or that is a position selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, and/or 298 of the wild type or reference MPG, wherein the position is numbered according to SEQ ID NO: 1.

In some embodiments, the amino acid mutation confers an ability to excise a base on the MPG. In some embodiments, the base is guanine.

In some embodiments, the amino acid mutation leads to increased base excising ability as compared to an otherwise identical control MPG without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation leads to increased guide sequence-specific base editing efficiency of the fusion protein as compared to an otherwise identical control fusion protein without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation comprises an amino acid substitution at a position that is corresponding to a position or that is a position selected from the group consisting of G163, N169, D175, C178, S198, K202, G203, S206, K210, and/or Q294 of the wild type or reference MPG, wherein the position is numbered according to SEQ ID NO: 1.

In some embodiments, the amino acid mutation comprises an amino acid substitution at a position that is corresponding to a position or that is a position selected from the group consisting of N169, D175, C178, and/or Q294 of the wild type or reference MPG, wherein the position is numbered according to SEQ ID NO: 1. In some embodiments, the wild type or reference MPG comprises the amino acid sequence of SEQ ID NO: 7.

In some embodiments, the amino acid substitution is a conservative amino acid substitution or a non-conservative amino acid substitution. In some embodiments, the amino acid substitution is an amino acid substitution with an amino acid residue that is different from the amino acid residue at the position of the wild type or reference MPG. In some embodiments, the amino acid substitution is an amino acid substitution with (1) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/1), Methionine (Met/M), Tryptophan (Trp/W), Phenylalanine (Phe/F); (2) a polar amino acid residue (such as, Serine (Ser/S), Threonine (Thr/T), Tyrosine (Tyr/Y), Asparagine (Asn/N), Glutamine (Gln/Q)); (3) a positively charged amino acid residue (such as, Lysine (Lys/K), Arginine (Arg/R), Histidine (His/H)); or (4) a negatively charged amino acid residue (such as, Aspartic Acid (Asp/D), Glutamic Acid (Glue/E)). In some embodiments, the amino acid substitution is a substitution with R, A, N, or G.

In some embodiments, the amino acid mutation comprises an amino acid substitution that is corresponding to a substitution or that is a substitution selected from the group consisting of G163R, N169G, D175R, C178N, S198A, K202A, G203A, S206A, K210A, Q294R, and a combination of any two or more substitutions thereof, wherein the position is numbered according to SEQ ID NO: 1.

In some embodiments, the MPG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the wild type or reference MPG.

In some embodiments, the amino acid mutation comprises an amino acid substitution that is corresponding to a substitution or that is a substitution selected from the group consisting of N169G, D175R, C178N, Q294R, and a combination of any two or more substitutions thereof, wherein the position is numbered according to SEQ ID NO: 1. In some embodiments, the wild type or reference MPG comprises the amino acid sequence of SEQ ID NO: 7.

In some embodiments, the amino acid mutation comprises a combination substitution that is corresponding to a combination substitution of N169G, D175R, C178N, and Q294R, wherein the position is numbered according to SEQ ID NO: 1. In some embodiments, the wild type or reference MPG comprises the amino acid sequence of SEQ ID NO: 7.

In some embodiments, the amino acid mutation comprises a combination substitution that is corresponding to a combination substitution of G163R, N169G, D175R, C178N, S198A, K202A, G203A, S206A, K210A, Q294R, wherein the position is numbered according to SEQ ID NO: 1. In some embodiments, the wild type or reference MPG comprises the amino acid sequence of SEQ ID NO: 1 or 9.

In some embodiments, the MPG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 8, 16, 18, 20, 22, 24, 26, 28, 30, 32, 34, 36, or 38.

In some embodiments, the MPG is (substantially) capable of excising guanine of dG. In some embodiments, the MPG is (substantially) incapable of excising thymine of dT. In some embodiments, the MPG is (substantially) incapable of excising cytosine of dC. In some embodiments, the MPG is (substantially) incapable of excising adenine of dA.

In some embodiments, the MPG is not wild type or naturally-occurring.

UNG

In some embodiments, the base excising domain comprises an uracil-DNA glycosylase (UNG). In some embodiments, the UNG comprises a motif GQDPYH. In some embodiments, the UNG is obtained from a species selected from Table C. In some embodiments, the UNG comprises an amino acid mutation relative to (compared to; with reference to) a wild type or reference UNG. In some embodiments, the wild type or reference UNG is human UNG1 (SEQ ID NO: 54) or human UNG2 (SEQ ID NO: 133) or an UNG obtained from a species selected from Table C or any UNG as set forth in Table D or a homology or mutant thereof or an N-terminal truncation thereof lacking the most N-terminal Methionine (Met) (coded by start codon ATG). In some embodiments, the wild type or reference UNG comprises the amino acid sequence of SEQ ID NO: 56, 58, 135, or 137.

In some embodiments, the amino acid mutation comprises an amino acid substitution at a position that is corresponding to a position or that is a position selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, and/or 313 of the wild type or reference UNG, wherein the position is numbered according to SEQ ID NO: 133.

Alternative splicing as well as transcription from two distinct start sites leads to two different human UNG isoforms, mitochondrial UNG1 (304 amino acids, aa) (SEQ ID NO: 54) and nuclear UNG2 (313 aa) (SEQ ID NO: 133), each possessing an unique N-terminus that mediates translocation to mitochondria or nucleus¹⁶ (FIG. 25). The sequence alignment of human UNG1 and human UNG2 shows that the amino acid residues at positions 1-35 of UNG1 are different from the amino acid residues at positions 1-44 of UNG2, and the remaining parts of UNG1 and UNG2 are identical. The corresponding relationship between the positions of UNG1 (SEQ ID NO: 54) and UNG2 (SEQ ID NO: 133) can be determined by sequence alignment. For example, the residues Y156 and N213 of UNG2 are corresponding to the residues Y147 and N204 of UNG1, respectively.

In some embodiments, the amino acid mutation confers an ability to excise a base on the UNG. In some embodiments, the base is thymine. In some embodiments, the base is cytosine.

In some embodiments, the amino acid mutation leads to increased base excising ability as compared to an otherwise identical control UNG without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation leads to increased guide sequence-specific base editing efficiency of the fusion protein as compared to an otherwise identical control fusion protein without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation comprises an amino acid substitution at a position that is corresponding to a position or that is a position selected from the group consisting of Y156, K184, N213, A214, Q259, and/or Y284 of the wild type or reference UNG, wherein the position is numbered according to SEQ ID NO: 133.

In some embodiments, the amino acid mutation comprises an amino acid substitution at a position that is corresponding to a position or that is a position selected from the group consisting of K184, A214, Q259, and/or Y284 of the wild type or reference UNG, wherein the position is numbered according to SEQ ID NO:133. In some embodiments, the wild type or reference UNG comprises the amino acid sequence of SEQ ID NO: 135 or 137.

In some embodiments, the amino acid substitution is a conservative amino acid substitution or a non-conservative amino acid substitution. In some embodiments, the amino acid substitution is an amino acid substitution with an amino acid residue that is different from the amino acid residue at the position of the wild type or reference UNG. In some embodiments, the amino acid substitution is an amino acid substitution with (1) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F); (2) a polar amino acid residue (such as, Serine (Ser/S), Threonine (Thr/T), Tyrosine (Tyr/Y), Asparagine (Asn/N), Glutamine (Gln/Q)); (3) a positively charged amino acid residue (such as, Lysine (Lys/K), Arginine (Arg/R), Histidine (His/H)); or (4) a negatively charged amino acid residue (such as, Aspartic Acid (Asp/D), Glutamic Acid (Glue/E)). In some embodiments, the amino acid substitution is a substitution with A, D, V, or T.

In some embodiments, the amino acid mutation comprises an amino acid substitution that is corresponding to a substitution or that is a substitution selected from the group consisting of Y156A, K184A, N213D, A214V, A214T, Q259A, Y284D, and a combination of any two or more substitutions thereof, wherein the position is numbered according to SEQ ID NO: 133.

In some embodiments, the amino acid mutation comprises an amino acid substitution that is corresponding to a substitution or that is a substitution selected from the group consisting of A214T, Q259A, Y284D, and a combination of any two or more substitutions thereof, wherein the position is numbered according to SEQ ID NO: 133. In some embodiments, the wild type or reference UNG comprises the amino acid sequence of SEQ ID NO: 135.

In some embodiments, the amino acid mutation comprises an amino acid substitution that is corresponding to a substitution or that is a substitution selected from the group consisting of K184A, A214V, and a combination of the two substitutions, wherein the position is numbered according to SEQ ID NO: 133. In some embodiments, the wild type or reference UNG comprises the amino acid sequence of SEQ ID NO: 137.

In some embodiments, the amino acid mutation comprises a deletion of amino acids at positions that are corresponding to positions or that are positions 1-65, 1-66, 1-67, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of the wild type or reference UNG, wherein the position is numbered according to SEQ ID NO: 133.

In some embodiments, the amino acid mutation comprises an amino acid substitution that is corresponding to a substitution or that is a substitution selected from the group consisting of A214T, Q259A, Y284D, and a combination of any two or more substitutions thereof, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 135.

In some embodiments, the amino acid mutation comprises an amino acid substitution that is corresponding to a substitution or that is a substitution selected from the group consisting of K184A, A214V, and a combination of the two substitutions, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 137.

In some embodiments, the UNG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the wild type or reference UNG.

In some embodiments, the amino acid mutation comprises a combination substitution that is corresponding to a combination substitution of A214T, Q259A, and Y284D, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 135.

In some embodiments, the amino acid mutation comprises a combination substitution that is corresponding to a combination substitution of K184A and A214V, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 137.

In some embodiments, the UNG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 56, 58, 60, 62, 135, 137, 139, 141, 144, 146, 148, 150, 152, 155, 157, and 159 or an N-terminal truncation thereof lacking the most N-terminal Methionine (M) (coded by start codon ATG).

In some embodiments, the UNG is (substantially) capable of excising thymine of dT. In some embodiments, the UNG is (substantially) capable of excising cytosine of dC. In some embodiments, the UNG is (substantially) incapable of excising thymine of dT. In some embodiments, the UNG is (substantially) incapable of excising cytosine of dC. In some embodiments, the UNG is (substantially) incapable of excising adenine of dA. In some embodiments, the UNG is (substantially) incapable of excising guanine of dG.

In some embodiments, the UNG is not wild type or naturally-occurring.

### Additional BED

In some embodiments, the base excising domain comprises TDG, SMUG1, OGG1, MBD4, MUTYH, NEIL1, NEIL2, NEIL3, or NTHL1. In some embodiments, the TDG, SMUG1, OGG1, MBD4, MUTYH, NEIL1, NEIL2, NEIL3, or NTHL1 comprises an amino acid mutation relative to (compared to; with reference to) a wild type or reference TDG, SMUG1, OGG1, MBD4, MUTYH, NEIL1, NEIL2, NEIL3, or NTHL1. In some embodiments, the wild type or reference TDG, SMUG1, OGG1, MBD4, MUTYH, NEIL1, NEIL2, NEIL3, or NTHL1 is human TDG, SMUG1, OGG1, MBD4, MUTYH, NEIL1, NEIL2, NEIL3, or NTHL1 (SEQ ID NO: 64, 65, 66, 67, 68, 69, 70, 71, or 72, respectively) or a homology or mutant thereof or an N-terminal truncation thereof lacking the most N-terminal Methionine (Met) (coded by start codon ATG). In some embodiments, the wild type or reference TDG, SMUG1, OGG1, MBD4, MUTYH, NEIL1, NEIL2, NEIL3, or NTHL1 comprises an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 64-72, respectively.

In some embodiments, the amino acid mutation comprises an amino acid substitution at a position that is corresponding to a position or that is a position selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, and/or 604 of the wild type or reference TDG, SMUG1, OGG1, MBD4, MUTYH, NEIL1, NEIL2, NEIL3, or NTHL1, wherein the position is numbered according to any one of SEQ ID NOs: 64-72.

In some embodiments, the amino acid mutation confers an ability to excise a base on the TDG, SMUG1, OGG1, MBD4, MUTYH, NEIL1, NEIL2, NEIL3, or NTHL1. In some embodiments, the base is guanine, thymine, cytosine, adenine, uracil, or hypoxanthine.

In some embodiments, the amino acid mutation leads to increased base excising ability as compared to an otherwise identical control TDG, SMUG1, OGG1, MBD4, MUTYH, NEIL1, NEIL2, NEIL3, or NTHL1 without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation leads to increased guide sequence-specific base editing efficiency of the fusion protein as compared to an otherwise identical control fusion protein without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid substitution is a conservative amino acid substitution or a non-conservative amino acid substitution. In some embodiments, the amino acid substitution is an amino acid substitution with an amino acid residue that is different from the amino acid residue at the position of the wild type or reference TDG, SMUG1, OGG1, MBD4, MUTYH, NEIL1, NEIL2, NEIL3, or NTHL1. In some embodiments, the amino acid substitution is an amino acid substitution with (1) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F); (2) a polar amino acid residue (such as, Serine (Ser/S), Threonine (Thr/T), Tyrosine (Tyr/Y), Asparagine (Asn/N), Glutamine (Gln/Q)); (3) a positively charged amino acid residue (such as, Lysine (Lys/K), Arginine (Arg/R), Histidine (His/H)); or (4) a negatively charged amino acid residue (such as, Aspartic Acid (Asp/D), Glutamic Acid (Glue/E)).

In some embodiments, the TDG, SMUG1, OGG1, MBD4, MUTYH, NEIL1, NEIL2, NEIL3, or NTHL1 comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the wild type or reference TDG, SMUG1, OGG1, MBD4, MUTYH, NEIL1, NEIL2, NEIL3, or NTHL1, respectively.

In some embodiments, the TDG, SMUG1, OGG1, MBD4, MUTYH, NEIL1, NEIL2, NEIL3, or NTHL1 is (substantially) capable of excising guanine of dG. In some embodiments, the TDG, SMUG1, OGG1, MBD4, MUTYH, NEIL1, NEIL2, NEIL3, or NTHL1 is (substantially) capable of excising thymine of dT. In some embodiments, the TDG, SMUG1, OGG1, MBD4, MUTYH, NEIL1, NEIL2, NEIL3, or NTHL1 is (substantially) capable of excising cytosine of dC. In some embodiments, the TDG, SMUG1, OGG1, MBD4, MUTYH, NEIL1, NEIL2, NEIL3, or NTHL1 is (substantially) capable of excising adenine of dA.

In some embodiments, the TDG, SMUG1, OGG1, MBD4, MUTYH, NEIL1, NEIL2, NEIL3, or NTHL1 is (substantially) incapable of excising thymine of dT. In some embodiments, the TDG, SMUG1, OGG1, MBD4, MUTYH, NEIL1, NEIL2, NEIL3, or NTHL1 is (substantially) incapable of excising cytosine of dC. In some embodiments, the TDG, SMUG1, OGG1, MBD4, MUTYH, NEIL1, NEIL2, NEIL3, or NTHL1 is (substantially) incapable of excising adenine of dA. In some embodiments, the TDG, SMUG1, OGG1, MBD4, MUTYH, NEIL1, NEIL2, NEIL3, or NTHL1 is (substantially) incapable of excising guanine of dG.

In some embodiments, the TDG, SMUG1, OGG1, MBD4, MUTYH, NEIL1, NEIL2, NEIL3, or NTHL1 is not wild type or naturally-occurring.

napDNAbd

As used herein, the term “napDNAbd” is used interchangeably with “nucleic acid programmable DNA binding protein (napDNAbp)”. In some embodiments, the napDNAbd is RNA programmable DNA binding protein. Various napDNAbd are known in the art, including, for example, those listed in WO2020/181195, which is incorporated herein by reference in its entirety. Representative napDNAbd include, for example, CRISPR-associated (Cas) proteins, IscB, IsrB, Argonaute, and TnpB.

In some embodiments, the napDNAbd substantially lacks dsDNA cleavage activity (endonuclease activity). In some embodiments, the napDNAbd substantially lacks dsDNA cleavage activity (endonuclease activity) and nickase activity. In some embodiments, the napDNAbd is nuclease-inactive, for example, a dead Cas. In some embodiments, the napDNAbd is endonuclease-inactive, for example, a dead Cas.

In some embodiments, the napDNAbd is a nickase. In some embodiments, the napDNAbd has nickase activity. In some embodiments, the napDNAbd has nickase activity to nick the target strand. In some embodiments, the napDNAbd nicks the target strand. In some embodiments, the method comprising nicking the target strand. In some embodiments, the nick on the target strand or nicking the target strand incorporates an indel (insertion and/or deletion) into the target strand.

In some embodiments, the napDNAbd is capable of inducing strand separation of the target dsDNA.

In some embodiments, the napDNAbd comprises a Cas domain. In some embodiments, the napDNAbd comprises a Cas nickase (nCas) or a dead (nuclease-inactive) Cas (dCas) of a Cas protein.

In some embodiments, the Cas protein is selected from a group consisting of a Cas9 protein (such as, SpCas9, SaCas9, GeoCas9, CjCas9, Cas9-KKH, circularly permuted Cas9, Argonaute (Ago), SmacCas9, Spy-macCas9, xCas9, SpCas9-NG, SpG Cas9); a Cas12 protein (such as, Cas12a (Cpf1), AsCas12a, LbCas12a, Cas12b, Cas12c, Cas12d, Cas12e, Cas12f (Cas14), Cas12g, Cas12h, Cas12i, xCas12i, Cas12Max, hfCas12Max, Cas12j, Cas12k, Cas12l, Cas12m, Cas12n, Cas12o, Cas12p, Cas12q, Cas12r, Cas12s, Cas12t, Cas12u, Cas12v, Cas12w, Cas12x, Cas12y, Cas12z); a Cas13 protein (such as, Cas13a, Cas13b, Cas13c, Cas13d, Cas13e, Cas13f, Cas13x, Cas13y); Csn2; and a mutant thereof. xCas12i, Cas12Max, and hfCas12Max are listed in PCT/CN2022/129376, which is incorporated herein by reference in its entirety.

In some embodiments, the Cas nickase is a Cas9 nickase (nCas9), such as SpCas9 nickase (SpCas9-D10A).

In some embodiments, the dead Cas is a dead Cas9 (dCas9), such as dead SpCas9 (SpCas9-D10A+H840A).

In some embodiments, the Cas nickase is a Cas12i nickase (nCas12i) or dead Cas12i (dCas12i), such as a deadCas12i of xCas12i polypeptide.

In some embodiments, the napDNAbd comprises an IscB nickase (nIscB) or a dead IscB (dIscB) of an IscB protein (e.g., OgeuIscB) or an IscB protein described in PCT/CN2023/129167, PCT/CN2023/142506, PCT/CN2024/071744, and PCT/CN2023/125069, which are incorporated herein by reference in their entireties.

In some embodiments, the napDNAbd comprise an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 2, 48, 50, 52, or 163.

In some embodiments, the napDNAbd comprises a TnpB nickase or a dead TnpB of a TnpB protein.

### BE Configuration

In some embodiments, the fusion protein comprises, from N-terminal to C-terminal, (1) the napDNAbp and the base excising domain; or (2) the base excising domain and the napDNAbp.

In some embodiments, the napDNAbd (e.g., Cas9) is a two-part napDNAbd, for example, a two-part split Cas9, comprising a N-terminal portion and a C-terminal portion, and wherein the fusion protein comprises, from N-terminal to C-terminal, (1) the N-terminal portion of the napDNAbd, the base excising domain, and the C-terminal portion of the napDNAbd; (2) the C-terminal portion of the napDNAbd, the base excising domain, and the N-terminal portion of the napDNAbd; or (3) the base excising domain, the C-terminal portion of the napDNAbd (e.g., amino acids at positions 1249-1368), and the N-temrinal portion (e.g., amino acids at positions 1-1248) of the napDNAbd.

In some embodiments, the napDNAbd is SpCas9 (e.g., a SpCas9 nickase) or a mutant thereof (e.g., a SpG Cas9 nickase). In some embodiments, the N-terminal portion of the napDNAbd is the amino acids of the napDNAbp at positions 1 or 2 to 1012, 1028, 1041, 1046, 1047, 1248, 1249, or 1300. In some embodiments, the C-terminal portion of the napDNAbd is the amino acids of the napDNAbp at positions 1013, 1029, 1042, 1047, 1048, 1249, 1063, 1064, 1230, 1249, or 1301 to 1368.

For example, in some embodiments, the fusion protein comprises the base excising domain embedded between positions 2-1248 of nCas9 (SEQ ID NO: 2) and positions 1249-1368 of nCas9 (SEQ ID NO: 2), wherein the first amino acid residue D of nCas9 (SEQ ID NO: 2) was designated as position 2; or embedded between positions 2-1047 of nCas9 (SEQ ID NO: 2) and positions 1064-1368 of nCas9 (SEQ ID NO: 2), wherein the first amino acid residue D of nCas9 (SEQ ID NO: 2) was designated as position 2.

A typical protein would usually have a N-terminal Met at its most N-terminal (position 1), since it requires to be translated from a polynucleotide containing a start codon ATG (encoding Met) at its most 5′ end. However, if such a protein is fused at the C-terminal of a second protein (e.g., an NLS, a napDNAbd), the start codon ATG may not be necessary for the protein since there would typically be a start codon upstream of the second protein for the translation of the fusion protein as a whole, and thus the N-terminal Met of the protein could be removed. Any protein described in the disclosure refers to both the protein per se and a N-terminal truncation thereof with its most N-terminal Met (if present) removed.

In some embodiments, the fusion protein comprises an NLS at the N-terminal and/or C-terminal of the napDNAbp. In some embodiments, the fusion protein comprises an NLS at the N-terminal and/or C-terminal of the base excising domain. In some embodiments, the NLS is or comprises a SV40 NLS, a bpSV40 NLS (e.g., SEQ ID NO: 10 or 11), or a NP NLS (Xenopus laevis Nucleoplasmin NLS, nucleoplasmin NLS). Additional NLS suitable for the disclosure or the way of linking an NLS to any of the components of the fusion protein of the disclosure include, for example, a linker of SGGS, or those listed in WO2020/181195, which is incorporated herein by reference in its entirety.

In some embodiments, the components (e.g., the napDNAbp and the base excising domain, the NLS and the napDNAbp, or the NLS and the base excising domain) of the fusion protein are fused to each other with or without a linker. Suitable linkers include, for example, SGGS, the linker of SEQ ID NO: 134, and those listed in WO2020/181195, which is incorporated herein by reference in its entirety.

In some embodiments, the fusion protein comprises an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 12, 14, 15, 17, 19, 21, 23, 25, 27, 29, 31, 33, 35, 37, 39, 55, 57, 59, 61, 63, 136, 138, 140, 142, 143, 145, 147, 149, 151, 153, 154, 156, 158, 160, 161, 162, and 164.

### Deaminase Domain

Notwithstanding the advantage of omissing a deamianse domain in the base editor of the disclosure, deamination may be allowed after the excision of the base of the target deoxyribonucleotide. In some aspects, the fusion protein of the disclosure may be used in combination with a deaminase domain for various purposes, e.g., improved outcome purity. By “purity” it means the percentage/proportion of an outcome among all possible outcomes. For example, purity of dT means the percentage/proportion of dT as an outcome among all possible outcomes including, for example, dA, dT, dG, and dC. As an example, the introduction of a deaminase domain may contribute to further conversion of an undesired deoxyribonucleotide as a byproduct (e.g., dC) to a desired deoxyribonucleotide (e.g., dT) by A-to-T base editing. So in summary, there could be a two-stage conversion if dG-to-dT is desired, first, the target dG is converted, in part, to dC by the base editing without deamination as described herein, and second, the dC is converted to dT by the C-to-T base editing with deamination, thereby achieving high purity dG-to-dT. Therefore, in some embodiments, the fusion protein further comprises a deaminase domain. The deaminase domain may be fused to a component of the fusion protein without or with a linker as described herein.

Various deaminases are known in the art, including, for example, those listed in WO2020/181195, which is incorporated herein by reference in its entirety. Representative adenine deaminases include, for example, TadA and homologs and variants thereof, and APOBEC and homologs and variants thereof.

In some embodiments, the deaminase domain is a deaminase domain (substantially) capable of deaminating adenine, guanine, hypoxanthine, cytidine, thymine, and/or uracil. In some embodiments, the deaminase domain is an adenine deaminase domain or a cytosine deaminase domain.

In some embodiments, the deaminase domain comprises a tRNA adenosine deaminase (TadA) or a functional variant or fragment thereof, e.g., TadA8e (SEQ ID NO: 3), TadA8.17, TadA8.20, TadA9, TadA8E^{V106W}, TadA8E^{V106W+D108Q} TadA-CDa, TadA-CDb, TadA-CDc, TadA-CDd, TadA-CDe, TadA-dual, T_{AD}AC-1.2, T_{AD}AC-1.14, T_{AD}AC-1.17, T_{AD}AC-1.19, T_{AD}AC-2.5, T_{AD}AC-2.6, T_{AD}AC-2.9, T_{AD}AC-2.19, T_{AD}AC-2.23, TadA8e-N46L, TadA8e-N46P.

In some embodiments, the deaminase domain comprises an apolipoprotein B mRNA-editing complex (APOBEC) family deaminase, an activation induced deaminase (AID), a cytidine deaminase 1 from Petromyzon marinus (pmCDA1), or a functional variant or fragment thereof, e.g., APOBEC1, APOBEC2, APOBEC3A, APOBEC3B, APOBEC3C, APOBEC3D, APOBEC3F, APOBEC3G, APOBEC3H.

### Protospacer Sequence

In some embodiments, the protospacer sequence comprises about, at least about, or at most about 14 contiguous nucleotides of the target dsDNA, e.g., about, at least about, or at most about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the nontarget strand of the target dsDNA, or in a numerical range between any two of the preceding values, e.g., from about 16 to about 50, or from about 17 to about 22 contiguous nucleotides of the target dsDNA. In some embodiments, the protospacer sequence comprises about 20, 30, or 50 contiguous nucleotides on the nontarget strand of the target dsDNA.

In some embodiments, the protospacer sequence is a stretch of about, at least about, or at most about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the nontarget strand of the target dsDNA, or a stretch of contiguous nucleotides on the nontarget strand of the target dsDNA in a numerical range between any two of the preceding values, e.g., a stretch of from about 16 to about 50 contiguous nucleotides. In some embodiments, the protospacer sequence is a stretch of about 20, 30, or 50 contiguous nucleotides on the nontarget strand of the target dsDNA.

In some embodiments, the protospacer sequence is immediately 5′ or 3′ to a protospacer adjacent motif (PAM) comprises sequence 5′-NN-3′, 5′-NNN-3′, 5′-NNNN-3′, 5′-NNNNN-3′, or 5′-NNNNNN-3′, wherein N is A, T, G, or C. For example, in some embodiments, the protospacer sequence is immediately 5′ to a protospacer adjacent motif (PAM) comprises sequence 5′-NGG-3′ or 5′-NTN-3′, wherein N is A, T, G, or C. In some embodiments, the protospacer sequence is immediately 3′ to a protospacer adjacent motif (PAM) comprises sequence 5′-TTN-3′, wherein N is A, T, G, or C.

### Guide Sequence

In some embodiments, the guide sequence is in a length of about, at least about, or at most about 14 nucleotides, e.g., about, at least about, or at most about 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides, or in a length of nucleotides in a numerical range between any two of the preceding values, e.g., in a length of from about 16 to about 50 nucleotides. In some embodiments, the guide sequence is in a length of about 20, 30, or 50 nucleotides.

In some embodiments, (1) the guide sequence is at least about 50%, 55%, 60%, 65%, 70%, 75%, 80%, 85%, 90%, 95%, or 100% (fully), optionally about 100% (fully), reversely complementary to the target sequence; (2) the guide sequence contains no more than 5, 4, 3, 2, or 1 mismatch or contains no mismatch with the target sequence; or (3) the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence when the PAM is immediately 5′ to the protospacer sequence or at the 3′ end of the guide sequence when the PAM is immediately 3′ to the protospacer sequence. In some embodiments, the guide sequence is about 100% (fully), reversely complementary to the target sequence.

In some embodiments, the guide sequence contains 1 mismatch with the target sequence. In some embodiments, the guide sequence is about 98% reversely complementary to the target sequence. In some embodiments, the 1 mismatch in the guide sequence is at a position corresponding the nucleotide of the target sequence that is intended to be substituted.

In some embodiments, the guide sequence comprises (1) a sequence of SEQ ID NO: 75-103 and 165-3030 (excluding the PAM if present) or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: SEQ ID NO: 75-103 and 165-3030 (excluding the PAM if present) or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: SEQ ID NO: 75-103 and 165-3030 (excluding the PAM if present).

In some embodiments, the guide sequence comprises a sequence of any one of SEQ ID NOs: SEQ ID NO: 75-103 and 165-3030 (excluding the PAM if present).

In some aspects, the disclosure provides a guide nucleic acid comprising a guide sequence as described herein and a scaffold sequence capable of forming a complex with a napDNAbd. The scaffold sequence and the napDNAbd may be as described herein.

### Scaffold Sequence

For the purpose of the disclosure, the scaffold sequence is compatible with the napDNAbd of the disclosure and is capable of complexing with the napDNAbd. The scaffold sequence may be a naturally occurring scaffold sequence identified along with the napDNAbd, or a variant thereof maintaining the ability to complex with the napDNAbd. Generally, the ability to complex with the napDNAbd is maintained as long as the secondary structure of the variant is substantially identical to the secondary structure of the naturally occurring scaffold sequence. A nucleotide deletion, insertion, or substitution in the primary sequence of the scaffold sequence may not necessarily change the secondary structure of the scaffold sequence (e.g., the relative locations and/or sizes of the stems, bulges, and loops of the scaffold sequence do not significantly deviate from that of the original stems, bulges, and loops). For example, the nucleotide deletion, insertion, or substitution may be in a bulge or loop region of the scaffold sequence so that the overall symmetry of the bulge and hence the secondary structure remains largely the same. The nucleotide deletion, insertion, or substitution may also be in the stems of the scaffold sequence so that the lengths of the stems do not significantly deviate from that of the original stems (e.g., adding or deleting one base pair in each of two stems correspond to 4 total base changes). In some embodiments, the scaffold sequence is 5′ or 3′ to the guide sequence.

In some embodiments, the scaffold sequence has substantially the same secondary structure as the secondary structure of the sequence of SEQ ID NO: 40, 73, or 74. In some embodiments, the scaffold sequence comprises (1) a sequence of SEQ ID NO: 40, 73, or 74 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 40, 73, or 74 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 40, 73, or 74. In some embodiments, the scaffold sequence comprises the sequence of SEQ ID NO: 40, 73, or 74.

### Translesion Synthesis (TLS) Polymerase

In some aspects, the fusion protein of the disclosure may be used in combination with a translesion synthesis (TLS) polymerase for improved outcome purity. By “purity” it means the percentage/proportion of an outcome among all possible outcomes. For example, purity of dT means the percentage/proportion of dT as an outcome among all possible outcomes including, for example, dA, dT, dG, and dC. TLS polymerases may have their own inclination of incorporating various deoxyribonucleotide opposite an abasic site during polymerization, as listed in Table 5. By taking advantage of such inclination, the base editing outcome may be intentionally controlled to improve outcome purity. For example, human Polη (SEQ ID NO: 118) is a TLS polymerase preferentially incorporating dA opposite an abasic site. With combination use of human Pohl, the base editing outcome may be adjusted toward dT, thereby increasing purity of dT product.

TABLE 5
DNA polymerases for incorporating
perfect base opposite abasic sites.

| DNA polymerases | Perfect base opposite abasic sites |
|---|---|
| Polα (alpha) | dA |
| Polδ (delta)/PCNA | dA |
| Polγ (gamma) | dA |
| Polη (eta) | dT and dA |
| Polι (iota) | dT, dG, and dA |
| Polκ (kappa) | dC and dA |
| Polθ (theta) | dA |
| REV1 | dC |

In some embodiments, the fusion protein or system of the disclosure further comprises a translesion synthesis (TLS) polymerase or a recruiting domain or component capable of recruiting a TLS polymerase. In some embodiments, the TLS polymerase or the recruiting domain or component is fused to a component of the fusion protein without or with a linker as described herein.

Non-limiting examples of the TLS polymerase is selected from the group consisting of Polα (alpha), Polβ (beta), Polδ (delta) (PCNA), Polγ (gamma), Polη (eta), Polι (iota), Polκ (kappa), Polλ (lamda), Polμ (mu), Polν (nu), Polθ (theta), and REV1.

In some embodiments, the TLS polymerase comprises an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 118. In some embodiments, the TLS polymerase comprises the amino acid sequence of SEQ ID NO: 118 (Pohl).

In some embodiments, the fusion protein or system further comprising the translesion synthesis (TLS) polymerase or a recruiting domain capable of recruiting a TLS polymerase leads to conversion of the first deoxyribonucleotide to dG, dC, dT, or dA.

### MPG

In yet another aspect, the disclosure provides an MPG described herein, or of the disclosure.

In some embodiments, the MPG comprises a motif GxxYxxxxYGxxxxxN, wherein x represents any amino acid. In some embodiments, the MPG is obtained from a species selected from Table A. In some embodiments, the MPG comprises an amino acid mutation relative to (compared to; with reference to) a wild type or reference MPG. In some embodiments, the wild type or reference MPG is human MPG (SEQ ID NO: 9) or an MPG obtained from a species selected from Table A or any MPG as set forth in Table D or a homology or mutant (e.g., comprising an amino acid sequence of SEQ ID NO: 5, 6, or 7) thereof or an N-terminal truncation thereof lacking the most N-terminal Methionine (Met) (coded by start codon ATG) (e.g., SEQ ID NO: 1). In some embodiments, the wild type or reference MPG comprises the amino acid sequence of SEQ ID NO: 7.

In some embodiments, the amino acid mutation comprises an amino acid substitution at a position that is corresponding to a position or that is a position selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, and/or 298 of the wild type or reference MPG, wherein the position is numbered according to SEQ ID NO: 1.

In some embodiments, the amino acid mutation confers an ability to excise a base on the MPG. In some embodiments, the base is guanine.

In some embodiments, the amino acid mutation leads to increased base excising ability as compared to an otherwise identical control MPG without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation leads to increased guide sequence-specific base editing efficiency of the fusion protein as compared to an otherwise identical control fusion protein without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation comprises an amino acid substitution at a position that is corresponding to a position or that is a position selected from the group consisting of G163, N169, D175, C178, S198, K202, G203, S206, K210, and/or Q294 of the wild type or reference MPG, wherein the position is numbered according to SEQ ID NO: 1.

In some embodiments, the amino acid mutation comprises an amino acid substitution at a position that is corresponding to a position or that is a position selected from the group consisting of N169, D175, C178, and/or Q294 of the wild type or reference MPG, wherein the position is numbered according to SEQ ID NO:1. In some embodiments, the wild type or reference MPG comprises the amino acid sequence of SEQ ID NO: 7.

In some embodiments, the amino acid substitution is a conservative amino acid substitution or a non-conservative amino acid substitution. In some embodiments, the amino acid substitution is an amino acid substitution with an amino acid residue that is different from the amino acid residue at the position of the wild type or reference MPG. In some embodiments, the amino acid substitution is an amino acid substitution with (1) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F); (2) a polar amino acid residue (such as, Serine (Ser/S), Threonine (Thr/T), Tyrosine (Tyr/Y), Asparagine (Asn/N), Glutamine (Gln/Q)); (3) a positively charged amino acid residue (such as, Lysine (Lys/K), Arginine (Arg/R), Histidine (His/H)); or (4) a negatively charged amino acid residue (such as, Aspartic Acid (Asp/D), Glutamic Acid (Glue/E)). In some embodiments, the amino acid substitution is a substitution with R, A, N, or G.

In some embodiments, the amino acid mutation comprises an amino acid substitution that is corresponding to a substitution or that is a substitution selected from the group consisting of G163R, N169G, D175R, C178N, S198A, K202A, G203A, S206A, K210A, Q294R, and a combination of any two or more substitutions thereof, wherein the position is numbered according to SEQ ID NO: 1.

In some embodiments, the MPG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the wild type or reference MPG.

In some embodiments, the amino acid mutation comprises an amino acid substitution that is corresponding to a substitution or that is a substitution selected from the group consisting of N169G, D175R, C178N, Q294R, and a combination of any two or more substitutions thereof, wherein the position is numbered according to SEQ ID NO: 1. In some embodiments, the wild type or reference MPG comprises the amino acid sequence of SEQ ID NO: 7.

In some embodiments, the amino acid mutation comprises a combination substitution that is corresponding to a combination substitution of N169G, D175R, C178N, and Q294R, wherein the position is numbered according to SEQ ID NO: 1. In some embodiments, the wild type or reference MPG comprises the amino acid sequence of SEQ ID NO: 7.

In some embodiments, the amino acid mutation comprises a combination substitution that is corresponding to a combination substitution of G163R, N169G, D175R, C178N, S198A, K202A, G203A, S206A, K210A, Q294R, wherein the position is numbered according to SEQ ID NO: 1. In some embodiments, the wild type or reference MPG comprises the amino acid sequence of SEQ ID NO: 1 or 9.

In some embodiments, the MPG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 8, 16, 18, 20, 22, 24, 26, 28, 30, 32, 34, 36, or 38.

In some embodiments, the MPG is (substantially) capable of excising guanine of dG. In some embodiments, the MPG is (substantially) incapable of excising thymine of dT. In some embodiments, the MPG is (substantially) incapable of excising cytosine of dC. In some embodiments, the MPG is (substantially) incapable of excising adenine of dA.

In some aspects, the disclosure provides a fusion protein comprising the MPG described herein, or of the disclosure, and a functional domain, such as, a napDNAbd.

In some aspects, the disclosure provides use of the MPG described herein, or of the disclosure, for base editing as described herein.

In some embodiments, the MPG is not wild type or naturally-occurring.

### UNG

In yet another aspect, the disclosure provides an UNG described herein, or of the disclosure.

In some embodiments, the UNG comprises a motif GQDPYH. In some embodiments, the UNG is obtained from a species selected from Table C. In some embodiments, the UNG comprises an amino acid mutation relative to (compared to; with reference to) a wild type or reference UNG. In some embodiments, the wild type or reference UNG is human UNG1 (SEQ ID NO: 54) or human UNG2 (SEQ ID NO: 133) or an UNG obtained from a species selected from Table C or any UNG as set forth in Table D or a homology or mutant thereof or an N-terminal truncation thereof lacking the most N-terminal Methionine (Met) (coded by start codon ATG). In some embodiments, the wild type or reference UNG comprises the amino acid sequence of SEQ ID NO: 56, 58, 135, or 137.

In some embodiments, the amino acid mutation comprises an amino acid substitution at a position that is corresponding to a position or that is a position selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, and/or 313 of the wild type or reference UNG, wherein the position is numbered according to SEQ ID NO: 133.

Alternative splicing as well as transcription from two distinct start sites leads to two different human UNG isoforms, mitochondrial UNG1 (304 amino acids, aa) (SEQ ID NO: 54) and nuclear UNG2 (313 aa) (SEQ ID NO: 133), each possessing an unique N-terminus that mediates translocation to mitochondria or nucleus¹⁶ (FIG. 25). The sequence alignment of human UNG1 and human UNG2 shows that the amino acid residues at positions 1-35 of UNG1 are different from the amino acid residues at positions 1-44 of UNG2, and the remaining parts of UNG1 and UNG2 are identical. The corresponding relationship between the positions of UNG1 (SEQ ID NO: 54) and UNG2 (SEQ ID NO: 133) can be determined by sequence alignment. For example, the residues Y156 and N213 of UNG2 are corresponding to the residues Y147 and N204 of UNG1, respectively.

In some embodiments, the amino acid mutation confers an ability to excise a base on the UNG. In some embodiments, the base is thymine. In some embodiments, the base is cytosine.

In some embodiments, the amino acid mutation leads to increased base excising ability as compared to an otherwise identical control UNG without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation leads to increased guide sequence-specific base editing efficiency of the fusion protein as compared to an otherwise identical control fusion protein without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation comprises an amino acid substitution at a position that is corresponding to a position or that is a position selected from the group consisting of Y156, K184, N213, A214, Q259, and/or Y284 of the wild type or reference UNG, wherein the position is numbered according to SEQ ID NO: 133.

In some embodiments, the amino acid mutation comprises an amino acid substitution at a position that is corresponding to a position or that is a position selected from the group consisting of K184, A214, Q259, and/or Y284 of the wild type or reference UNG, wherein the position is numbered according to SEQ ID NO:133. In some embodiments, the wild type or reference UNG comprises the amino acid sequence of SEQ ID NO: 135 or 137.

In some embodiments, the amino acid substitution is a conservative amino acid substitution or a non-conservative amino acid substitution. In some embodiments, the amino acid substitution is an amino acid substitution with an amino acid residue that is different from the amino acid residue at the position of the wild type or reference UNG. In some embodiments, the amino acid substitution is an amino acid substitution with (1) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/1), Methionine (Met/M), Tryptophan (Trp/W), Phenylalanine (Phe/F); (2) a polar amino acid residue (such as, Serine (Ser/S), Threonine (Thr/T), Tyrosine (Tyr/Y), Asparagine (Asn/N), Glutamine (Gln/Q)); (3) a positively charged amino acid residue (such as, Lysine (Lys/K), Arginine (Arg/R), Histidine (His/H)); or (4) a negatively charged amino acid residue (such as, Aspartic Acid (Asp/D), Glutamic Acid (Glue/E)). In some embodiments, the amino acid substitution is a substitution with A, D, V, or T.

In some embodiments, the amino acid mutation comprises an amino acid substitution that is corresponding to a substitution or that is a substitution selected from the group consisting of Y156A, K184A, N213D, A214V, A214T, Q259A, Y284D, and a combination of any two or more substitutions thereof, wherein the position is numbered according to SEQ ID NO: 133.

In some embodiments, the amino acid mutation comprises an amino acid substitution that is corresponding to a substitution or that is a substitution selected from the group consisting of A214T, Q259A, Y284D, and a combination of any two or more substitutions thereof, wherein the position is numbered according to SEQ ID NO: 133. In some embodiments, the wild type or reference UNG comprises the amino acid sequence of SEQ ID NO: 135.

In some embodiments, the amino acid mutation comprises an amino acid substitution that is corresponding to a substitution or that is a substitution selected from the group consisting of K184A, A214V, and a combination of the two substitutions, wherein the position is numbered according to SEQ ID NO: 133. In some embodiments, the wild type or reference UNG comprises the amino acid sequence of SEQ ID NO: 137.

In some embodiments, the amino acid mutation comprises a deletion of amino acids at positions that are corresponding to positions or that are positions 1-65, 1-66, 1-67, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of the wild type or reference UNG, wherein the position is numbered according to SEQ ID NO: 133.

In some embodiments, the amino acid mutation comprises an amino acid substitution that is corresponding to a substitution or that is a substitution selected from the group consisting of A214T, Q259A, Y284D, and a combination of any two or more substitutions thereof, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 135.

In some embodiments, the amino acid mutation comprises an amino acid substitution that is corresponding to a substitution or that is a substitution selected from the group consisting of K184A, A214V, and a combination of the two substitutions, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 137.

In some embodiments, the UNG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the wild type or reference UNG.

In some embodiments, the amino acid mutation comprises a combination substitution that is corresponding to a combination substitution of A214T, Q259A, and Y284D, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 135.

In some embodiments, the amino acid mutation comprises a combination substitution that is corresponding to a combination substitution of K184A and A214V, and comprises a deletion of amino acids at positions that are corresponding to positions or that are positions 1-88 of the wild type or reference UNG, wherein the position is numbered according to SEQ ID NO: 133. In some embodiments, the wild type or reference UNG comprises the amino acid sequence of SEQ ID NO: 137.

In some embodiments, the UNG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 56, 58, 60, 62, 135, 137, 139, 141, 144, 146, 148, 150, 152, 155, 157, and 159 or an N-terminal truncation thereof lacking the most N-terminal Methionine (M) (coded by start codon ATG).

In some embodiments, the UNG is (substantially) capable of excising thymine of dT. In some embodiments, the UNG is (substantially) capable of excising cytosine of dC. In some embodiments, the UNG is (substantially) incapable of excising thymine of dT. In some embodiments, the UNG is (substantially) incapable of excising cytosine of dC. In some embodiments, the UNG is (substantially) incapable of excising adenine of dA. In some embodiments, the UNG is (substantially) incapable of excising guanine of dG.

In some aspects, the disclosure provides a fusion protein comprising the UNG described herein, or of the disclosure, and a functional domain, such as, a napDNAbd.

In some aspects, the disclosure provides use of the UNG described herein, or of the disclosure, for base editing as described herein.

In some embodiments, the UNG is not wild type or naturally-occurring.

### Additional BEP

In yet another aspect, the disclosure provides a TDG, SMUG1, OGG1, MBD4, MUTYH, NEIL1, NEIL2, NEIL3, or NTHL1 described herein, or of the disclosure.

In some embodiments, the TDG, SMUG1, OGG1, MBD4, MUTYH, NEIL1, NEIL2, NEIL3, or NTHL1 comprises an amino acid mutation relative to (compared to; with reference to) a wild type or reference TDG, SMUG1, OGG1, MBD4, MUTYH, NEIL1, NEIL2, NEIL3, or NTHL1. In some embodiments, the wild type or reference TDG, SMUG1, OGG1, MBD4, MUTYH, NEIL1, NEIL2, NEIL3, or NTHL1 is human TDG, SMUG1, OGG1, MBD4, MUTYH, NEIL1, NEIL2, NEIL3, or NTHL1 (SEQ ID NO: 64, 65, 66, 67, 68, 69, 70, 71, or 72, respectively) or a homology or mutant thereof or an N-terminal truncation thereof lacking the most N-terminal Methionine (Met) (coded by start codon ATG). In some embodiments, the wild type or reference TDG, SMUG1, OGG1, MBD4, MUTYH, NEIL1, NEIL2, NEIL3, or NTHL1 comprises an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 64-72, respectively.

In some embodiments, the amino acid mutation comprises an amino acid substitution at a position that is corresponding to a position or that is a position selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, and/or 604 of the wild type or reference TDG, SMUG1, OGG1, MBD4, MUTYH, NEIL1, NEIL2, NEIL3, or NTHL1, wherein the position is numbered according to any one of SEQ ID NOs: 64-72.

In some embodiments, the amino acid mutation confers an ability to excise a base on the TDG, SMUG1, OGG1, MBD4, MUTYH, NEIL1, NEIL2, NEIL3, or NTHL1. In some embodiments, the base is guanine, thymine, cytosine, adenine, uracil, or hypoxanthine.

In some embodiments, the amino acid mutation leads to increased base excising ability as compared to an otherwise identical control TDG, SMUG1, OGG1, MBD4, MUTYH, NEIL1, NEIL2, NEIL3, or NTHL1 without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid mutation leads to increased guide sequence-specific base editing efficiency of the fusion protein as compared to an otherwise identical control fusion protein without said amino acid mutation, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, 400%, 500%, 600%, 700%, 800%, 900%, 1000%, 1100%, 1200%, 1300%, 1400%, 1500%, 1600%, 1700%, 1800%, 1900%, 2000%, or more.

In some embodiments, the amino acid substitution is a conservative amino acid substitution or a non-conservative amino acid substitution. In some embodiments, the amino acid substitution is an amino acid substitution with an amino acid residue that is different from the amino acid residue at the position of the wild type or reference TDG, SMUG1, OGG1, MBD4, MUTYH, NEIL1, NEIL2, NEIL3, or NTHL1. In some embodiments, the amino acid substitution is an amino acid substitution with (1) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F); (2) a polar amino acid residue (such as, Serine (Ser/S), Threonine (Thr/T), Tyrosine (Tyr/Y), Asparagine (Asn/N), Glutamine (Gln/Q)); (3) a positively charged amino acid residue (such as, Lysine (Lys/K), Arginine (Arg/R), Histidine (His/H)); or (4) a negatively charged amino acid residue (such as, Aspartic Acid (Asp/D), Glutamic Acid (Glue/E)).

In some embodiments, the TDG, SMUG1, OGG1, MBD4, MUTYH, NEIL1, NEIL2, NEIL3, or NTHL1 comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the wild type or reference TDG, SMUG1, OGG1, MBD4, MUTYH, NEIL1, NEIL2, NEIL3, or NTHL1, respectively.

In some embodiments, the TDG, SMUG1, OGG1, MBD4, MUTYH, NEIL1, NEIL2, NEIL3, or NTHL1 is (substantially) capable of excising guanine of dG. In some embodiments, the TDG, SMUG1, OGG1, MBD4, MUTYH, NEIL1, NEIL2, NEIL3, or NTHL1 is (substantially) capable of excising thymine of dT. In some embodiments, the TDG, SMUG1, OGG1, MBD4, MUTYH, NEIL1, NEIL2, NEIL3, or NTHL1 is (substantially) capable of excising cytosine of dC. In some embodiments, the TDG, SMUG1, OGG1, MBD4, MUTYH, NEIL1, NEIL2, NEIL3, or NTHL1 is (substantially) capable of excising adenine of dA.

In some embodiments, the TDG, SMUG1, OGG1, MBD4, MUTYH, NEIL1, NEIL2, NEIL3, or NTHL1 is (substantially) incapable of excising thymine of dT. In some embodiments, the TDG, SMUG1, OGG1, MBD4, MUTYH, NEIL1, NEIL2, NEIL3, or NTHL1 is (substantially) incapable of excising cytosine of dC. In some embodiments, the TDG, SMUG1, OGG1, MBD4, MUTYH, NEIL1, NEIL2, NEIL3, or NTHL1 is (substantially) incapable of excising adenine of dA. In some embodiments, the TDG, SMUG1, OGG1, MBD4, MUTYH, NEIL1, NEIL2, NEIL3, or NTHL1 is (substantially) incapable of excising guanine of dG.

In some aspects, the disclosure provides a fusion protein comprising the TDG, SMUG1, OGG1, MBD4, MUTYH, NEIL1, NEIL2, NEIL3, or NTHL1 described herein, or of the disclosure, and a functional domain, such as, a napDNAbd.

In some aspects, the disclosure provides use of the TDG, SMUG1, OGG1, MBD4, MUTYH, NEIL1, NEIL2, NEIL3, or NTHL1 described herein, or of the disclosure, for base editing as described herein.

In some embodiments, the TDG, SMUG1, OGG1, MBD4, MUTYH, NEIL1, NEIL2, NEIL3, or NTHL1 is not wild type or naturally-occurring.

### Regulation of Guide Nucleic Acid

Also provided in the disclosure is a polynucleotide comprising or encoding the guide nucleic acid.

In some embodiments, the polynucleotide comprising or encoding the guide nucleic acid is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the guide nucleic acid is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

### Regulation of Fusion Protein

In yet another aspect, the disclosure provides a polynucleotide encoding the fusion protein of the disclosure and optionally the guide nucleic acid of the disclosure.

In some embodiments, the polynucleotide encoding the fusion protein is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the polynucleotide encoding the napDNAbd is a mRNA.

In some embodiments, the polynucleotide encoding the napDNAbd comprises a sequence encoding the napDNAbd and a promoter operably linked to the sequence encoding the napDNAbd.

In some embodiments, the polynucleotide encoding the napDNAbd is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a human synapsin (hSyn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a OTOF promoter, a GRK1 promoter, a CRX promoter, a NRL promoter, a MECP2 promoter, a mMECP2 promoter, a hMECP2 promoter, an APP promoter, and a RCVRN promoter.

### Delivery

Various ways of delivery can be applied to the fusion protein of the disclosure or the system of the disclosure as needed in practices.

In yet another aspect, the disclosure provides a delivery system comprising (1) the fusion protein of the disclosure, the polynucleotide of the disclosure, or the system of the disclosure; and (2) a delivery vehicle.

In yet another aspect, the disclosure provides a vector comprising the polynucleotide of the disclosure. In some embodiments, the vector encodes a guide nucleic acid of the disclosure. In some embodiments, the vector is a plasmid vector, a recombinant AAV (rAAV) vector (vector genome), or a recombinant lentivirus vector.

In yet another aspect, the disclosure provides a recombinant AAV (rAAV) particle comprising the rAAV vector genome of the disclosure. A simple introduction of AAV for delivery may refer to “Adeno-associated Virus (AAV) Guide” (addgene. org/guides/aav/).

Adeno-associated virus (AAV), when engineered to delivery, e.g., a protein-encoding sequence of interest, may be termed as a (r) AAV vector, a (r) AAV vector particle, or a (r) AAV particle, where “r” stands for “recombinant”. And the genome packaged in AAV vectors for delivery may be termed as a (r) AAV vector genome, vector genome, or vg for short, while viral genome may refer to the original viral genome of natural AAVs.

The serotypes of the capsids of rAAV particles can be matched to the types of target cells. For example, Table 2 of WO2018002719A1 lists exemplary cell types that can be transduced by the indicated AAV serotypes (incorporated herein by reference).

In some embodiments, the rAAV particle comprising a capsid with a serotype suitable for delivery into a desired target cell. In some embodiments, the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV. PHP. eB, a member of the Clade to which any of the AAV1-AAV13 belong, or a functional variant (e.g., a functional truncation) thereof, encapsidating the rAAV vector genome. In some embodiments, the serotype of the capsid is wild type serotype or a functional variant thereof.

General principles of rAAV particle production are known in the art. In some embodiments, rAAV particles may be produced using the triple transfection method (described in detail in U.S. Pat. No. 6,001,650).

The vector titers are usually expressed as vector genomes per ml (vg/ml). In some embodiments, the vector titer is above 1×10⁹, above 5×10¹⁰, above 1×10¹¹, above 5×10¹¹, above 1×10², above 5×10¹², or above 1×10¹³ vg/ml.

Instead of packaging a single strand (ss) DNA sequence as a vector genome of a rAAV particle, systems and methods of packaging an RNA sequence as a vector genome into a rAAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of rAAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be a functional RNA sequence for use, or an RNA sequence can be produced from the RNA coding sequence, e.g., by RNA processing, or a protein can be translated from the RNA coding sequence.

For example, a fusion protein coding sequence encoding a fusion protein covers either a fusion protein DNA coding sequence from which a fusion protein is expressed (indirectly via transcription and translation) or a fusion protein RNA coding sequence from which a fusion protein is translated (directly).

For example, a gRNA coding sequence encoding a gRNA covers either a gRNA DNA coding sequence from which a gRNA is transcribed or a gRNA RNA coding sequence (1) which per se is the functional gRNA for use, or (2) from which a gRNA is produced, e.g., by RNA processing.

In some embodiments for rAAV RNA vector genomes, 5′-ITR and/or 3′-ITR as DNA packaging signals may be unnecessary and can be omitted at least partly, while RNA packaging signals can be introduced.

In some embodiments for rAAV RNA vector genomes, a promoter to drive transcription of DNA sequences may be unnecessary and can be omitted at least partly.

In some embodiments for rAAV RNA vector genomes, a sequence encoding a polyA signal may be unnecessary and can be omitted at least partly, while a polyA tail can be introduced.

Similarly, other DNA elements of rAAV DNA vector genomes can be either omitted or replaced with corresponding RNA elements and/or additional RNA elements can be introduced, in order to adapt to the strategy of delivering an RNA vector genome by rAAV particles.

In yet another aspect, the disclosure provides a complex comprising the fusion protein or the polynucleotide (e.g., a mRNA) encoding the fusion protein of the disclosure and a guide nucleic acid (e.g., a gRNA) of the disclosure.

In yet another aspect, the disclosure provides a ribonucleoprotein (RNP) comprising the fusion protein of the disclosure and a guide nucleic acid of the disclosure.

In yet another aspect, the disclosure provides a lipid nanoparticle (LNP) comprising an RNA (e.g., mRNA) encoding the fusion protein of the disclosure and a guide nucleic acid of the disclosure.

### Pharmaceutical Composition

In yet another aspect, the disclosure provides a pharmaceutical composition comprising (1) the system of the disclosure, the vector of the disclosure, the ribonucleoprotein of the disclosure, the lipid nanoparticle of the disclosure, or the cell of the disclosure; and (2) a pharmaceutically acceptable excipient.

In some embodiments, the pharmaceutical composition comprises the rAAV particle in a concentration selected from the group consisting of about 1×10¹⁰ vg/mL, 2×10¹⁰ vg/mL, 3×10¹⁰ vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical range between any of two preceding values, e.g., in a concentration of from about 9×10¹⁰ vg/mL to about 8×10¹¹ vg/mL. In some embodiments, the pharmaceutical composition is an injection formulation.

In some embodiments, the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

### Cells

The methods of the disclosure can be used to introduce the systems of the disclosure into a cell and cause the cell to alter the production of one or more cellular produces, such as antibody, starch, ethanol, or any other desired products. Such cells and progenies thereof are within the scope of the disclosure.

In yet another aspect, the disclosure provides a cell or a progeny thereof comprising the system of the disclosure. In some embodiments, the cell is a eukaryote. In some embodiments, the cell is a human cell.

In yet another aspect, the disclosure provides a cell or a progeny thereof modified by the system of the disclosure or the method of the disclosure. In some embodiments, the cell is a eukaryote. In some embodiments, the cell is a human cell. In some embodiments, the cell is modified in vitro, in vivo, or ex vivo.

In some embodiments, the cell is a stem cell. In some embodiments, the cell is not a human embryonic stem cell. In some embodiments, the cell is not a human germ cell.

In some embodiments, the cell is a prokaryotic cell.

In some embodiments, the cell is a eukaryotic cell (e.g., an animal cell, a vertebrate cell, a mammalian cell, a non-human mammalian cell, a non-human primate cell, a rodent (e.g., mouse or rat) cell, a human cell, a plant cell, or a yeast cell) or a prokaryotic cell (e.g., a bacteria cell).

In some embodiments, the cell is from a plant or an animal.

In some embodiments, the plant is a dicotyledon. In some embodiments, the dicotyledon is selected from the group consisting of soybean, cabbage (e.g., Chinese cabbage), rapeseed, brassica, watermelon, melon, potato, tomato, tobacco, eggplant, pepper, cucumber, cotton, alfalfa, eggplant, grape.

In some embodiments, the plant is a monocotyledon. In some embodiments, the monocotyledon is selected from the group consisting of rice, corn, wheat, barley, oat, sorghum, millet, grasses, Poaceae, Zizania, Avena, Coix, Hordeum, Oryza, Panicum (e.g., Panicum miliaceum), Secale, Setaria (e.g., Setaria italica), Sorghum, Triticum, Zea, Cymbopogon, Saccharum (e.g., Saccharum officinarum), Phyllostachys, Dendrocalamus, Bambusa, Yushania.

In some embodiments, the animal is selected from the group consisting of pig, ox, sheep, goat, mouse, rat, alpaca, monkey, rabbit, chicken, duck, goose, fish (e.g., zebra fish).

In some embodiments, the cell is a eukaryotic cell, such as a mammalian cell, including a human cell (a primary human cell or an established human cell line). In some embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc.). In some embodiments, the cell is from fish (such as salmon), bird (such as poultry bird, including chick, duck, goose), reptile, shellfish (e.g., oyster, claim, lobster, shrimp), insect, worm, yeast, etc. In some embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum;

cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the cell is not within the body of an organism, such as, human or animal. In some embodiments, the cell is not a human embryonic stem cell. In some embodiments, the cell is not a human germ cell.

### Method

In yet another aspect, the disclosure provides a method for modifying a target dsDNA, comprising contacting the target DNA with the system of the disclosure, wherein the guide sequence is capable of hybridizing to a target sequence of the target dsDNA, wherein the target dsDNA is modified by the complex.

In yet another aspect, the disclosure provides a method for diagnosing, preventing, and/or treating a disease in a subject in need thereof, comprising administering to the subject (e.g., a therapeutically effective amount/dose of) the system of the disclosure, the vector of the disclosure, the ribonucleoprotein of the disclosure, the lipid nanoparticle of the disclosure, the cell of the disclosure, or the pharmaceutical composition of the disclosure, wherein the disease is associated with a target dsDNA, wherein the guide sequence is capable of hybridizing to a target sequence of the target dsDNA, wherein the target dsDNA is modified by the complex, and wherein the modification of the target dsDNA diagnose, prevents, and/or treats the disease.

In some embodiments, the disease is selected from the group consisting of Angelman syndrome (AS), Alzheimer's disease (AD), transthyretin amyloidosis (ATTR), transthyretin amyloid cardiomyopathy (ATTR-CM), cystic fibrosis (CF), hereditary angioedema, diabetes, progressive pseudohypertrophic muscular dystrophy, Duchenne muscular dystrophy (DMD), Becker muscular dystrophy (BMD), spinal muscular atrophy (SMA), alpha-1-antitrypsin deficiency, Pompe disease, myotonic dystrophy, Huntington's disease (HTT), fragile X syndrome, Friedreich ataxia, amyotrophic lateral sclerosis (ALS), frontotemporal dementia, hereditary chronic kidney disease, hyperlipidemia, Leber congenital amaurosis (LCA), sickle cell disease, thalassemia (e.g., β-thalassemia), Parkinson's disease (PD), myelodysplastic syndrome (MDS), retinitis pigmentosa (RP), age-related macular degeneration (AMD), Hepatitis B, nonalcoholic fatty liver disease (NAFLD), Acquired Immune Deficiency Syndrome, corneal dystrophy (CD), hypercholesterolemia, familial hypercholesterolemia (FH), heart disease (e.g., hypertrophic cardiomyopathy (HCM)), and cancer.

In some embodiments, the target dsDNA encodes a mRNA, a tRNA, a ribosomal RNA (rRNA), a microRNA (miRNA), a non-coding RNA, a long non-coding (lnc) RNA, a nuclear RNA, an interfering RNA (iRNA), a small interfering RNA (siRNA), a ribozyme, a riboswitch, a satellite RNA, a microswitch, a microzyme, or a viral RNA.

In some embodiments, the target dsDNA is a eukaryotic DNA.

In some embodiments, the eukaryotic DNA is a mammal DNA, such as a non-human mammalian DNA, a non-human primate DNA, a human DNA, a plant DNA, an insect DNA, a bird DNA, a reptile DNA, a rodent (e.g., mouse, rat) DNA, a fish DNA, a nematode DNA, or a yeast DNA.

In some embodiments, the target dsDNA is in a eukaryotic cell, for example, a human cell, a non-human primate cell, or a mouse cell.

In some embodiments, the administrating comprises local administration or systemic administration.

In some embodiments, the administrating comprises intrathecal administration, intramuscular administration, intravenous administration, transdermal administration, intranasal administration, oral administration, mucosal administration, intraperitoneal administration, intracranial administration, intracerebroventricular administration, or stereotaxic administration.

In some embodiments, the administration is injection or infusion.

In some embodiments, the subject is a human, a non-human primate, or a mouse.

In some embodiments, the level of the transcript (e.g., mRNA) of the target dsDNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target dsDNA in the subject prior to the administration.

In some embodiments, the level of the transcript (e.g., mRNA) of the target dsDNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target dsDNA in the subject prior to the administration.

In some embodiments, the level of the expression product (e.g., protein) of the target dsDNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target dsDNA in the subject prior to the administration.

In some embodiments, the level of the expression product (e.g., protein) of the target dsDNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target dsDNA in the subject prior to the administration. In some embodiments, the expression product is a functional mutant of the expression product of the target dsDNA.

In some embodiments, the median survival of the subject suffering from the disease but receiving the administration is 5 days, 10 days, 20 days, 30 days, 2 months, 3 months, 4 months, 5 months, 6 months, 7 months, 8 months, 9 months, 10 months, 11 months, 12 months, 1.5 year, 2 years, 2.5 years, 3 years, 4 years, 5 years, 6 years, 7 years, 8 years, 9 years, 10 years or more longer than that of a subject or a population of subjects suffering from the disease and not receiving the administration.

The therapeutically effective dose may be either via a single dose, or multiple doses. One skilled in the art understands that the actual dose may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

For example, the therapeutically effective dose of the rAAV particle may be about 1.0E+8, 2.0E+8, 3.0E+8, 4.0E+8, 6.0E+8, 8.0E+8, 1.0E+9, 2.0E+9, 3.0E+9, 4.0E+9, 6.0E+9, 8.0E+9, 1.0E+10, 2.0E+10, 3.0E+10, 4.0E+10, 6.0E+10, 8.0E+10, 1.0E+11, 2.0E+11, 3.0E+11, 4.0E+11, 6.0E+11, 8.0E+11, 1.0E+12, 2.0E+12, 3.0E+12, 4.0E+12, 6.0E+12, 8.0E+12, 1.0E+13, 2.0E+13, 3.0E+13, 4.0E+13, 6.0E+13, 8.0E+13, 1.0E+14, 2.0E+14, 3.0E+14, 4.0E+14, 6.0E+14, 8.0E+14, 1.0E+15, 2.0E+15, 3.0E+15, 4.0E+15, 6.0E+15, 8.0E+15, 1.0E+16, 2.0E+16, 3.0E+16, 4.0E+16, 6.0E+16, 8.0E+16, or 1.0E+17 vg, or within a range of any two of the those point values. vg stands for vector genomes of rAAV particles for administration.

In yet another aspect, the disclosure provides a method of detecting a target dsDNA, comprising contacting the target dsDNA with the system of the disclosure, wherein the target dsDNA is modified by the complex, and wherein the modification detects the target dsDNA. In some embodiments, the modification generates a detectable signal, e.g., a fluorescent signal.

### Kits

In yet another aspect, the disclosure provides a kit comprising the fusion protein of the disclosure, the system of the disclosure, the polynucleotide of the disclosure, the vector of the disclosure, the RNP of the disclosure, the LNP of the disclosure, the delivery system of the disclosure, the cell of the disclosure, or the pharmaceutical composition of the disclosure, or any one, two, or all components of the same.

In some embodiments, the kit further comprises an instruction to use the component (s) contained therein, and/or instructions for combining with additional component (s) that may be available or necessary elsewhere.

In some embodiments, the kit further comprises one or more buffers that may be used to dissolve any of the component (s) contained therein, and/or to provide suitable reaction conditions for one or more of the component (s). Such buffers may include one or more of PBS, HEPES, Tris, MOPS, Na₂CO₃, NaHCO₃, NaB, or combinations thereof. In some embodiments, the reaction condition includes a proper pH, such as a basic pH. In some embodiments, the pH is between 7-10.

In some embodiments, any one or more of the kit components may be stored in a suitable container or at a suitable temperature, e.g., 4 degree Celsius.

Further embodiments are illustrated in the following Examples which are given for illustrative purposes only and are not intended to limit the scope of the disclosure.

### EXAMPLES

The following examples are provided to further illustrate some embodiments of the disclosure but are not intended to limit the scope of the invention; it will be understood by their exemplary nature that other procedures, methodologies, or techniques known to those skilled in the art may alternatively be used.

### Example 1. Development of Deaminase-Free, Glycosylase-Based Base Editors

It was intended to develop a novel base editing system by harnessing the BER pathway triggered by depurination of G or A. By fusing human MPG at the C-terminus of Cas9-D10A nickase (nCas9) (SEQ ID NO: 2), a prototype version of a deaminase-free, glycosylase-based base editor (gBE) was generated, in which the MPG was intended to remove normal G or A, two purines with similar structure of hypoxanthine (Hx), to generate AP sites, with nCas9 nicking the opposite non-edited strand. This would allow the damaged DNA to preferentially use the edited strand as a template for DNA repair and/or DNA replication. Therefore, nucleotide incorporation opposite the AP site via TLS would lead to diverse base editing outcomes (FIG. 1a).

To conveniently evaluate the editing efficiency of the gBE, a simple intron-split EGFP reporter system was developed as reported previously [11]. Disruptive point mutations (AG to CG or TG) were introduced in the intron boundary to generate inactive splicing acceptor (SA) signals, and G-to-T or A-to-T conversion (C-to-A or T-to-A conversion in the opposite strand) was required to correct the mutation for proper splicing of the EGFP coding sequence, thus activating EGFP expression and generating EGFP signals (FIG. 1b and FIG. 10a). The EGFP fluorescence intensity could be detected with flow cytometry (FIG. 10d). The G-to-T reporter was to be used for evaluating the guanine base editing efficiency of the gBE (for this purpose, termed as glycosylase-based guanine base editor (gGBE)), and the A-to-T reporter for evaluating the adenine base editing efficiency of the gBE (for this purpose, termed as glycosylase-based adenine base editor (gABE)).

gBE expression vectors with wild type human MPG (SEQ ID NO: 1) (without first N-terminal Methionine (M) as compared to the full length wild type human MPG (SEQ ID NO: 9)) or a distinctive version of human MPG mutants (i.e., MPGv0.2 (SEQ ID NO: 4), MPGv1 (SEQ ID NO: 5), MPGv2 (SEQ ID NO: 6), and MPGv3 (SEQ ID NO: 7)) that were reported in a previous study [11] were constructed. After co-transfecting the G-to-T or A-to-T reporter vector with the gBE expression vector encoding a targeting single guide RNA (targeting sgRNA) that targeted the intronic mis-splicing mutation, it was found that all the gBE showed G-to-T but not A-to-T conversion activity (FIG. 1c and FIG. 10b). The differences between the normal G and A substrate recognition capability of the tested MPGs might account for the failure of A editing in this system.

As used herein, the term “conversion activity” refers to the activity of the gBE of the disclosure to convert a target deoxyribonucleotide to an outcome deoxyribonucleoti de, and the outcome deoxyribonucleotide may be or may not be specified as a specific type of deoxyribonucleotide, e.g., G-to-T. As used herein the term “(base) editing efficiency” refers to the activity of the gBE of the disclosure to convert a target deoxyribonucleotide to an outcome deoxyribonucleotide, and the outcome deoxyribonucleotide may be or may not be specified as a specific type of deoxyribonucleotide, e.g., G-to-T. In the case that both the outcome deoxyribonucleotides for conversion activity and (base) editing efficiency are not specified, or both the outcome deoxyribonucleotides for conversion activity and (base) editing efficiency are specified as the same one or more specific types of deoxyribonucleotide, they may refer to the same performance of the gBE of the disclosure and can be used interchangeably.

For G base editing, the gBE (hereafter referred to as gGBEv3) (SEQ ID NO: 15) containing MPGv3 (SEQ ID NO: 7) exhibited the highest G-to-T base editing efficiency (4.33%) in cultured HEK293T cells (FIG. 1c) as compared to that of the gGBE (SEQ ID NO: 14) with WT hMPG (SEQ ID NO: 1) (0.03%), showing a striking 144-fold enhancement in G base editing efficiency. As two negative controls, no conversion activity was found for the gBE (SEQ ID NO: 13) with dead MPG (SEQ ID NO: 3) (dMPG, carrying E125A, Y127A, and H136A mutations) or gGBEv3 together with a nontargeting sgRNA (“NT”) (FIG. 10c). Thus, the G-to-T conversion by gGBEv3 was demonstrated to depend on the catalytic G excision domain of the MPG guided by the specific sgRNA.

### Example 2. Further Enhancement of G Editing Efficiency of gGBE

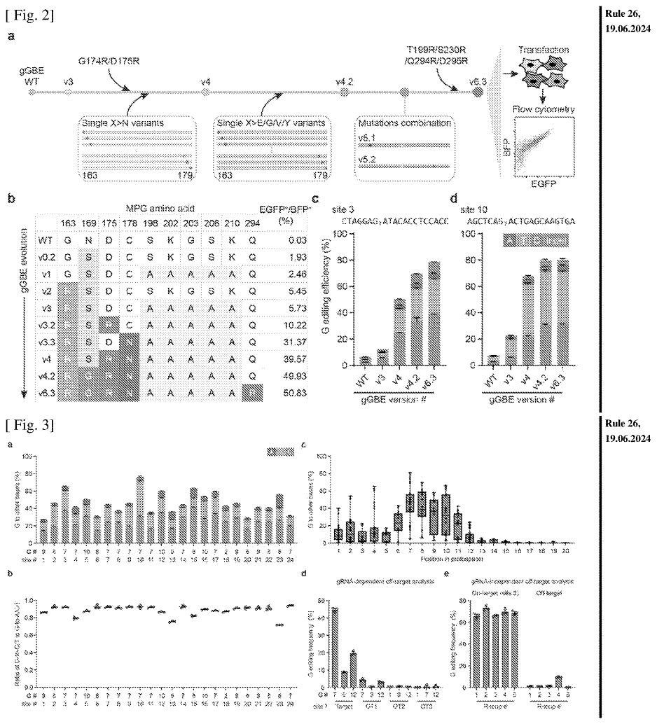

To further increase the G-to-T activity of gGBEv3, protein engineering was performed by several rounds of rational mutagenesis of MPGv3 contained in gGBEv3, and the G-to-T reporter in Example 1 was used to evaluate the G editing efficiency (FIG. 2a). Based on structural analysis of MPG [32, 33], the 17-aa-long region from R163 to V179 (R163-V179) of MPGv3 was selected, which forms a pocket around the targeted G base in a model of MPG-DNA complex (FIG. 11a).

On one hand, gGBEv3 was mutated with G174R or D175R, generating new base editors gGBEv3.1 (SEQ ID NO: 17) (containing MPGv3 carrying additional substitution G174R; termed as MPGv3.1 (SEQ ID NO: 16)) and gGBEv3.2 (SEQ ID NO: 19) (containing MPGv3 carrying additional substitution D175R; termed as MPGv3.2 (SEQ ID NO: 18)) (FIG. 2a). It was found that the G-to-T conversion activity of gGBEv3.2 (10.22%) was about 1.78-fold of gGBEv3 (5.73%) (FIG. 2b and FIG. 11b).

On the other hand, the R163-V179 region was scanned with sequential substitution to Asparagine (Asn/N) (X-to-N). Interestingly, gGBEv3.3 (SEQ ID NO: 21) (containing MPGv3 carrying additional substitution C178N; termed as MPGv3.3 (SEQ ID NO: 20)) (31.37%) was obtained with substantially elevated G-to-T conversion activity of about 5.5-fold compared to gGBEv3 (5.73%) (FIG. 2a and FIG. 11c).

Together, gGBEv4 (SEQ ID NO: 23) (containing MPGv3 carrying additional substitutions of both D175R and C178N; termed as MPGv4 (SEQ ID NO: 22)) (39.57%) achieved a synergistic enhancement of G-to-T editing efficiency of about 6.9-fold compared to gGBEv3 (5.73%) (FIG. 2b and FIG. 11b).

In addition, the orientation of nCas9 and MPG was changed to see whether the editing efficiency would be associated with the positional relationship of nCas9 and MPG. It was found that gGBEv4 with MPG fused at the C-terminus of nCas9 had slightly higher editing efficiency than gGBEv4 with MPG fused at the N-terminus of nCas9 (34.6% vs. 25.9%, FIG. 11d).

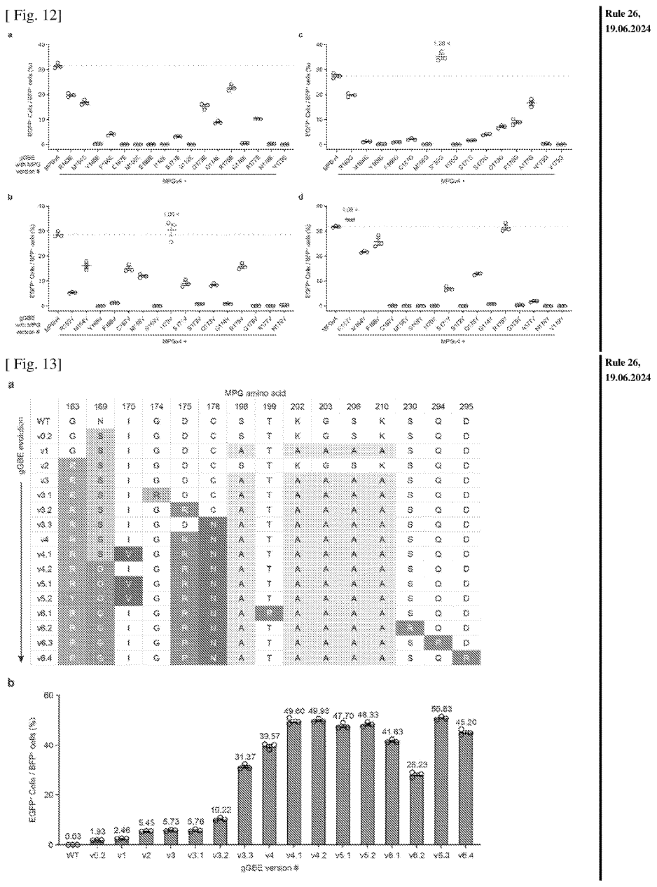

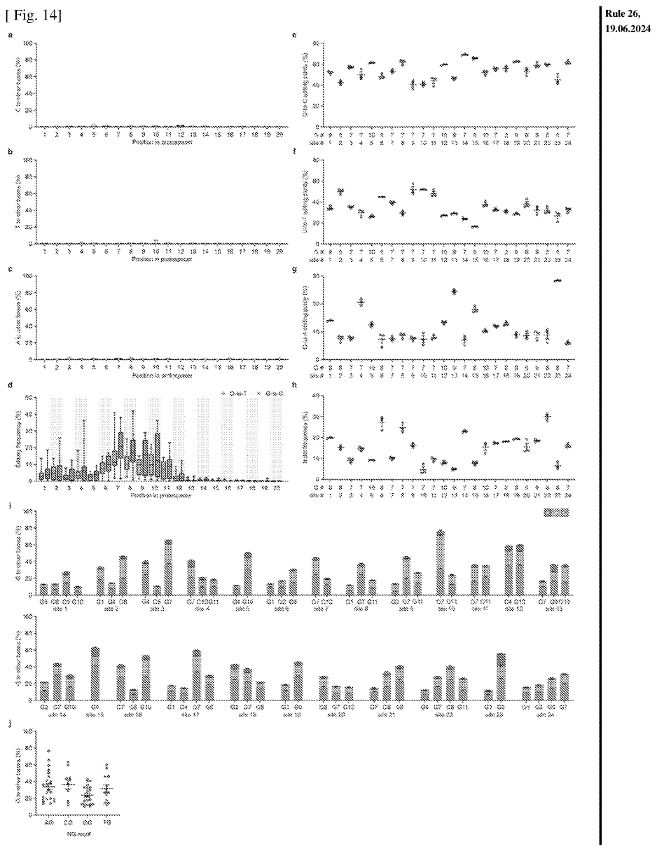

Furthermore, another round of mutagenesis and screening was performed based on gGBEv4 to elevate G editing efficiency. The R163-V179 region of MPGv4 was mutated by sequential replacement with amino acids having distinct properties, including glutamic acid (with negative charged side chain), valine (with small hydrophobic side chain), glycine (with no side chain), or tyrosine (with large hydrophobic side chain) (X-to-E, V, G, or Y). Three base editors, gGBEv4.1 (SEQ ID NO: 25) (containing MPGv4 carrying additional substitution T170V; termed as MPGv4.1 (SEQ ID NO: 24)), gGBEv4.2 (SEQ ID NO: 27) (containing MPGv4 carrying additional substitution S169G (or N169G if compared with WT MPG); termed as MPGv4.2 (SEQ ID NO: 26)), and gGBEv4.3 (SEQ ID NO: 29) (containing MPGv4 carrying additional substitution R163Y (or G163Y if compared with WT MPG); termed as MPGv4.3 (SEQ ID NO: 28)), were found to show elevated editing efficiency of 1.06-, 1.28-, and 1.09-fold, respectively (FIG. 12), as compared with that of gGBEv4. Two additional base editors (gGBEv5.1 with S169G and I170V and gGBEv5.2 with R163Y, S169G, and I170Y) were generated by combining the above three effective mutations, but no further enhancement was found compared with gGBEv4.1 and gGBEv4.2 (FIG. 13).

Encouraged by the above finding on gGBEv3.2, T199R, S230R, Q294R, or D295R was further introduced individually into gGBEv4.2. It was found that gGBEv6.3 (SEQ ID NO: 12) (containing MPGv4.2 plus additional substitution Q294R; termed as MPGv6.3 (SEQ ID NO: 8)) (50.83%) had further increased editing efficiency by about 1.02- and 1.694-folds, as compared with gGBEv4.2 (SEQ ID NO: 27) (49.93%) and gGBE (SEQ ID NO: 14) (0.03%) with WT MPG, respectively (FIG. 2b and FIG. 13). The amino acid sequences of gGBEv5.1, v5.2, v6.1, v6.2, and v6.4 are set forth in SEQ ID NOs: 31, 33, 35, 37, and 39, respectively, and the corresponding MPGv5.1, v5.2, v6.1, v6.2, and v6.4 are set forth in SEQ ID NOs: 30, 32, 34, 36, and 38, respectively.

The enhancement of G editing efficiency of the gGBEs (v3, v4, v4.2, v6.3, and gGBE with WT MPG) obtained above was next validated at two endogenous genomic sites in cultured HEK293T cells. The cells were transfected with a construct encoding each gGBE, together with mCherry and sgRNA that targeted site 3 or site 10, and mCherry⁺ cells were FACS-sorted for target deep sequencing analysis. A gradual elevation of overall G editing efficiency was obtained at G7 from 6.4% to 78.5% for site 3, and from 7.5% to 80.3% for site 10, respectively (FIGS. 2c and 2d), confirming that gGBEv6.3 was indeed the best version of gGBE. The G-to-C and G-to-T conversions were the predominant events at these two sites, and a small percentage of G-to-A conversion (4.6% for site 3, 5.0% for site 10) and a few insertions and deletions (indels, 8.5% for site 3; 4.3% for site 10) were detected for gGBEv6.3. These results indicate that the four rounds of mutagenesis described above had effectively optimized gGBE activity for G-to-C and G-to-T base editing. Taken together, the engineered gGBEv6.3 (SEQ ID NO: 12) (carrying G163R, N169G, D175R, C178N, S198A, K202A, G203A, S206A, K210A, Q294R mutations on MPGv6.3 (SEQ ID NO: 8)) had the highest G editing efficiency and was used in the following studies.

### Example 3. Characterization of gGBEv6.3 at Human Genomic DNA Sites

The editing profiles of gGBEv6.3 was characterized by targeting 24 endogenous genomic loci, most of which were used in previous base editing studies [9, 35, 36]. It was found that gGBEv6.3 achieved efficient G base editing efficiency (ranged from 27.6% to 76.5%), with predominately G-to-C and G-to-T conversions, and essentially no A, C, or T editing at all 24 sites examined (FIGS. 3a and 3c and FIG. 14a-c). Among all the cells examined, the ratio of G-to-C/T (G-to-Y, Y=C and T) to G-to-A/T/C conversion was high (ranging from 0.72 to 0.94, FIG. 3b). A small percentage of G-to-A conversion was detected (FIG. 3a and FIG. 14e-g), which is consistent with previous results with AYBE [11] and CGBEs [6-10]. It was found that gGBEv6.3 also induced indel at the 24 edited sites with frequency ranging from 4.7% to 30.0% (FIG. 14h). Furthermore, it was found that the editable range of gGBEv6.3 was positions 1 to 14, and the optimal editing window with high efficiency of G conversion covered protospacer positions 6 to 11 (FIG. 3c), with the highest editing efficiency at position 7 (FIG. 3c, FIG. 14d and S6i). The analysis of on-target editing and sequences of all the sites showed that gGBEv6.3 performed no obvious NG motif preference for G conversions (FIG. 14j).

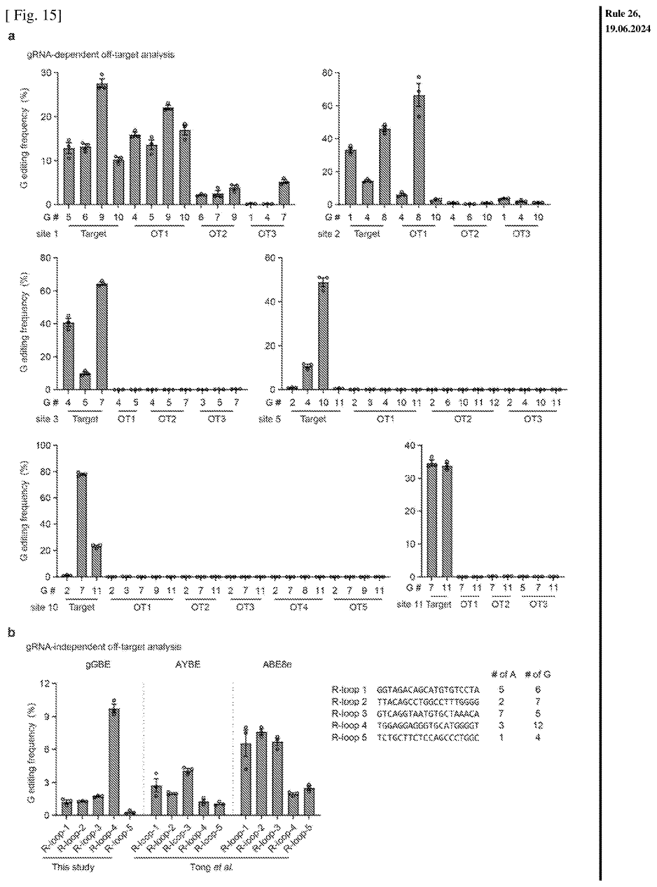

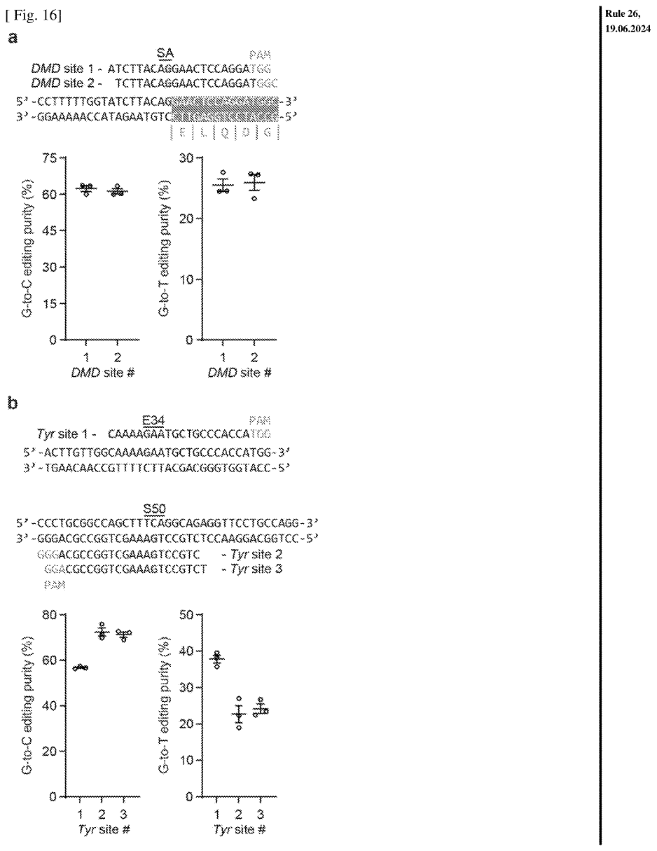

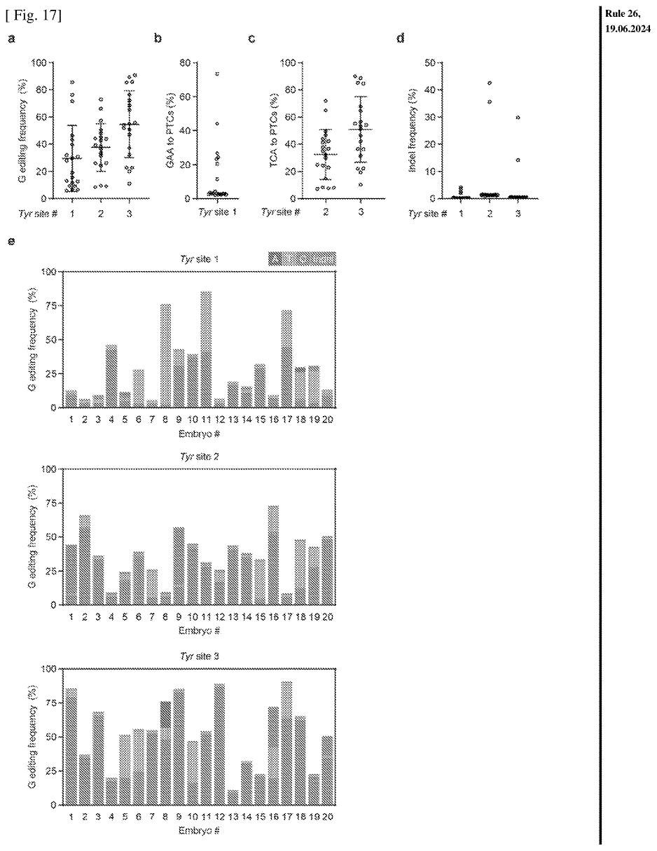

The guide sequence-dependent off-target base editing efficiency of gGBEv6.3 was analyzed at several previously reported [11, 35] and in silico-predicted [37] guide sequence-dependent off-target sites, and the ability of gGBEv6.3 to mediate guide sequence-independent off-target DNA editing was characterized using orthogonal R-loop assay in five dSaCas9 R-loops, as reported in previous studies [11, 35]. Similar or lower percentage of editing at the guide sequence-dependent off-target loci (FIG. 3d and FIG. 15a) was found, as compared with that of adenine base editors found previously [11, 35]. Moreover, among five guide sequence-independent off-target sites, very low frequencies (1.1% on average) were detected at four sites (FIG. 3e and FIG. 15b), and a slightly higher frequency was detected at one site, which harbored 12 Gs across the entire protospacer. Taken together, it was demonstrated that gGBEv6.3 represents a highly efficient G-to-Y base editor with a low off-target effect in mammalian cells.

On the other hand, gGBEv6.3 was also tested with A-to-T reporter, C-to-G reporter, and T-to-G reporter, and the editing efficiency was 0.68%, 0.58%, and 1.81%, respectively, demonstrating its specificity for G editing, which is desired for targeted base editing application and reduced unwanted off-target editing.

### Example 4. Potential Gene-Editing Applications of gGBE

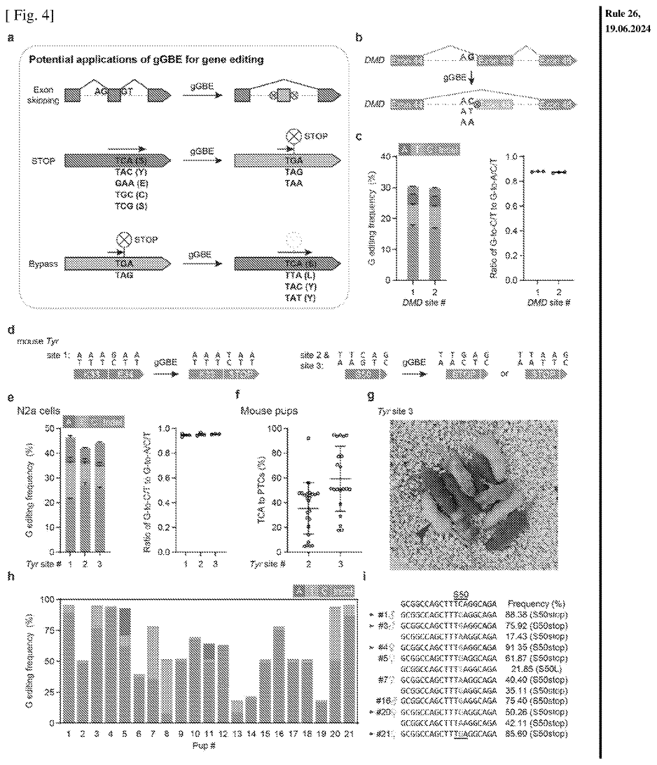

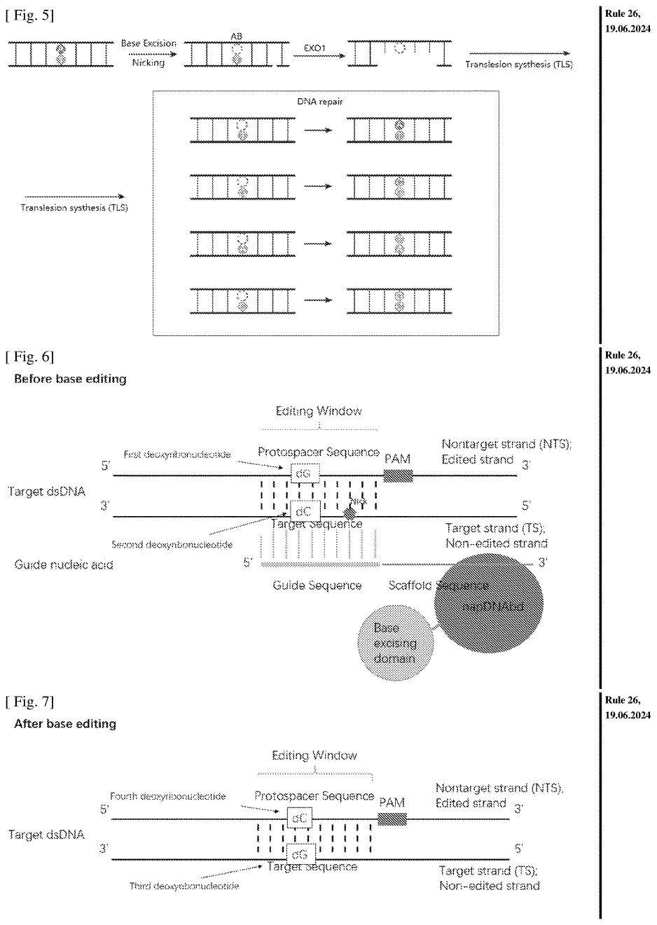

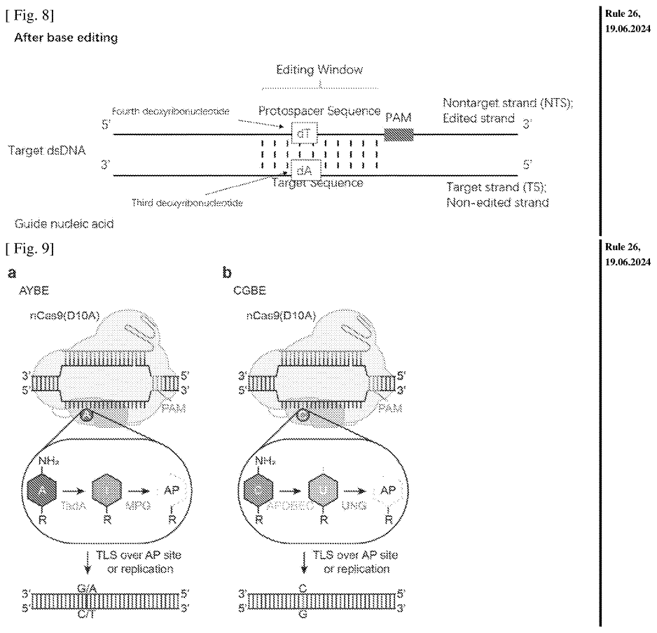

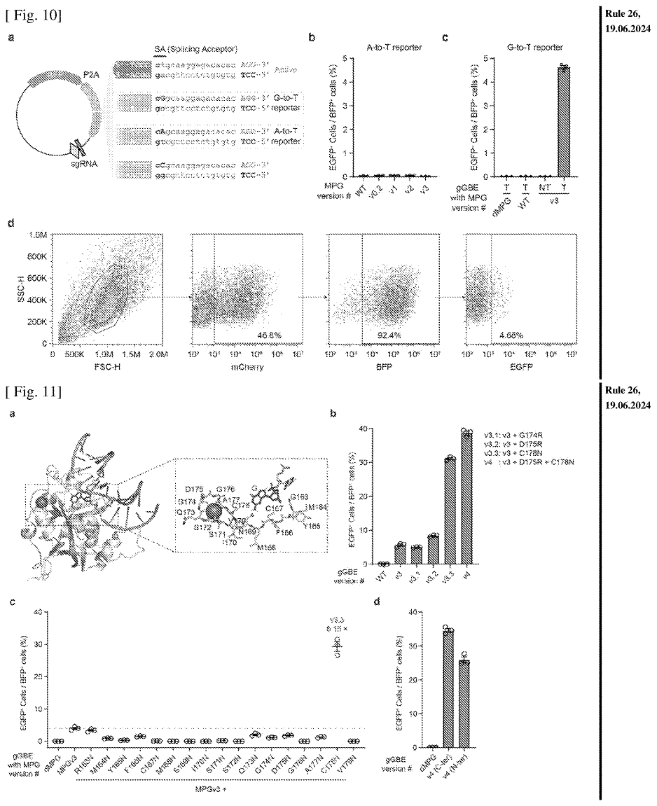

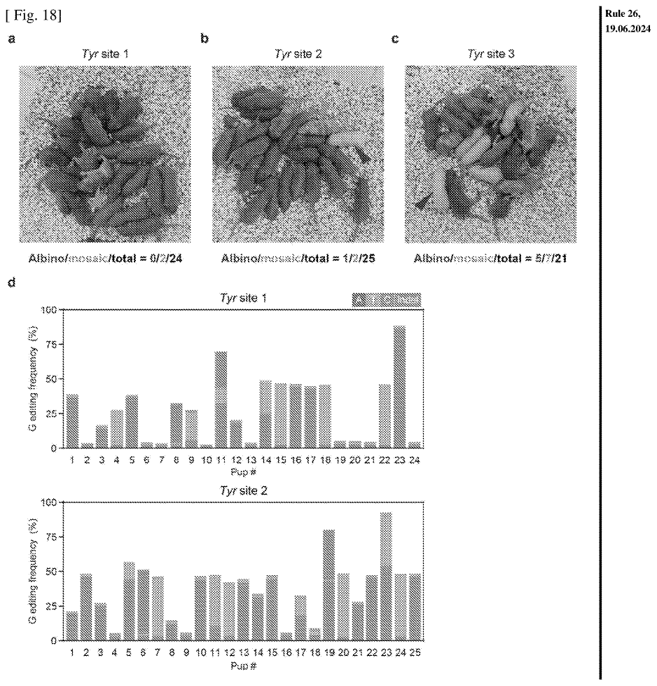

The G-to-Y conversion ability of gGBE allows for a variety of gene-editing applications, including editing splicing sites, introduction of premature termination codons (PTCs), as well as editing that bypasses PTCs (FIG. 4a). The inactive splicing acceptor (SA) signal with disruptive point mutations, exemplified by the intron-split EGFP reporter system used above, could be remediated with gGBE (FIG. 1b). Conversely, gGBE could be used for disrupting the splicing signal by converting G within a splicing donor site (“GT”) or splicing acceptor site (“AG”) to other bases, resulting in exon skipping. To illustrate this application, the splice acceptor site of DMD (Duchenne muscular dystrophy) exon 45 was edited with gGBEv6.3, and a high efficiency of G editing (up to 30.3%) was achieved with a high G-to-Y ratio (up to 0.88) when targeting DMD site 1 (FIG. 4b, c and FIG. 16a).

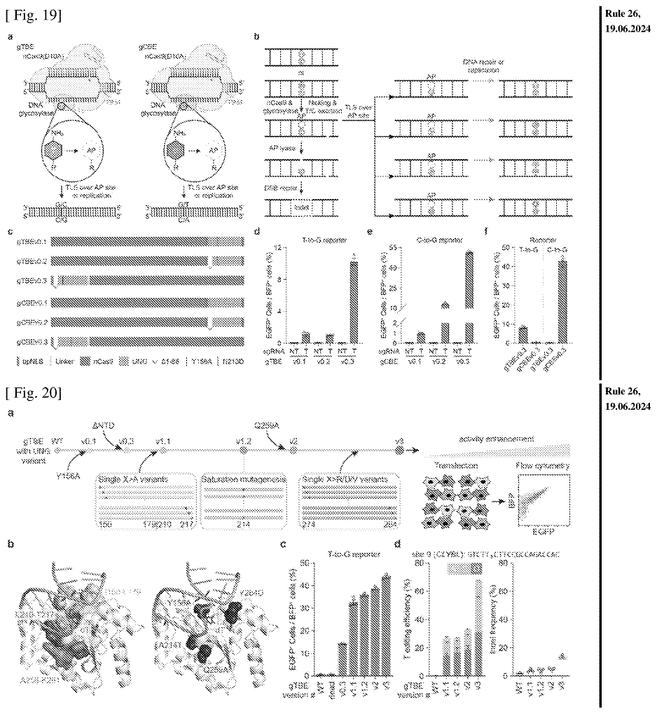

Another application of gGBE is to introduce PTCs to disrupt gene expression by converting TCA, TAC, or GAA codon into a stop codon TGA, TAG, or TAA. Note that PTC by GAA to TAA conversion could be introduced only by using gGBEv6.3, no other current base editor could induce this type of PTC. By targeting three sites in the mouse Tyr (Tyrosinase, associated with coat color) gene with gGBEv6.3 to create PTCs (FIG. 4d) in cultured N2a cells, a high efficiency of G editing (up to 46.3%) was achieved with a high G-to-Y ratio (up to 0.95) (FIG. 4e and FIG. 16b). To further illustrate the potential in vivo application, gGBEv6.3-encoding mRNA and Tyr-targeting sgRNA were co-injected into mouse zygotes of C57BL6 background (black coat color), with 20 mouse embryos used for each of the three Tyr-targeting sgRNAs. Highly efficient G editing (FIG. 17a) was found for two of the three sgRNAs, with an average of 50.9% PTC introduction efficiency when targeting the Tyr site 3 (FIG. 17b-c). Similar to the data obtained above in cultured cell lines (FIG. 4e), gGBE induced very few indels in mouse embryos (FIG. 17d-e). When targeting the Tyr site 3 with gGBEv6.3, all the 21 F0 pups had G conversions with an average of 59.4% efficiency (FIG. 4f-h). This gGBEv6.3-induced G-to-Y editing resulted in the albino or mosaic phenotype in the coat color of the F0 mice (FIG. 18), suggesting efficient disruption of the tyrosinase activity (FIG. 4i). Thus, gGBEv6.3 was demonstrated to be an efficient G base editor not only in cell lines but also in mouse embryos.

### DISCUSSION

Two major classes of deaminase-based base editors (dBE), ABE and CBE, as well as their derivatives (such as AYBE and CGBE), perform base editing with deamination of A or C as the first key step [3-11]. In the disclosure, deaminase-free base editors were designed based on engineered MPG, and a gGBE editor that could achieve highly efficient G-to-C and G-to-T conversion in both cultured human cells and mouse embryos was generated. The engineered MPG demonstrates that DNA glycosylases could be engineered into proteins that selectively excise a specific nucleotide base, such as, G. The high editing efficiency of the gGBE of the disclosure could be attributed to the mutations in the MPG moiety that may facilitate its specific substrate selection or DNA-binding activity, or both.

In all human pathogenic SNPs (60 372 total), there are about 10% C-to-G and 5% T-to-G SNPs [11]. Although C-to-G SNPs could be corrected by CGBE [6-10], an efficient sgRNA was not usually found due to the PAM limitation and narrow editing window. The gGBE of the disclosure could increase the opportunity to find an efficient sgRNA by targeting the opposite strand compared with CGBE. For T-to-G SNPs, no current base editor could efficiently induce G-to-T (or C-to-A in the opposite strand) conversion. Therefore, the gGBE of the disclosure greatly broadens the targeting scope of base editors.

### Methods

### Molecular Cloning

Base editor constructs used in this study were cloned into a mammalian expression plasmid backbone under the control of a EF1α promoter by standard molecular cloning techniques. Intron-split EGFP reporters were engineered as previously described [11]. In brief, corresponding mutations at the splice acceptor site were made to construct A-to-T reporter or G-to-T reporter via site-directed mutagenesis by PCR, respectively. Mutations at the splice acceptor site led to inactive EGFP production by non-spliced EGFP transcripts. Encouraged by the findings from previous base editors [7, 10], modification of the 68^{th} base (G-to-C) was made in the intron sequence for introducing artificial PAM on the template strand, thus the corresponding mutations at the splice acceptor site were at position 6 across the protospacer. Transversion corrections in A-to-T reporter or G-to-T reporter were required for proper splicing of EGFP-coding sequence. MPG mutagenesis libraries were designed and generated as previously described [46]. The 17aa-long region from R163 to V179 (R163-V179) of MPGv3 was selected for protein engineering. BpiI-harboring MPG mutants, MPG-G174R/D175R/T199R/S230R/Q294R/D295R mutants or corresponding combinations, were constructed via site-directed mutagenesis by PCR. Sequential asparagine/glutamic acid/valine/glycine/tyrosine substitutions (X-to-N, E, V, G, or Y) were designed, with oligos coding for the mutants annealed and ligated into corresponding BpiI-digested backbone vectors. The gRNA oligos were annealed and ligated into BpiI sites. Unless otherwise indicated, each and every mutation in MPG is numbered based on the full-length wild type human MPG (SEQ ID NO: 9) with the first N-terminal Met.

### Analysis of Target Sequencing Data

Target sequencing data analysis was described previously [11]. In brief, the targeted amplicon sequencing reads were processed using fastp with default parameters [47]. The cleaned pairs were then merged using FLASH v1.2.11. The amplified sequences from individual targets were demultiplexed using fastx barcode splitter. pl from fastx_toolkit (0.0.14). Further amplicon sequencing analysis was performed by CRTSPResso2 [48]. A 10-bp window was used to quantify modifications centered around the middle of the 20-bp gRNA. Otherwise, the default parameters were used for analysis. G-to-C purity was calculated as G-to-C editing efficiency/(G-to-C editing efficiency+G-to-T editing efficiency+G-to-A editing efficiency). G-to-Y conversion ratio was calculated as (G-to-C editing efficiency+G-to-T editing efficiency)/(G-to-C editing efficiency+G-to-T editing efficiency+G-to-A editing efficiency).

### Cell Culture, Transfection, and Flow Cytometry Analysis

HEK293T cells were cultured with DMEM (Catalog #11995065, Gibco) supplemented with 10% fetal bovine serum (Catalog #04-001-1ACS, BI) and 0.1 mM non-essential amino acids (Catalog #11140-050, Gibco). Cells were grown in an incubator at 37° C. with 5% CO₂. MPG mutant screening was conducted in 48-well plates. The day before transfection, 3×10⁴ HEK293T cells per well were plated in 250 μL of complete growth medium in the 48-well plates. After 12 h, 500 ng gGBE plasmids and 500 ng A-to-T or G-to-T reporter plasmids were co-transfected into cells with 2 g Polyethylenimine (PEI) (DNA/PEI ratio of 1:2) per well. For cell transfection of HEK293T for FACS, 5×10⁵ cells per well were plated in 12-well plates with 1 ml complete growth medium the day before transfection. After 14-16h, 2 g gGBE-sgRNA plasmids were transfected into cells using PEI (DNA/PEI ratio of 1:2). For targeting DMD site 2 with an NG PAM, a PAM-flexible Cas9 variant SpG was used. Orthogonal R-loop assays were performed as described previously [1, 2]. In brief, 1 g of gGBE plasmid with sgRNA targeting site 3 and 1 g of dSaCas9 plasmid with corresponding sgRNA targeting five off-target sites to generate R-loops were co-transfected into HEK293T cells in 12-well plates using PEI (DNA/PEI ratio of 1:2). 48 h after transfection, expression of mCherry, BFP and EGFP fluorescence were analyzed by BD FACS Aria III or Beckman CytoFLEX S. Flow cytometry results were analyzed with FlowJo V10.5.3. The gating strategy in the identification of mCherry⁺, BFP⁺ and EGFP⁺ cells for on-target editing efficiency evaluation was supplied in FIG. 10d.

### Animals

Experiments involving mice were approved by the Biomedical Research Ethics Committee of Center for HuidaGene Therapeutics Co. Ltd. Super ovulated C57BL/6 females (4 weeks old) were mated with C57BL/6 males (8 weeks old), and females from the ICR strain were used as foster mothers. Mice were maintained in a specific pathogen-free facility under a 12-hour dark-light cycle, and constant temperature (20-26° C.) and humidity (40-60%) maintenance.

In Vitro Transcription of gGBE mRNA and Tyr-sgRNAs

The gGBE plasmids were structured by standard PCR amplification with Phanta Max Super-Fidelity DNA Polymerase (Vazyme Biotech Co., Ltd), assembly with Gibson Assembly Master Mix (NEB E2611L), and transformation into chemically competent DH5a cells. The gGBE plasmids were linearized by the FastDigest KpnI restriction enzyme (Thermo Fisher), purified using Gel Extraction Kit (Omega), and used as the template for in vitro transcription (IVT) using the mMESSAGE mMACHINE T7 Ultra kit (Life Technologies). For Tyr-sgRNAs preparation, T7 promoter sequence was added to the sgRNA template by PCR amplification of px330 (Addgene, #42230). The T7-Tyr-sgRNA PCR product was purified using Gel Extraction Kit (Omega) and used as the template for IVT of sgRNAs using the MEGAshortscript T7 kit (Life Technologies). The gGBE mRNA and Tyr-sgRNAs were purified using the MEGAclear kit (Life Technologies) and eluted in RNase-free water. In vitro transcribed RNAs were aliquoted and stored at −80° C. until use. Prior to microinjection, the mixture of gGBE mRNA and Tyr-sgRNA was prepared by centrifuge for 10 min at 14,000 rpm at 4° C. and supernatant transferred to 0.2 mL fresh PCR tubes for injection.

Microinjection of Mouse Zygotes with gGBE mRNA and Tyr-sgRNA

Super-ovulated C57BL/6 females (4 weeks old) were mated with C57BL/6 males, and fertilized embryos were collected from oviducts 21 h post hCG injection. For zygote injection, the mixture of gGBE mRNA (100 ng/μL) and Tyr-sgRNAs (100 ng/μL) was injected into the cytoplasm of 1-cell embryo in a droplet of M2 medium using a FemtoJet microinjector (Eppendorf) with constant flow settings. The injected embryos were cultured in M16 medium with amino acids at 37° C. under 5% CO2 in air for 2 hours and then transferred into oviducts of pseudo-pregnant ICR foster mothers at 0.5-d. p. c.

### Target Sequencing of Endogenous Sites

At 72 h post-transfection, 10,000 mCherry positive cells were isolated by FACS. Genomic DNA was extracted by addition of 40 μl of lysis buffer and 1 μL Proteinase K (Catalog #PD101-01, Vazyme) directly into each tube of sorted cells. The genomic DNA/lysis buffer mixture was incubated at 55° C. for 45 min, followed by a 95° C. enzyme inactivation step for 10 min. The regions of interest for target sites were amplified by PCR using site-specific primers. The PCR reaction was performed at 95° C. for 5 min, 30 cycles at 95° cfor 15 s, 60° C. for 15 s, 72° C. 30 s, and a final extension at 72° C. for 5 min using Phanta @Max Super-Fidelity DNA Polymerase (Catalog #P505-d3, Vazyme). PCR products were purified using universal DNA purification kit (TIANGEN) according to the manufacturer's instructions, and analyzed by Sanger sequencing (Genewiz). The amplicons were ligated to adapters and sequencing was performed on the Illumina MiSeq platforms. Protospacer sequences used for each genomic locus are listed in Table 1.

### Statistical Analysis

Statistical tests performed by Graphpad Prism 8 included the two-tailed unpaired two-sample t-test or Dunnett's multiple comparisons test after one-way ANOVA.

### REFERENCES

1. Porto E M, Komor A C, Slaymaker I M, et al.; Base editing: advances and therapeutic opportunities. Nat Rev Drug Discov 2020; 19 (12): 839-859. doi: 10.1038/s41573-020-0084-6.
2. Rees H A, Liu D R; Base editing: precision chemistry on the genome and transcriptome of living cells. Nat Rev Genet 2018; 19 (12): 770-788. doi: 10.1038/s41576-018-0059-1.
3. Komor A C, Zhao K T, Packer M S, et al.; Improved base excision repair inhibition and bacteriophage Mu Gam protein yields C: G-to-T: Abase editors with higher efficiency and product purity. Sci Adv 2017; 3 (8): eaao4774. doi: 10.1126/sciadv. aao4774.
4. Komor A C, Kim Y B, Packer M S, et al.; Programmable editing of a target base in genomic DNA without double-stranded DNA cleavage. Nature 2016; 533 (7603): 420-4. doi: 10.1038/nature17946.
5. Gaudelli N M, Komor A C, Rees H A, et al.; Programmable base editing of A*T to G*C in genomic DNA without DNA cleavage. Nature 2017; 551 (7681): 464-471. doi: 10.1038/nature24644.
6. Zhao D, Li J, Li S, et al.; Glycosylase base editors enable C-to-A and C-to-G base changes. Nat Biotechnol 2021; 39 (1): 35-40. doi: 10.1038/s41587-020-0592-2.
7. Kurt I C, Zhou R, Iyer S, et al.; CRISPR C-to-G base editors for inducing targeted DNA transversions in human cells. Nat Biotechnol 2021; 39 (1): 41-46. doi: 10.1038/s41587-020-0609-x.
8. Koblan L W, Arbab M, Shen M W, et al.; Efficient C*G-to-G*C base editors developed using CRISPRi screens, target-library analysis, and machine learning. Nat Biotechnol 2021; 39 (11): 1414-1425. doi: 10.1038/s41587-021-00938-z.
9. Chen L, Park J E, Paa P, et al.; Programmable C: G to G: C genome editing with CRISPR-Cas9-directed base excision repair proteins. Nat Commun 2021; 12 (1): 1384. doi: 10.1038/s41467-021-21559-9.
10. Yuan T, Yan N, Fei T, et al.; Optimization of C-to-G base editors with sequence context preference predictable by machine learning methods. Nat Commun 2021; 12 (1): 4902. doi: 10.1038/s41467-021-25217-y.
11. Tong H, Wang X, Liu Y, et al.; Programmable A-to-Y base editing by fusing an adenine base editor with an N-methylpurine DNA glycosylase. Nat Biotechnol 2023. doi: 10.1038/s41587-022-01595-6.
12. Mok B Y, de Moraes M H, Zeng J, et al.; A bacterial cytidine deaminase toxin enables CRISPR-free mitochondrial base editing. Nature 2020; 583 (7817): 631-637. doi: 10.1038/s41586-020-2477-4.
13. Lei Z, Meng H, Liu L, et al.; Mitochondrial base editor induces substantial nuclear off-target mutations. Nature 2022; 606 (7915): 804-811. doi: 10.1038/s41586-022-04836-5.
14. Cho S I, Lee S, Mok Y G, et al.; Targeted A-to-G base editing in human mitochondrial DNA with programmable deaminases. Cell 2022; 185 (10): 1764-1776 e12. doi: 10.1016/j. cell. 2022.03.039.
15. Ladstatter S, Tachibana-Konwalski K; A Surveillance Mechanism Ensures Repair of DNA Lesions during Zygotic Reprogramming. Cell 2016; 167 (7) 1774-1787 e13. doi: 10.1016/j. cell. 2016.11.009.
16. Marrone A, Ballantyne J; Hydrolysis of DNA and its molecular components in the dry state. Forensic Sci Int Genet 2010; 4 (3): 168-77. doi: 10.1016/j. fsigen. 2009.08.007.
17. Lindahl T; Instability and decay of the primary structure of DNA. Nature 1993; 362 (6422): 709-15. doi: 10.1038/362709a0.
18. Lee S J, Choi M Y, Miller R E; Vibrational spectroscopy of xanthine in superfluid helium nanodroplets. Chemical Physics Letters 2009; 475 (1): 24-29. doi: 10.1016/j. cplett. 2009.05.016.
19. Hindi N N, Elsakrmy N, Ramotar D; The base excision repair process: comparison between higher and lower eukaryotes. Cell Mol Life Sci 2021; 78 (24): 7943-7965. doi: 10.1007/s00018-021-03990-9.
20. Thompson P S, Cortez D; New insights into abasic site repair and tolerance. DNA Repair (Amst) 2020; 90: 102866. doi: 10.1016/j. dnarep. 2020.102866.
21. Bauer N C, Corbett A H, Doetsch P W; The current state of eukaryotic DNA base damage and repair. Nucleic Acids Res 2015; 43 (21): 10083-101. doi: 10.1093/nar/gkv1136.
22. Jacobs A L, Schar P; DNA glycosylases: in DNA repair and beyond. Chromosoma 2012; 121 (1): 1-20. doi: 10.1007/s00412-011-0347-4.
23. Robertson A B, Klungland A, Rognes T, et al.; DNA repair in mammalian cells: Base excision repair: the long and short of it. Cell Mol Life Sci 2009; 66 (6): 981-93. doi: 10.1007/s00018-009-8736-z.
24. Gallagher P E, Brent T P; Partial purification and characterization of 3-methyladenine-DNA glycosylase from human placenta. Biochemistry 1982; 21 (25): 6404-9. doi: 10.1021/bi00268a013.
25. Asaeda A, Ide H, Asagoshi K, et al.; Substrate specificity of human methylpurine DNA N-glycosylase. Biochemistry 2000; 39 (8): 1959-65. doi: 10.1021/bi9917075.
26. Lau A Y, Wyatt M D, Glassner B J, et al.; Molecular basis for discriminating between normal and damaged bases by the human alkyladenine glycosylase, AAG. Proc Natl Acad Sci USA 2000; 97 (25): 13573-8. doi: 10.1073/pnas. 97.25.13573.
27. Wyatt M D, Samson L D; Influence of DNA structure on hypoxanthine and 1, N (6)-ethenoadenine removal by murine 3-methyladenine DNA glycosylase. Carcinogenesis 2000; 21 (5): 901-8. doi: 10.1093/carcin/21.5.901.
28. Saparbaev M, Laval J; Excision of hypoxanthine from DNA containing dIMP residues by the Escherichia coli, yeast, rat, and human alkylpurine DNA glycosylases. Proc Natl Acad Sci USA 1994; 91 (13): 5873-7. doi: 10.1073/pnas. 91.13.5873.
29. O'Brien P J, Ellenberger T; Dissecting the broad substrate specificity of human 3-methyladenine-DNA glycosylase. J Biol Chem 2004; 279 (11): 9750-7. doi: 10.1074/jbc. M312232200.
30. Berdal K G, Johansen R F, Seeberg E; Release of normal bases from intact DNA by a native DNA repair enzyme. EMBO J 1998; 17 (2): 363-7. doi: 10.1093/emboj/17.2.363.
31. Kay J E, Corrigan J J, Armijo A L, et al.; Excision of mutagenic replication-blocking lesions suppresses cancer but promotes cytotoxicity and lethality in nitrosamine-exposed mice. Cell Rep 2021; 34 (11): 108864. doi: 10.1016/j. celrep. 2021.108864.
32. Vallur A C, Maher R L, Bloom L B; The efficiency of hypoxanthine excision by alkyladenine DNA glycosylase is altered by changes in nearest neighbor bases. DNA Repair (Amst) 2005; 4 (10): 1088-98. doi: 10.1016/j. dnarep. 2005.05.008.
33. Lau A Y, Scharer O D, Samson L, et al.; Crystal structure of a human alkylbase-DNA repair enzyme complexed to DNA: mechanisms for nucleotide flipping and base excision. Cell 1998; 95 (2): 249-58. doi: 10.1016/s0092-8674 (00) 81755-9.
34. Connor E E, Wyatt M D; Active-site clashes prevent the human 3-methyladenine DNA glycosylase from improperly removing bases. Chem Biol 2002; 9 (9): 1033-41. doi: 10.1016/s1074-5521 (02) 00215-6.
35. Richter M F, Zhao K T, Eton E, et al.; Phage-assisted evolution of an adenine base editor with improved Cas domain compatibility and activity. Nat Biotechnol 2020; 38 (7): 883-891. doi: 10.1038/s41587-020-0453-z.
36. Doman J L, Raguram A, Newby G A, et al.; Evaluation and minimization of Cas9-independent off-target DNA editing by cytosine base editors. Nat Biotechnol 2020; 38 (5): 620-628. doi: 10.1038/s41587-020-0414-6.
37. Bae S, Park J, Kim J S; Cas-OFFinder: a fast and versatile algorithm that searches for potential off-target sites of Cas9 RNA-guided endonucleases. Bioinformatics 2014; 30 (10): 1473-5. doi: 10.1093/bioinformatics/btu048.
38. Zuo E, Sun Y, Wei W, et al.; GOTI, a method to identify genome-wide off-target effects of genome editing in mouse embryos. Nat Protoc 2020; 15 (9): 3009-3029. doi: 10.1038/s41596-020-0361-1.
39. Zuo E, Sun Y, Wei W, et al.; Cytosine base editor generates substantial off-target single-nucleotide variants in mouse embryos. Science 2019; 364 (6437): 289-292. doi: 10.1126/science. aav9973.
40. Yan N, Feng H, Sun Y, et al.; Cytosine base editors induce off-target mutations and adverse phenotypic effects in transgenic mice. Nat Commun 2023; 14 (1): 1784. doi: 10.1038/s41467-023-37508-7.
41. Lei Z, Meng H, Lv Z, et al.; Detect-seq reveals out-of-protospacer editing and target-strand editing by cytosine base editors. Nat Methods 2021; 18 (6): 643-651. doi: 10.1038/s41592-021-01172-w.
42. Chen L, Zhang S, Xue N, et al.; Engineering a precise adenine base editor with minimal bystander editing. Nat Chem Biol 2023; 19 (1): 101-110. doi: 10.1038/s41589-022-01163-8.
43. Kim Y B, Komor A C, Levy J M, et al.; Increasing the genome-targeting scope and precision of base editing with engineered Cas9-cytidine deaminase fusions. Nat Biotechnol 2017; 35 (4): 371-376. doi: 10.1038/nbt. 3803.
44. Wang Y, Zhao D, Sun L, et al.; Engineering of the Translesion DNA Synthesis Pathway Enables Controllable C-to-G and C-to-A Base Editing in Corynebacterium glutamicum. ACS Synth Biol 2022; 11 (10): 3368-3378. doi: 10.1021/acssynbio. 2c00265.
45. Sun N, Zhao D, Li S, et al.; Reconstructed glycosylase base editors GBE2.0 with enhanced C-to-G base editing efficiency and purity. Mol Ther 2022; 30 (7): 2452-2463. doi: 10.1016/j. ymthe. 2022.03.023.
46. Tong H, Huang J, Xiao Q, et al.; High-fidelity Cas13 variants for targeted RNA degradation with minimal collateral effects. Nat Biotechnol 2023; 41 (1): 108-119. doi: 10.1038/s41587-022-01419-7.
47. Chen S, Zhou Y, Chen Y, et al.; fastp: an ultra-fast all-in-one FASTQ preprocessor. Bioinformatics 2018; 34 (17): i884-i890. doi: 10.1093/bioinformatics/bty560.
48. Clement K, Rees H, Canver M C, et al.; CRISPResso2 provides accurate and rapid genome editing sequence analysis. Nat Biotechnol 2019; 37 (3): 224-226. doi: 10.1038/s41587-019-0032-3.

### Example 5. Development of Orthogonal Base Editors Based on Engineered Glycosylases

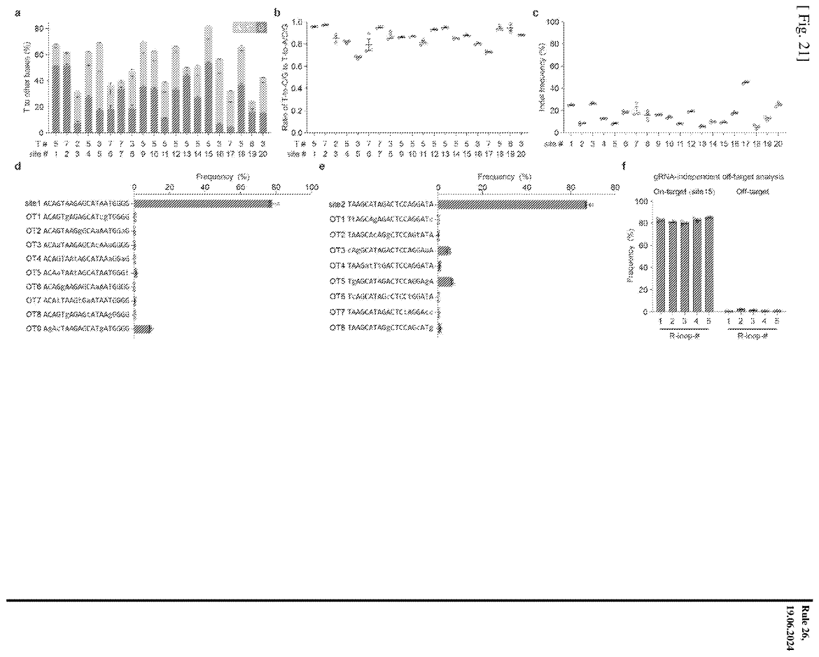

Encouraged by the development of gGBE in the above Examples, thymine and cytosine base editor were developed using the deaminase-free glycosylase-based strategy. Since the three pyrimidine bases (i.e., T, C, and U) are structurally similar, it was speculated that the excision of canonical T or C could be achieved by engineering certain uracil DNA glycosylase (UNG). The excision of T or C would generate an apurinic/apyrimidinic (AP) site, then trigger base excision repair (BER) pathway and facilitate direct T editing or C editing (FIG. 19a-b).

Alternative splicing as well as transcription from two distinct start sites leads to two different human UNG isoforms, mitochondrial UNG1 (304 amino acids, aa) (SEQ ID NO: 54) and nuclear UNG2 (313 aa) (SEQ ID NO: 133), each possessing an unique N-terminus that mediates translocation to mitochondria or nucleus¹⁶ (FIG. 25). The sequence alignment of human UNG1 and human UNG2 shows that the amino acid residues at positions 1-35 of UNG1 are different from the amino acid residues at positions 1-44 of UNG2, and the remaining parts of UNG1 and UNG2 are identical.

Two human UNG1 variants, UNG1-Y147A and UNG1-N204D, have been engineered to excise T and C in DNA, respectively¹⁷. The residues Y156 and N213 of UNG2 are corresponding to the residues Y147 and N204 of UNG1, respectively, as determined by sequence alignment of UNG1 and UNG2. To edit nuclear DNA, two prototype gBEs, a deaminase-free glycosylase-based thymine base editor (gTBE) (gTBEv0.1; SEQ ID NO: 136) and a deaminase-free glycosylase-based cytosine base editor (gCBE) (gCBEv0.1; SEQ ID NO:138), were generated by fusing UNG2-Y156A (SEQ ID NO: 135) and UNG2-N213D (SEQ ID NO: 137) (wherein Y156A and N213D of human UNG2 are equivalent to Y147A and N204D of human UNG1, respectively) (with its N-termial Met retained) at the C-terminus of Cas9-D10A nickase (nCas9) (SEQ ID NO:2) via a linker (SEQ ID NO: 134), respectively (FIG. 19a-c).

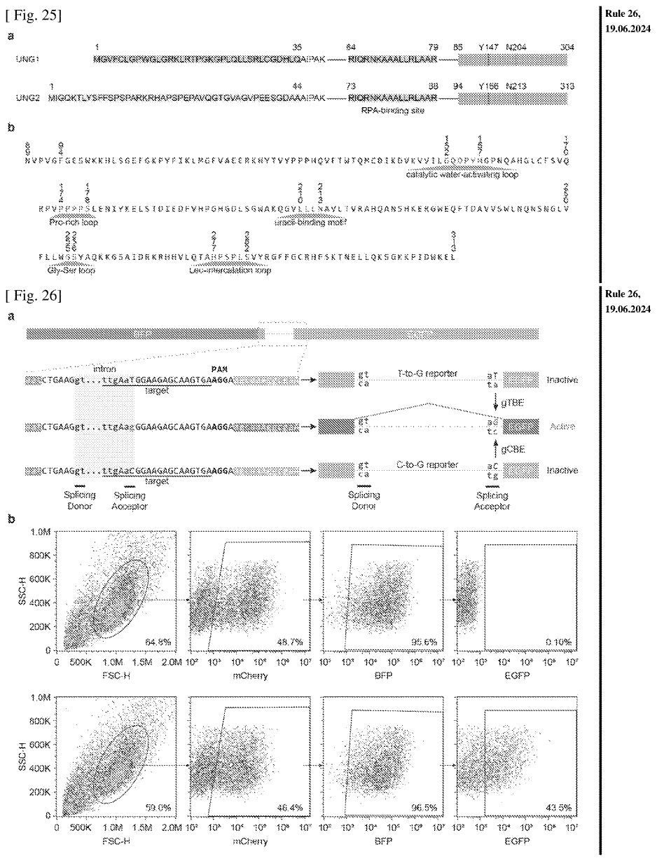

T-to-G reporter and C-to-G reporter, two similar intron-split EGFP reporter systems as reported previously⁹, were established to evaluate the editing efficiency of gTBE and gCBE, respectively (FIG. 26a). In these reporters, the AG-to-AT or AG-to-AC inactive splicing acceptor (SA) could only be remediated with T-to-G or C-to-G conversion, which leads to correct splicing of EGFP-coding sequence and EGFP expression and emission of EGFP signals (FIG. 26b). The gTBE or gCBE encoding vector was co-transfected with the T-to-G or C-to-G reporter vector containing a targeting single-guide RNA (targeting sgRNA) that targeted the corresponding mis-splicing mutations.

It was observed that as compared to a negative control, gTBEv0.1 showed T-to-G conversion activity, and gCBEv0.1 showed C-to-G conversion activity (FIG. 19c-e).

Given the disordered N-terminal domain (NTD) of UNG contains protein binding motifs and sites for post-translational modifications¹⁸, which might constrain targeted excision activity of the glycosylase domain in ssDNA^{19,20}, gTBEv0.2 (SEQ ID NO: 140) (with UNG2Δ88-Y156A (SEQ ID NO: 139) fused at the C-terminus of nCas9) and gCBEv0.2 (SEQ ID NO: 142) (with UNG2Δ88-N213D (SEQ ID NO: 141) fused at the C-terminus of nCas9) were constructed (FIG. 19c) by deleting 88 amino acid residues (denoted as “Δ88”) at the N-terminal of UNG2 to eliminate potential undesired protein-protein interactions²⁰⁻²². As noted, with the N-terminal deletion of 88 amino acid residues, the remaining part of human UNG2 would be identical to the remaining part of human UNG1 with corresponding N-terminal deletion of 79 amino acid residues, and in such a case, both UNG2 and UNG1 may be termed as UNG unless it is necessary for the context to distinguish UNG2 from UNG1. Unless otherwise indicated, the amino acid mutation on UNG is numbered based on full length human UNG2.

gTBEv0.2 exhibited comparable T-to-G conversion activity with gTBEv0.1 (1.0% vs. 1.1%, FIG. 19d), while gCBEv0.2 exhibited significantly increased C-to-G conversion activity compared with gCBEv0.1 (13.3% vs. 1.0%, FIG. 19e). Moreover, both gTBEv0.3 (SEQ ID NO: 143) (with UNG2Δ88-Y156A fused at the N-terminus of nCas9) and gCBEv0.3 (SEQ ID NO: 154) (with UNG2Δ88-N213D fused at the N-terminus of nCas9) showed much higher editing efficiency than gTBEv0.2 and gCBEv0.2 with UNG2 mutant fused at the C-terminus of nCas9 (10.2% vs. 1.0% for gTBE and 51.4% vs. 13.3% for gCBE; FIG. 19c-e), about 10- and 3.9-fold enhancement in editing efficiency, respectively. This indicated that it would be desired to have N′-UNG-nCas9-C′ configuration for both gTBE and gCBE. Unless otherwise indicated, this configuration was used in all the subsequent Examples.

For negative control, no editing efficiency was found for all the above-mentioned gTBE and gCBE together with a nontargeting sgRNA (FIG. 19d-e).

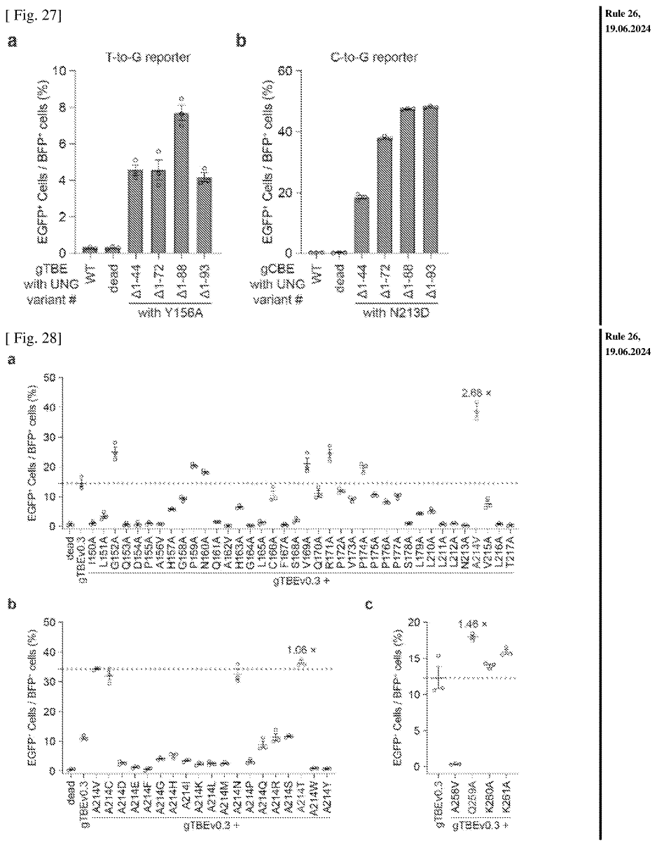

In addition, various versions of N-terminal deletion were applied to UNG2 of gTBE and gCBE. For gTBE, it was demonstrated that the deletion of 88 amino acid residues in gTBEv0.3 achieved significantly increased editing efficiency than the deletion of 44, 72, and 93 amino acid residues; and for gCBE, it was demonstrated that the deletion of 88 amino acid residues in gCBEv0.3 achieved comparable editing efficiency to the deletion of 93 amino acid residues and significantly increased editing efficiency than the deletion of 44 and 72 amino acid residues (FIG. 27).

Furthermore, the orthogonality of gTBE and gCBE for base editing was examined. Although engineered from the same original DNA glycosylase UNG, no C editing efficiency was observed for gTBEv0.3 and no T editing efficiency was observed for gCBEv0.3 (FIG. 19f). Thus, two orthogonal base editors were developed, gTBE for direct T editing and gCBE for C editing.

### Example 6. Evolution of gTBE with Enhanced Editing Efficiency

To further increase the T-to-G conversion activity of gTBEv0.3, rational mutagenesis was performed for engineering the UNG moiety, using the T-to-G reporter to evaluate the editing efficiency in cultured mammalian cells (HEK293T cells) (FIG. 20a). Based on structural and functional analysis, WT UNG contains five conserved motifs required for efficient glycosylase activity: the catalytic water-activating loop, the proline-rich loop, the uracil-binding motif, the glycine-serine motif, and the leucine loop²³⁻²⁵ (FIG. 25b). Since Y156 in the catalytic water-activating loop and N213 in the uracil-binding motif are critical for activity switch from U excision to T or C excision, sequential and spatial neighbors of these two residues were selected for examination of their roles in the regulation of base excision activity (FIG. 20a-b). Alanine-scanning mutagenesis was conducted by replacing all non-alanine with alanine (X-to-A) and alanine with valine (A-to-V) to cover all the residues in the regions of I150-L179 and L210-T217.

Interestingly, gTBEv1.1 (SEQ ID NO: 145) (v0.3 plus A214V) with UNG2Δ88-Y156A+A214V (SEQ ID NO: 144) was obtained with largely elevated T-to-G conversion activity of about 2.68-fold as compared with gTBEv0.3 (FIG. 28a). To check whether there was any amino acid at position 214 that would perform better than valine, site-saturation mutagenesis focusing on the residue at position 214 was further performed. gTBEv1.2 (SEQ ID NO: 147) (v0.3 plus A214T) with UNG2Δ88-Y156A+A214T (SEQ ID NO:146) was obtained with elevated editing efficiency of about 1.06-fold in comparison with the T editing efficiency of gTBEv1.1 (FIG. 28b).

On the other hand, the spatial neighbors of A214, nearby the Gly-Ser loop that compresses the DNA backbone 3′ to the lesion (FIG. 20b), was examined, and gTBEv1.3 (SEQ ID NO: 149) (v0.3 plus Q259A) with UNG2Δ88-Y156A+Q259A (SEQ ID NO: 148) was obtained with increased editing efficiency of about 1.46-fold as compared with gTBEv0.3 (FIG. 28c).

Furthermore, a synergistic enhancement of T-to-G editing efficiency of about 2.7-fold in comparison with the T editing efficiency of gTBEv0.3 was found for gTBEv2 (SEQ ID NO: 151) (v0.3 plus combination of A214T and Q259A) with UNG2Δ88-Y156A+A214T+Q259A (SEQ ID NO: 150) (FIG. 20c).

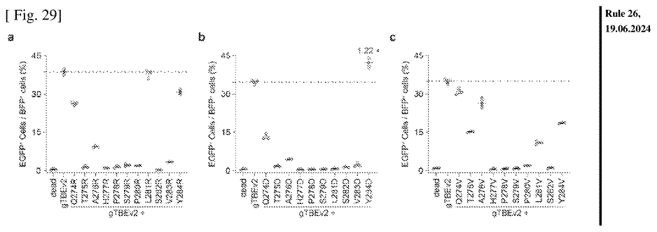

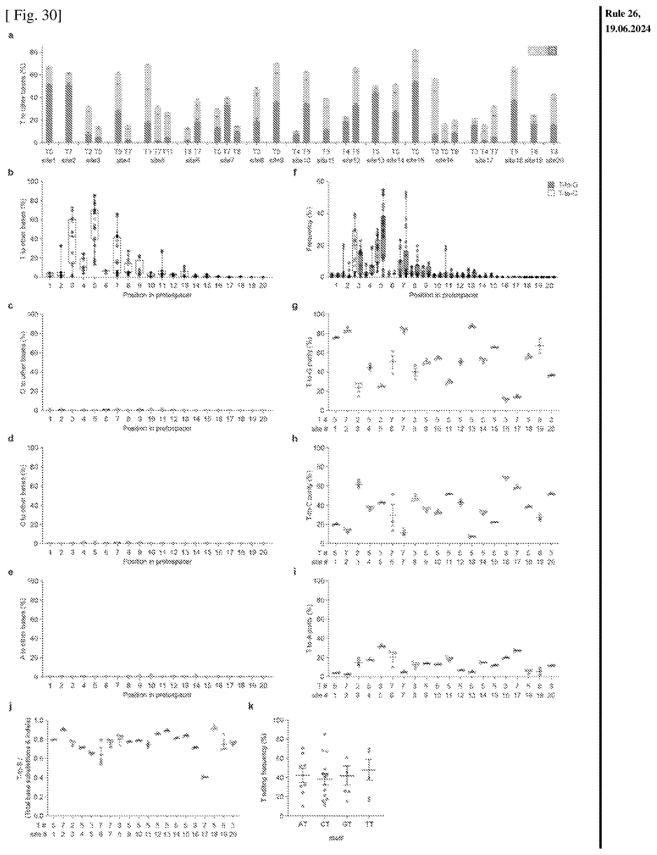

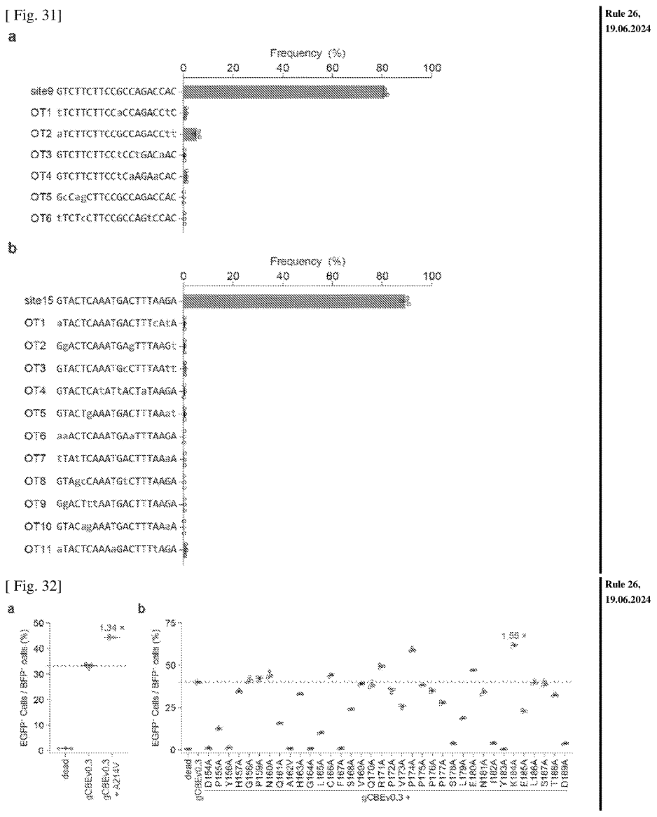

The residues in the regions of Q274-Y284, in or nearby the Leu-intercalation loop, was also scanned by sequential replacement with amino acids of distinct properties, including arginine (with positive charged side chain), aspartic acid (with negative charged side chain), or valine (with small hydrophobic side chain) (X-to-R, D, or V). gTBEv3 (SEQ ID NO: 153) (v2 plus Y284D) with UNG2Δ88-Y156A+A214T+Q259A+Y284D (SEQ ID NO: 152) was found to show elevated editing efficiency of about 1.22-fold as compared with gTBEv2 (FIG. 29b) and of about 3.09-fold as compared with gTBEv0.3 (FIG. 20c).

The improvement of T editing efficiency across different gTBE was validated at one endogenous genomic site in cultured mammalian cells (HEK293T cells). After transfected with all-in-one constructs encoding each gTBE, together with a targeting sgRNA that targeted site 9 in CLYBL gene and mCherry for fluorescence-activated cell sorting (FACS), mCherry-positive cells were FACS-sorted. Through target deep sequencing analysis, a gradual increase of overall T editing efficiency was obtained at T5 from 26.9% for gTBEv1.1 to 67.4% for gTBEv3, confirming that gTBEv3 was the best version for base editing at endogenous targets, with T-to-S (i.e., T-to-C or T-to-G; S=C or G base) conversions as the predominant events at this site (FIG. 20d).

These results indicate that the mutagenesis described above effectively optimized the performance of gTBE for T-to-C and T-to-G base editing. gTBEv3 having the highest T editing efficiency was used in the subsequent Examples.

### Example 7. Characterization of gTBEv3 at Human Genomic DNA Sites

The editing profiles of gTBEv3 was characterized by targeting 20 endogenous genomic loci, most of which were used in previous base editing studies^{11, 12, 26, 27}. It was found that gTBEv3 achieved efficient T base editing efficiency (ranged from 24.3% to 81.5%; FIG. 21a and FIG. 30a-b), and essentially no A, C, or G editing at all the examined sites (FIG. 30c-e). The T-to-C or T-to-G conversions were the predominant events (FIG. 30f-h), only a low percentage of T-to-A conversion were detected (FIG. 21a and FIG. 30i), consistent with the previous findings for gGBE³, AYBE⁹ and CGBEs¹¹⁻¹⁵. The ratios of T-to-Sto T conversion ranged from 0.68 to 0.97 (without indels, FIG. 21b) and from 0.41 to 0.92 (with indels, FIG. 30j). It was found that gTBEv3 also induced indels with frequency ranging from 5.2% to 45.2% at the 20 edited sites (FIG. 21c). Furthermore, it was found that the editable range of gTBEv3 was positions 2 to 11, and the optimal editing window with high efficiency of T conversion covered protospacer positions 3 to 7, with the highest editing efficiency at position 5 (FIG. 30b). No obvious motif preference was found for T conversions with gTBEv3 by analyzing the on-target editing and sequences of all the tested sites (FIG. 30k).

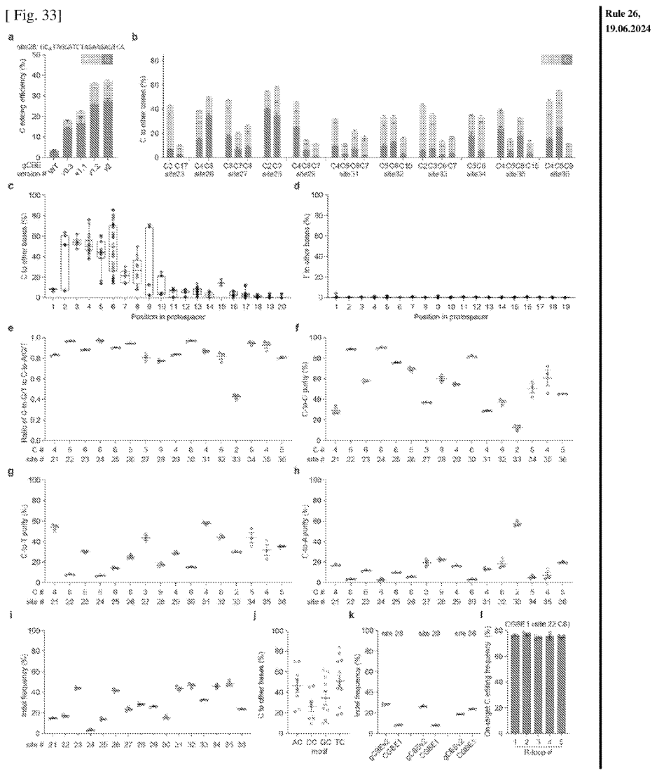

The off-target activity of gTBEv3 was analyzed at several in silico-predicted²¹ guide sequence-dependent off-target sites, and the ability of gTBEv3 to mediate guide sequence-independent off-target DNA editing was characterized by using orthogonal R-loop assay in five previously reported dSaCas9 R-loops^{9,29}. Very low percentage of editing was found at all the guide sequence-dependent off-target loci (FIG. 21d-e and FIG. 31), and very low frequencies (1.1% in average) was detected at all five guide sequence-independent off-target sites (FIG. 21f). Taken together, gTBEv3 represents a highly efficient T-to-S base editor with low off-target effects in mammalian cells.

### Example 8. Enhancement of C Editing Efficiency of gCBE

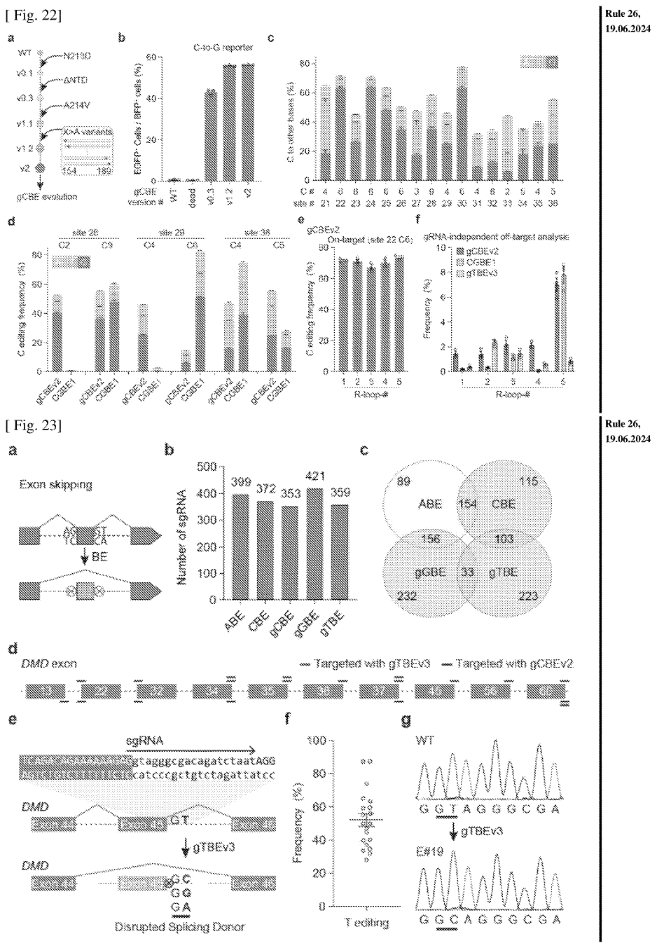

To examine whether the mutations emerged from the engineering of gTBE would benefit the enhancement of gCBE activity, gCBEv1.1 was generated by introducing A214V into gCBEv0.3 (FIG. 22a). It was found that gCBEv1.1 (SEQ ID NO: 156) with UNG2Δ88-N213D+A214V (SEQ ID NO: 155) had largely elevated C-to-G conversion activity of about 1.34-fold as compared to gCBEv0.3 when evaluated using the C-to-G reporter (FIG. 32a).

On the other hand, alanine-scanning mutagenesis was conducted on the region of D154-D189 of UNG2 to examine its role in the regulation of base excision activity, and gCBEv1.2 (SEQ ID NO: 158) (v0.3 plus K184A) with UNG2Δ88-N213D+K184A (SEQ ID NO: 157) was obtained with largely elevated C-to-G conversion activity by about 1.55-fold as compared with gCBEv0.3 (FIG. 32b).

The combination of A214V and K184A was further investigated by combining these two mutations to generate gCBEv2 (SEQ ID NO: 160) with UNG2Δ88-N213D+K184A+A214V (SEQ ID NO: 159), achieving C-to-G editing efficiency of about 1.3-fold compared with gCBEv0.3 (FIG. 22b). The improvement of C editing efficiency across different gCBE was further validated by targeting an endogenous genomic site, and a gradual increase of overall C editing efficiency from 18.2% to 37.2% at C2 of the site 28 was observed (FIG. 33a).

By targeting 16 endogenous genomic loci, the editing profiles of gCBEv2 was characterized, showing efficient C base editing efficiency ranging from 31.8% to 77.7% (FIG. 22c and FIG. 33b-d). It was found that gCBEv2 induced predominant C-to-G conversions as well as C-to-T conversions, with the ratios of C-to-G/T to C-to-A/G/T conversion reaching up to 0.97, and there were very few C-to-A conversions detected (FIG. 22c, FIG. 33e-h). gCBEv2 induced indels with frequency ranged from 3.1% to 48.3% at the examined sites (FIG. 33i). After analyzing the sequences of all the tested sites, it was found that the editable range of gCBEv2 was positions 2 to 9 (FIG. 33c), and gCBEv2 showed preferences for editing at AC or TC motifs with a higher efficiency than other motifs (FIG. 33j).

When compared to CGBE1¹², a C-to-G base editor, it was found that gCBEv2 showed higher editing efficiency at certain positions towards the distal end of the target sequence (FIG. 22d and FIG. 33c), indicating its positional preference within different optimal editing windows (positions 2 to 6 for gCBEv2 vs. positions 5 to 7 for CGBE1¹²). gCBEv2 induced fewer indels at site 36, and more indels at site 28 and site 29 than CGBE1 (FIG. 33k). To be noted, using the orthogonal R-loop assay^{9, 29} mentioned above, it was found that gCBEv2 showed comparably frequencies with CGBE1 at two guide sequence-independent off-target sites (FIG. 22e-f and FIG. 33l).

Moreover, it was found that gCBEv2 could solely facilitate C editing, and there was essentially no T editing at all the examined sites (FIG. 33c-d). The editing specificity of gCBEv2, together with that of gTBEv3 (FIG. 30b-e), consolidated the orthogonality of these two base editors for direct base editing.

### Example 9. Applications of gTBE and gCBE

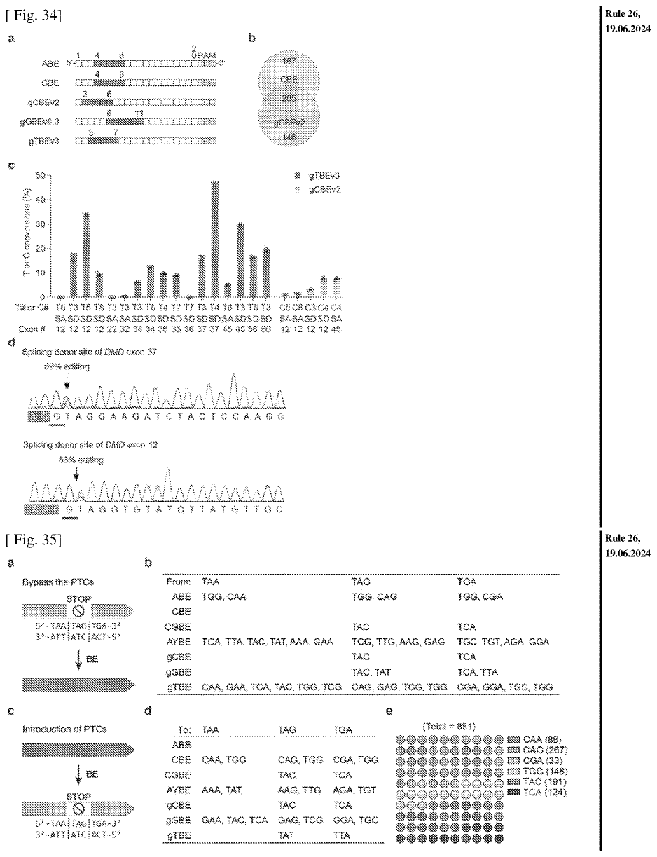

The potential applications of gTBE and gCBE were further evaluated. The gTBE could not only remediate inactive splicing signals in the intron-split EGFP reporter systems used above (FIG. 19-20 and FIG. 26), but also be used for exon skipping by disrupting splicing signals at splicing donor (SD) or splicing acceptor (SA) sites (FIG. 23a). After analyzing the splicing sites in 16 well-studied genes for gene and cell therapy research³⁰⁻³², it was found that gTBE and gCBE, together with other existing base editors, provide 1904 sgRNA candidates (protospacer sequence/guide sequence shown in Table 3) with the SD or SA sites located in each optimal editing window (FIG. 23b and FIG. 34a). Among the 771 sgRNA candidates for ABE and CBE targeting, 156 and 103 candidates overlapped with those for gGBE and gTBE, respectively (FIG. 23c). Moreover, 232 and 223 sgRNA candidates could only be screened by gGBE or gTBE targeting, respectively (FIG. 23c). For gCBE, apart from 205 sgRNA candidates overlapped with those for CBE, there were 148 unique sgRNA candidates (FIG. 34b). The availability of these base editors could largely expand the scope of guide RNA screening for efficient editing at splicing sites (FIG. 34). In addition, these newly developed base editors could be utilized for bypassing premature termination codons (PTCs) and introduction of PTCs (FIG. 35). The gTBE and gCBE could provide more versatile codon outcomes from PTCs editing (FIG. 35b) and introduce PTCs by editing more codons encoding various amino acids (FIG. 35d). To potentially disrupt gene function by introduction of PTCs, 851 sgRNA candidates (protospacer sequence/guide sequences shown in Table 4) targeting various codons for PTCs introduction in 15 genes were analyzed with gGBE and CBE, with 191 TAC and 124 TCA for gGBE targeting (FIG. 35e).

To illustrate these applications, editing the splicing sites in human DMD gene (Duchenne muscular dystrophy, encoding dystrophin) that cannot be targeted with ABE or CBE was developed. A series of sgRNAs specifically targeting SD or SA sites was designed and screened with gTBEv3 or gCBEv2 (FIG. 23d and FIG. 34c), including three sgRNAs targeting the SD sites of DMD exon 45 (FIG. 23e), 12, and 37 (FIG. 34d) uniquely targeted by gTBEv3. Disruption of the SD site of exon 45, thus leading to exon skipping, would be applicable to restore dystrophin expression in 9% DMD patients³³. Thus, gTBEv3-encoding mRNA and sgRNA targeting the SD site of DMD exon 45 were co-injected into zygotes of humanized mice to explore the potential application of gTBE. It was found that 100% (20/20) mouse embryos harbored efficient base conversion (ranged from 35.0% to 97.0%) at the desired position T3 (FIG. 23f-g), indicating the great potential of gTBE for human disease modeling and gene therapy. Overall, gBEs, including gTBE, gCBE, and gGBE, provide more options for the sites that deaminase-based base editors could not target, largely expanding the targeting scope of base editors.

### Example 10. Comparison of Different Base Editing Systems

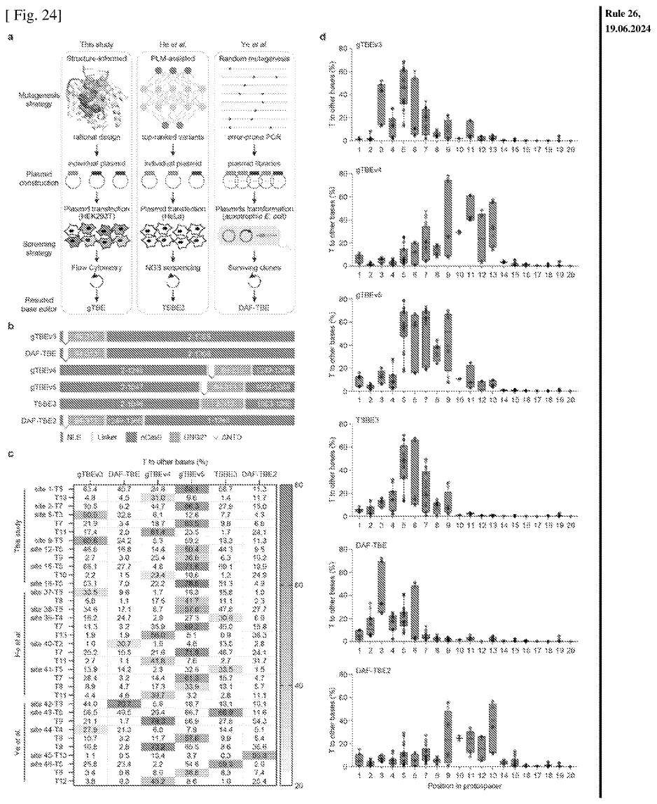

gTBEv4 (SEQ ID NO: 161) and gTBEv5 (SEQ ID NO: 162) were generated by inserting the UNG2 mutant (SEQ ID NO: 152) contained in gTBEv3 (SEQ ID NO: 153) into split nCas9 domains at different locations (FIG. 24b). For gTBEv4 (SEQ ID NO: 161), the UNG2 mutant (SEQ ID NO: 152) was embedded between positions 2-1248 of nCas9 (SEQ ID NO: 2) and positions 1249-1368 of nCas9 (SEQ ID NO: 2). For gTBEv5 (SEQ ID NO: 162), the UNG2 mutant (SEQ ID NO: 152) was embedded between positions 2-1047 of nCas9 (SEQ ID NO: 2) and positions 1064-1368 of nCas9 (SEQ ID NO: 2). Note that the first amino acid residue D of nCas9 (SEQ ID NO: 2) was numbered as position 2 instead of position 1.

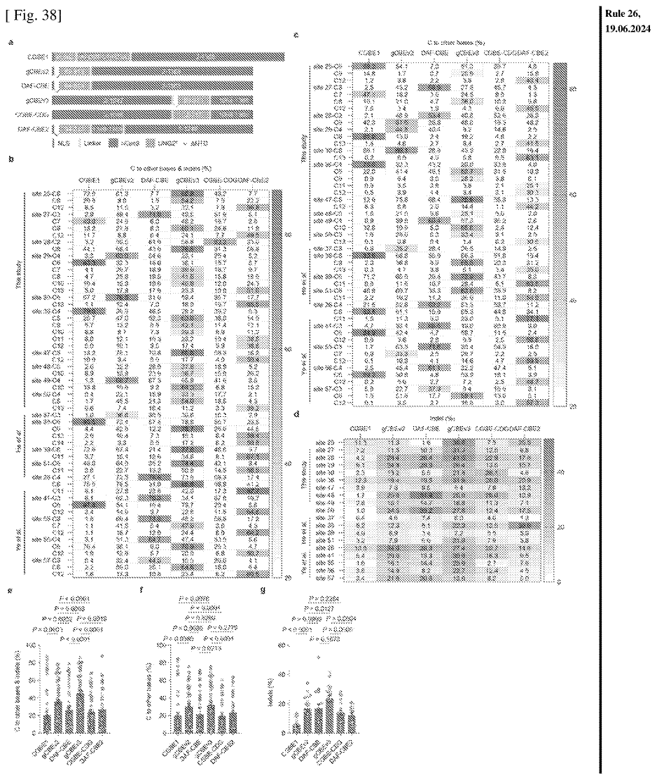

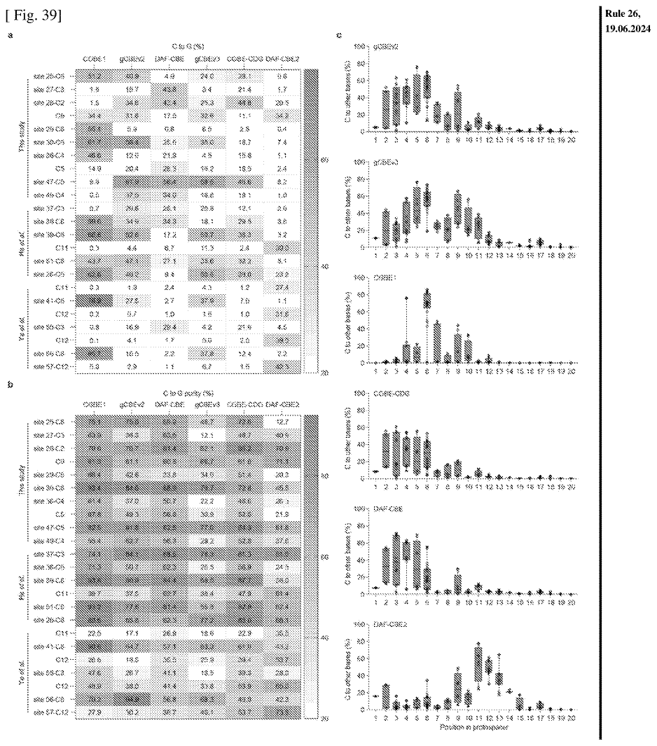

Similarly, gCBEv3 (SEQ ID NO: 164) (FIG. 38a) was generated by replacing the UNG mutant (SEQ ID NO: 152) in gTBEv5 (SEQ ID NO: 162) with the UNG mutant (SEQ ID NO: 159) in gCBEv2 (SEQ ID NO: 160).

To better characterize the performance of various deaminase-free base editors, a side-by-side comparison of base editors in this study and those from the other two studies in He et al. developing a TSBE3 for T-to-G/C substitutions using protein language model (PLM)-assisted strategy³⁴ and Ye et al. conducting rounds of random mutagenesis by error-prone PCR for directed evolution in Escherichia coli and obtaining several deaminase-free base editors (DAF-TBEs and DAF-CBEs)³⁵ were made (FIG. 24a). The basic architectures of the above-mentioned base editors are different, for instance, TSBE3 was constructed using an embedding strategy and DAF-TBE2 using a circularly permuted strategy (FIG. 24b).

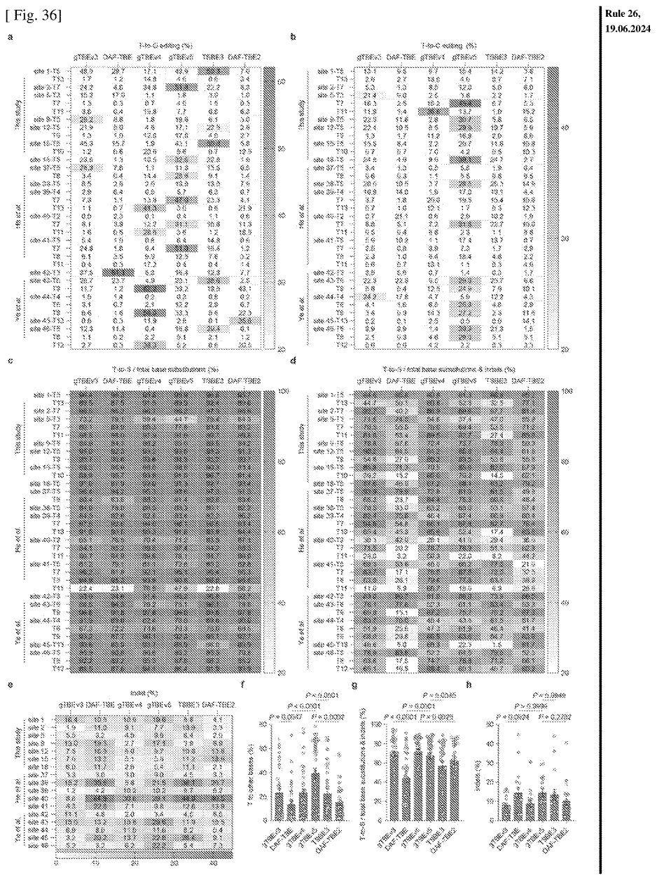

The T editing efficiency of various thymine base editors was compared at 17 endogenous sites, including five sites from He's study³⁴ and five sites from Ye's study³⁵ (FIG. 24c and FIG. 36). For the base editors with UNG mutants fused at the N-terminus of nCas9, gTBEv3 showed higher editing efficiency than DAF-TBE at the overwhelming majority of Ts (29 out of 35) of tested sites (FIG. 24c, FIG. 36f), indicating that UNG mutants generated herein by rational mutagenesis are superior to those by random mutagenesis.

gTBEv3 was also compared with gTBEv4 and gTBEv5, two base editors constructed using the embedding strategy. gTBEv4 showed a shifted editing window of positions 7-13 from positions 3-7 (FIG. 24d), with no significant difference in average editing efficiency from gTBEv3 (23.2% vs. 23.1%, FIG. 36f). For gTBEv5, the editing efficiency was largely increased compared to that of gTBEv3 (averaging 39.3% vs. 23.1%, FIG. 36f) and gTBEv4 and others, with the same predominant T-to-S conversions (FIG. 36a-d and g), and the optimal editing window covered protospacer positions 5 to 9 (FIG. 24d).

TSBE3 (carrying L83Q and G116E mutations, equivalent to L74Q and G107E in UNG1) is an nCas9-embedded base editor with almost the same insertion position as gTBEv5 (FIG. 24c). gTBEv5 showed higher editing efficiency than TSBE3 (39.3% vs. 22.5%, FIG. 36f) at the overwhelming majority of Ts (29 out of 35) of the tested sites (FIG. 24c), indicating that the UNG mutant generated herein by rational mutagenesis are superior to those generated by PLM-assisted mutagenesis. The optimal editing window of TSBE3 covered protospacer positions 4 to 9 (FIG. 24d).

The circularly permuted DAF-TBE2 showed low average editing efficiency and an editing window of positions 9-13, different from the editing window (positions 2-6) of DAF-TBE (FIG. 24d).

Despite showing the highest average editing efficiency, gTBEv5 induced comparable indel rates to that of DAF-TBE (14.4% vs. 14.4%), DAF-TBE2 (14.4% vs. 10.3%), and TSBE3 (14.4% vs. 13.5%, FIG. 36e-g). To be noted, gTBEs induced much fewer unintended T editing than TSBE3 and DAF-TBEs in the proximal DNA sequence upstream from two sites (site 38 and site 44) harboring unintended edits (FIG. B13), consistent with the finding that the NTD of UNG could promote targeting the enzyme to ssDNA-dsDNA junctions¹⁹.

Similarly, the C editing efficiency of various base editors (FIG. 38a) were compared at 19 endogenous sites, including five sites from He's study³⁴ and five sites from Ye's study³⁵ (FIG. 38b-d). It was found that both gCBEv2 and gCBEv3 showed higher overall average editing efficiency than all the other base editors (FIG. 38e-f), especially for gCBEv3. gCBEv2 outperformed DAF-CBE (30.1% vs. 21.3%) and CGBE-CDG (30.1% vs. 19.3%) for the average efficiency of base conversion (FIG. 38e-f), indicating that the UNG mutant generated herein by rational mutagenesis are superior to those by random mutagenesis. gCBEv2 induced comparable average indel rates with other deaminase-free base editors, including DAF-CBE (16.8% vs. 16.9%), DAF-CBE2 (16.8% vs. 12.1%), and CGBE-CDG (16.8% vs. 13.6%, FIG. 38d-g). The C-to-G editing frequency and purity of different base editors showed respective advantages for CGBE1 and various deaminase-free base editors at different cytosine position across the protospacer (FIG. 39a-b). Each base editor can edit its target base within a certain editable window, that is, positions 2 to 9 for gCBEv2, positions 2 to 11 for gCBEv3, positions 4 to 10 for CGBE1, positions 2 to 9 for CGBE-CDG, positions 2 to 9 for DAF-CBE, and positions 9 to 12 for DAF-CBE2 (FIG. 39c).

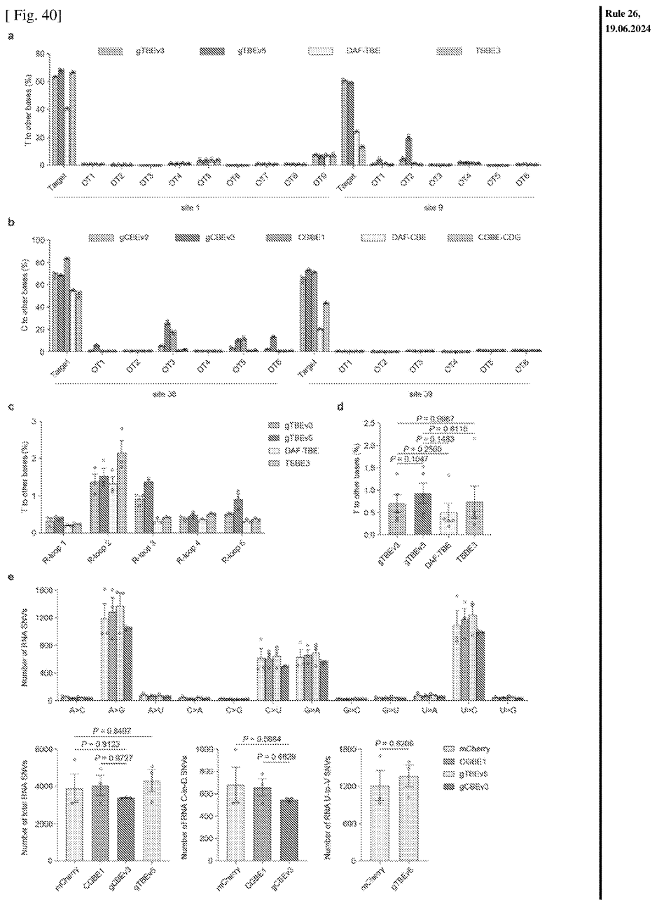

By analyzing the off-target effects both at some guide sequence-dependent and guide sequence-independent off-target sites, it was found that gTBEs and gCBEs induced comparable low-level off-target edits similar to that of other base editors at most sites (FIG. 40a-c). Moreover, by performing transcriptome-wide RNA analysis, it was found that gTBEv5 and gCBEv3 did not exhibit significant off-target RNA editing or impact the cell's inherent DNA repair processes (FIG. 40d), consistent with those of DAF-TBE, DAF-CBE, CGBE-CDG, and TSBE3^{34,35}.

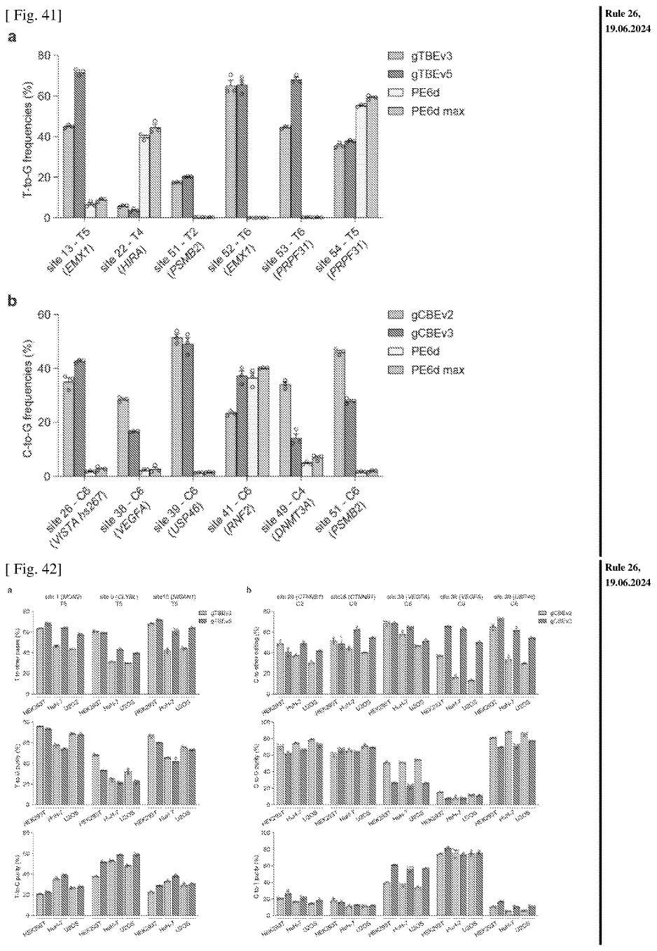

Prime editing (PE) system could theoretically mediate all types of base substitution, including T-to-G conversion and C-to-G conversion³⁹. gTBEv3 and gTBEv5 were compared with the recently evolved PE6d system⁴⁰ at six previously reported endogenous sites³⁵ in HEK293T cells. gTBEv3 and gTBEv5 outperformed PE6d or PE6d max for T-to-G conversion at four tested sites (FIG. 41a). gCBEv2 and gCBEv3 outperformed PE6d or PE6d max for C-to-G conversion at five tested sites (FIG. 41b). These findings indicate that base editing and prime editing offer complementary strengths, and base editors generally show more efficient editing if the target base is positioned optimally. In addition, gTBEs and gCBEs exhibited efficient T and C editing efficiency across three different human cell lines (HEK293T, U2OS, and HuH-7 cells), with slight perturbations of the product purity for gTBEs and comparable substitution frequency of certain base for gCBEs in different cell lines (FIG. 42).

Taken together, it was found that gTBEs and gCBEs in this study outperformed other base editors, including DAF-TBEs, DAF-CBE, TSBE3, and CGBE-CDG from the other two studies. And the alternative editing windows of different base editors would provide more choices for proper base conversion.

### DISCUSSION

The deaminase-based base editor (dBE) and derivatives thereof enable direct editing of adenine (A) and cytosine (C), but not thymine (T). In human, about 19% of the pathogenic single nucleotide polymorphism (SNP) could be corrected through T-to-G conversion⁹. In this study, two orthogonal base editors, gTBE and gCBE, that could achieve highly efficient T and C editing in both cultured human cells and mouse embryos were developed. The gTBE and gCBE could greatly broaden the targeting scope of base editors by breaking the limitations of PAM and narrow editing window, thus increasing the opportunity to obtain an efficient strategy for further research. The T-to-S conversion ability of gTBE allows for a variety of gene editing applications, including editing splicing sites, as well as editing that bypass PTCs.

It has been shown that the same original DNA glycosylase could be engineered into enzymes that selectively excise different specific nucleotide bases and harnessed to develop novel base editors using the deaminase-free glycosylase-based strategy of the disclosure. The enhanced editing efficiency could be attributed to the mutations in the UNG moiety that facilitate its specific substrate preference or ssDNA-binding activity, or both. The high editing efficiency of gTBEv5 indicates that insertion of UNG mutant into split nCas9 might enhance target DNA accessibility by modulate the interaction between the UNG moiety and the target DNA.

In this study, the glycosylase-mediated base editors developed in different studies were systematically compared. Structure-informed rational design was used in the disclosure, successfully engineering gTBE and gCBE for highly efficient T and C editing, respectively. He et al. utilized PLM to assist the engineering of TSBE3, while Ye et al. obtained DAF-TBE and DAF-CBE by performing random mutagenesis (FIG. 24a). It was found that gTBE/gCBE in the disclosure outperformed DAF-TBE, DAF-CBE, TSBE3, and CGBE-CDG, with higher average editing efficiency and alternative editing windows (FIG. 24c-d and FIG. 38-39).

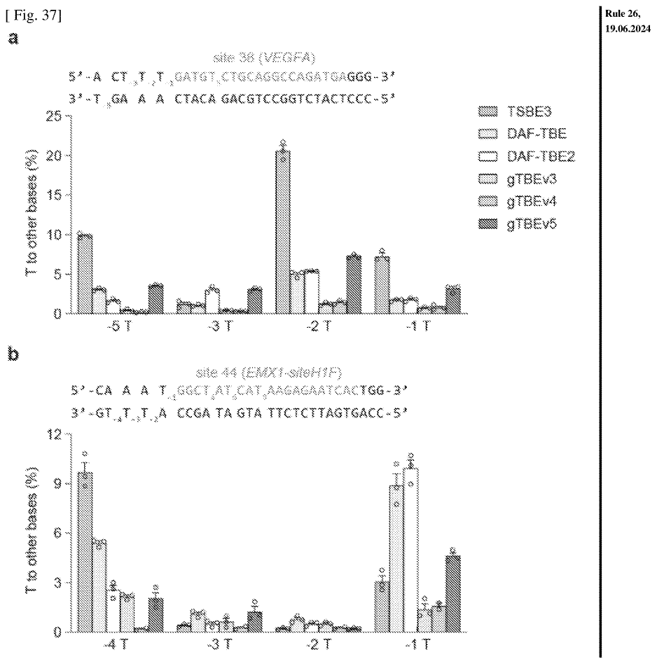

Wild-type UNG proteins are highly specific against uracil in both ssDNA and dsDNA, with a preference for ssDNA⁴³. The NTD of UNG containing motifs and sites for undesired protein-protein interactions and post-translational modifications could promote targeting the enzyme to ssDNA-dsDNA junctions^{19, 20} TSBE3, with full length UNG2, and DAF-TBEs induced more undesired edits than gTBEs in the proximal DNA sequence upstream from two sites harboring unintended edits (FIG. 37).

In summary, two orthogonal base editors based on the same original DNA glycosylase have been engineered for direct T editing and direct C editing, and structure-informed rational design represents an efficient and efficacious protein engineering strategy, providing reference and solving thought for the subsequent evolution of other proteins.

### Methods

### Molecular Cloning

Base editor constructs used in this study were cloned into a mammalian expression plasmid backbone under the control of a EF1α promoter by standard molecular cloning techniques, and the two intron-split EGFP reporters were constructed similar to those described previously⁹, except that the engineered sequence containing the last 86 base pairs (bp) intron of human RPS5 was inserted between BFP and EGFP coding sequences. And the corresponding mutations at the splice acceptor site were made to construct T-to-G reporter or C-to-G reporter via site-directed mutagenesis by PCR, respectively. Mutations at the splice acceptor site led to inactive EGFP production. Encouraged by the findings from previous base editors^{12,15}, the corresponding mutations at the splice acceptor site were put at position 6 across the protospacer.

The wild-type UNG2 sequence (313 amino acids long) (SEQ ID NO: 133) was PCR-amplified from cDNA of HEK293T, UNG2-Y156A, UNG2-N213D, UNG-NTD-truncated mutants, and corresponding combinations were constructed via site-directed mutagenesis by PCR. The UNG mutants were fused at different orientations with respect to nCas9 via Gibson Assembly method. PE6d architecture harbored a human codon-optimized RNaseH-truncated evolved and engineered M-MLV variant with R221K/N394K/H840A mutations in SpCas9. The nick sgRNA and epegRNA with tevoPreQ₁ motif were cloned into PE6d construct using Golden Gate assembly, resulting in an all-in-one plasmid. For PE6d max, the codon-optimized hMLH1dn was co-expressed with PE6d.

UNG mutagenesis libraries were designed and generated as previously described ⁵² with some modification. In brief, the region of 98-313 aa in UNG2 were divided into 8 aa long segments. BpiI-harboring mutants containing Y156A or N213D were introduced via site-directed mutagenesis by PCR. For evolution of gTBE, the regions of I150-L179, A158-K261, L210-T217, and Q274-Y284 were selected for rounds of sequential alanine/arginine/aspartic acid/valine substitutions (X-to-A, R, D, or V). And site-saturation mutagenesis of the residue 214 was conducted to check whether there is any amino acid at this position that would perform better than the valine. For evolution of gCBE, the region of D154-D189 was selected for sequential alanine substitutions (X-to-A). To cover all the residues in the corresponding segments for sequential alanine substitutions, alanine was mutated to valine (A-to-V). Oligos coding for the mutants annealed and ligated into corresponding BpiI-digested backbone vectors.

The Cas-OFFinder²⁸ was used to search for potential guide sequence-dependent off-target sites of Cas9 RNA-guided endonucleases with a maximum of 3 mismatches (with no bulges). For sgRNAs targeting DMD splicing sites with an NGN PAM, a PAM-flexible Cas9 variant SpG (SEQ ID NO: 163) was used in place of nCas9 (SEQ ID NO: 2). The sgRNA oligos were annealed and ligated into BpiI sites.

### Cell Culture, Transfection, and Flow Cytometry Analysis

HEK293T, HuH-7, and U2OS cells were cultured with DMEM (Catalog #11995065, Gibco) supplemented with 10% fetal bovine serum (Catalog #04-001-1ACS, BI) and 0.1 mM non-essential amino acids (Catalog #11140-050, Gibco) in an incubator at 37° C. with 5% CO₂.

Mutant screening was conducted in 48-well plates, with 3×10⁴ HEK293T cells per well plated in 250 μL of complete growth medium the day before transfection. Between 16 and 24 h after seeding, cells were co-transfected with 250 ng gTBE (or gCBE) plasmids, 250 ng T-to-G (or C-to-G) reporter plasmids, and 1 μg Polyethylenimine (PEI) (DNA/PEI ratio of 1:2) per well. For cell transfection of HEK293T, HuH-7, and U2OS for FACS, 5×10⁵ cells per well were plated in 12-well plates with 1 ml complete growth medium the day before transfection. After 14-16h, 2 μg all-in-one plasmids expressing gTBE or gCBE and corresponding sgRNA were transfected into cells using PEI (DNA/PEI ratio of 1:2) or FuGENE HD transfection reagent (DNA: FuGENE ratio of 1: 3; E2311, Promega). Orthogonal R-loop assays were performed as described previously^{9,29}. In brief, 1 μg of gTBE or gCBE expression plasmid with sgRNA targeting the corresponding site (with mCherry as reporter) and 1 g of dSaCas9 expression plasmid with corresponding sgRNA targeting five off-target sites to generate R-loops (with EGFP as reporter) were co-transfected into HEK293T cells using PEI (DNA/PEI ratio of 1:2). For prime editing, 2 g all-in-one plasmids containing PE6d, nick sgRNA, and epegRNA, or 1 g all-in-one plasmid and 1 g of hMLH1dn plasmid were co-transfected into cells using PEI (DNA/PEI ratio of 1:2).

At 48 h post-transfection, expression of mCherry, BFP, and EGFP fluorescence were analyzed by BD FACS Aria III or Beckman CytoFLEX S. Flow cytometry results were analyzed with FlowJo V10.5.3. The gating strategy in the identification of mCherry⁺, BFP⁺ and EGFP⁺ cells for on-target editing efficiency evaluation was supplied in FIG. 26b.

### Target Sequencing of Endogenous Sites and Data Analysis

Endogenous target sites of interest were amplified from genomic DNA as previously described⁹. Briefly, 10,000 positive cells with mCherry were isolated by FACS after 72 h of transfection, then genomic DNA was extracted and the regions of interest for target sites were amplified by PCR using site-specific primers. The purified PCR products were analyzed by Sanger sequencing (Genewiz).

Target sequencing data analysis was described in the previous paper³. In brief, the amplicons were ligated to adapters and sequencing was performed on the Illumina MiSeq platforms, then the targeted amplicon sequencing reads were processed using fastp with default parameters⁵³, and further amplicon sequencing analysis were performed by CRISPResso2⁵⁴. T-to-G purity was calculated as T-to-G editing efficiency/(T-to-C editing efficiency+T-to-G editing efficiency+T-to-A editing efficiency). T-to-S conversion ratio was calculated as (T-to-C editing efficiency+T-to-G editing efficiency)/(T-to-C editing efficiency+T-to-G editing efficiency+T-to-A editing efficiency). Protospacer sequences guide sequence are shown in Table 2.

In Vitro Transcription of gTBEv3 mRNA and DMD sgRNA

The mRNA and sgRNA preparations were performed as previously described⁹. The gTBEv3 expression plasmid was linearized by the FastDigest KpnI restriction enzyme (Catalog #FD0524, Thermo Fisher), purified using Gel Extraction Kit (Catalog #D2500-03, Omega), and used as the template for in vitro transcription (IVT) using the mMESSAGE mMACHINE T7 Ultra kit (Catalog #AM1345, Thermo Ambion). For DMD-sgRNA preparation, T7 promoter sequence was added to the sgRNA template by PCR amplification. The T7-DMD-sgRNA PCR product was purified using Gel Extraction Kit (Catalog #D2500-03, Omega) and used as the template for IVT of sgRNAs using the MEGAshortscript T7 kit (Catalog #AM1354, invitrogen). The gTBEv3-encoding mRNA and DMD-sgRNA were purified using the MEGAclear kit (Catalog #AM1908, invitrogen), eluted in RNase-free water and stored at −80° C. until use.

### Animals and Microinjection of Mouse Zygotes

Animal manipulations were consistent with those reported previously³. Experiments involving mice were approved by the Biomedical Research Ethics Committee of Center for HuidaGene Therapeutics Co. Ltd. Mice were maintained in a specific pathogen-free facility under a 12-hour dark-light cycle, and constant temperature (20-26° C.) and humidity (40-60%) maintenance.

Super ovulated humanized DMD females with human DMD exon 45 in C57BL/6 background (4 weeks old) were mated with C57BL/6 males (8 weeks old), and females from the ICR strain were used as foster mothers. Fertilized embryos were collected from oviducts 21 h post hCG injection. For zygote injection, the mixture of gTBEv3-encoding mRNA (250 ng/μL) and DMD-sgRNA (100 ng/μL) was injected into the cytoplasm of 1-cell embryo in a droplet of M2 medium using a FemtoJet microinjector (Eppendorf) with constant flow settings. The injected embryos were cultured in M16 medium with amino acids to blastocysts for three days (37° C. and 5% CO2) before genomic DNA extraction and target amplification.

### RNA Sequencing Experiments

HEK293T cells were plated in 12-well plates as above and transfected with 2 g of gTBEv5, gCBEv3, CGBE1, or mCherry plasmids using PEI (DNA/PEI ratio of 1:2). At 48 hours after transfection, around 5×10⁶ cells were collected. Total RNA was extracted with a TRIzol-based method, fragmented, and reverse transcribed to cDNAs with HiScript Q RT SuperMix according to the manufacturer's instructions. Total RNA integrity was quantified using an Agilent 2100 Bioanalyzer. The RNA-seq library was qualified using the Illumina NovaSeq 6000 platform (performed by GENEWIZ). Trimmomatic (v. 0.39-2)⁵⁵ was used to filter the RNAseq raw data. The clean reads were aligned to the hg38 reference genome with Hisat2 (v. 2.2.1)⁵⁶. RNA editing sites were calculated using REDItools2⁵⁷ with default parameters. The dbSNP (v. 146) database downloaded from NCBI was used to filter the sites overlapped with common single nucleotide variants (SNVs). The sites with less than five mutated or nonmutated reads were further filtered.

StringTie⁵⁸ was used to calculate expression value. DESeq2⁵⁹ was used to calculate differentially expressed genes with FDR<0.05 and Fold change>1.

### Statistical Analysis

Statistical tests performed by Graphpad Prism 8 included the two-tailed unpaired two-sample t-test or Dunnett's multiple comparisons test after one-way ANOVA.

### REFERENCES

1. Porto, E. M., Komor, A. C., Slaymaker, I. M. & Yeo, G. W. Base editing: advances and therapeutic opportunities. Nat Rev Drug Discov 19, 839-859 (2020).
2. Rees, H. A. & Liu, D. R. Base editing: precision chemistry on the genome and transcriptome of living cells. Nat Rev Genet 19, 770-788 (2018).
3. Tong, H. et al. Programmable deaminase-free base editors for G-to-Y conversion by engineered glycosylase. Natl Sci Rev 10, nwad143 (2023).
4. Gaudelli, N. M. et al. Programmable base editing of A*T to G*C in genomic DNA without DNA cleavage. Nature 551, 464-471 (2017).
5. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
6. Mok, B. Y. et al. A bacterial cytidine deaminase toxin enables CRISPR-free mitochondrial base editing. Nature 583, 631-637 (2020).
7. Lei, Z. et al. Mitochondrial base editor induces substantial nuclear off-target mutations. Nature 606, 804-811 (2022).
8. Zhang, X. et al. Dual base editor catalyzes both cytosine and adenine base conversions in human cells. Nat Biotechnol 38, 856-860 (2020).
9. Tong, H. et al. Programmable A-to-Y base editing by fusing an adenine base editor with an N-methylpurine DNA glycosylase. Nat Biotechnol 41, 1080-1084 (2023).
10. Chen, L. et al. Adenine transversion editors enable precise, efficient A*T-to-C*G base editing in mammalian cells and embryos. Nat Biotechnol (2023).
11. Zhao, D. et al. Glycosylase base editors enable C-to-A and C-to-G base changes. Nat Biotechnol 39, 35-40 (2021).
12. Kurt, I. C. et al. CRISPR C-to-G base editors for inducing targeted DNA transversions in human cells. Nat Biotechnol 39, 41-46 (2021).
13. Koblan, L. W. et al. Efficient C*G-to-G*C base editors developed using CRISPRi screens, target-library analysis, and machine learning. Nat Biotechnol 39, 1414-1425 (2021).
14. Chen, L. et al. Programmable C: G to G: C genome editing with CRISPR-Cas9-directed base excision repair proteins. Nat Commun 12, 1384 (2021).
15. Yuan, T. et al. Optimization of C-to-G base editors with sequence context preference predictable by machine learning methods. Nat Commun 12, 4902 (2021).
16. Nilsen, H. et al. Nuclear and mitochondrial uracil-DNA glycosylases are generated by alternative splicing and transcription from different positions in the UNG gene. Nucleic Acids Res 25, 750-755 (1997).
17. Kavli, B. et al. Excision of cytosine and thymine from DNA by mutants of human uracil-DNA glycosylase. EMBO J 15, 3442-3447 (1996).
18. Rodriguez, G. et al. Disordered N-Terminal Domain of Human Uracil DNA Glycosylase (hUNG2) Enhances DNA Translocation. ACS Chem Biol 12, 2260-2263 (2017).
19. Weiser, B. P., Rodriguez, G., Cole, P. A. & Stivers, J. T. N-terminal domain of human uracil DNA glycosylase (hUNG2) promotes targeting to uracil sites adjacent to ssDNA-dsDNA junctions. Nucleic Acids Res 46, 7169-7178 (2018).
20. Perkins, J. L. & Zhao, L. The N-terminal domain of uracil-DNA glycosylase: Roles for disordered regions. DNA Repair (Amst) 101, 103077 (2021).
21. Nagelhus, T. A. et al. A sequence in the N-terminal region of human uracil-DNA glycosylase with homology to XPA interacts with the C-terminal part of the 34-kDa subunit of replication protein A. J Biol Chem 272, 6561-6566 (1997).
22. Torseth, K. et al. The UNG2 Arg88Cys variant abrogates RPA-mediated recruitment of UNG2 to single-stranded DNA. DNA Repair (Amst) 11, 559-569 (2012).
23. Schormann, N., Ricciardi, R. & Chattopadhyay, D. Uracil-DNA glycosylases-structural and functional perspectives on an essential family of DNA repair enzymes. Protein Sci 23, 1667-1685 (2014).
24. Parikh, S. S. et al. Uracil-DNA glycosylase-DNA substrate and product structures: conformational strain promotes catalytic efficiency by coupled stereoelectronic effects. Proc Natl Acad Sci USA 97, 5083-5088 (2000).
25. Parikh, S. S. et al. Base excision repair initiation revealed by crystal structures and binding kinetics of human uracil-DNA glycosylase with DNA. EMBO J 17, 5214-5226 (1998).
26. Chen, L. et al. Re-engineering the adenine deaminase TadA-8e for efficient and specific CRISPR-based cytosine base editing. Nat Biotechnol 41, 663-672 (2023).
27. Jeong, Y. K. et al. Adenine base editor engineering reduces editing of bystander cytosines. Nat Biotechnol 39, 1426-1433 (2021).
28. Bae, S., Park, J. & Kim, J. S. Cas-OFFinder: a fast and versatile algorithm that searches for potential off-target sites of Cas9 RNA-guided endonucleases. Bioinformatics 30, 1473-1475 (2014).
29. Richter, M. F. et al. Phage-assisted evolution of an adenine base editor with improved Cas domain compatibility and activity. Nat Biotechnol 38, 883-891 (2020).
30. Uddin, F., Rudin, C. M. & Sen, T. CRISPR Gene Therapy: Applications, Limitations, and Implications for the Future. Front Oncol 10, 1387 (2020).
31. Nordestgaard, B. G., Nicholls, S. J., Langsted, A., Ray, K. K. & Tybjaerg-Hansen, A. Advances in lipid-lowering therapy through gene-silencing technologies. Nat Rev Cardiol 15, 261-272 (2018).
32. Zhang, X. et al. Gene knockout in cellular immunotherapy: Application and limitations. Cancer Lett 540, 215736 (2022).
33. Bladen, C. L. et al. The TREAT-NMD DMD Global Database: analysis of more than 7,000 Duchenne muscular dystrophy mutations. Hum Mutat 36, 395-402 (2015).
34. He, Y. et al. Protein language models-assisted optimization of a uracil-N-glycosylase variant enables programmable T-to-G and T-to-C base editing. Mol Cell, Online ahead of print (2024).
35. Ye, L. et al. Glycosylase-based base editors for efficient T-to-G and C-to-G editing in mammalian cells. Nat Biotechnol, Online ahead of print (2024).
36. Li, S. et al. Docking sites inside Cas9 for adenine base editing diversification and RNA off-target elimination. Nat Commun 11, 5827 (2020).
37. Liu, Y. et al. A Cas-embedding strategy for minimizing off-target effects of DNA base editors. Nat Commun 11, 6073 (2020).
38. Nguyen Tran, M. T. et al. Engineering domain-inlaid SaCas9 adenine base editors with reduced RNA off-targets and increased on-target DNA editing. Nat Commun 11, 4871 (2020).
39. Anzalone, A. V. et al. Search-and-replace genome editing without double-strand breaks or donor DNA. Nature 576, 149-157 (2019).
40. Doman, J. L. et al. Phage-assisted evolution and protein engineering yield compact, efficient prime editors. Cell 186, 3983-4002 e3926 (2023).
41. Zuo, E. et al. Cytosine base editor generates substantial off-target single-nucleotide variants in mouse embryos. Science 364, 289-292 (2019).
42. Yan, N. et al. Cytosine base editors induce off-target mutations and adverse phenotypic effects in transgenic mice. Nat Commun 14, 1784 (2023).
43. Slupphaug, G. et al. Properties of a recombinant human uracil-DNA glycosylase from the UNG gene and evidence that UNG encodes the major uracil-DNA glycosylase. Biochemistry 34, 128-138 (1995).
44. Chen, L. et al. Engineering a precise adenine base editor with minimal bystander editing. Nat Chem Biol 19, 101-110 (2023).
45. Kim, Y. B. et al. Increasing the genome-targeting scope and precision of base editing with engineered Cas9-cytidine deaminase fusions. Nat Biotechnol 35, 371-376 (2017).
46. Huang, M. E. et al. C-to-G editing generates double-strand breaks causing deletion, transversion and translocation. Nat Cell Biol 26, 294-304 (2024).
47. Hindi, N. N., Elsakrmy, N. & Ramotar, D. The base excision repair process: comparison between higher and lower eukaryotes. Cell Mol Life Sci 78, 7943-7965 (2021).
48. Thompson, P. S. & Cortez, D. New insights into abasic site repair and tolerance. DNA Repair (Amst) 90, 102866 (2020).
49. Wang, Y. et al. Engineering of the Translesion DNA Synthesis Pathway Enables Controllable C-to-G and C-to-A Base Editing in Corynebacterium glutamicum. ACS Synth Biol 11, 3368-3378 (2022).
50. Sun, N. et al. Reconstructed glycosylase base editors GBE2.0 with enhanced C-to-G base editing efficiency and purity. Mol Ther 30, 2452-2463 (2022).
51. Komor, A. C. et al. Improved base excision repair inhibition and bacteriophage Mu Gam protein yields C: G-to-T: A base editors with higher efficiency and product purity. SCI ADV 3, eaao4774 (2017).
52. Tong, H. et al. High-fidelity Cas13 variants for targeted RNA degradation with minimal collateral effects. Nat Biotechnol 41, 108-119 (2023).
53. Chen, S., Zhou, Y., Chen, Y. & Gu, J. fastp: an ultra-fast all-in-one FASTQ preprocessor. Bioinformatics 34, i884-i890 (2018).
54. Clement, K. et al. CRISPResso2 provides accurate and rapid genome editing sequence analysis. Nat Biotechnol 37, 224-226 (2019).
55. Bolger, A. M., Lohse, M. & Usadel, B. Trimmomatic: a flexible trimmer for Illumina sequence data. Bioinformatics 30, 2114-2120 (2014).
56. Kim, D., Paggi, J. M., Park, C., Bennett, C. & Salzberg, S. L. Graph-based genome alignment and genotyping with HISAT2 and HISAT-genotype. Nat Biotechnol 37, 907-915 (2019).
57. Flati, T. et al. HPC-REDItools: a novel HPC-aware tool for improved large scale RNA-editing analysis. BMC Bioinformatics 21, 353 (2020).
58. Pertea, M. et al. StringTie enables improved reconstruction of a transcriptome from RNA-seq reads. Nat Biotechnol 33, 290-295 (2015).
59. Anders, S. & Huber, W. Differential expression analysis for sequence count data. Genome Biol 11, R106 (2010).
60. Krusong, K., Carpenter, E. P., Bellamy, S. R., Savva, R. & Baldwin, G. S. A comparative study of uracil-DNA glycosylases from human and herpes simplex virus type 1. J Biol Chem 281, 4983-4992 (2006).

EXEMPLARY SEQUENCES

SEQ ID NO: 1, wild type human MPG without first N-terminal M, 297 aa
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMEMKPGTLYVYIIYGMYFCMNISSQGDGACVLLRALEPLEGLETMRQLRSTLRKGTASRVLK
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 2, SpCas9-D10A, Cas9 nickase, nCas9, without first N-terminal Met, 1367 aa
DKKYSIGLAIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERMINF
DKNLPNEKVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDREMIEERLKTYA
HLEDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEII
EQISEFSKRVILADANLDKVLSAYNKHRDKPIREQAENIIHLFTLTNLGAPAAFKYFDTTIDRKRYTSTKEV
LDATLIHQSITGLYETRIDLSQLGGD
SEQ ID NO: 3, dMPG (dead MPG, inactivated MPG-E125A + Y127A + H136A mutant)
VTPALQMKKPKQFCRRMGQKKQRPARAGQPHSSSDAAQAPAEQPHSSSDAAQAPCPRERCLGPPTTPGP
YRSIYESSPKGHLTRLGLEFFDQPAVPLARAFLGQVLVRRLPNGTELRGRIVETAAALGPEDEAAASRGGR
QTPRNRGMEMKPGTLYVYIIYGMYFCMNISSQGDGACVLLRALEPLEGLETMRQLRSTLRKGTASRVLK
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 4, MPGv0.2 (MPG-N169S)
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYGMYFCMSISSQGDGACVLLRALEPLEGLETMRQLRSTLRKGTASRVLK
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 5, MPGv1 (MPG-N169S + S198A + K202A + G203A + S206A + K210A)
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYLYGMYFCMSISSQGDGACVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 6, MPGv2 (MPG-G163R + N169S)
VTPALQMKKPKQFCRRMGQKKQRPARAGQPHSSSDAAQAPAEQPHSSSDAAQAPCPRERCLGPPTTPGP
YRSIYESSPKGHLTRLGLEFFDQPAVPLARAFLGQVLVRRLPNGTELRGRIVETEAYLGPEDEAAHSRGGR
QTPRNRGMFMKPGTLYVYIIYRMYFCMSISSQGDGACVLLRALEPLEGLETMRQLRSTLRKGTASRVLKD
RELCSGPSKLCQALAINKSFDQRDLAQDEAVWLERGPLEPSEPAVVAAARVGVGHAGEWARKPLRFYVR
GSPWVSVVDRVAEQDTQA
SEQ ID NO: 7, MPGv3 (MPG-G163R + N169S + S198A + K202A + G203A + S206A + K210A)
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYITYRMYFCMSISSQGDGACVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 8, MPGv6.3 (MPG-
G163R + N169G + D175R + C178N + S198A + K202A + G203A + S206A + K210A + Q294R)
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYITYRMYFCMGISSQGRGANVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAERDTQA
SEQ ID NO: 9, wild type human MPG with first N-terminal M, 298 aa
MVTPALQMKKPKQFCRRMGQKKQRPARAGQPHSSSDAAQAPAEQPHSSSDAAQAPCPRERCLGPPTTPG
PYRSTYFSSPKGHLTRLGLEFFDQPAVPLARAFLGQVLVRRLPNGTELRGRIVETEAYLGPEDEAAHSRGG
RQTPRNRGMEMKPGTLYVYiIYGMYFCMNISSQGDGACVLLRALEPLEGLETMRQLRSTLRKGTASRVL
KDRELCSGPSKLCQALAINKSFDQRDLAQDEAVWLERGPLEPSEPAVVAAARVGVGHAGEWARKPLRFY
VRGSPWVSVVDRVAEQDTQA
SEQ ID NO: 10, bpSV40 NLS 1
KRTADGSEFESPKKKRKV
SEQ ID NO: 11, bpSV40 NLS 2
KRTADGSEFEPKKKRKV
SEQ ID NO: 12, gGBEv6.3
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTPNEKSNFDLAE
DAKLQLSKDTYDDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEELLVKLNREDLLR.
KQRTEDNGSIPHQIHLGELHAILRRQEDFYPELKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKIIKDKDELDNEE
NEDILEDIVLTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPVENTQLQNEKLYLYYLQNGRD
MYVDQELDINRLSDYDVDHIVPQSELKDDSIDNKVLTRSDKNRGKSDNVPSEEVVKKMKNYWRQLLNA
KLITQRKFDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELALPSKYVNFLYL
ASHYEKLKGSPEDNEQKQLFVEQHKHYLDETTEQISEFSKRVILADANLDKVLSAYNKHRDKPIREQAENI
IHLFTLTNLGAPAAFKYFDTTIDRKRYTSTKEVLDATLIHQSITGLYETRIDLSQLGGDSGGSKRTADGSEF
EPKKKRKVLGGDSGGSGGSGGSVTPALQMKKPKQFCRRMGQKKQRPARAGQPHSSSDAAQAPAEQPH
SSSDAAQAPCPRERCLGPPTTPGPYRSIYESSPKGHLTRLGLEFFDQPAVPLARAFLGQVLVRRLPNGTEL
RGRIVETEAYLGPEDEAAHSRGGRQTPRNRGMEMKPGTLYVYIIYRMYFCMGISSQGRGANVLLRALEP
LEGLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAERDTQASGGSKRTADGSEFEPKKKRKV
SEQ ID NO: 13, gGBE-dMPG, full length, as a negative control
MSGGSDKKYSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDNLL
AQIGDQYADLFLAAKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFKTNRKVTVKQ
LKEDYFKKIECFDSVEISGVEDRFNASLGTYHDLLKIIKDKDELDNEENEDILEDIVLTLTLFEDREMIEERL
KTYAHLFDDKVMKQLKRRRYTGWGRLSRKLINGIRDKQSGKTILDFLKSDGFANRNFMQLIHDDSLTFK
EDIQKAQVSGQGDSLHEHIANLAGSPAIKKGILQTVKVVDELVKVMGRHKPENIVIEMARENQTTQKGQ
KNSRERMKRIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDERKDFQFYKVREINNYHHA
HDAYLNAVVGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDELEAKGYKEVKKDLII
KLPKYSLFELENGRKRMLASAGELQKGNELALPSKYVNFLYLASHYEKLKGSPEDNEQKQLFVEQHKHY
LDELIEQISEFSKRVILADANLDKVLSAYNKHRDKPIREQAENIIHLFTLTNLGAPAAFKYFDTTIDRKRYTS
TKEVLDATLIHQSITGLYETRIDLSQLGGDSGGSKRTADGSEFEPKKKRKVLGGDSGGSGGSGGSVTPALQ
MKKPKQFCRRMGQKKQRPARAGQPHSSSDAAQAPAEQPHSSSDAAQAPCPRERCLGPPTTPGPYR
SIYFSSPKGHLTRLGLEFFDQPAVPLARAFLGQVLVRRLPNGTELRGRIVETAAALGPEDEAAASRG
GRQTPRNRGMFMKPGTLYVYIIYGMYFCMNISSQGDGACVLLRALEPLEGLETMRQLRSTLRKGT
ASRVLKDRELCSGPSKLCQALAINKSFDQRDLAQDEAVWLERGPLEPSEPAVVAAARVGVGHAGE
WARKPLRFYVRGSPWVSVVDRVAEQDTQASGGSKRTADGSEFEPKKKRKV
SEQ ID NO: 14, gGBEv0.1 with WT.hMPG, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMITNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKLIKDKDFLDNEENEDI
LEDIVLTLTLFEDREMIEERLKTYAHLEDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYGMYFCMNISSQGDGACVLLRALEP
LEGLETMRQLRSTLRKGTASRVLKDRELCSGPSKLCQALAINKSFDQRDLAQDEAVWLERGPLEPS
EPAVVAAARVGVGHAGEWARKPLRFYVRGSPWVSVVDRVAEQDTQASGGSKRTADGSEFEPKKKRK
V
SEQ ID NO: 15, gGBEv3, full length
MKRTADGSEFESPKKKRKVSGGSDKKYSIGLAIGTNSVGWAVITDEYKVPSKKEKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLEKTNRKVTVKQLKEDYFKKIECFDSVEISGVEDRFNASLGTYHDLLKLIKDKDFLDNEENEDI
LEDIVLTLILFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSELKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSEEK
NPIDELEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMSISSQGDGACVLLRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 16, MPG-G163R, N169S, G174R, S198A, K202A, G203A, S206A, K210A (MPGv3.1),
8 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMSISSQRDGACVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 17, gGBEv3.1, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYEDTTIDRKRYTSTKEVLDATLIHQSITGLYETRIDLSQLGGDSGGSKRTADGSEFEPKKKRKVL
GGDSGQSGQSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMSISSQRDGACVLLRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 18, MPG-G163R, N169S, D175R, S198A, K202A, G203A, S206A, K210A (MPGv3.2),
8 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMSISSQGRGACVLLRALEPLEGLETMRQLRATLRAATAARVLA
DRELCSGPSKLCQALAINKSEDQRDLAQDEAVWLERGPLEPSEPAVVAAARVGVGHAGEWARKPLRFYV
RGSPWVSVVDRVAEQDTQA
SEQ ID NO: 19, gGBEv3.2, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLILFEDREMIEERLKTYAHLEDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIEINGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMSISSQGRGACVLLRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 20, MPG-G163R, N169S, C178N, S198A, K202A, G203A, S206A, K210A (MPGv3.3),
8 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMSISSQGDGANVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 21, gGBEv3.3, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKLIKDKDELDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDELIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMSISSQGDGANVLLRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 22, MPG-G163R, N169S, D175R, C178N, S198A, K202A, G203A, S206A, K210A
(MPGv4), 9 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMSISSQGRGANVLLRALEPLEGLETMRQLRATLRAATAARVLA
DRELCSGPSKLCQALAINKSFDQRDLAQDEAVWLERGPLEPSEPAVVAAARVQVGHAGEWARKPLRFYV
RGSPWVSVVDRVAEQDTQA
SEQ ID NO: 23, gGBEv4, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNSDVDKLFIQLVQ
TYNQLFEENPINASGVDAKAILSARLSKSRRLENLIAQLPGEKKNGLFGNLLALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKUNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNEDKNLPNEKVLPKHSLLYEYFTVYNELTKVKYVTEGMRKPAELSGEQ
KKAIVDLLFKTNRKVTVKQLKEDYFKKTECFDSVEISGVEDRFNASLGTYHDLLKIIKDKDELDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSELKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDETTEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMSISSQGRGANVLLRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 24, MPG-G163R, N169S, 1170V, D175R, C178N, S198A, K202A, G203A, S206A,
K210A (MPGv4.1), 10 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAELGQVLVRRLPNGTELRGRIVETEAYLGPEDEAAHSRGGR
QTPRNRGMFMKPGTLYVYIIYRMYFCMSVSSQGRGANVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 25, gGBEv4, 1, full length
MKRTADGSEFESPKKKRKVSGGSDKKYSIGLAIGTNSVGWAVITDEYKVPSKKEKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFELSGEQ
KKAIVDLLFKTNRKVTVKQLKEDYFKKIECFDSVEISGVEDRFNASLGTYHDLLKIIKDKDELDNEENEDI
LEDIVLTLILFEDREMIEERLKTYAHLFDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMSVSSQGRGANVLLRALEPL
EGLETMRQLRATLRAATAARVLADRELCSGPSKLCQALAINKSFDQRDLAQDEAVWLERGPLEPSEP
AVVAAARVGVGHAGEWARKPLREYVRGSPWVSVVDRVAEQDTQASGGSKRTADGSEFEPKKKRKV
SEQ ID NO: 26, MPG-G163R, S169G, D175R, C178N, S198A, K202A, G203A, S206A, K210A
(MPGv4.2), 9 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAELGQVLVRRLPNGTELRGRIVETEAYLGPEDEAAHSRGGR
QTPRNRGMFMKPGTLYVYIIYRMYFCMGISSQGRGANVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 27, gGBEv4.2, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLIPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLINL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSTYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMGISSQGRGANVLLRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 28, MPG-RI63Y, N169S, D175R, C178N, S198A, K202A, G203A, S206A, K210A
(MPGv4.3), 9 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYYMYFCMSISSQGRGANVLLRALEPLEGLETMRQLRATLRAATAARVLA
DRELCSGPSKLCQALAINKSEDQRDLAQDEAVWLERGPLEPSEPAVVAAARVGVGHAGEWARKPLRFYV
RGSPWVSVVDRVAEQDTQA
SEQ ID NO: 29, gGBEv4.3, full length
MKRTADGSEFESPKKKRKVSGGSDKKYSIGLAIGTNSVGWAVITDEYKVPSKKEKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYYMYFCMSISSQGRGANVLLRALEPLE
GLETMRQLRATLRAATAARVLADRELCSGPSKLCQALAINKSEDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 30, MPG-G163R, S169G, I170V, D175R, C178N, S198A, K202A, G203A, S206A,
K210A (MPGv5.1), 10 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMGVSSQGRGANVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 31, gGBEv5.1, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLIPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMINFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGEDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMGVSSQGRGANVLLRALEPL
EGLETMRQLRATLRAATAARVLADRELCSGPSKLCQALAINKSFDQRDLAQDEAVWLERGPLEPSEP
AVVAAARVGVGHAGEWARKPLRFYVRGSPWVSVVDRVAEQDTQASGGSKRTADGSEFEPKKKRKV
SEQ ID NO: 32, MPG-R163Y, S169G, 1170V, D175R, C178N, S198A, K202A, G203A, S206A,
K210A (MPGv5.2), 10 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYYMYFCMGVSSQGRGANVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQDTQA
SEQ ID NO: 33, gGBEv5.2, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMIRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLIRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNEFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYYMYFCMGVSSQGRGANVLLRALEPL
EGLETMRQLRATLRAATAARVLADRELCSGPSKLCQALAINKSFDQRDLAQDEAVWLERGPLEPSEP
AVVAAARVGVGHAGEWARKPLRFYVRGSPWVSVVDRVAEQDTQASGGSKRTADGSEFEPKKKRKV
SEQ ID NO: 34, MPG-G163R, S169G, D175R, C178N, S198A, T199R, K202A, G203A, S206A,
K210A (MPGv6.1), 9 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMGISSQGRGANVLLRALEPLEGLETMRQLRARLRAATAARVLA
DRELCSGPSKLCQALAINKSEDQRDLAQDEAVWLERGPLEPSEPAVVAAARVGVGHAGEWARKPLRFYV
RGSPWVSVVDRVAEQDTQA
SEQ ID NO: 35, gGBEv6.1, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGBQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHIVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENITHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMGISSQGRGANVLLRALEPLE
GLETMRQLRARLRAATAARVLADRELCSGPSKLCQALAINKSFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 36, MPG-G163R, S169G, D175R, C178N, S198A, K202A, G203A, S206A, K210A
(MPGv6.2), 9 mutations
VTPALQMKKPKQFCRRMGQKKQRPARAGQPHSSSDAAQAPAEQPHSSSDAAQAPCPRERCLGPPTTPGP
YRSIYFSSPKGHLTRLGLEFFDQPAVPLARAFLGQVLVRRLPNGTELRGRIVETEAYLGPEDEAAHSRGGR
QTPRNRGMFMKPGTLYVYITYRMYFCMGISSQGRGANVLLRALEPLEGLETMRQLRATLRAATAARVLA
DRELCSGPSKLCQALAINKRFDQRDLAQDEAVWLERGPLEPSEPAVVAAARVGVGHAGEWARKPLRFYV
RGSPWVSVVDRVAEQDTQA
SEQ ID NO: 37, gGBEv6.2, full length
MKRTADGSEFESPKKKRKVSGGSDKKYSIGLAIGTNSVGWAVITDEYKVPSKKEKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKTEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDELDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDETTEQISEFSKRVILADANLDKVLSAYNKHRDKPIREQAENIIHLETLINL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGTLYVYIIYRMYFCMGISSQGRGANVLLRALEPLE
GLETMRQLRATLRAATAARVLADRELCSGPSKLCQALAINKRFDQRDLAQDEAVWLERGPLEPSEPA
VVAAARVGVGHAGEWARKPLRFYVRGSPWVSVVDRVAEQDTQASGGSKRTADGSEFEPKKKRKV
SEQ ID NO: 38, MPG-G163R, S169G, D175R, C178N, S198A, K202A, G203A, S206A, K210A,
D295R (MPGv6.4), 9 mutations
VTPALQMKKPKQFCRRMGQKKQRPARAGQPHSSSDAAQAPAEQPHSSSDAAQAPCPRERCLGPPTTPGP
YRSTYFSSPKGHLTRLGLEFFDQPAVPLARAFLGQVLVRRLPNGTELRGRIVETEAYLGPEDEAAHSRGGR
QTPRNRGMFMKPGTLYVYIIYRMYFCMGISSQGRGANVLLRALEPLEGLETMRQLRATLRAATAARVLA
DRELCSGPSKLCQALAINKSFDQRDLAQDEAVWLERGPLEPSEPAVVAAARVGVGHAGEWARKPLRFYV
RGSPWVSVVDRVAEQRTQA
SEQ ID NO: 39, gGBEv6.4, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLEDDKVMKQLKRRRYTGWGRLSRKLINGIRDKQSGKTILDFLKSD
GFANRNFMQLIHDDSLTFKEDIQKAQVSGQGDSLHEHLANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLIRSDKNRGKSDNVPSEEVVKKMKNYWRQLLNAKLITQ
RKEDNLTKAERGGLSELDKAGFIKRQLVETRQTTKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQIGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLINL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAYPLARAFLGQVLVRRLPNGTELRGRI
VETEAYLGPEDEAAHSRGGRQTPRNRGMFMKPGTLYVYIIYRMYFCMGISSQGRGANVILRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAEQRTQASGGSKRTADGSEFEPKKKRKV
SEQ ID NO: 40, Cas9 gRNA scaffold
gttttagagctaGAAAtagcaagttaaaataaggctagtccgttatcaacttgaaaaagtggcacogagtcggtgc
SEQ ID NO: 41, A-to-T reporter, insertion seq
gtgggtgagggcactccggttggggggtcttaagttgggcatttgtgggggtccttcagaattcagaCctgtgtgtctccttgcTg
SEQ ID NO: 42, G-to-T reporter, insertion seq
gtgggtgagggcactccggttggggggtcttaagttgggcatttgtgggggtccttcagaattcagaCctgtgtgtctccttgcCg
SEQ ID NO: 43, C-to-G reporter, insertion seq
CtgggtgagggcactccggttggggggtcttaagttgggcatttgtgggggtccttcagaattcagaCctgtgtgtctccttgcag
SEQ ID NO: 44, T-to-G reporter, insertion seq
Ttgggtgagggcactccggttggggggtcttaagttgggcatttgtgggggtccttcagaattcagagctgtgtgtctccttgcag
SEQ ID NO: 45, U6 promoter
gagggcctatttcccatgattccttcatatttgcatatacgatacaaggctgttagagagataattggaattaatttgactgtaaaca
caaagatattagtacaaaatacgtgacgtagaaagtaataatttcttgggtagtttgcagttttaaaattatgttttaaaatggacta
tcatatgcttaccgtaacttgaaagtatttcgatttcttggctttatatatcttGTGGAAAGGACGAAACACC
SEQ ID NO: 46, EF-1α promoter
GGCTCCGGTGCCCGTCAGTGGGCAGAGCGCACATCGCCCACAGTCCCCGAGAAGTTGGGGGGAGGG
GTCGGCAATTGAACCGGTGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTAGTCGCCGTGAACGTTCTT
TTTCGCAACGGGTTTGCCGCCAGAACACAGGTAAGIGCCGTGTGTGGTTCCCGCGGGCCTGGCCTCT
TTACGGGTTATGGCCCTTGCGTGCCTTGAATTACTTCCACCTGGCTGCAGTACGTGATTCTTGATCCCG
AGCTTCGGGTTGGAAGTGGGTGGGAGAGTTCGAGGCCTTGCGCTTAAGGAGCCCCTTCGCCTCGTGC
TTGAGTTGAGGCCTGGCCTGGGCGCTGGGGCCGCCGCGTGCGAATCTGGTGGCACCTTCGCGCCTGT
CTCGCTGCTTTCGATAAGTCTCTAGCCATTTAAAATTTTTGATGACCTGCTGCGACGCTTTTTTTCTGGC
AAGATAGTCTTGTAAATGCGGGCCAAGATCTGCACACTGGTATTTCGGTTTTTGGGGCCGCGGGCGGC
GACGGGGCCCGTGCGTCCCAGCGCACATGTTCGGCGAGGCGGGGCCTGCGAGCGCGGCCACCGAGA
ATCGGACGGGGGTAGTCTCAAGCTGGCCGGCCTGCTCTGGTGCCTGGCCTCGCGCCGCCGTGTATCG
CCCCGCCCTGGGCGGCAAGGCTGGCCCGGTCGGCACCAGTTGCGTGAGCGGAAAGATGGCCGCTTC
CCGGCCCTGCTGCAGGGAGCTCAAAATGGAGGACGCGGCGCTCGGGAGAGCGGGCGGGTGAGTCAC
CCACACAAAGGAAAAGGGCCTTTCCGTCCTCAGCCGTCGCTTCATGTGACTCCACGGAGTACCGGGC
GCCGTCCAGGCACCTCGATTAGTTCTCGAGCTTTTGGAGTACGTCGTCTTTAGGTTGGGGGGAGGGGT
TTTATGCGATGGAGTTTCCCCACACTGAGTGGGTGGAGACTGAAGTTAGGCCAGCTTGGCACTTGATG
TAATTCTCCTTGGAATTTGCCCTTTTTGAGTTTGGATCTTGGTTCATTCTCAAGCCTCAGACAGTOGTT
CAAAGTTTTTTTCTTCCATTTCAGGTGTCGTGAGGAATTAGCTTGGTACtaatacgactcactatagggagagcegccac
C
SEQ ID NO: 47, SV40 poly(A) signal
ACGCGTCGAACTTGTTTATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGTATCTTATCATGTCTG
GATC
gGBEv6.3-dCas9
SEQ ID NO: 48, dCas9
DKKYSIGLAIGTNSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNEKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERMTNF
DKNLPNEKVLPKHSLLYEYETVYNELTKVKYVTEGMRKPAFLSGEQKKAIVDLLEKTNRKVTVKQLKED
YFKKIECFDSVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVEQHKHYLDEIT
EQISEFSKRVILADANLDKVLSAYNKHRDKPIREQAENIIHLFTLINLGAPAAFKYFDTTIDRKRYTSTKEV
LDATLIHQSITGLYETRIDLSQLGGD
SEQ ID NO: 49, gGBEv6, 3-dCas9, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLIPNFKSNEDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPELKDNREKIEKILTFRIPYYVGPLARGNSRFAWMIRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLEDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDAIVPQSELKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGE
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDETTEQISEFSKRVILADANLDKVLSAYNKHRDKPIREQAENIIHLFTLINL
GAPAAFKYFDTTIDRKRYTSTKEVLDATLIHQSITGLYETRIDLSQLGGDSGGSKRTADGSEFEPKKKRKVL
GGDSGGSGGSGGSVTPALQMKKPKQFCRRMGQKKQRPARAGQPHSSSDAAQAPAEQPHSSSDAAQ
APCPRERCLGPPTTPGPYRSIYFSSPKGHLTRLGLEFFDQPAVPLARAFLGQVLVRRLPNGTELRGRI
VETEAYLGPEDEAAHSRGGRQTPRNRGMFMKPGILYVYIIYRMYFCMGISSQGRGANVLLRALEPLE
GLETMRQLRATLRAATAARVLADRELCSGPSKLCQALAINKSFDQRDLAQDEAVWLERGPLEPSEPA
VVAAARVGVGHAGEWARKPLRFYVRGSPWVSVVDRVAERDTQASGGSKRTADGSEFEPKKKRKV
gGBEv6.3-nCas12imax
SEQ ID NO: 50, nCas12Max (xCas12i-N243R + E336R + W896R)
MSSDVVRPYNTKLLPDNRKHNMFLQTFKRLNSISLNHFDLLICLYAAITNKKAEEYKSEKEAHVTADSLC
AINWERPMSKRYSKYATTTFNMLELFKEYSGHEPDAYSKNYLMSNIDSDRFVWVDCRKFAKDFAYQMEL
GFHEFTVLAETLLANSILVLNESTKANWAWGTVSALYGGGDKEDSTLKSKILLAFVDALNNHELKTKREI
LNQVCESLKYQSYQDMYVDFRSVVDENGNKKSPRGSMPIVTKFETDDLISDNQRKAMISNFTKNAAAK
AAKKPIPYLDRLKEHMVSLCDEYNVYAWAAAITNSNADVTARNTRNLTFIGEQNSRRKRLSVLQTTTNE
KAKDILNKINDNLIQEVRYTPAPKHLGRDLANLFDTLKEKDINNIENEEEKQNVINDCIEQYVDDCRSLNR
NPIAALLKHISRYYEDFSAKNFLDGAKLNVLTEVVNRQKAHPTIWSEKAYTWISKFDKNRRQANSSLVG
WVVPPEEVHKEKIAGQQSMMWVTLTLLDDGKWVKHHIPFSDSRYYSEVYAYNPNLPYLDGGIPRQSKFG
NKPTTNLTAESQALLANSKYKKANKSELRAKENATHNVRVSPNTSLCIRLLKDSAGNQMEDKIGNVLFG
MQINHKITVGKPNYKIEVGDRFLGFDQNQSENHTYAVLQRVSESSHDTHHFNGWDVKVLEKGKVTSDVI
VRDEVYDQLSYEGVPYDSSKFAEWRDKRRRFVLENLSIQLEEGKTFLTEFDKLNKDSLYRWNMNYLKLL
RKAIRAGGKEFAKIAKTEIFELAVERFGPINLGSLSQISLKMIASFKGVVQSYFSVSGCVDDASKKAHDSM
LFTFMCAAEEKRTNKREEKTNRAASFILQKAYLHGCKMIVCEDDLPVADGKTGKAQNADRMDRCARAL
AKKVNDGCVAMSICYRAIPAYMSSHQDPFVHMQDKKTSVLRPRFMEVNKDSIRDYHVAGLRRMLNSKS
DAGTSVYYRQAALHFCEALGVSPELVKNKKTHAAELGKHMGSAMLMPWRGGRVYIASKKLTSDAKSV
KYCGEDMWQYHADEIAAVNIAMYEVCCQTGAFGKKQKKSDELPG
SEQ ID NO: 51, gGBEv6.3-nCas12Max, full length
MKRTADGSEFESPKKKRKVSGGSMSSDVVRPYNTKLLPDNRKHNMFLQTFKRLNSISLNHFDLLICLYAA
ITNKKAEEYKSEKEAHVTADSLCAINWFRPMSKRYSKYATTTFNMLELFKEYSGHEPDAYSKNYLMSNID
SDRFVWVDCRKFAKDFAYQMELGFHEFTVLAETLLANSILVLNESTKANWAWGTVSALYGGGDKEDSTL
KSKILLAFVDALNNHELKTKREILNQVCESLKYQSYQDMYVDFRSVVDENGNKKSPRGSMPIVTKFETD
DLISDNQRKAMISNFTKNAAAKAAKKPIPYLDRLKEHMVSLCDEYNVYAWAAAITNSNADVTARNTRNL
TFIGEQNSRRKRLSVLQTTTNEKAKDILNKINDNLIQEVRYTPAPKHLGRDLANLEDTLKEKDINNIENEEE
KQNVINDCIEQYVDDCRSLNRNPIAALLKHISRYYEDFSAKNFLDGAKLNVLTEVVNRQKAHPTIWSEKA
YTWISKFDKNRRQANSSLVGWVVPPEEVHKEKIAGQQSMMWVTLTLLDDGKWVKHHIPFSDSRYYSEV
YAYNPNLPYLDGGIPRQSKFGNKPTTNLTAESQALLANSKYKKANKSFLRAKENATHNVRVSPNTSLCIRL
LKDSAGNQMFDKIGNVLFGMQINHKITVGKPNYKIEVGDRFLGFDQNQSENHTYAVLQRVSESSHDTHH
FNGWDVKVLEKGKVTSDVIVRDEVYDQLSYEGVPYDSSKFAEWRDKRRRFVLENLSIQLEEGKTFLTEF
DKLNKDSLYRWNMNYLKLLRKAIRAGGKEFAKIAKTEIFELAVERFGPINLGSLSQISLKMIASFKGVVQS
YFSVSGCVDDASKKAHDSMLFTFMCAAEEKRTNKREEKTNRAASFILQKAYLHGCKMIVCEDDLPVADG
KTGKAQNADRMDRCARALAKKVNDGCVAMSICYRAIPAYMSSHQDPFVHMQDKKTSVLRPRFMEVNK
DSIRDYHVAGLRRMLNSKSDAGTSVYYRQAALHFCEALGVSPELVKNKKTHAAELGKHMGSAMLMPW
RGGRVYIASKKLTSDAKSVKYCGEDMWQYHADELAAVNIAMYEVCCQTGAFGKKQKKSDELPGSGGSK
RTADGSEFEPKKKRKVLGGDSGGSGGSGGSVTPALQMKKPKQFCRRMGQKKQRPARAGQPHSSSDA
AQAPAEQPHSSSDAAQAPCPRERCLGPPTTPGPYRSIYFSSPKGHLTRLGLEFFDQPAVPLARAFLGQ
VLVRRLPNGTELRGRIVETEAYLGPEDEAAHSRGGRQTPRNRGMEMKPGTLYVYIIYRMYFCMGIS
SQGRGANVLLRALEPLEGLETMRQLRATLRAATAARVLADRELCSGPSKLCQALAINKSFDQRDLAQD
EAVWLERGPLEPSEPAVVAAARVGVGHAGEWARKPLRFYVRGSPWVSVVDRVAERDTQASGGSKRT
ADGSEFEPKKKRKV
gGBEv6.3-OgeuIscB(enIscB-D61A)
SEQ ID NO: 52, gGBEv6.3-OgeuIscB(enIscB-D61A), full length
MMAVVYVISKSGKPLMPTTRCGHVRILLKEGKARVVERKPFTIQLTYESAEETQPLVLGIAPGRINIGMSV
VTESGESVFNAQIRTRNKDVPKLMKDRKQYRMAHRRLKRRCKRRRRAKAAGTAFEEGEKQRLLPGCFK
PITCKSIRNKEARFNNRKRPVGWLTPTANHLLVTHLNVVKKVQKILPVAKVVLELNRFSFMAMNNPKVQ
RWQYQRGPLYGKGSVEEAVSMQQDGHCLFCKHGIDHYHHVVPRRKNGSETLENRVGLCEEHHRLVHTD
KEWEANLASKKSGMNKKYHALSVLNQIIPYLADQLADMFPGNFCVTSGQDTYLFREEHGIPKDHYLDA
YCIACSALTDAKKVSSPKGRPYMVRQFRRHDRQACHKANLNRRYYMGGKLVATNRHKAMDQKTDSLE
EYRAAHSAADVSKLTVKHPSAQYKDMSRIMPGSILVSGEGKLFTLRRSEGRNKGQVNYFVSTEGIKYWA
RKCQYLRNNGGLQIYV
SEQ ID NQ: 53, gGBEv6.3-QgeuIscB(enIscB-D61A), full length
MKRTADGSEFESPKKKRKVSGGSMMAVVYVISKSGKPLMPTTRCGHVRILLKEGKARVVERKPFTIQLTY
ESAEETQPLVLGIAPGRTNIGMSVVTESGESVFNAQIRTRNKDVPKLMKDRKQYRMAHRRLKRRCKRRR
RAKAAGTAFEEGEKQRLLPGCFKPITCKSIRNKEARFNNRKRPVGWLTPTANHLLVTHLNVVKKVQKILP
VAKVVLELNRFSFMAMNNPKVQRWQYQRGPLYGKGSVEEAVSMQQDGHCLFCKHGIDHYHHVVPRRK
NGSETLENRVGLCEEHHRLVHTDKEWEANLASKKSGMNKKYHALSVLNQIIPYLADQLADMFPGNFCV
TSGQDTYLFREEHGIPKDHYLDAYCIACSALTDAKKVSSPKGRPYMVRQFRRHDRQACHKANLNRRYY
MGGKLVATNRHKAMDQKTDSLEEYRAAHSAADVSKLTVKHPSAQYKDMSRIMPGSILVSGEGKLFTLRR
SEGRNKGQVNYFVSTEGIKYWARKCQYLRNNGGLQIYVSGGSKRTADGSEFEPKKKRKVLGGDSGGSG
GSGGSVTPALQMKKPKQFCRRMGQKKQRPARAGQPHSSSDAAQAPAEQPHSSSDAAQAPCPRERC
LGPPTTPGPYRSIYFSSPKGHLTRLGLEFFDQPAVPLARAFLGQVLVRRLPNGTELRGRIVETEAYLG
PEDEAAHSRGGRQTPRNRGMFMKPGTLYVYIIYRMYFCMGISSQGRGANVLLRALEPLEGLETMRQL
RATLRAATAARVLADRELCSGPSKLCQALAINKSFDQRDLAQDEAVWLERGPLEPSEPAVVAAARVG
VGHAGEWARKPLRFYVRGSPWVSVVDRVAERDTQASGGSKRTADGSEFEPKKKRKV
SEQ ID NO: 54, wild-type human UNG1 (UniProt P13051-2)
(https://www.uniprot.org/uniprotkb/P13051/entry#sequences), 304 aa
MGVFCLGPWGLGRKLRTPGKGPLQLLSRLCGDHLQAIPAKKAPAGQEEPGTPPSSPLSAEQLDRIQRNKA
AALLRLAARNVPVGFGESWKKHLSGEFGKPYFIKLMGFVAEERKHYTVYPPPHQVFTWTQMCDIKDVK
VVILGQDPYHGPNQAHGLCFSVQRPVPPPPSLENIYKELSTDIEDFVHPGHGDLSGWAKQGVLLLNAVLT
VRAHQANSHKERGWEQFTDAVVSWLNQNSNGLVFLLWGSYAQKKGSAIDRKRHHVLQTAHPSPLSVYR
GFFGCRHFSKTNELLQKSGKKPIDWKEL
SEQ ID NO: 55, gTBEv0.1, full length (with wt UNGI)
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENED]
LEDIVLTLTLFEDREMIEERLKTYAHLFDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDETTEQISEFSKRVILADANLDKVLSAYNKHRDKPIREQAENIIHLETLTNL
GAPAAFKYFDTTIDRKRYTSTKEVLDATLIHQSITGLYETRIDLSQLGGDSGGSKRTADGSEFEPKKKRKVL
GGDSGGSGGSGGSGVFCLGPWGLGRKLRTPGKGPLQLLSRLCGDHLQAIPAKKAPAGQEEPGTPPSSPLS
AEQLDRIQRNKAAALLRLAARNVPVGFGESWKKHLSGEFGKPYFIKLMGFVAEERKHYTVYPPPHQVFT
WTQMCDIKDVKVVILGQDPYHGPNQAHGLCFSVQRPVPPPPSLENTYKELSTDIEDFVHPGHGDLSGWA
KQGVLLLNAVLTVRAHQANSHKERGWEQFTDAVVSWLNQNSNGLVFLLWGSYAQKKGSAIDRKRHHV
LQTAHPSPLSVYRGFFGCRHFSKTNELLQKSGKKPIDWKELSGGSKRTADGSEFEPKKKRKV
SEQ ID NO: 56, UNG1-Y147A
GVFCLGPWGLGRKLRTPGKGPLQLLSRLCGDHLQAIPAKKAPAGQEEPGTPPSSPLSAEQLDRIQRNKAA
ALLRLAARNVPVGFGESWKKHLSGEFGKPYFIKLMGFVAEERKHYTVYPPPHQVFTWTQMCDIKDVKV
VILGQDPAHGPNQAHGLCFSVQRPVPPPPSLENIYKELSTDIEDFVHPGHGDLSGWAKQGVLLLNAVLTVR
AHQANSHKERGWEQFTDAVVSWLNQNSNGLVFLLWGSYAQKKGSAIDRKRHHVLQTAHPSPLSVYRGF
FGCRHFSKTNELLQKSGKKPIDWKEL
SEQ ID NO: 57, gTBEv0.2, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQHIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKLIKDKDELDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGQFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSGVFCLGPWGLGRKLRTPGKGPLQLLSRLCGDHLQAIPAKKAPAGQEEPGTPPSSPLS
AEQLDRIQRNKAAALLRLAARNVPVGFGESWKKHLSGEFGKPYFIKLMGFVAEERKHYTVYPPPHQVFT
WTQMCDIKDVKVVILGQDPAHGPNQAHGLCFSVQRPVPPPPSLENTYKELSTDIEDFVHPGHGDLSGWAK
QGVLLLNAVLTVRAHQANSHKERGWEQFTDAVVSWLNQNSNGLVFLLWGSYAQKKGSAIDRKRHHVL
QTAHPSPLSVYRGFFGCRHFSKTNELLQKSGKKPIDWKELSGGSKRTADGSEFEPKKKRKV
SEQ ID NO: 58, UNG1-N204D
GVFCLGPWGLGRKLRTPGKGPLQLLSRLCGDHLQAIPAKKAPAGQEEPGTPPSSPLSAEQLDRIQRNKAA
ALLRLAARNVPVGFGESWKKHLSGEFGKPYFIKLMGFVAEERKHYTVYPPPHQVFTWTQMCDIKDVKV
VILGQDPYHGPNQAHGLCFSVQRPVPPPPSLENIYKELSTDIEDFVHPGHGDLSGWAKQGVLLLDAVLIV
RAHQANSHKERGWEQFTDAVVSWLNQNSNGLVFLLWGSYAQKKGSAIDRKRHHVLQTAHPSPLSVYRG
FFGCRHFSKTNELLQKSGKKPIDWKEL
SEQ ID NO: 59, gTBEv0.3, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLELAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTADGSEFEPKKKRKVL
GGDSGGSGGSGGSGVFCLGPWGLGRKLRTPGKGPLQLLSRLCGDHLQAIPAKKAPAGQEEPGTPPSSPLS
AHQLDRIQRNKAAALLRLAARNVPVGFGESWKKHLSGEFGKPYFIKLMGFVAEERKHYTVYPPPHQVFT
WTQMCDIKDVKVVILGQDPYHGPNQAHGLCFSVQRPVPPPPSLENIYKELSTDIEDFVHPGHGDLSGWA
KQGVLLLDAVLTVRAHQANSHKERGWEQFTDAVVSWLNQNSNGLVFLLWGSYAQKKGSAIDRKRHHV
LQTAHPSPLSVYRGFFGCRHFSKTNELLQKSGKKPIDWKELSGGSKRTADGSEFEPKKKRKV
SEQ ID NO: 60, UNG1-Δ1-59, Y147A
EQLDRIQRNKAAALLRLAARNVPVGFGESWKKHLSGEFGKPYFIKLMGFVAEERKHYTVYPPPHQVFTW
TQMCDIKDVKVVILGQDPAHGPNQAHGLCFSVQRPVPPPPSLENIYKELSTDIEDFVHPGHGDLSGWAKQ
GVLLLNAVLTVRAHQANSHKERGWEQFTDAVVSWLNQNSNGLVFLLWGSYAQKKGSAIDRKRHHVLQT
AHPSPLSVYRGFFGCRHFSKTNELLQKSGKKPIDWKEL
SEQ ID.NO: 61, gTBEv0.4, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTETTKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKLIKDKDELDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDELEAKGYKEVKKDLIIKLPKYSLFELENGRKRMLASAGELQKGNELALPSKYVNFLYLASHYEKLK
GSPEDNEQKQLFVEQHKHYLDEHIEQISEFSKRVILADANLDKVLSAYNKHRDKPIREQAENIIHLFILTNL
GAPAAFKYFDTTIDRKRYTSTKEVLDATLIHQSITGLYETRIDLSQLGGDSGGSKRTADGSEFEPKKKRKVL
GGDSGGSGGSGGSEQLDRIQRNKAAALLRLAARNVPVGFGESWKKHLSGEFGKPYFIKLMGFVAEERKH
YTVYPPPHQVFTWTQMCDIKDVKVVILGQDPAHGPNQAHGLCFSVQRPVPPPPSLENIYKELSTDIEDFV
HPGHGDLSGWAKQGVLLLNAVLTVRAHQANSHKERGWEQFTDAVVSWLNQNSNGLVELLWGSYAQKK
GSAIDRKRHHVLQTAHPSPLSVYRGFFGCRHFSKTNELLQKSGKKPIDWKELSGGSKRTADGSEFEPKKK
RKV
SEQ ID NO: 62, UNG1-Δ1-59, N204D
EQLDRIQRNKAAALLRLAARNVPVGFGESWKKHLSGEFGKPYFIKLMGFVAEERKHYTVYPPPHQVFTW
TQMCDIKDVKVVILGQDPYHGPNQAHGLCESVQRPVPPPPSLENIYKELSTDIEDFVHPGHGDLSGWAKQ
GVLLLDAVLTVRAHQANSHKERGWEQFTDAVVSWLNQNSNGLVELLWGSYAQKKGSAIDRKRHHVLQT
AHPSPLSVYRGFFGCRHFSKTNELLQKSGKKPIDWKEL
SEQ ID NO: 63, gTBEv0.5, full length
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQTTKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAENIIHLFILINL
GAPAAFKYFDTTIDRKRYTSTKEVLDATLIHQSITGLYETRIDLSQLGGDSGGSKRTADGSEFEPKKKRKVL
GGDSGGSGGSGGSEQLDRIQRNKAAALLRLAARNVPVGFGESWKKHLSGEFGKPYFIKLMGFVAEERKH
YTVYPPPHQVFTWTQMCDIKDVKVVILGQDPYHGPNQAHGLCFSVQRPVPPPPSLENIYKELSTDIEDFV
HPGHGDLSGWAKQGVLLLDAVLTVRAHQANSHKERGWEQFTDAVVSWLNQNSNGLVFLLWGSYAQKK
GSAIDRKRHHVLQTAHPSPLSVYRGFFGCRHFSKTNELLQKSGKKPIDWKELSGGSKRTADGSEFEPKKK
RKV
SEQ ID NO: 64, TDG
EAENAGSYSLQQAQAFYTFPFQQLMAEAPNMAVVNEQQMPEEVPAPAPAQEPVQEAPKGRKRKPRTTEP
KQPVEPKKPVESKKSGKSAKSKEKQEKITDTFKVKRKVDRENGVSEAELLTKTLPDILTFNLDIVIIGINPG
LMAAYKGHHYPGPGNHFWKCLFMSGLSEVQLNHMDDHTLPGKYGIGFTNMVERTTPGSKDLSSKEFRE
GGRILVQKLQKYQPRIAVFNGKCIYEIFSKEVFGVKVKNLEFGLQPHKIPDTETLCYVMPSSSARCAQFPR
AQDKVHYYIKLKDLRDQLKGIERNMDVQEVQYTFDLQLAQEDAKKMAVKEEKYDPGYEAAYGGAYGE
NPCSSEPCGESSNGLIESVELRGESAFSGIPNGQWMTQSFTDQIPSFSNHCGTQEQEEESHA
SEQ ID NO: 65, SMUG1
PQAFLLGSIHEPAGALMEPQPCPGSLAESFLEEELRLNAELSQLQFSEPVGIIYNPVEYAWEPHRNYVTRYC
QGPKEVLFLGMNPGPFGMAQTGVPFGEVSMVRDWLGIVGPVLTPPQEHPKRPVLGLECPQSEVSGARFW
GFFRNLCGQPEVFFHHCFVHNLCPLLFLAPSGRNLTPAELPAKQREQLLGICDAALCRQVQLLGVRLVVG
VGRLAEQRARRALAGLMPEVQVEGLLHPSPRNPQANKGWEAVAKERLNELGLLPLLLK
SEQ ID NO: 66, OGG1
PARALLPRRMGHRTLASTPALWASIPCPRSELRLDLVLPSGQSFRWREQSPAHWSGVLADQVWTLTQTEE
QLHCTVYRGDKSQASRPTPDELEAVRKYFQLDVTLAQLYHHWGSVDSHFQEVAQKFQGVRLLRQDPIEC
LFSFICSSNNNIARITGMVERLCQAFGPRLIQLDDVTYHGFPSLQALAGPEVEAHLRKLGLGYRARYVSAS
ARAILEEQGGLAWLQQLRESSYEEAHKALCILPGVGTKVADCICLMALDKPQAVPVDVHMWHIAQRDYS
WHPTTSQAKGPSPQTNKELGNFFRSLWGPYAGWAQAVLFSADLRQSRHAQEPPAKRRKGSKGPEG
SEQ ID NO: 67, MBD4
GTTGLESLSLGDRGAAPTVTSSERLVPDPPNDLRKEDVAMELERVGEDEEQMMIKRSSECNPLLQEPIASA
QFGATAGTECRKSVPCGWERVVKQRLFGKTAGREDVYFISPQGLKFRSKSSLANYLHKNGETSLKPEDED
FTVLSKRGIKSRYKDCSMAALTSHLQNQSNNSNWNLRTRSKCKKDVEMPPSSSSELQESRGLSNFTSTHL
LLKEDEGVDDVNFRKVRKPKGKVTILKGIPIKKTKKGCRKSCSGEVQSDSKRESVQNKADAESEPVAQKS
QLDRTVCISDAGACGETLSVTSEENSLVKKKERSLSSGSNFCSEQKTSGIINKFCSAKDSEHNEKYEDTFLE
SEEIGTKVEVVERKEHLHTDILKRGSEMDNNCSPTRKDFTGEKIFQEDTIPRTQIERRKTSLYFSSKYNKEA
LSPPRRKAFKKWTPPRSPFNLVQETLFHDPWKLLIATIFLNRTSGKMAIPVLWKFLEKYPSAEVARTADWR
DVSELLKPLGLYDLRAKTIVKESDEYLTKQWKYPIELHGIGKYGNDSYRIFCVNEWKQVHPEDHKLNKY
HDWLWENHEKLSLS
SEQ ID NO: 68, MUTYH
TPLVSRLSRLWAIMRKPRAAVGSGHRKQAASQEGRQKHAKNNSQAKPSACDGMIAECPGAPAGLARQPE
EVVLQASVSSYHLFRDVAEVTAFRGSLLSWYDQEKRDLPWRRRAEDEMDLDRRAYAVWVSEVMLQQT
QVATVINYYTGWMQKWPTLQDLASASLEEVNQLWAGLGYYSRGRRLQEGARKVVEELGGHMPRTAET
LQQLLPGVGRYTAGAIASIAFGQATGVVDGNVARVLCRVRAIGADPSSTLVSQQLWGLAQQLVDPARPGD
FNQAAMELGATVCTPQRPLCSQCPVESLCRARQRVEQEQLLASGSLSGSPDVEECAPNTGQCHLCLPPSE
PWDQTLGVVNFPRKASRKPPREESSATCVLEQPGALGAQILLVQRPNSGLLAGLWEFPSVTWEPSEQLQR
KALLQELQRWAGPLPATHLRHLGEVVHTFSHIKLTYQVYGLALEGQTPVTTVPPGARWLTQEEFHTAAVS
TAMKKVFRVYQGQQPGTCMGSKRSQVSSPCSRKKPRMGQQVLDNFFRSHISTDAHSLNSAAQ
SEQ ID NO: 69, NEIL1
PEGPELHLASQFVNEACRALVFGGCVEKSSVSRNPEVPFESSAYRISASARGKELRLILSPLPGAQPQQEPL
ALVFRFGMSGSFQLVPREELPRHAHLRFYTAPPGPRLALCFVDIRRFGRWDLGGKWQPGRGPCVLQEYQ
QFRENVLRNLADKAFDRPICEALLDQRFFNGIGNYLRAEILYRLKIPPFEKARSVLEALQQHRPSPELTLSQ
KIRTKLQNPDLLELCHSVPKEVVQLGGKGYGSESGEEDFAAFRAWLRCYGMPGMSSLQDRHGRTIWFQG
DPGPLAPKGRKSRKKKSKATQLSPEDRVEDALPPSKAPSRTRRAKRDLPKRTATQRPEGTSLQQDPEAPTV
PKKGRRKGRQAASGHCRPRKVKADIPSLEPEGTSAS
SEQ ID NO: 70, NEIL2
PEGPLVRKFHHLVSPFVGQQVVKTGGSSKKLQPASLQSLWLQDTQVHGKKLFLRFDLDEEMGPPGSSPTP
EPPQKEVQKEGAADPKQVGEPSGQKTLDGSSRSAELVPQGEDDSEYLERDAPAGDAGRWLRVSFGLFGS
VWVNDFSRAKKANKRGDWRDPSPRLVLHFGGGGFLAFYNCQLSWSSSPVVTPTCDILSEKFHRGQALEA.
LGQAQPVCYTLLDQRYFSGLGNIIKNEALYRAGIHPLSLGSVLSASRREVLVDHVVEFSTAWLQGKFQGR
PQHTQVYQKEQCPAGHQVMKEAFGPEDGLQRLTWWCPQCQPQLSEEPEQCQFS
SEQ ID NO: 71, NEIL3
VEGPGCTLNGEKIRARVLPGQAVTGVRGSALRSLQGRALRLAASTVVVSPQAAALNNDSSQNVLSLENG
YVYSGVETLGKELFMYFGPKALRIHFGMKGFIMINPLEYKYKNGASPVLEVQLTKDLICFFDSSVELRNS
MESQQRIRMMKELDVCSPEFSFLRAESEVKKQKGRMLGDVLMDQNVLPGVGNIIKNEALFDSGLHPAVK
VCQLTDEQTHHLMKMIRDFSILFYRCRKAGLALSKHYKVYKRPNCGQCHCRITVCRFGDNNRMTYFCPH
CQKENPQHVDICKLPTRNTIISWTSSRVDHVMDSVARKSEEHWTCVVCTLINKPSSKACDACLTSRPIDSV
LKSEENSTVFSHLMKYPCNTFGKPHTEVKINRKTAFGTTTLVLTDFSNKSSTLERKTKQNQILDEEFQNSPP
ASVCLNDIQHPSKKTTNDITQPSSKVNISPTISSESKLFSPAHKKPKTAQYSSPELKSCNPGYSNSELQINMT
DGPRTLNPDSPRCSKHNRLCILRVVGKDGENKGRQFYACPLPREAQCGFFEWADLSFPFCNHGKRSTMKT
VLKIGPNNGKNFFVCPLGKEKQCNFFQWAENGPGIKIIPGC
SEQ ID NO: 72, NTHL1
CSPQESGMTALSARMLTRSRSLGPGAGPRGCREEPGPLRRREAAAEARKSHSPVKRPRKAQRLRVAYEGS
DSEKGEGAEPLKVPVWEPQDWQQQLVNIRAMRNKKDAPVDHLGTEHCYDSSAPPKVRRYQVLLSLMLS
SQTKDQVTAGAMQRLRARGLTVDSILQTDDATLGKLIYPVGFWRSKVKYIKQTSAILQQHYGGDIPASVA
ELVALPGVGPKMAHLAMAVAWGTVSGIAVDTHVHRIANRLRWTKKATKSPEETRAALEEWLPRELWHEI
NGLLVGFGQQTCLPVHPRCHACLNQALCPAAQGL
SEQ ID NO: 73, Cas12i scaffold sequence
AGAAATGTGTCCCCAGTTGACAC
SEQ ID NO: 74, IscB scaffold sequence
GGCTCGTCCAACTGCGGTTGAACGAGCACAGGCTGAGACATTCGTAAGGCCGAAAGGCCGGACGCA
CCCTGGGATTTCCCCAGTCCCCGGAACTGCATAGCGGATGCCAGTTGATGGAGCAATCTATCAGATAA
GCCAGGGGGAACAATCACCTCTCTGTATCAGAGAGAGTTTTACAAAAGGAGGAACGG
SEQ ID NO: 118, human Polη
MATGQDRVVALVDMDCFFVQVEQRQNPHLRNKPCAVVQYKSWKGGGITAVSYEARAFGVTRSMWADD
AKKLCPDLLLAQVRESRGKANLIKYREASVEVMEIMSRFAVIERASIDEAYVDLTSAVQERLQKLQGQPIS
ADLLPSTYIEGLPQGPTTAEETVQKEGMRKQGLFQWLDSLQIDNLTSPDLQLTVGAVIVEEMRAAIERETG
FQCSAGISHNKVLAKLACGLNKPNRQTLVSHGSVPQLFSQMPIRKIRSLGGKLGASVIEILGIEYMGELTQF
TESQLQSHFGEKNGSWLYAMCRGIEHDPVKPRQLPKTIGCSKNFPGKTALATREQVQWWLLQLAQELEE
RLTKDRNDNDRVATQLVVSIRVQGDKRLSSLRRCCALTRYDAHKMSHDAFTVIKNCNTSGIQTEWSPPLT
MLFLCATKFSASAPSSSTDITSFLSSDPSSLPKVPVTSSEAKTQGSGPAVTATKKATTSLESFFQKAAERQKV
KEASLSSLTAPTQAPMSNSPSKPSLPFQTSQSTGTEPFFKQKSLLLKQKQLNNSSVSSPQQNPWSNCKALP
NSLPTEYPGCVPVCEGVSKLEESSKATPAEMDLAHNSQSMHASSASKSVLEVTQKATPNPSLLAAEDQVP
CEKCGSLVPVWDMPEHMDYHFALELQKSFLQPHSSNPQVVSAVSHQGKRNPKSPLACTNKRPRPEGMQT
LESFFKPLTH
SEQ ID NO: 133, Wild type human UNG2 (UniProt P13051-1)
(https://www.uniprot.org/uniprotkb/P13051/entry#sequences), 313 aa
MIGQKTLYSFFSPSPARKRHAPSPEPAVQGTGVAGVPEESGDAAAIPAKKAPAGQEEPGTPPSSPLSAEQLD
RIQRNKAAALLRLAARNVPVGFGESWKKHLSGEFGKPYFIKLMGFVAEERKHYTVYPPPHQVFTWTQM
CDIKDVKVVILGQDPYHGPNQAHGLCESVQRPVPPPPSLENIYKELSTDIEDFVHPGHGDLSGWAKQGVL
LLNAVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 134, linker
SGGSSGGSSGSETPGTSESATPESSGGSSGGS
SEQ ID NO: 135, UNG2-Y156A
MIGQKTLYSFESPSPARKRHAPSPEPAVQGTGVAGVPEESGDAAAIPAKKAPAGQEEPGTPPSSPLSAEQLD
RIQRNKAAALLRLAARNVPVGFGESWKKHLSGEFGKPYFIKLMGFVAEERKHYTVYPPPHQVFTWTQM
CDIKDVKVVILGQDPAHGPNQAHGLCFSVQRPVPPPPSLENIYKELSTDIEDFVHPGHGDLSGWAKQGVL
LLNAVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 136, gTBEv0.1 (M-bpSV40 NLS-nCas9-linker-UNG2-Y156A-linker-bpSV40 NLS)
MKRTADGSEFESPKKKRKVDKKYSIGLAIGTNSVGWAVITDEYKVPSKKEKVLGNTDRHSIKKNLIGALL
FDSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDLLRKQRTEDNGS
IPHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQELDIN
RLSDYDVDHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKLKGSPED
NEQKQLFVEQHKHYLDEITEQISEFSKRVILADANLDKVLSAYNKHRDKPIREQAENIIHLETLTNLGAPAA
FKYFDTTIDRKRYTSTKEVLDATLIHQSITGLYETRIDLSQLGGDSGGSSGGSSGSETPGTSESATPESSGGS
SGGSMIGQKTLYSFFSPSPARKRHAPSPEPAVQGTGVAGVPEESGDAAAIPAKKAPAGQEEPGTPPSSPLSA
EQLDRIQRNKAAALLRLAARNVPVGFGESWKKHLSGEFGKPYFIKLMGFVAEERKHYTVYPPPHQVFTW
TQMCDIKDVKVVILGQDPAHGPNQAHGLCESVQRPVPPPPSLENIYKELSTDIEDFVHPGHGDLSGWAKQ
GVLLLNAVLTVRAHQANSHKERGWEQFTDAVVSWLNQNSNGLVFLLWGSYAQKKGSAIDRKRHHVLQT
AHPSPLSVYRGFFGCRHFSKTNELLQKSGKKPIDWKELSGGSKRTADGSEFEPKKKRKV
SEQ ID NO: 137, UNG2-N213D
MIGQKTLYSFFSPSPARKRHAPSPEPAVQGTGVAGVPEESGDAAAIPAKKAPAGQEEPGTPPSSPLSAEQLD
RIQRNKAAALLRLAARNVPVGFGESWKKHLSGEFGKPYFIKLMGFVAEERKHYTVYPPPHQVFTWTQM
CDIKDVKVVILGQDPYHGPNQAHGLCFSVQRPVPPPPSLENTYKELSTDIEDFVHPGHGDLSGWAKQGVL
LLDAVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 138, gCBEv0.1 (M-bpSV40 NLS-nCas9-linker-UNG2- N213D-linker-bpSV40 NLS)
MKRTADGSEFESPKKKRKVDKKYSIGLAIGTNSVGWAVITDEYKVPSKKEKVLGNTDRHSIKKNLIGALL
FDSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDLLRKQRTFDNGS
IPHQIHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQELDIN
RLSDYDVDHIVPQSFLKDDSIDNKVLTRSDKNRGKSDNVPSEEVVKKMKNYWRQLLNAKLITQRKEDNL
TKAERGGLSELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGDSGGSSGGSSGSETPGTSESATPESSGGS
SGGSMIGQKTLYSFFSPSPARKRHAPSPEPAVQGTGVAGVPEESGDAAAIPAKKAPAGQEEPGTPPSSPLSA
EQLDRIQRNKAAALLRLAARNVPVGFGESWKKHLSGEFGKPYFIKLMGFVAEERKHYTVYPPPHQVFTW
TQMCDIKDVKVVILGQDPYHGPNQAHGLCFSVQRPVPPPPSLENIYKELSTDIEDFVHPGHGDLSGWAKQ
GVLLLDAVLTVRAHQANSHKERGWEQFTDAVVSWLNQNSNGLVFLLWGSYAQKKGSAIDRKRHHVLQT
AHPSPLSVYRGFFGCRHFSKTNELLQKSGKKPIDWKELSGGSKRTADGSEFEPKKKRKV
SEQ ID NO: 139, UNG2-Y156A + N-trucation 88aa (NA88), the double strikethrough indicates
deleted amino acids. (“Δ88”)

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPAHGPNQAHGLCFSVQRPVPPPPSLENTYKELSTDIEDFVHPGHGDLSGWAKQGVL
LLNAVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 140, gTBEv0.2 (M-bpSV40 NLS-nCas9-linker-UNG2-Y156A + NA88-linker-bpSV40 NLS)
MKRTADGSEFESPKKKRKVDKKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKSNFDLAEDAKLQLS
KDTYDDDLDNLLAQIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNREDLLRKQRTEDNGS
IPHQIHLGELHAILRRQEDFYPELKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQELDIN
RLSDYDVDHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKLKGSPED
NEQKQLFVEQHKHYLDEITEQISEFSKRVILADANLDKVLSAYNKHRDKPIREQAENIIHLFTLTNLGAPAA
FKYFDTTIDRKRYTSTKEVLDATLIHQSITGLYETRIDLSQLGGDSGGSSGGSSGSETPGTSESATPESSGGS
SGGSNVPVGFGESWKKHLSGEFGKPYFIKLMGFVAEERKHYTVYPPPHQVFTWTQMCDIKDVKVVILGQ
DPAHGPNQAHGLCFSVQRPVPPPPSLENIYKELSTDIEDFVHPGHGDLSGWAKQGVLLLNAVLTVRAHQA
NSHKERGWEQFTDAVVSWLNQNSNGLVELLWGSYAQKKGSAIDRKRHHVLQTAHPSPLSVYRGFFGCR
HFSKTNELLQKSGKKPIDWKELSGGSKRTADGSEFEPKKKRKV
SEQ ID NO: 141, UNG2A88-N213D, the double strikethrough indicates deleted amino acids
(“Δ88”)

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPYHGPNQAHGLCFSVQRPVPPPPSLENIYKELSTDIEDFVHPGHGDLSGWAKQGVL
LLDAVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 142, gCBEv0.2 (M-bpSV40 NLS-nCas9-linker-UNG2A88-N213D-linker-bpSV40 NLS)
MKRTADGSEFESPKKKRKVDKKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKERGHFLIEGDLNPDNSDVDKLFIQLVQTYNQL
FEENPINASGVDAKAILSARLSKSRRLENLIAQLPGEKKNGLFGNLIALSLGLIPNFKSNFDLAEDAKLQLS
KDTYDDDLDNLLAQIGDQYADLFLAAKNLSDAILLSDILRVNTEITKAPLSASMIKRYDEHHQDLILLKAL
VRQQLPEKYKEIFFDQSKNGYAGYIDGGASQEEFYKFIKPILEKMDGTEELLVKLNREDLLRKQRTFDNGS
IPHQIHLGELHAILRRQEDFYPELKDNREKIEKILTFRIPYYVGPLARGNSREAWMTRKSEETITPWNFEEV
VDKGASAQSFIERMTNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQELDIN
RLSDYDVDHIVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKSKLVSDFRKDFQ
FYKVREINNYHHAHDAYLNAVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMPQVNIVKKTEVQTGGFSKESILP
KRNSDKLIARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKLKGSPED
NEQKQLFVEQHKHYLDEITEQISEFSKRVILADANLDKVLSAYNKHRDKPIREQAENIIHLFTLTNLGAPAA
FKYFDTTIDRKRYTSTKEVLDATLIHQSITGLYETRIDLSQLGGDSGGSSGGSSGSETPGTSESATPESSGGS
SGGSNVPVGFGESWKKHLSGEFGKPYFIKLMGFVAEERKHYTVYPPPHQVFTWTQMCDIKDVKVVILGQ
DPYHGPNQAHGLCFSVQRPVPPPPSLENTYKELSTDIEDFVHPGHGDLSGWAKQGVLLLDAVLTVRAHQA
NSHKERGWEQFTDAVVSWLNQNSNGLVFLLWGSYAQKKGSAIDRKRHHVLQTAHPSPLSVYRGFFGCR
HFSKTNELLQKSGKKPIDWKELSGGSKRTADGSEFEPKKKRKV
SEQ ID NO: 143, gTBEv0.3 (M-bpSV40.NLS-UNG2A88-Y156A-linker--nCas9-linker-bpSV40 NLS)
MKRTADGSEFESPKKKRKVNVPVGFGESWKKHLSGEFGKPYFIKLMGFVAEERKHYTVYPPPHQVFTWT
QMCDIKDVKVVILGQDPAHGPNQAHGLCESVQRPVPPPPSLENIYKELSTDIEDFVHPGHGDLSGWAKQG
VLLLNAVLTVRAHQANSHKERGWEQFTDAVVSWLNQNSNGLVFLLWGSYAQKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQETFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYFTVYNELTKVKYVTEGMRKPAFLSGEQKKAIVDLLEKTNRKVTVKQLKEDYFK
KIECFDSVEISGVEDRFNASLGTYHDLLKIIKDKDELDNEENEDILEDIVLTLTLFEDREMIEERLKTYAHLF
DDKVMKQLKRRRYTGWGRLSRKLINGIRDKQSGKTILDELKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 144, UNG2A88-Y156A + A214V, the double strikethrough indicates deleted amino
acids (“Δ88”)

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPAHGPNQAHGLCFSVQRPVPPPPSLENIYKELSTDIEDFVHPGHGDLSGWAKQGVL
LLNVVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 145, gTBEv1.1 (M-bpSV40 NLS-UNG2A88-Y156A + A214V-linker--nCas9-linker-
bpSV40 NLS)
MKRTADGSEFESPKKKRKVNVPVGFGESWKKHLSGEFGKPYFIKLMGFVAEERKHYTVYPPPHQVFTWT
QMCDIKDVKVVILGQDPAHGPNQAHGLCFSVQRPVPPPPSLENIYKELSTDIEDFVHPGHGDLSGWAKQG
VLLLNVVLTVRAHQANSHKERGWEQFTDAVVSWLNQNSNGLVFLLWGSYAQKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDST
DKADLRLIYLALAHMIKERGHFLIEGDLNPDNSDVDKLFIQLVQTYNQLFEENPINASGVDAKAILSARLS
KSRRLENLIAQLPGEKKNGLEGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTEDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYFTVYNELTKVKYVTEGMRKPAFLSGEQKKAIVDLLFKINRKVTVKQLKEDYFK
KIECFDSVEISGVEDRENASLGTYHDLLKIIKDKDELDNEENEDILEDIVLTLILFEDREMIEERLKTYAHLF
DDKVMKQLKRRRYTGWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGETVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGQSKRTADGSEFEPKKKRKV
SEQ ID NO: 146, UNG2A88-Y156A + A214T, the double strikethrough indicates deleted amino
acids (“Δ88”)

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPAHGPNQAHGLCFSVQRPVPPPPSLENIYKELSTDIEDFVHPGHGDLSGWAKQGVL
LLNTVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 147, gTBEv1.2 (M-bpSV40 NLS-UNG2488-Y156A + A214T-linker--nCas9-linker-
bpSV40 NLS)
MKRTADGSEFESPKKKRKVNVPVGFGESWKKHLSGEFGKPYFIKLMGFVABERKHYTVYPPPHQVFTWT
QMCDIKDVKVVILGQDPAHGPNQAHGLCFSVQRPVPPPPSLENIYKELSTDIEDFVHPGHGDLSGWAKQG
VLLLNTVLTVRAHQANSHKERGWEQFTDAVVSWLNQNSNGLVELLWGSYAQKKGSAIDRKRHHVLQTA
HPSPLSVYRGFFGCRHFSKTNELLQKSGKKPIDWKELSGGSSQGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR.
RKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYETVYNELTKVKYVTEGMRKPAFLSGEQKKAIVDLLEKTNRKVTVKQLKEDYFK
KIECFDSVEISGVEDRENASLGTYHDLLKIIKDKDELDNEENEDILEDIVLTLTLFEDREMIEERLKTYAHLF
DDKVMKQLKRRRYTGWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERM
KRTEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDE?IEQI
SEFSKRVILADANLDKVLSAYNKHRDKPIREQAENIIHLFTLTNLGAPAAFKYEDTTIDRKRYTSTKEVLDA
TLIHQSITGLYETRIDLSQLGGDSGGSKRTADGSEFEPKKKRKV
SEQ ID NO: 148, UNG2A88-Y156A + Q259A, the double strikethrough indicates deleted amino
acids (“Δ88”)

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPAHGPNQAHGLCFSVQRPVPPPPSLENIYKELSTDIEDFVHPGHGDLSGWAKQGVL
LLNAVLTVRAHQANSHKERGWEQFTDAVVSWLNQNSNGLVFLLWGSYAAKKGSAIDRKRHHVLQTAHP
SPLSVYRGFFGCRHFSKTNELLQKSGKKPIDWKEL
SEQ ID NO: 149, gTBEv1.3 (M-bpSV40 NLS-UNG2488-Y156A + Q259A-linker-nCas9-linker-bpSV40
NLS)
MKRTADGSEFESPKKKRKVNVPVGFGESWKKHLSGEFGKPYFIKLMGFVAEERKHYTVYPPPHQVFTWT
QMCDIKDVKVVILGQDPAHGPNQAHGLCESVQRPVPPPPSLENIYKELSTDIEDFVHPGHGDLSGWAKQG
VLLLNAVLTVRAHQANSHKERGWEQFTDAVVSWLNQNSNGLVFLLWGSYAAKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYFTVYNELTKVKYVTEGMRKPAFLSGEQKKAIVDLLEKTNRKVTVKQLKEDYFK.
KIECFDSVEISGVEDRENASLGTYHDLLKIIKDKDELDNEENEDILEDIVLTLTLFEDREMIEERLKTYAHLF
DDKVMKQLKRRRYTGWGRLSRKLINGIRDKQSGKTILDELKSDGFANRNEMQLIHDDSLTFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 150, UNG2A88-Y156A + A214T + Q259A, the double strikethrough indicates
deleted amino acids (“Δ88”)

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPAHGPNQAHGLCFSVQRPVPPPPSLENIYKELSTDIEDFVHPGHGDLSGWAKQGVL
LLNTVLTVRAHQANSHKERGWEQFTDAVVSWLNQNSNGLVFLLWGSYAAKKGSAIDRKRHHVLQTAHP
SPLSVYRGFFGCRHFSKTNELLQKSGKKPIDWKEL
SEQ ID NO: 151, gTBEv2 (M-bpSV40 NLS-UNG2A88-Y156A + A214T + Q259A -nCas9-linker-bpSV40
NLS)
MKRTADGSEFESPKKKRKVNVPVGFGESWKKHLSGEFGKPYFIKLMGFVAEERKHYTVYPPPHQVFTWT
QMCDIKDVKVVILGQDPAHGPNQAHGLCFSVQRPVPPPPSLENIYKELSTDIEDFVHPGHGDLSGWAKQG
VLLLNTVLTVRAHQANSHKERGWEQFTDAVVSWLNQNSNGLVFLLWGSYAAKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLEDSGETAEATRLKRTARRRYTR
RKNRICYLQEIFSNEMAKVDDSFFHRLEESELVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNRKVTVKQLKEDYFK
KIECFDSVEISGVEDRENASLGTYHDLLKIIKDKDFLDNEENEDILEDIVLTLTLFEDREMIEERLKTYAHLF
DDKVMKQLKRRRYTGWGRLSRKLINGIRDKQSGKTILDELKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 152, UNG2A88-Y156A + A214T + Q259A, the double strikethrough indicates
deleted amino acids (“Δ88”)

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPAHGPNQAHGLCFSVQRPVPPPPSLENIYKELSTDIEDFVHPGHGDLSGWAKQGVL
LLNTVLTVRAHQANSHKERGWEQFTDAVVSWLNQNSNGLVFLLWGSYAAKKGSAIDRKRHHVLQTAHP
SPLSVDRGFFGCRHFSKTNELLQKSGKKPIDWKEL
SEQ ID NO: 153, gTBEv3 (M-bpSV40 NLS-UNG2A88-Y156A + A214T + Q259A-nCas9-linker-bpSV40
NLS)
MKRTADGSEFESPKKKRKVNVPVGFGESWKKHLSGEFGKPYFIKLMGFVAEERKHYTVYPPPHQVFTWT
QMCDIKDVKVVILGQDPAHGPNQAHGLCFSVQRPVPPPPSLENIYKELSTDIEDFVHPGHGDLSGWAKQG
VLLLNTVLTVRAHQANSHKERGWEQFTDAVVSWLNQNSNGLVFLLWGSYAAKKGSAIDRKRHHVLQTA
HPSPLSVDRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNEDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTEDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYFTVYNELTKVKYVTEGMRKPAFLSGEQKKAIVDLLFKTNRKVTVKQLKEDYFK
KIECEDSVEISGVEDRENASLGTYHDLLKIIKDKDELDNEENEDILEDIVLTLTLFEDREMIEERLKTYAHLF
DDKVMKQLKRRRYTGWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGETVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEITEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 154, gCBEv0.3 (M-bpSV40 NLS-UNG2A88-N213D-linker--nCas9-linker-bpSV40 NLS)
MKRTADGSEFESPKKKRKVNVPVGFGESWKKHLSGEFGKPYFIKLMGFVAEERKHYTVYPPPHQVFTWT
QMCDIKDVKVVILGQDPYHGPNQAHGLCFSVQRPVPPPPSLENIYKELSTDIEDFVHPGHGDLSGWAKQG
VLLLDAVLTVRAHQANSHKERGWEQFTDAVVSWLNQNSNGLVFLLWGSYAQKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDST
DKADLRLIYLALAHMIKERGHFLIEGDLNPDNSDVDKLFIQLVQTYNQLFEENPINASGVDAKAILSARLS
KSRRLENLIAQLPGEKKNGLFGNLIALSLGLTPNFKSNEDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSREAWMTRKSEETITPWNFEEVVDKGASAQSFIERMTNFDKN
LPNEKVLPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKTYAHLE
DDKVMKQLKRRRYTGWGRLSRKLINGIRDKQSGKTILDFLKSDGFANRNFMQLIHDDSLIFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 155, UNG2Δ88-N213D + A214V

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPYHGPNQAHGLCFSVQRPVPPPPSLENIYKELSTDIEDFVHPGHGDLSGWAKQGVL
LLDVVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 156, gCBEv1.1 (M-bpSV40 NLS-UNG2A88-N213D + A214V-linker--nCas9-linker-bpSV40
NLS)
MKRTADGSEFESPKKKRKVNVPVGFGESWKKHLSGEFGKPYFIKLMGFVAEERKHYTVYPPPHQVFTWT
QMCDIKDVKVVILGQDPYHGPNQAHGLCFSVQRPVPPPPSLENIYKELSTDIEDFVHPGHGDLSGWAKQG
VLLLDVVLTVRAHQANSHKERGWEQFTDAVVSWLNQNSNGLVFLLWGSYAQKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYFTVYNELTKVKYVTEGMRKPAFLSGEQKKAIVDLLFKTNRKVTVKQLKEDYFK
KIECEDSVEISGVEDRENASLGTYHDLLKIIKDKDELDNEENEDILEDIVLTLTLFEDREMIEERLKTYAHLF
DDKVMKQLKRRRYTGWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGETVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEITEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 157, UNG2Δ88-N213D + K184A

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPYHGPNQAHGLCFSVQRPVPPPPSLENIYAELSTDIEDFVHPGHGDLSGWAKQGVL
LLDAVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 158, gCBEv1.2 (M-bpSV40 NLS-UNG2488-N213D + K184A-linker--nCas9-linker-
bpSV40 NLS)
MKRTADGSEFESPKKKRKVNVPVGFGESWKKHLSGEFGKPYFIKLMGFVAEERKHYTVYPPPHQVFTWT
QMCDIKDVKVVILGQDPYHGPNQAHGLCFSVQRPVPPPPSLENIYAELSTDIEDFVHPGHGDLSGWAKQG
VLLLDAVLTVRAHQANSHKERGWEQFTDAVVSWLNQNSNGLVFLLWGSYAQKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS.
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYFTVYNELTKVKYVTEGMRKPAFLSGEQKKAIVDLLFKTNRKVTVKQLKEDYFK
KIECFDSVEISGVEDRENASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGEANRNFMQLIHDDSLTFKEDIQKAQ
VSGQGDSLHEHIANLAGSPAIKKGILQTVKVVDELVKVMGRHIKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 159, UNG2Δ88-N213D + K184A + A214V

[symbol]

[symbol]

[symbol]

[symbol]

[symbol]

[symbol]

NVPVGFGESWKKHLSGEFGKPYFIKLMGFVAEERKHYTVYPPPHQVFTWTQM
CDIKDVKVVILGQDPYHGPNQAHGLCFSVQRPVPPPPSLENIYAELSTDIEDFVHPGHGDLSGWAKQGVL
LLDVVLTVRAHQANSHKERGWEQFTDAVVSWLNQNSNGLVFLLWGSYAQKKGSAIDRKRHHVLQTAHP
SPLSVYRGFFGCRHFSKTNELLQKSGKKPIDWKEL
SEQ ID NO: 160, gCBEv2 (M-bpSV40 NLS-UNG2488-N213D + K184A + A214V-nCas9-linker-bpSV40
NLS)
MKRTADGSEFESPKKKRKVNVPVGFGESWKKHLSGEFGKPYFIKLMGFVAEERKHYTVYPPPHQVFTWT
QMCDIKDVKVVILGQDPYHGPNQAHGLCFSVQRPVPPPPSLENIYAELSTDIEDFVHPGHGDLSGWAKQG
VLLLDVVLTVRAHQANSHKERGWEQFTDAVVSWLNQNSNGLVFLLWGSYAQKKGSAIDRKRHHVLQTA
HPSPLSVYRGFFGCRHFSKTNELLQKSGKKPIDWKELSGGSSGGSSGSETPGTSESATPESSGGSSGGSDK
KYSIGLAIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASGVDAKAILSARLS
KSRRLENLIAQLPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQSFIERMTNEDKN
LPNEKVLPKHSLLYEYFTVYNELTKVKYVTEGMRKPAFLSGEQKKAIVDLLFKTNRKVTVKQLKEDYFK
KIECFDSVEISGVEDRENASLGTYHDLLKIIKDKDFLDNEENEDILEDIVLTLTLFEDREMIEERLKTYAHLE
DDKVMKQLKRRRYTGWGRLSRKLINGIRDKQSGKTILDFLKSDGFANRNFMQLIHDDSLTFKEDIQKAQ
VSGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREINNYHHAHDAYLN
AVVGTALIKKYPKLESEFVYGDYKVYDVRKMTAKSEQEIGKATAKYFFYSNIMNFEKTEITLANGEIRKRP
LIETNGETGETVWDKGRDFATVRKVLSMPQVNTVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
SEQ ID NO: 161, gTBEv4 (M-bpSV40 NLS-linker-SpCas9-D10A(2-1248)-linker-UNG2Δ88-
Y156A + A214T + Q259A-linker-SpCas9-D10A(1249-1368)-linker-bpSV40 NLS)
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNEDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPELKDNREKIEKILTFRIPYYVGPLARGNSRFAWMIRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEENEDI
LEDIVLTLTLFEDREMIEERLKTYAHLEDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTGGF
SKESILPKRNSDKLIARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYVNFLYLASHYEKLK
GSSGGSSGGSSGSETPGTSESATPESSGGSSGGSNVPVGFGESWKKHLSGEFGKPYFIKLMGFVAEERKHY
TVYPPPHQVFTWTQMCDIKDVKVVILGQDPAHGPNQAHGLCFSVQRPVPPPPSLENIYKELSTDIEDFVHP
GHGDLSGWAKQGVLLLNTVLTVRAHQANSHKERGWEQFTDAVVSWLNQNSNGLVELLWGSYAAKKGS
AIDRKRHHVLQTAHPSPLSVDRGFFGCRHESKTNELLQKSGKKPIDWKELSGGSSGGSSGSETPGTSESAT
PESSGGSSGGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGDSGGSKRTADGSEFE
PKKKRKV
SEQ ID NO: 162, gTBEv5 (M-bpSV40 NLS-linker-SpCas9-D10A(2-1047)-linker-UNG2Δ88-
Y156A + A214T + Q259A-linker-SpCas9-D10A(1064-1368)-linker-bpSV40 NLS)
MKRTADGSEFESPKKKRKVSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKLIKDKDELDNEENEDI
LEDIVLTLTLFEDREMIEERLKTYAHLFDDKVMKQLKRRRYTGWGRLSRKLINGIRDKQSGKTILDELKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQTTKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKSGGSSGGSSGSETPGTSESATPESSGGSSGGSNVPVGFGESWKKHLSGEFGKPYFIKL
MGFVAEERKHYTVYPPPHQVFTWTQMCDIKDVKVVILGQDPAHGPNQAHGLCESVQRPVPPPPSLENTY
KELSTDIEDFVHPGHGDLSGWAKQGVLLLNTVLTVRAHQANSHKERGWEQFTDAVVSWLNQNSNGLVF
LLWGSYAAKKGSAIDRKRHHVLQTAHPSPLSVDRGFFGCRHESKTNELLQKSGKKPIDWKELSGGSSGGS
SGSETPGTSESATPESSGGSSGGSETNGETGEIVWDKGRDFATVRKVLSMPQVNIVKKTEVQTGGESKESI
LPKRNSDKLIARKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEKLKGSPED
NEQKQLFVEQHKHYLDEITEQISEFSKRVILADANLDKVLSAYNKHRDKPIREQAENITHLFTLTNLGAPAA
FKYFDTTIDRKRYTSTKEVLDATLIHQSITGLYETRIDLSQLGGDSGGSKRTADGSEFEPKKKRKV
SEQ ID NO: 163, SpG nCas9-D10A amino acid sequence, 1367 aa
DKKYSIGLAIGTNSVGWAVITDEYKVPSKKEKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPINASGVDAKAILSA
RLSKSRRLENLIAQLPGEKKNGLFGNLIALSLGLTPNFKSNEDLAEDAKLQLSKDTYDDDLDNLLAQIGD
QYADLFLAAKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGELHAILRRQE
DFYPFLKDNREKIEKILTFRIPYYVGPLARGNSRFAWMTRKSEETITPWNFEEVVDKGASAQSFIERMTNF
DKNLPNEKVLPKHSLLYEYFTVYNELTKVKYVTEGMRKPAFLSGEQKKAIVDLLFKTNRKVTVKQLKED
YFKKIECEDSVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTFKEDIQK
AQVSGQGDSLHEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKEDNLTKAERGGLSELDKAGFIK
RQLVETRQITKHVAQILDSRMNTKYDENDKLIREVKVITLKSKLVSDFRKDFQFYKVREINNYHHAHDAY
LNAVVGTALIKKYPKLESEFVYGDYKVYDVRKMIAKSEQEIGKATAKYFFYSNIMNFFKTEITLANGEIRK
RPLIETNGETGEIVWDKGRDFATVRKVLSMPQVNIVKKTEVQTGGFSKESILPKRNSDKLIARKKDWDPK
KYGGFLWPTVAYSVLVVAKVEKGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPEDNEQKQLEVEQHKHYLDEI
IEQISEFSKRVILADANLDKVLSAYNKHRDKPIREQAENIIHLFTLTNLGAPAAFKYEDTTIDRKQYRSTKE
VLDATLIHQSITGLYETRIDLSQLGGD
SEQ ID NO: 164, gCBEv3 (M-bpSV40 NLS-linker-SpCas9-D10A(2-1047)-linker-UNG2Δ88-
K184A + N213D + A214V-linker-SpCas9-D10A(1064-1368)-linker-bpSV40 NLS)
MKRTADGSEFESPKKKRKVSGGSDKKYSIGLAIGTNSVGWAVITDEYKVPSKKEKVLGNTDRHSIKKNLI
GALLFDSGETAEATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKERGHFLIEGDLNPDNSDVDKLFIQLVQ
TYNQLFEENPINASGVDAKAILSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDELDNEENEDI
LEDIVLTLTLFEDREMIEERLKTYAHLEDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWRQLLNAKLITQ
RKFDNLTKAERGGLSELDKAGFIKRQLVETRQITKHVAQILDSRMNTKYDENDKLIREVKVITLKSKLVSD
FRKDFQFYKVREINNYHHAHDAYLNAVVGTALIKKYPKLESEFVYGDYKVYDVRKMIAKSEQEIGKATA
KYFFYSNIMNFFKSGGSSGGSSGSETPGTSESATPESSGGSSGGSNVPVGFGESWKKHLSGEFGKPYFIKL
MGFVAEERKHYTVYPPPHQVFTWTQMCDIKDVKVVILGQDPYHGPNQAHGLCFSVQRPVPPPPSLENIY
AELSTDIEDFVHPGHGDLSGWAKQGVLLLDVVLTVRAHQANSHKERGWEQFTDAVVSWLNQNSNGLVF
LLWGSYAQKKGSAIDRKRHHVLQTAHPSPLSVYRGFFGCRHFSKTNELLQKSGKKPIDWKELSGGSSGGS.
SGSETPGTSESATPESSGGSSGGSETNGETGEIVWDKGRDFATVRKVLSMPQVNIVKKTEVQTGGFSKESI
LPKRNSDKLIARKKDWDPKKYGGEDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEKLKGSPED
NEQKQLFVEQHKHYLDEITEQISEFSKRVILADANLDKVLSAYNKHRDKPIREQAENIIHLFTLTNLGAPAA
FKYFDTTIDRKRYTSTKEVLDATLIHQSITGLYETRIDLSQLGGDSGGSKRTADGSEFEPKKKRKV

TABLE A
Homo sapiens; Pan troglodytes; Pongo abelii; Theropithecus gelada; Gorilla gorilla gorilla; Cercocebus atys;
Mandrillus leucophaeus; Hylobates moloch; Macaca nemestrina; Rhinopithecus bieti; Chlorocebus sabaeus;
Sapajus apella; Callithrix jacchus; Aotus nancymaae; Cebus imitator; Propithecus coquereli; Otolemur
garnettii; Microcebus murinus; Ursus arctos; Ailuropoda melanoleuca; Molossus molossus; Equus caballus;
Hyaena hyaena; Panthera leo; Canis lupus familiaris; Castor canadensis; Puma concolor; Sciurus carolinensis;
Panthera tigris; Equus przewalskii; Canis lupus dingo; Hipposideros armiger; Puma yagouaroundi; Panthera
uncia; Suricata suricatta; Felis catus; Rangifer tarandus platyrhyncus; Ovis aries; Moschus berezovskii;
Miniopterus natalensis; Phacochoerus africanus; Rousettus aegyptiacus; Marmota marmota marmota;
Galeopterus variegatus; Marmota monax; Tupaia chinensis; Lynx pardinus; Odocoileus virginianus texanus;
Cervus elaphus; Meles meles; Myotis lucifugus; Sorex araneus; Bos mutus; Arvicola amphibius; Suncus
etruscus; Pteronotus parnellii mesoamericanus; Capra hircus; Nycticebus coucang; Myotis brandtii; Chinchilla
lanigera; Enhydra lutris kenyoni; Diceros bicornis minor; Heterocephalus glaber; Apodemus sylvaticus;
Vicugna pacos; Mus musculus; Mastomys coucha; Arvicanthis niloticus; Octodon degus; Cricetulus griseus;
Peromyscus leucopus; Acomys russatus; Eptesicus fuscus; Sus scrofa; Mustela putorius furo; Grammomys
surdaster; Eschrichtius robustus; Manis pentadactyla; Camelus ferus; Camelus bactrianus; Mesocricetus
auratus; Rattus rattus; Dipodomys spectabilis; Camelus dromedarius; Onychomys torridus; Choloepus
didactylus; Fukomys damarensis; Nannospalax galili; Oryctolagus cuniculus; Perognathus longimembris
pacificus; Lutra lutra; Orycteropus afer afer; Cavia porcellus; Manis javanica; Phodopus roborovskii; Mirounga
angustirostris; Dipodomys ordii; Talpa occidentalis; Pipistrellus kuhlii; Ochotona curzoniae; Antechinus
flavipes; Trichechus manatus latirostris; Sarcophilus harrisii; Trichosurus vulpecula; Phascolarctos cinereus;
Loxodonta africana; Gracilinanus agilis; Vombatus ursinus; Monodelphis domestica; Chrysochloris asiatica;
Meriones unguiculatus; Dromiciops gliroides; Echinops telfairi; Elephantulus edwardii; Rhinolophus sinicus;
Chrysemys picta bellii; Dermochelys coriacea; Malaclemys terrapin pileata; Trachemys scripta elegans;
Gopherus evgoodei; Chelonoidis abingdonii; Caretta caretta; Mauremys mutica; Mauremys reevesii; Apteryx
mantelli mantelli, Centropus unirufus; Nyctereutes procyonoides; Tachyglossus aculeatus; Alligator
mississippiensis; Acipenser ruthenus; Tympanuchus pallidicinctus; Pelodiscus sinensis; Centrocercus
urophasianus; Geotrypetes seraphini; Nipponia nippon; Latimeria chalumnae; Microcaecilia unicolor;
Meleagris gallopavo; Lagopus muta; Hirundo rustica rustica; Strigops habroptila; Coturnix japonica; Zosterops
borbonicus; Motacilla alba alba; Gavia stellata; Spizaetus tyrannus; Lamprotornis superbus; Apus apus;
Onychostruthus taczanowskii; Bufo bufo; Burhinus bistriatus; Lonchura striata domestica; Engystomops
pustulosus; Bufo gargarizans; Chloebia gouldiae; Rana temporaria; Scyliorhinus torazame; Brienomyrus
brachyistius; Xenopus tropicalis; Salvelinus alpinus; Carcharodon carcharias; Nanorana parkeri; Oncorhynchus
nerka; Pitangus sulphuratus; Podarcis muralis; Varanus komodoensis; Xenopus laevis; Thunnus albacares;
Bombina bombina; Protopterus annectens; Polypterus senegalus; Puntigrus tetrazona; Scomber japonicus;
Synchiropus splendidus; Clarias gariepinus; Megalops cyprinoides; Plectropomus leopardus; Chanos chanos;
Zootoca vivipara; Lacerta agilis; Cheilinus undulatus; Alosa alosa; Eublepharis macularius; Sinocyclocheilus
grahami; Tachysurus fulvidraco; Micropterus dolomieu; Lerista edwardsae; Paramormyrops kingsleyae;
Synaphobranchus kaupii; Cottoperca gobio; Colossoma macropomum; Siniperca chuatsi; Pangasius djambal;
Lates calcarifer; Bagarius yarrelli; Silurus meridionalis; Oryzias melastigma; Myripristis murdjan; Ictalurus
punctatus; Syngnathus scovelli; Gymnodraco acuticeps; Oryzias javanicus; Tetraodon nigroviridis;
Larimichthys crocea; Gymnothorax javanicus; Pangasianodon gigas; Epinephelus fuscoguttatus; Ictalurus
furcatus; Sphaerodactylus townsendi; Notechis scutatus; Monopterus albus; Rattus norvegicus; Labeo rohita;
Pristis pectinata; Danionella translucida; Betta splendens; Scleropages formosus; Denticeps clupeoides;
Hymenochirus boettgeri; Pantherophis guttatus; Acanthopagrus latus; Thalassophryne amazonica; Nibea
albiflora; Epinephelus lanceolatus; Phrynocephalus forsythii; Protobothrops mucrosquamatus; Cynoglossus
semilaevis; Hemicordylus capensis; Sceloporus undulatus; Spea bombifrons; Sparus aurata; Thamnophis
elegans; Crotalus tigris; Amblyraja radiata; Perca fluviatilis; Perca flavescens; Danio rerio; Rissa tridactyla;
Naja naja; Labrus bergylta; Takifugu rubripes; Toxotes jaculatrix; Sander lucioperca; Etheostoma spectabile;
Etheostoma cragini; Callipepla squamata; Anguilla anguilla; Mastacembelus armatus; Stegastes partitus;
Pseudonaja textilis; Electrophorus electricus; Pleurodeles waltl; Austrofundulus limnaeus; Conger conger;
Pleuronectes platessa; Pogona vitticeps; Callorhinchus milli; Nothobranchius furzeri; Oryzias latipes; Gadus
morhua; Scophthalmus maximus; Polyodon spathula; Araneus ventricosus; Anabarilius grahami; Anolis
carolinensis; Gekko japonicus; Dicentrarchus labrax; Clupea harengus; Lepisosteus oculatus; Notolabrus
celidotus; Takifugu flavidus; Nematolebias whitei; Uloborus diversus; Python bivittatus; Petromyzon marinus;
Asterias rubens; Seriola lalandi dorsalis; Sinocyclocheilus anshuiensis; Trichonephila clavata; Phocoena sinus;
Nephila pilipes; Trichonephila inaurata madagascariensis; Stegodyphus dumicola; Stegodyphus mimosarum;
Caerostris darwini; Saccoglossus kowalevskii; Lingula anatina; Branchiostoma floridae; Procambarus clarkii;
Cinara cedri; Penaeus monodon; Sipha flava; Crassostrea gigas; Penaeus vannamei; Crassostrea virginica;
Dreissena polymorpha; Portunus trituberculatus; Aplysia californica; Daktulosphaira vitifoliae; Zootermopsis
nevadensis; Holothuria leucospilota; Centruroides sculpturatus; Branchiostoma belcheri; Biomphalaria
glabrata; Pecten maximus; Apostichopus japonicus; Capitella teleta; Polistes dominula; Argiope bruennichi;
Oedothorax gibbosus; Owenia fusiformis; Exaiptasia diaphana; Ignelater luminosus; Vespula vulgaris; Haliotis
rufescens; Fopius arisanus; Aphis glycines; Rhopalosiphum maidis; Polistes canadensis; Mizuhopecten
yessoensis; Acropora millepora; Anneissia japonica; Coptotermes formosanus; Vespa crabro; Elysia chlorotica;
Parasteatoda tepidariorum; Bulinus truncatus; Medioppia subpectinata; Branchiostoma lanceolatum; Aphis
gossypii; Melanaphis sacchari; Zophobas morio; Gigantopelta aegis; Aphis craceivora; Soboliphyme baturini;
Cryptotermes secundus; Ixodes scapularis; Diachasma alloeum; Solenopsis invicta; Orbicella faveolata; Idotea
baltica; Lamprigera yunnana; Pomacea canaliculata; Haliotis rubra; Cephus cinctus; Stylophora pistillata;
Pogonomyrmex barbatus; Chelonus insularis; Nematostella vectensis; Coccinella septempunctata; Leptopilina
heterotoma; Homarus americanus; Cotesia congregata; Nezara viridula; Myzus persicae; Trichogramma
pretiosum; Nasonia vitripennis; Harmonia axyridis; Lamellibrachia satsuma; Tenebrio molitor; Desmophyllum
pertusum; Halyomorpha halys; Adelges cooleyi; Ctenocephalides felis; Cotesia typhae; Agrilus planipennis;
Dermacentor andersoni; Acyrthosiphon pisum; Planctomycetota bacterium; Ciona intestinalis; Osmia lignaria;
Neogale vison; Monomorium pharaonis; Patiria miniata; Osmia bicornis bicornis; Thioalkalivibrio sp. XN279;
Linepithema humile; Dendronephthya gigantea; Pocillopora damicornis; Acanthoscelides obtectus; Ostrea
edulis; Orussus abietinus; Thioalkalivibrio sp. XN8; Trichoplax sp. H2; Apis laboriosa; Trichoplax adhaerens;
Pediculus humanus corporis; Megachile rotundata; Acromyrmex charruanus; Temnothorax curvispinosus;
Eurytemora affinis; Frieseomelitta varia; Hypsibius exemplaris; Cotesia glomerata; Copidosoma floridanum;
Aethina tumida; Trichinella zimbabwensis; Armadillidium vulgare; Venturia canescens; Ooceraea biroi;
Wasmannia auropunctata; Trichinella spiralis; Octopus sinensis; Acromyrmex echinatior; Cyphomyrmex
costatus; Ceratina calcarata; Rhipicephalus sanguineus; Batillaria attramentaria; Dufourea novaeangliae;
Formica exsecta; Callosobruchus maculatus; Ixodes persulcatus; Hyalella azteca; Nomia melanderi; Lytechinus
variegatus; Psylliodes chrysocephala; Daphnia pulicaria; Gonioctena quinquepunctata; Microplitis demolitor;
Lytechinus pictus; Bemisia tabaci; Leptotrombidium deliense; Tribolium castaneum; Actinia tenebrosa;
Acanthaster planci; Haemaphysalis longicornis; Eufriesea mexicana; Cimex lectularius; Belonocnema kinseyi;
Daphnia pulex; Rhipicephalus microplus; Bombus pyrosoma; Terrapene carolina triunguis; Diuraphis noxia;
Trachymyrmex septentrionalis; Wenzhouxiangella sp. XN24; Dinothrombium tinctorium; Timema monikensis;
Phycisphaerales bacterium; Ceratosolen solmsi marchali; Trachymyrmex cornetzi; Timema californicum;
Eriocheir sinensis; Cloeon dipterum; Neodiprion pinetum; Xenia sp. Carnegie-2017; Gryllus bimaculatus;
Neodiprion virginianus; Phyllotreta striolata; Timema poppensis; Trichuris suis; Diprion similis; Mya arenaria;
Homalodisca vitripennis; Timema cristinae; Holotrichia oblita; Strongylocentrotus purpuratus; Apolygus
lucorum; Cataglyphis hispanica; Plakobranchus ocellatus; Timema tahoe; Timema douglasi; Hylaeus
anthracinus; Pseudomyrmex gracilis; Ampulex compressa; Leptinotarsa decemlineata; Notodromas monacha;
Trichuris trichiura; Anoplophora glabripennis; Tribolium madens; Periplaneta americana; Diabrotica virgifera
virgifera; Sitophilus oryzae; Atta colombica; Darwinula stevensoni; Phaedon cochleariae; Thrips palmi;
Cyanobacteria bacterium CYA; Schistocerca gregaria; Vollenhovia emeryi; Schistocerca serialis cubense;
Patella vulgata; Priapulus caudatus; Macrosteles quadrilineatus; Planctomycetales bacterium ZRK34;
Tropilaelaps mercedesae; Acidobacteriota bacterium; Bombus terrestris; Oppia nitens; Leptopilina boulardi;
Scortum barcoo; Bombus vosnesenskii; Phallusia mammillata; Dinoponera quadriceps; Phycisphaeraceae
bacterium; Ischnura elegans; Diaphorina citri; Eretmocerus hayati; Blattella germanica; Chrysoperla carnea;
Photinus pyralis; Oopsacas minuta; Diabrotica balteata; Armadillidium nasatum; Trinorchestia longiramus;
Mytilus galloprovincialis; Schistocerca nitens; Aphidius gifuensis; Nicrophorus vespilloides; Frankliniella
occidentalis; Gammaproteobacteria bacterium; Brassicogethes aeneus; Encephalitozoon hellem; Phycisphaerae
bacterium; Amphimedon queenslandica; Harpegnathos saltator; Mytilus coruscus; Vittaforma corneae ATCC
50505; Encephalitozoon romaleae SJ-2008; Galendromus occidentalis; Ramazzottius varieomatus; Daphnia
magna; Athalia rosae; Daphnia sinensis; Ladona fulva; Encephalitozoon cuniculi GB-MI; Callosobruchus
chinensis; Cyprinus carpio; Trachymyrmex zeteki; Fusobacterium necrogenes; Encephalitozoon intestinalis
ATCC 50506; Phycisphaera sp.; Tigriopus californicus; Brachionus calyciflorus; Onthophagus taurus;
Halotydeus destructor; Anaerolineae bacterium; Oppiella nova; Planctomycetaceae bacterium; Bradysia
odoriphaga; Planctomycetales bacterium; Camponotus floridanus; Propionispora vibrioides; Dimorphilus
gyrociliatus; Sarcoptes scabiei; Folsomia candida; Deltaproteobacteria bacterium RIFOXYA12_FULL_58_15;
Dermatophagoides farinae; Candidatus Parcubacteria bacterium; Methanocella sp. CWC-04; Leptolyngbya sp.
PLA1; Clostridium polyendosporum; bacterium; Rickettsiales bacterium; Nilaparvata lugens; Clostridium
perfringens; Asbolus verrucosus; Abscondita terminalis; Clostridium saudiense; Odontomachus brunneus;
Tepidimicrobium xylanilyticum; Megalopta genalis; Clostridium sp. DJ247; Abditibacteriota bacterium;
Candidatus Desantisbacteria bacterium CG1_02_38_46; Clostridium acidisoli; Rubeoparvulum massiliense;
Clostridium estertheticum; Chloroflexota bacterium; Gemmatimonadota bacterium; Colletes gigas; Clostridium
sp. CS001; Apis florea; Clostridium sp. Cult1; Propionigenium maris DSM 9537; Deltaproteobacteria
bacterium RIFCSPLOWO2_12_FULL_60_19; Menidia menidia; Caldilineae bacterium; Clostridium sp. CX1;
Methanocella conradii; Dermatophagoides pteronyssinus; Sporohalobacter salinus; Leptolyngbya sp.;
Muricomes intestini; Tetranychus urticae; Propionispora hippei; Planctomycetes bacterium Poly30; Candidatus
Portnoybacteria bacterium RIFCSPLOWO2_01_FULL_43_11; Clostridium manihotivorum;
Deltaproteobacteria bacterium; Candidatus Buchananbacteria bacterium RBG_13_39_9; Acidobacteria
bacterium 13_1_40CM_56_16; Propionispora hippei DSM 15287; uncultured Clostridium sp.; Apis dorsata;
Candidatus Portnoybacteria bacterium CG03_land_8_20_14_0_80_41_10; Paenibacillus dendritiformis;
Clostridium sp. C8-1-8; Bacillota bacterium; Tissierellia bacterium; Clostridium collagenovorans; Nitrospinota
bacterium; Candidatus Bathyarchaeota archaeon; Candidatus Nealsonbacteria bacterium; Paenibacillus
rhizolycopersici; Atta cephalotes; Candidatus Portnoybacteria bacterium; Methanocella arvoryzae; unclassified
Clostridium; Caloramator proteoclasticus; Pyrinomonadaceae bacterium; Methanocella paludicola; Candidatus
Binatia bacterium; Caloramator fervidus; Habropoda laboriosa; Panonychus citri; Patescibacteria group
bacterium; Propionispora sp. 2/2-37; Paenibacillus sp. ACRRX; Parcubacteria group bacterium; Clostridiales
bacterium; Clostridium tetani; Caerostris extrusa; Fusobacterium perfoetens; Clostridium tagluense;
Selenihalanaerobacter shriftii; Paenibacillus phocaensis; Anaeromicropila populeti; Caloramator sp.;
Clostridium; Eubacteriaceae bacterium; Acetohalobium arabaticum; unclassified Psychrilyobacter;
Psychrilyobacter sp. BL5; Candidatus Uhrbacteria bacterium; Sinanaerobacter chloroacetimidivorans; Blomia
tropicalis; Clostridium aestuarii; Orenia metallireducens; Clostridium botulinum; Clostridium liquoris;
Tepidisphaera sp.; Pseudomonadota bacterium; Candidimonas sp.; Parcubacteria group bacterium
RIFCSPHIGHO2_02_FULL_48_10b; Caloramator australicus; Clostridium cochlearium; candidate division
NC10 bacterium; Clostridium thailandense; Clostridium sp. C105KSO13; Caloramator sp. Dgby_cultured_2;
Eciton burchellii; Paenibacillus taiwanensis; Syntrophobotulus glycolicus; uncultured bacterium;
Natranaerobius thermophilus; Oxobacter pfennigii; Clostridium sp. D1-1; Anaerolineales bacterium;
Heterotrigona itama; Paenibacillus barengoltzii; Vulgatibacter incomptus; Paenibacillus sp. HMSSN-139;
Hypnocyclicus thermotrophus; Candidatus Nealsonbacteria bacterium
CG11_big_fil_rev_8_21_14_0_20_37_68; Ruminiclostridium cellobioparum; Clostridia bacterium;
Clostridium sp. Marseille-Q2269; Clostridium prolinivorans; Clostridium fungisolvens; Clostridium
yunnanense; Desulfitobacterium chlororespirans; Clostridium oryzae; Desulfitobacterium chlororespirans DSM
11544; Vespula germanica; Peptoniphilus phoceensis; Gottschalkia acidurici; Didymodactylos carnosus;
Verrucomicrobiales bacterium; Geotoga petraea; Parapusillimonas sp. SGNA-6; Polyangiaceae bacterium;
Alkaliphilus pronyensis; Ardenticatenia bacterium; Pusillimonas harenae; Pusillimonas soli; Ruminiclostridium
herbifermentans; Gemmatimonadales bacterium; Candidatus Latescibacteria bacterium ADurb.Bin168;
Castellaniella denitrificans; Paenibacillus anaericanus; Clostridium akagii; Pusillimonas sp. T7-7;
Desulfosporosinus sp. FKB; Eleutherodactylus coqui; Paenibacillus sp. MER TA 81-3; Thermoflexales
bacterium; Allacma fusca; Clostridium tepidum; Clostridium lundense; Desulfitobacterium dehalogenans;
Clostridium sp. JN-1; Kiritimatiellia bacterium; candidate division Zixibacteria bacterium; Gottschalkia
purinilytica; Clostridium rhizosphaerae; Polistes exclamans; Schnuerera ultunensis; Desulfosporosinus
youngiae; Verrucomicrobiota bacterium; Noviherbaspirillum aridicola; Pusillimonas ginsengisoli; Natronincola
ferrireducens; Culicoidibacter larvae; Lutispora thermophila; Clostridium cellulovorans; Parcubacteria
bacterium DG_74_3; Pusillimonas sp. M17; Moraxellaceae bacterium; Paenibacillus sp. 481;
Gemmatimonadetes bacterium 13_1_40CM_3_69_22; Thermobrachium celere; Clostridium aminobutyricum;
Candidatus Rokubacteria bacterium; Halanaerobium saccharolyticum; Parcubacteria group bacterium
Gr01-1014_38; Clostridium frigoris; Acanthopleuribacter pedis; Clostridium sporogenes; Clostridium mobile;
Paenibacillus sp. SC116; Oscillospiraceae bacterium; Candidatus Marinimicrobia bacterium; Paenibacillus
arenosi; Paenibacillus sp. FSL R7-0273; Sulfobacillus benefaciens; Clostridium septicum; Apis cerana cerana;
Oceanirhabdus sp. W0125-5; Sphaerochaeta coccoides; Clostridium sp.; Desulfosporosinus orientis;
Candidatus Portnoybacteria bacterium RBG_13_40_8; Vicinamibacterales bacterium; Tissierellales;
unclassified Paenibacillus; Sphingobacteriia bacterium; Gelria sp. Kuro-4; Candidatus Odinarchaeota archaeon;
Anaerolineaceae bacterium; Burkholderiales bacterium; Thermoflexia bacterium; Clostridium caldaquaticum;
Synergistaceae bacterium; Crassaminicella thermophila; Ignavibacteriota bacterium; Epulopiscium sp.;
Actinomycetia bacterium; Microgenomates group bacterium LiPW_16; Clostridium muellerianum;
Alcaligenaceae; Firmicutes bacterium HGW-Firmicutes-13; Methanobacterium; Limosa lapponica baueri;
Keratinibaculum paraultunense; Paenibacillus profundus; Tissierellaceae bacterium; Faecalicatena contorta;
Pusillimonas sp. TS35; Paenibacillus sp. J53TS2; Clostridium sp. BL-8; Clostridium hydrogenum; Clostridium
butyricum; Blastocatellia bacterium; Anaerococcus vaginalis; Aneurinibacillus aneurinilyticus; Clostridium
tunisiense; Paenibacillus apiarius; Acidobacteria bacterium ADurb.Bin051; Legionella sp.;; Candidatus
Niyogibacteria bacterium CG10_big_fil_rev_8_21_14_0_10_46_36; Garciella nitratireducens; Halobacteroides
halobius; Alkaliphilus flagellatus; Alcaligenaceae bacterium; Candidatus Aminicenantes bacterium; Orenia
marismortui; Alkaliphilus peptidifermentans; Paenibacillus tengchongensis; Clostridium chauvoei;
Fusobacteria bacterium ZRK30; Candidatus Nealsonbacteria bacterium CG_4_10_14_0_2_um_filter_35_20;
Chromatiales bacterium 21-64-14; Alkaliphilus transvaalensis; Myxococcales bacterium; Candidatus
Eremiobacteraeota bacterium; Clostridium cadaveris; Anaerococcus nagyae; Oxalobacteraceae bacterium OM1;
Peptoniphilus stercorisuis; Clostridium saccharobutylicum; Candidatus Egerieicola faecale; Paenibacillus
macerans; Clostridium baratii; Sporomusaceae bacterium; Candidatus Omnitrophica bacterium 4484_49;
Aneurinibacillus terranovensis; Clostridium sp. 29_15; Burkholderiaceae bacterium; Castellaniella defragrans;
Anaerococcus senegalensis; Gemmatimonadaceae bacterium; Mucisphaera calidilacus; Candidatus
Calescamantes bacterium; Methanocella sp. PtaU1.Bin125; Mischocyttarus mexicanus; Castellaniella
defragrans 65Phen; Alkaliphilus oremlandii; Haloimpatiens massiliensis; Clostridium sp. LS; Algisphaera
agarilytica; Candidatus Egerieicola pullicola; Clostridium acetobutylicum; Salicibibacter kimchii; Candidatus
Uhrbacteria bacterium RIFCSPHIGHO2_02_FULL_46_47; Paenibacillus agilis; Alcaligenaceae bacterium
CGII-47; Actinomycetota bacterium; Clostridium sp. TW13; Clostridium rectalis; Candidatus
Buchananbacteria bacterium; Bryobacterales bacterium; Clostridium sp. CAG: 221; Paenibacillus sp. P32E;
Clostridium sp. SM-530-WT-3G; Pseudobdellovibrionaceae bacterium; Galdieria sulphuraria;
Chloracidobacterium sp.; Desulfitobacterium sp. PCE1; Natribacillus halophilus; Ruminiclostridium hungatei;
Candidatus Niyogibacteria bacterium RIFCSPLOWO2_12_FULL_41_13; Castellaniella sp.; Clostridium
bowmanii; Paenibacillus senegalimassiliensis; Oscillibacter sp.; Paenibacillus auburnensis; Clostridium
taeniosporum; Paracandidimonas soli; Halonatronum saccharophilum; Candidatus Poribacteria bacterium;
Fusobacterium sp.; Sorangium cellulosum; Haloplasma contractile; Clostridium tetanomorphum;
Ignavibacteriales bacterium; Clostridium cavendishii; Tepiditoga spiralis; Geomonas silvestris;
Limnochordaceae bacterium; Clostridium sp. C2-6-12; Oceanotoga sp. DSM 15011; Gemmatimonadetes
bacterium 13_1_40CM_4_69_8; Clostridium fallax; Paenibacillus piscarius; Pseudalkalibacillus berkeleyi;
Pusillimonas sp. MFBS29; Orchesella cincta; Paenibacillus assamensis; Holophagales bacterium; Thiolapillus
brandeum; Phycisphaera mikurensis; Romboutsia sp. CE17; Anaeromicrobium sediminis; Parapusillimonas
granuli; Romboutsia; Paenibacillus segetis; Bacteroidota bacterium; Clostridium mediterraneense;
Halanaerobium sp. ST460_2HS_T2; Clostridium pasteurianum; Paenibacillus sp. P3E; Elusimicrobiota
bacterium; Clostridium hydrogeniformans; Geomicrobium sp. JCM 19055; Paenibacillus oryzae; Tissierella
simiarum; Candidatus Omnitrophota bacterium; Oscillibacter valericigenes; Halanaerobium kushneri;
Candidatus Niyogibacteria bacterium RIFCSPLOWO2_02_FULL_45_13; Xanthomonadales bacterium;
Sedimentisphaerales bacterium; Lentisphaerae bacterium GWF2_57_35; Candidatus Cellulosilyticum
pullistercoris; Candidatus Lokiarchaeota archaeon; Paenibacillus silagei; Geomonas limicola; Desnuesiella
massiliensis; Nitrospirota bacterium; Paenibacillus camerounensis; Clostridium sp. N3C; Candidatus
Niyogibacteria bacterium; Ruminiclostridium sufflavum; Acidobacteriaceae bacterium; Clostridium
carboxidivorans; Paenibacillus riograndensis; Clostridium gelidum; Pusillimonas noertemannii; Hortaea
werneckii; Paenibacillus sp. Marseille-P2973; Caldisericota bacterium; Marasmitruncus massiliensis; Aquicella
siphonis; Flavobacteriaceae bacterium CRH; Ignavibacteriaceae bacterium; Sporomusaceae bacterium FL31;
Niameybacter massiliensis; Willisornis vidua; Deltaproteobacteria bacterium GWC2 65 14; Sulfurifustis
variabilis; Hathewaya massiliensis; Betaproteobacteria bacterium GR16-43; Caloramator mitchellensis;
Poriferisphaera corsica; Paenibacillus sonchi; Herpetosiphon sp.; Cellulosilyticum sp. WCF-2; Peptoniphilus
timonensis; candidate division WWE3 bacterium CG06_land_8_20_14_3_00_42_16; Clostridium sp. DSM
17811; Candidatus Cloacimonadota bacterium; Saccharibacillus brassicae; Peptoniphilus sp. HMSC062D09;
Usitatibacter rugosus; Bdellovibrio sp.; Desulfotruncus arcticus; Paenibacillus sp. DMB5; Myxococcaceae
bacterium; Candidatus Kryptonium thompsoni; Paenibacillus sp. PK3_47; Crassaminicella profunda;
Ignavibacteriales bacterium CG07_land_8_20_14_0_80_59_12; Caldilineales bacterium; Sulfidibacter
corallicola; Paenibacillus tianjinensis; Pollutimonas subterranea; Herbinix luporum; Ignavibacteria bacterium;
Betaproteobacteria bacterium; Aneurinibacillus migulanus; Clostridium senegalense; Gemmatimonadetes
bacterium 13_1_20CM_4_69_16; Clostridium sulfidigenes; Candidatus Kryptobacter tengchongensis;
Galdieria partita; Paeniclostridium sordellii; Desulfosporosinus sp. BRH_c37; Thermoanaerobaculia bacterium;
Dethiobacter alkaliphilus; Gemmatimonas sp. SG8_28; Paenibacillus borealis; Saccharibacillus sp. WB 17;
Paenibacillus faecalis; Clostridium felsineum; Zhenhengia yiwuensis; Parcubacteria group bacterium
ADurb.Bin159; Rubrivivax sp.; Brockia lithotrophica; Paeniclostridium hominis; Saprospiraceae bacterium;
Solimonas fluminis; Clostridium bovifaecis; Candidatus Woesearchaeota archaeon; Portibacter marinus;
Paenibacillus sp. FSL R5-0912; Paenibacillus tepidiphilus; unclassified Candidatus Frackibacter; Solimonas sp.
K1W22B-7; Chlorobium sp.; Clostridium ljungdahlii; Paenibacillus woosongensis; Pedobacter sp.; Hathewaya
histolytica; Akkermansiaceae bacterium; Tyrophagus putrescentiae; Alkaliphilus sp.; Sebaldella sp.;
Clostridiaceae bacterium HFYG-1003; Prosthecochloris; Gemmatales bacterium; Treponema sp.; Candidatus
Eisenbacteria bacterium; Paenibacillus typhae; Paenibacillus graminis; Candidatus Moranbacteria bacterium
GW2011_GWF1_34_10; Anaerosolibacter carboniphilus; Halanaerobium sp.; Clostridium novyi;
Mesorhizobium sp. ES1-1; Halanaerobium congolense; Clostridium frigidicarnis; Steroidobacter
agaridevorans; Pedobacter mucosus; Clostridium luticellarii; Xanthomonadaceae bacterium; Pullulanibacillus
pueri; Steroidobacter gossypii; Lachnospiraceae bacterium NSJ-143; Legionella erythra; Candidatus Sumerlaea
chitinivorans; Tetrahymena thermophila SB210; Candidatus Kapabacteria bacterium; Candidatus Azambacteria
bacterium RBG_16_47_10; Noviherbaspirillum aerium; Rhodospirillales bacterium; Candidatus Moranbacteria
bacterium GW2011_GWE1_35_17; Aridibacter famidurans; Paenibacillus shirakamiensis; Ardenticatenales
bacterium; Clostridium colicanis; Natroniella sulfidigena; Legionella adelaidensis; Candidatus
Thermoplasmatota archaeon; Lutibacter sp. B2; Oncorhynchus mykiss; bacterium (Candidatus
Gribaldobacteria) CG02_land_8_20_14_3_00_41_15; Candidatus Hydrogenedentes bacterium; Pelodictyon
luteolum; Rhodanobacter sp.; Bacillus luti; Paenibacillus dakarensis; Clostridium butyricum 60E.3;
Cellulosilyticum sp.; Desulfobacterales bacterium; Halobacteriales archaeon; Chlorobiaceae bacterium;
candidate division BRC1 bacterium SM23_51; Calditrichota bacterium; Fusobacterium varium; Candidimonas
humi; Acetobacteraceae bacterium; candidate division Zixibacteria bacterium SM1_73; Geobacter sp. DSM
9736; Legionella yabuuchiae; Limnochordales bacterium; Janthinobacterium sp. 17J80-10; Syntrophaceae
bacterium; Pedobacter sp. SYSU D00535; uncultured Acidobacteriota bacterium; Herpetosiphon geysericola;
Paenibacillus tritici; Leptospirillum ferriphilum; Candidatus Heimdallarchaeota archaeon; Nevskia sp.;
Mesorhizobium sp. AR10; Beijerinckia sp. 28-YEA-48; Bdellovibrionales bacterium; Bacillus cereus;
bacterium HR10; Clarias magur; Clostridium sp. Marseille-P2415; Chlorobaculum thiosulfatiphilum;
Candidatus Saccharicenans sp.; Candidatus Manganitrophus noduliformans; Verrucomicrobia subdivision 3
bacterium; Desulfobacteraceae bacterium; Peptoniphilus sp. HMSC075B08; Peptoniphilus gorbachii;
Acetivibrio saccincola; Paenibacillus sp. S150; Parcubacteria group bacterium CG2_30_44_18;
Psychrilyobacter atlanticus; Lacrimispora saccharolytica; Aquisphaera sp. JC669; Singulisphaera acidiphila;
unclassified Romboutsia; Chitinophagia bacterium; Gammaproteobacteria bacterium
RIFCSPHIGHO2_12_FULL_41_20; Anaerococcus sp. mt242; Paenibacillus caui; Nevskia soli; Sorangium;
Paenibacillus ihumii; Lysobacter telluris; Acholeplasmataceae bacterium; Peptoniphilus sp. SAHP1;
Bradymonadaceae bacterium; Paenibacillus sp. FSL P4-0081; Aneurinibacillus sp. Ricciae_BoGa-3;
Peptoniphilus senegalensis; Bacteroidia bacterium; Clostridium sp. C8; Lachnospiraceae bacterium;
Acidobacteriia bacterium; Verrucomicrobiae bacterium; Castellaniella sp. S9; Candidatus Angelobacter sp.;
Saprospirales bacterium; Gammaproteobacteria bacterium RIFCSPHIGHO2_12_FULL_37_34;
Deltaproteobacteria bacterium CG2_30_66_27; Pedobacter frigidisoli; Clostridium algifaecis; Candidatus
Buchananbacteria bacterium RBG_13_36_9; Sporanaerobacter acetigenes; Pedobacter sp. Leaf176;
Parcubacteria group bacterium GW2011_GWA2_42_80; Mesorhizobium sp. B2-3-3; Fictibacillus
phosphorivorans; Paenibacillus vini; Parcubacteria group bacterium GW2011_GWF2_43_11;
Noviherbaspirillum malthae; Clostridium sp. ‘White wine YQ’; Deltaproteobacteria bacterium GWA2_65_63;
Pollutimonas nitritireducens; Deltaproteobacteria bacterium CG_4_9_14_3_um_filter_65_9; Chryseobacterium
carnipullorum; Flavobacterium johnsoniae; Chloroflexi bacterium ADurb.Bin360; Nannochloropsis salina
CCMP1776; Chryseobacterium sp. CY350; Sorangium cellulosum So ce56; Candidatus Azambacteria
bacterium; Gammaproteobacteria bacterium RIFCSPHIGHO2_12_FULL_37_14; Melioribacteraceae
bacterium; Natroniella acetigena; Salicibibacter cibarius; Paeniclostridium ghonii; Flavobacterium; Penaeicola
halotolerans; Clostridium sartagoforme AAU1; Pullulanibacillus camelliae; Portibacter lacus;
Acidaminococcaceae bacterium; Candidatus Falkowbacteria bacterium; Crassaminicella indica;
Gammaproteobacteria bacterium RIFCSPHIGHO2_12_38_15; Herpetosiphon llansteffanensis; Rhabdobacter
roseus; Hydrogenibacillus schlegelii; Pedobacter sp. AK013; Methanomassiliicoccales archacon PtaU1.Bin030;
Silvibacterium sp.; Bacillus cereus group; Clostridium formicaceticum; Chryseobacterium sp. LJ668;
Clostridium nigeriense; Thermoprotei archaeon; Candidatus Manganitrophaceae bacterium; Pedobacter
borealis; Leptospirillum ferriphilum YSK; Aliifodinibius roseus; Pedobacter terrae; Paenibacillus hunanensis;
Turicibacter sp.; Deltaproteobacteria bacterium GWA2_45_12; Flavobacterium frigidimaris; Succiniclasticum
ruminis; Anaeromyxobacter dehalogenans; Actinobacteria bacterium RBG_19FT_COMBO_70_19; Legionella
jordanis; Candidatus Korarchaeota archaeon; Acidipila sp. EB88; Platysternon megacephalum; Pedobacter sp.
BMA; Tepidibacter thalassicus; Clostridiaceae bacterium; Clostridium aceticum; Halomonas sp.; Castellaniella
caeni; Clostridium sp. MSJ-8; Clostridium sp. D43t1_170807_H7; Halarsenatibacter silvermanii;
Aneurinibacillus sp. B1; Sulfobacillus sp. hq2; Leptospirillum sp. Group II ‘CF-1’; Microgenomates group
bacterium GW2011_GWA2_39_19; Clostridium culturomicium; Peptoniphilus tyrrelliae; Candidatus
Krumholzibacteriota bacterium; Fictibacillus macauensis; Paenibacillus sp. FSL R7-277; Legionella
longbeachae; Legionella sp. 27cVA30; Inconstantimicrobium porci; Paenibacillus fonticola;
Geomesophilobacter sediminis; Paenibacillus zeisoli; Mesorhizobium sp. LNJC394B00; Neofamilia
massiliensis; Solimicrobium sp.; Clostridium carboxidivorans P7; Legionella lansingensis; Parcubacteria group
bacterium Gr01-1014_106; Noviherbaspirillum suwonense; Marinospirillum celere; Clostridium sp.
1001271B_151109_B4; Candidatus Aerophobetes bacterium; Aneurinibacillus sp. BA2021; Sulfobacillus
thermosulfidooxidans; Mesorhizobium; Noviherbaspirillum pedocola; Pedobacter rhizosphaerae; Anaerolineae
bacterium SM23_63; Paralcaligenes sp. KSB-10; Aquisphaera giovannonii; Candidatus Thermofonsia Clade 1
bacterium; Pedobacter roseus; Desulfitibacter sp. BRH c19; Bacillus marinisedimentorum; unclassified
Mesorhizobium; Legionella oakridgensis ATCC 33761 = DSM 21215; Clostridium ihumii; FCB group
bacterium; Luteimonas gilva; Clostridium simiarum; Fontibacillus phaseoli; Clostridium paridis;
Pseudoclostridium thermosuccinogenes; Anaerocolumna aminovalerica; bacterium HR08; Paenibacillus
cineris; Paenibacillus dokdonensis; Pedobacter sp. Leaf216; Bacillus rhizoplanae; Clostridium bornimense;
Flavobacterium sp. HSC-32F16; Papillibacter cinnamivorans; Chryseobacterium profundimaris; Paenibacillus
bouchesdurhonensis; Candidatus Pacearchaeota archaeon; Candidatus Peregrinibacteria bacterium;
Mesorhizobium sp. B2-4-17; Gracilibacteraceae bacterium; Spirochaetia bacterium; Nannochloropsis gaditana;
Candidatus Pacebacteria bacterium; Litoribacterium kuwaitense; Paenibacillus sp. DMB20; Halomonas
azerica; Mesorhizobium sp. INR15; Bacillaceae bacterium JMAK1; Pedobacter sp. HMF7647;
Chitinophagaceae bacterium; Mesorhizobium sp. B2-4-12; Legionella oakridgensis; Noviherbaspirillum soli;
Smithella sp.; candidate division Zixibacteria bacterium RBG-1; Peptoniphilus harei; Anaerovirgula
multivorans; Parcubacteria bacterium DG_72; Paraclostridium bifermentans; Pedobacter sp. SJ11;
Saccharibacillus sp. O16; Paenibacillus sp. PK4536; Pedobacter sp. B4-66; Flavobacterium phragmitis;
Daejeonella rubra; Paenibacillus lemnae; Pedobacter sp. HCMS5-2; Syntrophomonadaceae bacterium;
Paenibacillus albidus; Pseudacidobacterium ailaaui; Paenibacillus cellulositrophicus; Paenibacillus sp. YPG26;
Caulobacter sp.; Maledivibacter halophilus; Clostridium sardiniense; Polyangium aurulentum; Candidatus
Moranbacteria bacterium; Candidatus Dadabacteria bacterium; Chryseobacterium indoltheticum; Legionella
nagasakiensis; Leptospirillum sp. Group IV ‘UBA BS’; Chryseobacterium sp. OSA05B; Chryseobacterium
shigense; Gemmatimonas sp.; Bacillus thuringiensis; Candidatus Microgenomates bacterium; Bryobacteraceae
bacterium; Mesorhizobium sp. B2-4-15; Anaerococcus hydrogenalis; candidate division Kazan bacterium
RBG_13_50_9; Methylovirgula sp. 4M-Z18; Terrabacteria group bacterium ANGP1; Chryseobacterium sp.
MEBOG07; Candidatus Syntrophonatronum acetioxidans; Rhynchophorus ferrugineus; Halonatronomonas
betaini; Paenibacillus sp. S28; Anaerocolumna sp. AGMB13025; Sporobacter termitidis; candidate division
Zixibacteria bacterium RBG_16_40_9; Pedobacter suwonensis; Chromatiales bacterium; Acidobacteria
bacterium OLB17; Geomonas sp. RF6; Chlorobaculum parvum; Gemmatimonadetes bacterium
RIFCSPLOWO2_12_FULL_68_9; Clostridiaceae bacterium 14S0207; Paenibacillus sp.; Lysobacter terrigena;
Nitrospira sp. CG24E; Bacillus wiedmannii; Methylocapsa sp. S129; Pedobacter sp. SYSU D00382;
Gemmatimonadetes bacterium GWC2_71_10; Steroidobacter sp.; Mesorhizobium sp. AR02; Candidatus
Margulisbacteria bacterium GWE2_39_32; Cellulosilyticum ruminicola; Bdellovibrio sp. NC01; Paenibacillus
sp. J22TS3; Eubacteriales bacterium; Tatlockia sp.; Anaerococcus sp. Marseille-P9784; Mesorhizobium sp.
Root102; Mesorhizobium sp. B263B2A; Paenibacillus durus; Chryseobacterium cheonjiense; Candidatus
Thermokryptus mobilis; Steroidobacter soli; Mesorhizobium sophorae; unclassified Flavobacterium;
Flavobacterium crocinum; Terasakiispira papahanaumokuakeensis; Candidatus Bipolaricaulota bacterium;
Paralcaligenes ureilyticus; Clostridium sp. LY3-2; Ignavibacteriales bacterium
CG_4_9_14_3_um_filter_30_11; Desulfotomaculum sp. 46_80; Mesorhizobium sp. 113-3-3; Pedobacter
ginsenosidimutans; candidate division WOR-1 bacterium RIFCSPHIGHO2_01_FULL_53_15;
Armatimonadota bacterium; Peptoclostridium acidaminophilum; Bacillus cereus group sp. BfR-BA-01492;
Pelotomaculum sp.; Legionella taurinensis; Chryseobacterium sp. OV279; Chlorobium sp. N1; Paenibacillus
apii; Candidatus Flavonifractor merdavium; Dokdonella sp.; Armatimonadetes bacterium 13_1_40CM_3_65_7;
Mesorhizobium kowhaii; Desulfotomaculum sp.; Fundicoccus ignavus; Desulforamulus ruminis; Polaromonas
sp.; Lucifera butyrica; Mesorhizobium sangaii; Paenibacillus sp. CAA11; Pelotomaculum thermopropionicum
SI; Trueperaceae bacterium; Acidimicrobiales bacterium; Paenibacillus sabinae; Candidatus Kuenenbacteria
bacterium; Siphonobacter curvatus; Prosthecochloris sp. CIB 2401; Paenibacillus lentus; Clostridium
botulinum C; Chryseobacterium sp. Leaf201; Rufibacter sp. XAAS-G3-1; Methylovirgula ligni;
Dethiosulfovibrio peptidovorans; Roseisolibacter agri; Singulisphaera sp. GP187; Ferruginibacter sp.;
Intestinimonas massiliensis; Pusillimonas faecipullorum; Geomonas oryzisoli; Paucimonas lemoignei;
Clostridiales bacterium GWB2_37_7; Candidatus Lokiarchaeota archaeon Loki_b31; Natronincola
peptidivorans; Steroidobacter cummioxidans; Pirellulaceae bacterium; Bacillus cereus group sp.
BfR-BA-01383; Niallia circulans; Chlorobiota bacterium; Fictibacillus enclensis; Bacillaceae; Fusibacter
paucivorans; Deltaproteobacteria bacterium HGW-Deltaproteobacteria-6; Noviherbaspirillum sp. L7-7A;
Anaerococcus ihuae; Flavobacterium sp. S87F.05.LMB.W.Kidney.N; Paenibacillus sp. J23TS9;
Deltaproteobacteria bacterium HGW-Deltaproteobacteria-1; Armatimonadia bacterium; Solimonas aquatica;
Candidatus Saccharibacteria bacterium QS_5_54_17; Ignavibacterium album; Oligoflexia bacterium;
Paenibacillus algicola; Daejeonella lutea; Pedobacter petrophilus; Lewinellaceae bacterium; Candidatus
Buchananbacteria bacterium RIFCSPHIGHO2_02_FULL_38_8; Flavobacterium sp. JLP; Mesorhizobium loti;
bacterium HR36; Bacillus sp. HMF5848; Flavonifractor sp. An82; Mesorhizobium sp.; Noviherbaspirillum
saxi; Anaerococcus porci; Flavobacterium sp. 245; Saccharibacillus sacchari; Nitrospira sp.; Chryseobacterium
sp.; Deltaproteobacteria bacterium RIFCSPHIGHO2_02_FULL_40_28; Chlorobaculum sp. MV4-Y;
Romboutsia lituseburensis; Clostridium kluyveri DSM 555; Candidatus Saccharibacteria bacterium CPR2;
Chryseobacterium sp. 09-1422; Acidimicrobiia bacterium; Owenweeksia hongkongensis; Flavobacterium
reichenbachii; Paracidobacterium acidisoli; Paenibacillus rhizosphaerae; Chryseotalea sanaruensis;
Paludisphaera borealis; Methanomassiliicoccus sp.; Prosthecochloris sp. GSB1; Gemmatimonas aurantiaca
T-27; Gemmatimonas aurantiaca; Clostridium kluyveri NBRC 12016; Massilibacterium senegalense;
Clostridium sp. Ade.TY; Paramaledivibacter caminithermalis; Abyssisolibacter fermentans; Citrifermentans
bemidjiense; Desulfoscipio gibsoniae; Firmicutes bacterium ADurb.Bin373; unclassified Halomonas;
Flavobacterium hydrophilum; Parcubacteria group bacterium Athens0714_25; Calditrichia bacterium;
Calditrichaceae bacterium; Flavobacterium album; Pedobacter yulinensis; bacterium ADurb.Bin429;
Thermanaeromonas sp. C210; Paenibacillus; Burkholderiales bacterium RIFCSPLOWO2_02_FULL_57_36;
Pedobacter namyangjuensis; Candidatus Sumerlaeaceae bacterium; Clostridium kluyveri; Pelosinus
fermentans; Pedobacter sp. MR22-3; Noviherbaspirillum galbum; Thermanaeromonas sp.; Mucilaginibacter
auburnensis; Bacillus cytotoxicus; Peptococcaceae bacterium; Paenibacillus spiritus; Rhodanobacter sp. B04;
candidate division Zixibacteria bacterium RBG_16_48_11; Caulobacter sp. UNC358MFTsu5.1; Acidimicrobiia
bacterium EGI L10123; Armatimonadetes bacterium CG07_land_8_20_14_0_80_40_9; Bacteroidetes
bacterium SW_10_40_5; Spirochaetota bacterium; Hydrogenibacillus sp. N12; bacterium B17; Marivirga sp.
S37H4; Flammeovirgaceae bacterium; Scopulibacillus daqui; Saccharibacillus alkalitolerans; Hippocampus
comes; Runella rosea; Noviherbaspirillum sp. Root189; Clostridium tarantellae; Mesorhizobium sp. B2-3-4;
Candidatus Saccharibacteria bacterium GW2011_GWC2_48_9; miscellaneous Crenarchaeota group archaeon
SMTZ-80; Pelotomaculum propionicicum; Fictibacillus aquaticus; Rhodanobacter sp. C05; Clostridium
botulinum CFSAN002369; Pedobacter sp. Leaf250; Catalinimonas alkaloidigena; Mesorhizobium sp.
LSJC269B00; candidate division CPR3 bacterium 4484_211; Acidobacteria bacterium 13_1_20CM_4_56_7;
Paenibacillus uliginis; Mesorhizobium sp. L2C084A000; Mesorhizobium sp. ES1-4; Pedobacter foliorum;
Fictibacillus nanhaiensis; Chryseobacterium luteum; Pedobacter yonginense; Agrobacterium tumefaciens;
Planomicrobium sp. CPCC 101079; bacterium SGD-2; Flavobacterium sp. KB82; Candidimonas bauzanensis;
Chryseobacterium sp. 7; Terriglobales bacterium; Thermotogota bacterium; Minicystis rosea; Saccharibacillus
qingshengii; Candidatus Kryptonium sp.; Pedobacter endophyticus; Candidatus Tectomicrobia bacterium;
Pedobacter sp. SYSU D00823; Citrifermentans bremense; Mesorhizobium helmanticense; Pelotomaculum
thermopropionicum; Candidatus Saganbacteria bacterium; Chloroflexi bacterium
RBG_19FT_COMBO_62_14; Geomicrobium halophilum; Acidisarcina polymorpha; Ectobacillus funiculus;
Flavihumibacter sediminis; Thermoanaerobaculum sp.; unclassified Halanaerobium; Candidatus Flavonifractor
intestinigallinarum; Rufibacter hautae; Geomonas azotofigens; Prosthecochloris sp. HL-130-GSB; Candidatus
Latescibacteria bacterium; Halomonas malpeensis; Membranihabitans marinus; Chryseobacterium
shandongense; Desulfitibacter alkalitolerans; Paenalcaligenes niemegkensis; Desulfotomaculum nigrificans;
Desulforamulus aeronauticus; Anaerococcus urinomassiliensis; Melioribacter roseus; Nitrospiraceae bacterium;
Maledivibacter sp.; Thermanaeromonas toyohensis; Mesorhizobium sp. M8A.F.Ca.ET.057.01.1.1; Spiroplasma
sp. JKS002671; Saccharibacillus kuerlensis; Noviherbaspirillum sp. UKPF54; Peptostreptococcaceae
bacterium; Gemmatimonas phototrophica; Fictibacillus marinisediminis; Ohtaekwangia koreensis; Pedobacter
psychroterrae; Desulfatitalea sp. BRH_c12; Selenomonadaceae bacterium; Ignavibacteria bacterium
GWF2_33_9; Fictibacillus sp. 26RED30; Ignavibacteriales bacterium
CG18_big_fil_WC_8_21_14_2_50_31_20; Runella sp. S5

TABLE B
NP_002425.2; XP_016784459.1; NP_001015052.1; NP_001015054.1; PNJ02743.1; XP_025225159.1;
XP_055220685.1; XP_011891187.1; XP_011822258.1; XP_032001168.1; XP_032001167.1;
XP_011822257.1; XP_024649919.1; XP_017716223.1; XP_007978137.2; XP_011822259.1;
XP_032125324.1; XP_035122191.2; XP_012324157.1; XP_017366330.1; XP_032125322.1;
XP_012515057.1; XP_012661237.1; XP_012593217.1; XP_012593216.1; XP_026341206.1;
XP_034526637.1; KAF6490278.1; XP_001494845.3; XP_039079336.1; XP_042779002.1; XP_005621833.1;
XP_020032627.1; XP_005621832.1; XP_005621834.1; XP_025779098.1; XP_042779001.1;
XP_047389408.1; XP_042828520.1; XP_008536632.1; XP_042828518.1; XP_025273023.1;
XP_019504337.1; XP_040315543.1; XP_049495005.1; XP_029805927.1; XP_023102455.1;
XP_036135088.1; CAI9172501.1; KAG5194849.1; XP_055264946.1; XP_016071697.1; XP_047637115.1;
XP_055264947.1; XP_015992259.1; XP_015346550.1; XP_008585505.1; XP_046296760.1;
XP_014442491.1; VFV38375.1; XP_020755858.1; XP_043771623.1; XP_045850005.1; XP_014304681.1;
ELW67741.1; KAF7480741.1; XP_054990167.1; MXQ80907.1; XP_015346551.1; XP_038180989.1;
XP_049623945.1; XP_054442739.1; XP_017895328.1; XP_054990168.1; XP_053410140.1; EPQ10253.1;
XP_005391732.1; XP_022372452.1; KAF5921018.1; XP_022372451.1; EHB14148.1; XP_052053650.1;
XP_006204427.3; AAL32368.1; XP_031209386.1; XP_034361511.1; XP_023567264.1; XP_012924302.2;
ERE69542.1; XP_014398442.1; XP_028749167.1; XP_051023345.1; XP_008155903.2; XP_013843190.1;
XP_044928051.1; XP_028612280.1; MBV99522.1; KAI5228846.1; EPY85320.1; XP_010949223.1;
XP_040604337.1; XP_010949223.2; XP_032768024.1; XP_042524591.1; XP_032316608.1;
XP_031303599.1; XP_036054078.1; XP_014338435.1; XP_037669613.1; XP_010618935.1;
XP_036771927.1; XP_008849009.1; XP_051698168.1; XP_048187913.1; XP_047570167.1;
XP_007955662.1; XP_023416443.1; XP_017523794.2; XP_036857672.1; XP_051036977.1;
XP_045730506.1; XP_012866754.1; XP_037364022.1; XP_036316212.1; XP_040829612.1;
XP_051824617.1; XP_023584137.1; XP_023584136.1; XP_023353523.1; XP_023584135.1;
XP_036595076.1; XP_020862251.1; XP_023413367.1; XP_044513642.1; XP_036595077.1;
XP_031801540.1; XP_027718619.1; XP_007499243.1; XP_006873975.1; XP_021513279.1; ACG63682.1;
XP_043855868.1; XP_004706161.2; XP_006894972.1; XP_013373281.1; XP_037678606.1;
XP_019573642.1; KAF6490275.1; XP_055266486.1; XP_055280219.1; XP_042717497.1; XP_008160760.1;
XP_027718620.1; XP_038274169.1; XP_053898185.1; XP_038274168.1; XP_053898183.1;
XP_043349489.1; XP_034638960.1; XP_030434793.1; XP_032650542.1; XP_048721919.1;
XP_044834924.1; XP_039348635.1; XP_032650540.1; XP_013795360.1; NWR76136.1; XP_032650541.1;
CAD7674994.1; XP_038619306.1; XP_014461253.1; XP_033908328.2; XP_052536393.1; XP_052536391.1;
XP_014429784.1; XP_042683972.1; XP_006124598.1; KYO44572.1; XP_033770015.1; XP_009475529.1;
XP_005990872.1; XP_030068854.1; XP_010717698.1; XP_048817408.1; XP_042717498.1;
XP_005306213.1; RMC17816.1; XP_030338271.1; XP_015732803.1; TRZ24251.1; XP_038007644.1;
XP_009812794.1; NXJ51196.1; KAI1233858.1; XP_051488130.1; XP_041257475.1; XP_040296808.1;
XP_040296809.1; NWQ88916.1; OWK55395.1; KAG8558501.1; XP_044160264.1; XP_044160263.1;
RLW01589.1; XP_040212953.1; GCB80072.1; XP_048847494.1; XP_012825907.2; XP_017952613.2;
XP_023993446.1; XP_041044137.1; XP_018426287.1; XP_029491806.1; KAJ7410026.1; XP_028561546.1;
KAF7243101.1; XP_041433201.1; XP_044187891.1; XP_053550486.1; XP_043945379.1; XP_039630404.1;
XP_043091153.1; XP_053194454.1; XP_053708547.1; XP_053348122.1; XP_036409147.1;
XP_042360152.1; XP_030646734.1; XP_036409146.1; XP_034987466.1; XP_033022399.1;
XP_041633713.1; XP_048102419.1; XP_054849241.1; XP_016143145.1; XP_047663674.1;
XP_045922271.1; KAJ6655979.1; XP_023677646.1; KAJ8345015.1; XP_029293748.1; XP_036422733.1;
XP_044036522.1; MCJ8746490.1; XP_018560545.1; TSZ68964.1; XP_046690522.1; XP_024128040.1;
XP_029913726.1; XP_017310079.1; XP_049601576.1; XP_034075877.1; RVE69714.1; CAG12204.1;
KAE8283351.1; KAJ8251206.1; MCI4392275.1; XP_049417167.1; XP_029913723.1; XP_053468741.1;
XP_048349834.1; XP_026539152.1; XP_020465969.1; EDM04017.1; XP_053468740.1; RXN22755.1;
XP_051881318.1; TRY89240.1; XP_029014714.1; XP_018610296.1; XP_028841468.1; KAG8429658.1;
XP_018610294.1; XP_034297269.1; XP_036945314.1; XP_034047026.1; KAG8006851.1; XP_033501421.1;
KAJ7306076.1; XP_015667436.1; XP_008327456.1; XP_053132311.1; XP_042293839.1; XP_053328103.1;
XP_030264311.1; XP_032086387.1; XP_039216642.1; XP_053328101.1; XP_032877252.1;
XP_039679639.1; XP_028455931.1; NP_001076319.1; XP_054066824.1; KAG8145895.1; XP_020511554.1;
XP_011602413.1; XP_040918728.1; XP_031168853.1; XP_032393364.1; XP_034750113.1; OXB54910.1;
XP_035253115.1; XP_026179884.1; XP_008304671.1; XP_035253113.1; XP_026560323.1;
XP_026851288.2; KAJ1109397.1; XP_013879602.1; KAJ8253921.1; XP_053300099.1; XP_020667655.1;
XP_042188736.1; KAF7225650.1; XP_004071647.2; XP_030236282.1; KAF0032868.1; MBN3276840.1;
GBN10628.1; ROL51177.1; XP_003230501.2; KAG2455860.1; XP_015265378.1; XP_051243433.1;
XP_031420737.1; XP_015215875.1; XP_034567220.1; XP_029692893.1; TWW70145.1; CAI5675877.1;
XP_037544500.1; XP_054717128.1; XP_018610297.1; XP_007433930.1; XP_032832053.1;
XP_032832054.1; XP_033631247.1; XP_023269749.1; XP_016321102.1; GFR00254.1; GFR14856.1;
XP_032461981.1; XP_033631246.1; GFT86630.1; GFY70054.1; XP_035210800.1; KFM56773.1;
XP_030236291.1; GIY84848.1; XP_002734634.1; XP_013420197.1; XP_035696132.1; XP_045603965.1;
XP_045603964.1; VVC45432.1; XP_037798962.1; XP_045603963.1; XP_025425257.1; XP_011437056.2;
XP_027218029.1; XP_022317085.1; XP_052261707.1; XP_045131863.1; XP_005098790.1;
XP_050541131.1; XP_021942413.1; KAJ8037963.1; XP_023218940.1; KAI8501103.1; XP_021942412.1;
KAI8766451.1; XP_033749820.1; PIK42417.1; XP_033749821.1; ELU15282.1; XP_015172118.1;
KAF8773469.1; KAG8183705.1; CAH1794932.1; XP_020906283.1; KAF2899816.1; XP_050848968.1;
XP_048245971.1; XP_011307097.1; KAE9543129.1; XP_026806311.1; XP_011307098.1; XP_014607331.1;
XP_021362657.1; XP_029189721.2; XP_033120183.1; GFG40971.1; XP_046838869.1; RUS81070.1;
XP_015922971.1; KAH9504064.1; CAD7635358.1; CAH1274370.1; CAH1737706.1; XP_025193378.1;
KAJ3664310.1; XP_041363322.1; KAF0767464.1; VDO94504.1; XP_023701425.1; XP_041363451.1;
XP_042142330.1; XP_033606128.1; XP_023701424.1; XP_015117417.1; XP_039312569.1;
XP_020626047.1; MCL4122375.1; KAF5303965.1; XP_025087545.1; XP_046544346.1; XP_024940954.1;
XP_022799137.1; XP_025073356.1; OWF45987.1; XP_034942327.1; XP_048575316.1; XP_044763519.1;
XP_043479324.1; XP_042220361.1; PIK41027.1; CAG5100763.1; CAH1391019.1; XP_022171343.1;
XP_014226303.1; EEC03892.1; XP_032452040.1; CAH1391018.1; XP_045467208.1; KAI0217015.1;
XP_008213416.1; CAH1367731.1; KAJ7386224.1; XP_014276329.1; XP_050422713.1; XP_026465296.1;
KAG8042206.1; XP_018328288.1; XP_050043043.1; XP_008188248.1; RMH14586.1; XP_018666791.1;
XP_034180517.1; EEC03891.1; XP_044089689.1; XP_048576207.1; XP_012531196.1; XP_038069241.1;
XP_029038394.2; XP_022171345.1; XP_022171346.1; NHA14208.1; XP_012219475.1; XP_028413179.1;
XP_027043452.1; CAH1987048.1; XP_048757975.1; XP_012271922.1; KAJ3664309.1; NGP52021.1;
WP_240901301.1; RDD47663.1; XP_043793683.1; XP_002116268.1; XP_002429174.1; XP_012145446.1;
KAH9504065.1; CAH1737707.1; KAG5347081.1; XP_024892153.1; XP_023331833.1; XP_043526823.1;
OQV13712.1; XP_048757974.1; XP_044574840.1; XP_014215010.1; GFG40028.1; XP_042142356.1;
XP_019871875.1; KRZ15991.1; RXG55541.1; XP_043280100.1; XP_011338011.1; XP_011687355.1;
XP_044574839.1; XP_003375670.1; XP_029640826.1; XP_025087562.1; XP_011056122.1;
XP_018403602.1; XP_017884890.1; XP_037509822.1; KAG5713404.1; XP_015438929.1; XP_029677480.1;
VEN33459.1; KAG0425902.1; XP_018020384.1; XP_031837335.1; XP_041473622.1; CAH1099993.1;
XP_046644184.1; KAG5887409.1; XP_008549627.1; XP_054763943.1; XP_018915897.1; RWS22065.1;
XP_967967.1; XP_031554200.1; XP_022080263.1; XP_022080262.1; KAH9371247.1; XP_017752566.1;
XP_014249492.1; WP_240900858.1; XP_033214785.1; XP_046445489.1; XP_037285765.1;
XP_043598397.1; XP_026506571.1; XP_015377141.1; XP_018355523.1; NGX15980.1; XP_033214784.1;
RWS11353.1; CAD7432253.1; MCL4220174.1; XP_011498146.1; XP_018373329.1; CAD7570631.1;
XP_050740050.1; CAB3362084.1; XP_046490543.1; XP_046856270.1; GLG94577.1; XP_046628265.1;
XP_026464104.1; TVQ63452.1; CAG9857745.1; CAB3362083.1; CAD7408308.1; KFD57891.1;
XP_046750752.1; XP_052814312.1; XP_046672794.1; CAD7403950.1; KAI4471329.1; KAF2899815.1;
XP_786488.3; KAF6215819.1; CAD7408307.1; XP_021942418.1; XP_050461441.1; GFO16014.1;
PNF39933.1; XP_023701413.1; CAD7453771.1; XP_019871876.2; XP_021942416.1; CAD7201032.1;
XP_053997133.1; XP_020284364.1; KAG7209764.1; XP_023028411.1; CAD7277595.1; GLG98304.1;
CDW56157.1; XP_018569218.1; XP_044270088.1; XP_023701412.1; KAJ4443954.1; XP_028151354.1;
XP_030766092.1; XP_18047240.1; CAD7241138.1; CAG9822359.1; WP_206211863.1; XP_034242323.1;
KAA0213200.1; XP_049852321.1; XP_011875093.1; XP_049950098.1; XP_050393574.1; XP_018569225.1;
MBI1368773.1; XP_014672378.1; XP_054268013.1; QNN23585.1; OQR70227.1; RMG42883.1;
XP_012171887.3; XP_054155559.1; MCC6677046.1; XP_051161082.1; XP_050393573.1; KAI3359167.1;
XP_033365725.1; CAB3263928.1; XP_014476208.1; MCC7146123.1; XP_046385293.1; KAI5698255.1;
KAJ8667383.1; PSN39652.1; XP_032460867.1; XP_044737807.1; XP_031347571.1; XP_019615571.1;
KAI6652545.1; MBX2851576.1; CAH1281818.1; HCD34427.1; KAJ8038094.1; KAB7495034.1;
KAF2369236.1; VDI69304.1; XP_049799850.1; VDI69305.1; XP_044006431.1; XP_017781484.1;
XP_026281906.1; TVQ48961.1; CAH0547631.1; MBK7405029.1; UTX43260.1; GDX99003.1;
KAH0811256.1; TVS06949.1; KAG5859779.1; MCC6321698.1; XP_019853450.1; EFN81544.1;
CAC5402312.1; ROT84732.1; XP_007605385.1; XP_009264592.1; XP_014276395.1; MCC7205472.1;
EFX88790.1; CAG0998698.1; XP_003739583.1; GAV07795.1; XP_045025554.1; XP_012262224.2;
MCC5862068.1; XP_011144080.1; KAI9564548.1; KAG8222540.1; XP_025110441.1; XP_044223456.1;
NP_586320.1; MAX25709.1; CAH7751284.1; KAH7962001.1; MAY75699.1; XP_042588631.1;
XP_018314358.1; WP_115268644.1; KAA0196133.1; MBX3323842.1; MCC5787853.1; XP_043460798.1;
XP_003073865.1; MAT81836.1; XP_043460797.1; XP_007605579.1; MBI1371276.1; XP_043460795.1;
MCC7409368.1; TRY62801.1; MBL4701465.1; MCL4209960.1; MCC6227892.1; XP_054167211.1;
MBS0196656.1; CAF0845495.1; XP_022905691.1; KAI1303168.1; MCA9306591.1; CAG0973675.1;
CAD7650327.1; MBC7835230.1; MCL4741684.1; TVQ51605.1; MBX3351451.1; NUQ68035.1;
MCG3122578.1; MAE62470.1; KAG4076374.1; MCA9196371.1; XP_011263384.1; KAI4471566.1;
MBX3365941.1; MBX3376569.1; MBX3365424.1; TVQ33698.1; XP_032784219.1; WP_091748845.1;
MBX3362973.1; MCK4872181.1; CAD5112555.1; MCC6428735.1; KAF7490627.2; XP_021958864.1;
OGQ81693.1; XP_046908976.1; RJR30723.1; MCH2154346.1; MBM4108223.1; HBS29806.1;
WP_230742211.1; MCE7972834.1; WP_212903617.1; UXI20054.1; GFQ67279.1; MCR9074446.1;
XP_020284368.1; MBL9001768.1; MCC7192354.1; MBJ93217.1; XP_039290864.1; NNM25430.1;
XP_020284365.1; GJQ29937.1; MCA9304271.1; MBL8745391.1; WP_164800541.1; QYK48446.1;
MCA9280728.1; MBY0112128.1; MCB9845457.1; MCB9847677.1; MBX3404824.1; RZC37530.1;
KAF5304422.1; QKK09513.1; TVQ60234.1; XP_030236299.1; MBM6838090.1; XP_024940956.1;
RMH28342.1; QOI99514.1; XP_032674632.1; MCH8005347.1; WP_093752143.1; XP_043793684.1;
MBX3315857.1; XP_033336230.1; XP_029677489.1; WP_185652882.1; XP_024940955.1; MBP5737488.1;
MBX3392526.1; MBL8964303.1; QQS10476.1; CAD7628785.1; MBO6739829.1; XP_024892152.1;
MBS0188124.1; MCA9287928.1; OIN95852.1; WP_084114959.1; WP_231638353.1; MBZ9686730.1;
MCA9274493.1; HEC33330.1; PYO43024.1; MCE9589536.1; XP_043259089.1; NBC11645.1;
MCC6681845.1; WP_226260660.1; XP_031774959.1; WP_236910817.1; GLI56595.1; MCC6660455.1;
OGQ81321.1; CAG5897951.1; HEY80736.1; MCC7387393.1; WP_242367215.1; WP_261670454.1;
WP_174591786.1; WP_061429332.1; MBX3388764.1; XP_027205096.1; CAD7642519.1; WP_204990525.1;
MBC6954457.1; WP_274292477.1; WP_132379491.1; MBM4042199.1; MAO25048.1; XP_025016883.1;
XP_015786052.1; MBN8597573.1; WP_149733859.1; QDV08608.1; XP_051161073.1; OGZ37962.1;
WP_128211072.1; MBS1105336.1; MAA53098.1; OGY42125.1; XP_011687357.1; XP_012271926.1;
OLC36847.1; MBX3385975.1; SHI77557.1; SCJ61537.1; XP_031364394.1; PIV10374.1; HCT44834.1;
MBY0308484.1; MBG9794905.1; WP_160670170.1; NLC45912.1; NLW41057.1; MCC6951250.1;
MBS0190423.1; MCA9291151.1; WP_197259191.1; WP_072830443.1; MBI3326196.1; MCL1978163.1;
MBL0926033.1; MBL9032369.1; RMD66764.1; RLC39998.1; WP_193418736.1; XP_012062396.1;
HHD92008.1; MCH9057169.1; WP_012036110.1; WP_216302856.1; PYS54000.1; WP_073248251.1;
WP_014405253.1; NLY76915.1; MBD0325789.1; WP_012898785.1; MBL8763102.1; NLD17575.1;
GIW41706.1; WP_103896679.1; KOC69474.1; XP_053203364.1; MBZ9578394.1; EGT3615067.1;
NLF87936.1; WP_054261630.1; WP_239329420.1; PYS41515.1; MAF42745.1; TMB41141.1; HHX12711.1;
WP_129029654.1; XP_018047242.1; RLC98955.1; GIY91558.1; EZA54659.1; WP_176829508.1;
MCD6553490.1; MCD6115309.1; UCD74135.1; MCE2966512.1; WP_216277458.1; WP_078809583.1;
MCH7748195.1; WP_068784404.1; MCA9127743.1; WP_092559944.1; NMB07971.1; UCC64762.1;
MCA1617962.1; MBM4030660.1; MBZ4663795.1; WP_055263583.1; MCD6528622.1; HHT51406.1;
WP_013278049.1; WP_202922757.1; MCC6969354.1; NDI77599.1; MBI2551719.1; WP_227020203.1;
KAJ6222058.1; HDQ34739.1; WP_268039191.1; TMB02216.1; WP_068719165.1; EJO5347840.1;
TMA66795.1; WP_106063558.1; MBI1190664.1; RIL00882.1; MBF6617100.1; PYP52759.1;
XP_029677546.1; OHB21064.1; WP_008908098.1; WP_095177559.1; RLC89339.1; WP_089862934.1;
HBY94031.1; MBP2671468.1; MBV7272392.1; HEB60203.1; WP_089984058.1; MBL8761743.1;
WP_274766984.1; HHU50004.1; KAH0950838.1; WP_084159124.1; XP_033214933.1; RYZ75594.1;
WP_013624138.1; EKE11093.1; MCH8165522.1; NLJ99300.1; WP_012447186.1; WP_054873787.1;
MBC7772629.1; MCH8315553.1; WP_261852950.1; MBE7468524.1; MBU1146249.1; MCQ3974700.1;
TMA49204.1; CAD1473226.1; WP_127576204.1; WP_168179928.1; AKU92512.1; HEY63014.1;
GJM83970.1; MBL0922305.1; WP_246615214.1; MBM4464615.1; TDT72483.1; XP_017797699.1;
MBP5405940.1; MCH8152075.1; PIR01875.1; WP_027630518.1; MBS4026929.1; MBI4836903.1;
WP_251859830.1; WP_127837256.1; WP_183279399.1; WP_200270261.1; WP_072771651.1;
WP_079426545.1; SHN59978.1; PYR95918.1; HIP97136.1; KAF7417593.1; WP_062552570.1;
RMH26294.1; PYP38301.1; WP_014967375.1; WP_050726669.1; CAF1326176.1; MCC7374546.1;
WP_091403152.1; NGM88762.1; MCC6897444.1; WP_151861215.1; MCH7571428.1; XP_043280101.1;
WP_244946990.1; RME11084.1; UYV14214.1; WP_130040192.1; WP_129971401.1; WP_137698044.1;
MBA2627298.1; MCH7848535.1; WP_209020038.1; OQB38947.1; WP_269357957.1; WP_127194599.1;
MBI3335301.1; MBX3380977.1; WP_026882255.1; WP_013741469.1; QNU68739.1; WP_088227517.1;
KAG9463768.1; WP_251579913.1; MCS7060824.1; CAG7833985.1; WP_078025021.1; WP_027623626.1;
HHY26649.1; WP_123052680.1; MBL7075938.1; RME30577.1; WP_050353719.1; WP_202749142.1;
KAI4496517.1; WP_005587313.1; MCD6233286.1; NLV76016.1; WP_007785203.1; TDJ55687.1;
MCH7547420.1; MCK6629438.1; MCA1632151.1; MBI1337948.1; MCL4785899.1; WP_220806198.1;
WP_130007660.1; WP_090551104.1; MCC5830744.1; HHT64961.1; WP_138190752.1; WP_073025196.1;
WP_010076035.1; MCH7797917.1; KPJ71378.1; NLM25810.1; MBA3784845.1; NLY50522.1;
WP_264131648.1; MCC2637519.1; WP_232278650.1; MBM4106754.1; MBI2361352.1; OLD04782.1;
NPA90098.1; WP_018663798.1; WP_206582426.1; HDQ71389.1; PYO52526.1; WP_005488473.1;
TSC72121.1; HHY81685.1; MBA2605300.1; WP_246578088.1; WP_133381733.1; MBO1319816.1;
MCA9286561.1; PYM54917.1; PYO98594.1; RME75644.1; WP_163261621.1; MCH2133231.1;
MBH06728.1; WP_216440752.1; WP_258279069.1; MBR4703082.1; TAH34408.1; HHU79353.1;
MBA2621063.1; RUA03009.1; WP_133531373.1; MBD8499858.1; WP_047170985.1; PSR28342.1;
MBP1743155.1; WP_066678470.1; PBC32966.1; MBX3409232.1; MCC6667894.1; WP_271227724.1;
WP_013738983.1; MCI6276069.1; MBI4517240.1; AIQ44572.1; WP_014185990.1; WP_225445825.1;
MBK6723949.1; MBP9664812.1; OGZ32460.1; MCS7178447.1; MCH2254476.1; MCD6270551.1;
WP_071139351.1; WP_009226811.1; NCA94359.1; WP_221038514.1; WP_066021910.1; MCD6484158.1;
MCB9078614.1; MCD6242889.1; MBN9403123.1; RME34058.1; WP_252130342.1; MBQ7214892.1;
WP_148810639.1; MBP2690163.1; MBI5474552.1; NLI89411.1; MBI3648935.1; MCC5821971.1;
MCB1023794.1; TSC53375.1; WP_169296202.1; WP_202411711.1; PKM81377.1; WP_048081485.1;
MBI4527594.1; PKU36177.1; WP_132028624.1; WP_233695837.1; TMB58773.1; MBE6081100.1;
WP_109714626.1; MYN13408.1; GIP50915.1; MBN1875372.1; NLC42700.1; OOM80105.1;
WP_234117006.1; PYS98413.1; MCU1230778.1; MBE6062575.1; WP_243435971.1; HBB86127.1;
WP_279118650.1; MBC7249445.1; NME99414.1; WP_133516221.1; WP_017416274.1; PYP16026.1;
MBI2402517.1; WP_087431756.1; MAE62895.1; OQC42009.1; MBA4697311.1; MCL4300436.1;
WP_092478394.1; PIR69545.1; RBP46807.1; KAF3420782.1; WP_015327055.1; WP_216415041.1;
MBE7551242.1; MCP4537330.1; NYT59191.1; MCI4445220.1; MBA3443711.1; MBC7225722.1;
MBA2647788.1; WP_018247832.1; TET51458.1; RLC61444.1; WP_091539909.1; NLO46476.1;
WP_246188213.1; WP_021875990.1; UUV17866.1; MCM3869940.1; MCE5222397.1; NOX62846.1;
WP_163237678.1; PIZ90091.1; OYV76697.1; WP_026476103.1; MCA9626698.1; MBX9736552.1;
MBV8299792.1; MCF7885479.1; MBM4118496.1; NLA85541.1; WP_051196348.1; WP_087678506.1;
WP_134117621.1; WP_117522241.1; MBP7377213.1; MCP3902166.1; MBI5576314.1; NLF13123.1;
NLU36854.1; MBI2459678.1; XP_041085183.1; WP_035783131.1; MBM4263964.1; MCL2569392.1;
TFW10202.1; WP_210059978.1; WP_077865853.1; MCS7281965.1; HIU41296.1; NLH31329.1;
SFG23325.1; PYO66729.1; WP_240961381.1; HHW15573.1; MCD6550158.1; MBP1642876.1;
WP_251596377.1; MBK7933728.1; NJD63281.1; WP_054199652.1; MBC8015346.1; MCI0364766.1;
OQX84116.1; WP_027417978.1; NLK36460.1; PYP22401.1; MBV8517737.1; OKZ86273.1; MBP6020273.1;
MBI3414047.1; MBV9209840.1; MBN1955095.1; NQU99780.1; WP_084330854.1; PZN34639.1;
NLE43798.1; WP_040397677.1; MCH8825206.1; MBL0937418.1; WP_145446274.1; MCS7087473.1;
MBQ9902640.1; MBL9136649.1; PYQ64726.1; MCD6521593.1; OPY28901.1; WP_027127889.1;
KAI4499404.1; CDM25097.1; WP_012159016.1; WP_102399493.1; WP_008422804.1; MBA2705073.1;
MBV9620880.1; MBI1852822.1; MSP39821.1; WP_010966252.1; MBR6421744.1; MBB6430921.1;
MCL2827574.1; HHV53998.1; HIR41021.1; NOV87961.1; XP_014248457.1; OMG46231.1;
WP_114370573.1; OGL69065.1; TVX88429.1; TMA94058.1; HBF38381.1; MBY0312470.1;
MBV6273798.1; MCB9103364.1; MBV8545578.1; RLG99179.1; TMK35985.1; WP_261827211.1;
NLN11067.1; HHV45422.1; MBA3334667.1; TMK19505.1; PYP03965.1; WP_261378445.1;
WP_125154684.1; MBV9280475.1; MBD3359307.1; WP_097019371.1; MCC6859981.1; NLL74123.1;
HET89694.1; CDB16267.1; OKP86644.1; TKR56099.1; MCL2367768.1; WP_206151593.1; MCC6533641.1;
NME82333.1; MBC8263297.1; WP_055207239.1; MCA1592874.1; NJM09921.1; GJD11514.1;
MBK9215462.1; NLB41483.1; WP_019850172.1; WP_090395523.1; NLB22297.1; WP_080063508.1;
MBV9215798.1; OGZ32766.1; MBV2181944.1; WP_216156137.1; WP_059048677.1; WP_083612289.1;
MBQ8389468.1; MBX3041565.1; WP_236337573.1; MBP2683725.1; WP_069678481.1; WP_132473398.1;
WP_027338607.1; MBC7791696.1; MBI5417070.1; MBS5037637.1; AUX25053.1; MBM2804531.1;
WP_008826301.1; NRS86771.1; MBD3409240.1; MBO4699008.1; WP_072990027.1; WP_198423085.1;
HFB52463.1; WP_183354800.1; MBE3598845.1; WP_242515294.1; WP_160690060.1; WP_246403261.1;
PYP72262.1; MCF7837491.1; WP_264149663.1; MBM4113669.1; OLC68134.1; WP_072892453.1;
WP_238655861.1; WP_236334795.1; WP_227683755.1; MCA1602785.1; ODM95369.1; WP_245583628.1;
QQR75686.1; MBI4616617.1; GFO59978.1; QLQ05411.1; TAN30479.1; PYO34951.1; HEC06454.1;
MBE3583887.1; WP_014437298.1; WP_168572956.1; WP_095132772.1; WP_218953158.1; MBV9924196.1;
WP_153972893.1; MBW8839172.1; WP_188542643.1; MCC6396956.1; MBL7198787.1; MBK5236158.1;
WP_066874330.1; MBK7580845.1; WP_114490353.1; MBI4475012.1; HBG76488.1; MBN1811280.1;
MBN1922660.1; MBW2237200.1; GIU99696.1; TMA12353.1; WP_003445719.1; MCL5281191.1;
OKP75302.1; MBV9080065.1; UCG89291.1; WP_034849750.1; WP_042360397.1; WP_068679582.1;
NLT42347.1; MBV8369746.1; KAJ3636176.1; MBU5439924.1; NLW08213.1; MCD6080931.1;
NKB87257.1; MBS5315248.1; NLL88646.1; MBN2002489.1; MCR4405566.1; WP_235228933.1;
MBE6948729.1; NLY68132.1; WP_076543453.1; OGZ30788.1; MCA0351070.1; MCL2564081.1;
NIP27196.1; MCB1569085.1; MBN1359515.1; OGV45929.1; MBU3803894.1; PYS69883.1; NLM83887.1;
TFG01810.1; MCC6670508.1; WP_209879504.1; MCH7510832.1; MBI3852464.1; WP_183362498.1;
PZN90524.1; WP_055669232.1; NQU84060.1; MCI7759261.1; MCL4485388.1; WP_042202073.1;
WP_074364995.1; MBI4114303.1; WP_110460258.1; GFO69921.1; MCU1308164.1; WP_048030406.1;
MCJ7459014.1; WP_046506235.1; WP_224035742.1; WP_277472736.1; WP_017525166.1;
WP_278677106.1; MBE3110401.1; KAI7265333.1; WP_211024704.1; MCR4428626.1; MBC7911054.1;
MBC8030606.1; WP_020425645.1; MCL2843082.1; MBP5436084.1; WP_101909648.1; WP_205426792.1;
WP_148337824.1; MBI4470339.1; MCA1577648.1; KUJ61207.1; MBE2280471.1; GBG55416.1;
WP_242847643.1; HDQ22498.1; KAJ7426784.1; OGP34630.1; MBA3661527.1; PYS02629.1; BAU49210.1;
WP_142414719.1; WP_055225420.1; MBM2828976.1; MBP8863310.1; MBW3565377.1; APV50913.1;
MBF8257865.1; PYT06129.1; MBK6589453.1; WP_057979432.1; MCH3965335.1; NFG22394.1;
MBK8248216.1; PVY61913.1; MCI4444656.1; QDU34090.1; MBO4711200.1; MBK8147040.1;
MCS6806380.1; MBC7342782.1; MBC7241223.1; MCE3198113.1; HAF12516.1; HBW50259.1;
MBE7479327.1; NLL17531.1; WP_127067918.1; WP_145077652.1; MBS5884909.1; WP_040542443.1;
HEY76410.1; NPV08023.1; PIU69114.1; MBS0657291.1; WP_253201704.1; MCC7208381.1;
MBP7088961.1; MBM4401764.1; MBA3778205.1; WP_141447891.1; QQS43076.1; OFK81106.1;
WP_171092610.1; MBO9371695.1; PWU22088.1; MBP1646848.1; MBU0474797.1; WP_197703196.1;
WP_092471123.1; WP_068728831.1; MCA2981244.1; WP_075457728.1; MBV9027074.1; MBX7171173.1;
WP_249902101.1; WP_223037694.1; WP_083303719.1; PIU45079.1; MBE6811762.1; HHY35847.1;
MCO6453311.1; WP_237382739.1; MCP4680095.1; MBI2363493.1; KFM94113.1; WP_206102689.1;
MCC6849691.1; MCA9599255.1; WP_102072770.1; MBI4493612.1; WP_058258134.1; MBI1807296.1;
WP_233181196.1; MBI4206544.1; NVM16547.1; KIV51452.1; WP_216450865.1; MCD8211577.1;
MCQ2968635.1; OLD93133.1; MBE6061602.1; MBA2494072.1; WP_072149603.1; NLK21704.1;
MBQ1397564.1; GJQ15876.1; HHY90629.1; WP_055341036.1; KUO76353.1; MBL8887496.1;
WP_104984287.1; NLA87526.1; WP_043664444.1; MCI7691026.1; MCK4519855.1; MBQ9434159.1;
MCM2315453.1; WP_264700532.1; HBL26978.1; KPK05303.1; MBA2334414.1; MBU0704436.1;
MCD6178192.1; MBP7147638.1; NYT65868.1; MBL8980494.1; WP_042218738.1; WP_150235634.1;
WP_106769488.1; WP_077835680.1; MPZ43779.1; WP_249331725.1; MCU1244318.1; OQB44524.1;
MBC7933008.1; NJD09135.1; WP_121443823.1; MCE5245892.1; WP_270647395.1; MCB0691441.1;
MBI4545928.1; MBA3892258.1; WP_207766143.1; QGU94942.1; MBV8725243.1; MBI5066126.1;
WP_235296546.1; OAD60044.1; WP_042231506.1; MBV9959226.1; WP_150275769.1; AIQ38718_1;
NOT48852.1; WP_089749626.1; AXQ31618.1; MBZ4218666.1; NYT81523.1; MBQ6863441.1;
WP_013236885.1; WP_155613500.1; NLM35753.1; RZK21514.1; WP_138209679.1; EKD72108.1;
HHX61530.1; MBC8096944.1; RMD59102.1; KAH9389652.1; MCK9267462.1; MBP9479435.1;
UUM13225.1; WP_114607726.1; MCR4310558.1; KAH0540242.1; MBK7392785.1; MCS7166068.1;
MCL4514871.1; MBQ7263968.1; MCL2763061.1; MCA1590425.1; MBN1659702.1; RKZ28044.1;
WP_205527256.1; MBR3474179.1; TMQ53512.1; TEU16594.1; WP_221803714.1; RMG48518.1;
AIQ66312.1; KKP57765.1; MBM3877837.1; WP_184307252.1; WP_010297031.1; MBN1220360.1;
MBE0569636.1; PUU93680.1; WP_039253595.1; WP_224662908.1; WP_073155376.1; MBP7177838.1;
MBK6515487.1; WP_090040314.1; WP_161810121.1; MBX3357939.1; WP_238417156.1; PRR84240.1;
MBP2673371.1; MCA0375024.1; MCO5095333.1; WP_188499000.1; WP_203165936.1; MCJ7856474.1;
WP_058526080.1; NLT13368.1; AXA36306.1; MBI2213193.1; XP_001025685.1; MBL7989945.1;
MBU0600168.1; NLG63443.1; UZE92930.1; RMF03638.1; OGD23720.1; WP_151637601.1;
MBX3708647.1; MCC7018032.1; OMD38763.1; MBI1966541.1; MCC2667029.1; KKP68865.1;
MAV91806.1; MCO6510053.1; HID63917.1; MBE6071986.1; WP_083675927.1; MBQ9534887.1;
MBV8202933.1; WP_209866367.1; MBA3532139.1; WP_061857102.1; WP_248618273.1; RJP65857.1;
WP_058461528.1; MBS3781910.1; WP_061994293.1; MBF8982352.1; XP_036796953.1; PPE72542.1;
PIV46989.1; WP_089655110.1; HHT36740.1; MCA9149031.1; RMH24471.1; WP_106010061.1;
MYG81815.1; WP_011357084.1; TAL72089.1; WP_071711021.1; MCS6774795.1; HID87996.1;
MCL2558303.1; WP_054955410.1; MBS1794384.1; ENZ29615.1; QQE12148.1; MBQ1275065.1;
NLG76515.1; MBZ9577732.1; MCG6972537.1; MBR6461401.1; MBX3355242.1; MCA1813210.1;
NTU97430.1; MBI3082421.1; KPL08801.1; WP_057579100.1; RMI05071.1; BBA50130.1;
WP_217965740.1; MBV9250693.1; KPL03977.1; TLU58742.1; MBX3384319.1; WP_088535311.1;
WP_133129720.1; MBE3576729.1; WP_128900888.1; NTW78279.1; WP_207425478.1; BAL55034.1;
WP_054537480.1; MBE6023175.1; WP_058293144.1; RME16256.1; MBR6574137.1; MBX5465348.1;
MCE5264489.1; WP_173141090.I; MBA2275890.1; WP_014961429.1; MBL8125362.1; MBD3190878.1;
NLM48758.1; MBA3355997.1; MBL6751543.1; WP_008518324.1; MCU1265627.1; MCE1163986.1;
WP_258605798.1; SEB80022.1; MCC7404932.1; MCB2203714.1; WP_098252338.1; UCD78329.1;
GBC82590.1; KAF5908081.1; MCR4416871.1; MBC5815143.1; WP_077610354.1; WP_139456952.1;
MBP6059657.1; MCA9257724.1; WP_168058325.1; MCC6820266.1; REJ75671.1; RJQ68215.1;
OFO62937.1; WP_205051388.1; NLW25906.1; NOX36824.1; WP_219220884.1; WP_090698789.1;
OIP77463.1; WP_245584876.1; MCD6500583.1; WP_013273278.1; WP_011721816.1; KZK73489.1;
BDE07084.1; WP_165226081.1; PYS95793.1; TMQ57630.1; WP_061612682.1; HCT41220.1;
WP_015247934.1; WP_122638847.1; MBE7514954.1; NDC78974.1; OGT46816.1; MBS4956539.1;
WP_203126634.1; WP_223069335.1; WP_210400918.1; MBI3810930.1; WP_029922036.1; HHW90181.1;
NOZ71182.1; WP_129578573.1; WP_055105445.1; WP_166209404.1; MBL8757574.1; WP_077397608.1;
MBP1656600.1; OOH73632.1; HHX79775.1; WP_250342634.1; MBA2665359.1; MBI5020667.1;
WP_042132721.1; WP_272440820.1; WP_019106905.1; MBL9024965.1; NLC79356.1; MCS6904544.1;
WP_213595547.1; WP_047000818.1; HAU85397.1; MBW2275164.1; MBV8731684.1; MCS7049480.1;
MSV29102.1; WP_269497714.1; MCD6289791.1; UCD15917.1; MCU1309027.1; MCB0275684.1;
TVQ42994.1; OGT43571.1; OIP34757.1; WP_131562885.1; MBK9067681.1; MBW7931683.1;
MBX3020991.1; PYQ30721.1; MCB1161060.1; MBX5480786.1; WP_209701425.1; MCL8208171.1;
MCL4196048.1; PYP54988.1; OGY41777.1; MCI5628058.1; WP_072745160.1; MCD6528612.1;
NIM98768.1; MBV8783137.1; WP_056095747.1; KKS74015.1; TPN34615.1; RYZ06284.1;
WP_066244025.1; WP_213656510.1; NIO11490.1; MCA9283518.1; MCC7549846.1; MBN8727417.1;
MBN1975937.1; KKS91478.1; WP_194723407.1; NBW99213.1; WP_274228206.1; MBR5702463.1;
OGP19056.1; WP_102068556.1; PJB31608.1; MBA3317634.1; MBI3124815.1; MBX7061392.1;
WP_276733348.1; MBZ9572709.1; MCL5953707.1; WP_071637603.1; OQA18497.1; MBV8126958.1;
HHV06838.1; TFJ85644.1; MBN1260978.1; HEY89593.1; WP_267599971.1; CAN96343.1; MCH9014580.1;
MCR4322607.1; MBR5292278.1; OGT35465.1; WP_275935413.1; MCK5086134.1; WP_248624914.1;
WP_200123329.1; WP_250674091.1; MBW6502773.1; WP_144213347.1; MBR5485721.1;
WP_226389319.1; EOR20699.1; MCR4433143.1; NOZ26767.1; WP_188695842.1; MBR3955030.1;
WP_235291028.1; MBM6985669.1; MBN1326130.1; WP_218283615.1; HAK43090.1; KAF0151397.1;
OGT23430.1; WP_205701433.1; PYS32606.1; WP_184171494.1; MBS1645038.1; MBT9281979.1;
WP_184468136.1; MBU91299.1; OPY26440.1; MBV8630427.1; MCI8329159.1; MCB0078514.1;
WP_016113278.1; WP_070967570.1; WP_220179372.1; WP_066892700.1; RKY35266.1; RLE89319.1;
HIE38836.1; MBZ5678235.1; TAJ96682.1; WP_063555521.1; WP_029274354.1; AIA30774.1;
WP_073062089.1; WP_090496264.1; NYT78136.1; UCC87256.1; MCC7487838.1; WP_250345713.1;
MCL6522601.1; NTW56640.1; MBN1507962.1; MBV8265089.1; MBI2839698.1; MCL1951133.1;
OGP09484.1; WP_074657924.1; HHY59166.1; WP_093912856.1; WP_015935392.1; UCE52012.1;
NLN49791.1; OFW78065.1; KTD16249.1; RLG44779.1; UCG35210.1; MBE0643302.1; PZN26995.1;
MCL2128656.1; MCR9102818.1; MBI3182388.1; MBV8244343.1; WP_124847885.1; MCB0265946.1;
TFK07434.1; WP_047800489.1; WP_072723443.1; HHW47326.1; MCR4316839.1; MCC6618973.1;
HBN84858.1; WP_044823451.1; TVM05519.1; MCC7353135.1; MBP1697184.1; TAJ09672.1;
WP_084386375.1; WP_216390537.1; WP_195468087.1; MCB9127901.1; WP_089758804.1;
WP_272561395.1; WP_103374156.1; MBS4030095.1; AKS24695.1; MCC6283726.1; MCM8823472.1;
KKR10019.1; WP_040194434.1; MCL5883828.1; MBP8911302.1; NCQ16241.1; CAG7591085.1;
MCC5910906.1; MBN2169961.1; WP_007202072.1; MBV8491530.1; WP_036724434.1; PYO61056.1;
MBM4358201.1; WP_012978918.1; WP_253350515.1; WP_090043060.1; RMG64568.1; MBD3254879.1;
MBA3522585.1; WP_277644160.1; WP_019639947.1; MBJ6723879.1; WP_215907678.1; WP_246021945.1;
TLY24869.1; MBV8341127.1; WP_023744872.1; KAF0142743.1; WP_054252822.1; MBE3555618.1;
MCA9145836.1; MCD6027436.1; MBP2685379.1; MBN1179205.1; MBP1710577.1; MBK9155277.1;
EET85738.1; WP_028373544.1; MBW4052400.1; TSC63152.1; MBI1815593.1; MCU1348344.1;
SMP64906.1; WP_091961210.1; NLN18902.1; MCK7508137.1; WP_195999563.1; MBC7189982.1;
PYK57976.1; MBX7233738.1; TMB03153.1; NLZ81125.1; MBN6187189.1; WP_053957844.1;
MCC6585618.1; WP_136621782.1; MBX3006900.1; MBV8772433.1; WP_200595167.1; WP_090882512.1;
KPK93981.1; WP_255696721.1; WP_246196355.1; PJF36190.1; WP_187594956.1; CAG0967655.1;
RUA15927.1; TMC98318.1; KUO48860.1; MCF7871121.1; MBC8060216.1; WP_070120596.1;
NLL64100.1; WP_023726779.1; AHE67011.1; WP_040328658.1; TMQ55160.1; MBW6458041.1;
RLG40065.1; MBN2168046.1; RKY44736.1; WP_223266881.1; MBU5591988.1; WP_245955077.1;
WP_202766764.1; AUS95653.1; WP_091687135.1; GBC78163.1; WP_244879535.1; WP_136607614.1;
WP_055913731.1; WP_230575243.1; MCL2139976.1; PID56965.1; MCF2639694.1; MBO2488105.1;
WP_216463051.1; WP_253784995.1; WP_084233707.1; GIO58394.1; MXX33187.1; SMP35281.1;
MBI3609002.1; WP_253294761.1; WP_110929781.1; KYQ47640.1; RUT28078.1; MCB9874972.1;
MBP6908795.1; MCJ7568840.1; RME85912.1; MBI4501240.1; MCK5321572.1; MBP8600934.1;
MBI3331551.1; WP_023525273.1; WP_181168736.1; NLK35783.1; PHV71870.1; MCE1157664.1;
EWM27651.1; MCK5591991.1; WP_151618662.1; WP_165200766.1; MBP1563815.1; WP_046679737.1;
WP_171701960.1; WP_195179520.1; MBK5259782.1; EZH67389.1; WP_160843656.1; PLX32597.1;
MBA2249594.1; WP_181165641.1; WP_025385632.1; MCH7874918.1; MBP7137836.1; WP_194714420.1;
NMC91746.1; EQB63449.1; KXA30763.1; SNS03741.1; KPJ55115.1; WP_150886414.1; MBW3552241.1;
WP_269414633.1; OWA32971.1; MCI0530437.1; WP_273592898.1; MBA3586883.1; MBQ5695322.1;
WP_214226791.1; WP_091495338.1; WP_245704497.1; GJQ61605.1; WP_169506673.1; PYS82461.1;
MCG8540682.1; NLG86134.1; NPV66177.1; WP_269426853.1; MBD1209013.1; MBT9174093.1;
NLH42692.1; WP_041064930.1; WP_229696444.1; MBX6360317.1; NNG16361.1; WP_244364631.1;
WP_251376963.1; WP_098385998.1; MBO9559915.1; WP_079493923.1; WP_221860989.1;
WP_1369231.50.1; WP_000645926.1; HBI17318.1; RMG40534.1; MBR3771371.1; WP_201134445.1;
WP_133127856.1; EQD24757.1; WP_223607829.1; WP_228456258.1; MBO4930252.1; MBA3919334.1;
WP_193413709.1; MBI3984071.1; MCS6954090.1; NTU57420.1; TPK69672.1; MBI3787530.1;
WP_276876808.1; TMQ65905.1; OGB74306.1; WP_116398992.1; MCC6453224.1; MBL0890201.1;
TMJ11024.1; WP_236849965.1; UCB53628.1; RQD77253.1; MCF6270871.1; GFY72294.1; KAF7274075.1;
WP_270453263.1; MBJ9993545.1; WP_278271007.1; MBA3344923.1; WP_073080361.1; MBQ7155737.1;
RME45569.1; KAF0155529.1; MBV5302801.1; MCL4521383.1; RJP61988.1; MBI4101207.1; RPJ13399.1;
MBX2989589.1; OGC78404.1; TMK63772.1; WP_090987245.1; WP_098268474.1; MBL8201731.1;
TET96780.1; MBM4165558.1; MCB9584397.1; MBQ8830930.1; KXK04257.1; MBD5543211.1;
HAV41483.1; MCI9576011.1; MCD4738541.1; MCI1958282.1; MCF8382986.1; WP_230995512.1;
WP_012501607.1; OGU32050.1; AWZ48092.1; MCL6606162.1; TLY71469.1; NLN53922.1; MBI3965483.1;
WP_246022816.1; MBY9002427.1; MBC7928076.1; MBI5214544.1; MBV8067930.1; MCF0147710.1;
WP_039219087.1; PYP18383.1; MCG3149391.1; THJ20627.1; WP_137012809.1; WP_158814994.1;
WP_256006436.1; MBI3802175.1; NLG69483.1; MBI3613965.1; OGT99633.1; KYG11062.1; TAL78490.1;
MBS0037454.1; MBL8265334.1; WP_258588361.1; TFG01008.1; MCA9642629.1; MBV9359514.1;
HEY47771.1; OGI08217.1; WP_054739700.1; WP_142698001.1; MBV8455355.1; WP_244862769.1;
MCA9183294.1; MCE5236249.1; MBA3536642.1; HEC21758.1; MBS1934119.1; NMA54919.1;
PMP96069.1; MBC7360987.1; MCL4427075.1; WP_151409672.1; KQU92052.1; WP_224728674.1;
AIQ10695.1; WP_169230385.1; WP_235894723.1; WP_217705710.1; MTI83882.1; MBI4394904.1;
RLG50316.1; MBA3969371.1; NLK23788.1; WP_095083834.1; WP_244482825.1; WP_264526817.1;
MBK6771443.1; RLE91807.1; MBV9718445.1; HEY42411.1; MBR3059047.1; RYZ87136.1;
WP_109193176.1; RKY54987.1; WP_069000066.1; HAO46512.1; MBS3825915.1; WP_001148759.1;
RME60245.1; WP_243700812.1; MBR4018672.1; MBR9976907.1; WP_042208934.1; WP_257916941.1;
PJB00347.1; KUK65027.1; WP_004814036.1; MCK4383604.1; MBI2604617.1; WP_202357020.1;
HHT70832.1; MBX2995414.1; WP_057934685.1; MBS4804100.1; OGB90356.1; HHF98769.1;
MBY0490012.1; WP_154782579.1; MCD6359959.1; UCD02171.1; WP_025435886.1; WP_242199485.1;
MBK7876764.1; MBQ6207910.1; BCG77296.1; TMK30136.1; NPV74250.1; STY25448.1; SKC83347.1;
GIP24646.1; WP_072923824.1; UCD24252.1; WP_131356466.1; MBM4175359.1; MBV9506819.1;
MBA3959011.1; WP_238324644.1; TLU56739.1; KXA00138.1; WP_165104060.1; HHW06720.1;
HJB99157.1; MBO9662053.1; OLD48313.1; WP_111548052.1; MBE6967807.1; HAG12170.1;
MBK6305092.1; NUQ92933.1; MBI4259298.1; MBY9012917.1; MBI5477736.1; WP_153832592.1;
WP_013842452.1; MBC7671276.1; NYT25000.1; NLN38959.1; WP_122627091.1; MCS7222797.1;
MBD5105614.1; MBX3744282.1; WP_184872802.1; MBV8163365.1; WP_108463819.1; BAF60127.1;
TMQ01146.1; MBW6455630.1; MCU0275052.1; WP_025332649.1; NCO23113.1; WP_104713855.1;
MBE3603946.1; WP_068867852.1; MCR9192249.1; MBL0169239.1; MBP6004017.1; WP_125081600.1;
MCD3245064.1; WP_056220070.1; AUG58453.1; WP_181304597.1; MBK7643173.1; WP_115834715.1;
MAD78307.1; PIE55315.1; MBI5446182.1; GLC28504.1; WP_074304865.1; MCJ7825102.1; MCI6693313.1;
NLU08881.1; NOT92889.1; WP_050617728.1; RLE76115.1; MBA3658231.1; NLP36629.1; NBX77277.1;
WP_226953121.1; MBP2648726.1; MBS1917420.1; MBL7062761.1; MBL8983806.1; MCL5028132.1;
WP_275423163.1; WP_165973787.1; OGO79450.1; TKJ24953.1; WP_090446594.1; WP_116807868.1;
MCC7338284.1; PHV69870.1; HHH83815.1; HHW99441.1; PYV55525.1; WP_242213497.1;
WP_109768775.1; NOX18828.1; WP_061972410.1; WP_031537677.1; WP_213237714.1; PYO86852.1;
HIC89776.1; TET48490.1; PKN19254.1; MBM4194002.1; MBO2478621.1; HHB13388.1; WP_217346214.1;
TCT09792.1; MBC8022709.1; WP_236785190.1; WP_243839394.1; WP_213653266.1; GIU96586.1;
MCM8774206.1; TDX08388.1; PKN85107.1; MBD3174058.1; WP_093289010.1; PSO43001.1;
WP_014559487.1; MBX7137825.1; NMD32878.1; MBL7821018.1; QCT00836.1; WP_079701245.1;
QWV95396.1; WP_154280260.1; TMQ30166.1; PZR58594.1; HCC34060.1; XP_005702867.1;
MCB9275390.1; UCC27186.1; OGY47979.1; MCP4896985.1; WP_233281086.1; MBI5913373.1;
MBP7979088.1; WP_194566344.1; BAV49049.1; MBE6995622.1; MBA2264453.1; MBS3817210.1;
GBD36364.1; MCL5037541.1; WP_125906726.1; MCI9355065.1; MCK4813805.1; MSR85848.1;
MCB0417930.1; WP_087338179.1; TIY09155.1; MBO07203.1; WP_119771354.1; MCK5475765.1;
WP_154539131.1; WP_133524088.1; WP_224689260.1; MBS0578728.1; WP_051507027.1; MBL8072532.1;
RZJ91289.1; MBD3168735.1; OGQ06929.1; MBI4420569.1; WP_260533679.1; MSU76028.1;
MCP5024680.1; CEH34934.1; EDK35729.1; WP_044039286.1; MCE7936971.1; WP_264751773.1;
MCC6339582.1; PYS90121.1; NTV99414.1; WP_014202656.1; WP_035689749.1; WP_117300088.1;
WP_269190145.1; WP_246426965.1; MCL2243841.1; WP_127120906.1; WP_237170742.1; MBI0582604.1;
WP_020373972.1; MBP9106577.1; MCS7235644.1; MCI9586561.1; MBI64889.1; MBQ3087734.1;
RCK75934.1; WP_094083256.1; BAH37697.1; WP_197526041.1; MCH7714533.1; BAH08358.1;
NDC42418.1; MCK4891406.1; WP_062197697.1; MBB3132201.1; MBP7766367.1; MBI4693425.1;
MCS7017112.1; MBD3193662.1; WP_024615341.1; WP_073151358.1; WP_066500405.1; MSR06686.1;
WP_012530547.1; MSQ73494.1; WP_006522384.1; OQA10689.1; WP_096282015.1; MBR5248737.1;
WP_110346093.1; TSD01833.1; RMF37333.1; MBP6772095.1; NRA36053.1; MCK6620459.1; NUQ43289.1;
NIM48118.1; WP_108778369.1; PYO43899.1; WP_107215465.1; OPZ87079.1; OLZ10095.1;
WP_173299151.1; MBV9073566.1; WP_223088495.1; NTV25583.1; OGB22682.1; WP_113652683.1;
MBX7244036.1; MBI3004534.1; WP_041700869.1; MCL2190886.1; MCF7833433.1; MBK8380304.1;
WP_235333940.1; MBK7254673.1; MBX3126588.1; MCL2809964.1; WP_265837405.1; MBY9016348.1;
MBV8144620.1; MCL5259145.1; WP_163968490.1; MCG0277332.1; WP_100341417.1; MBP7341632.1;
TMA46369.1; MBI1957212.1; WP_048721514.1; NLI12027.1; MCI6488445.1; MBD3234295.1;
NLX50488.1; WP_150458240.1; MBS1732519.1; WP_077556911.1; OGC85582.1; MBT9131603.1;
NBQ53616.1; WP_081916455.1; MCO8128272.1; PIU67894.1; PSR04823.1; MCL1991492.1; QZA33405.1;
WP_204259824.1; OVE74398.1; WP_201430073.1; HHL53063.1; WP_239549276.1; UCF41175.1;
WP_166280144.1; HEY52065.1; NLA05579.1; NLN07838.1; MCS7027341.1; XP_019714689.1;
WP_114066199.1; MCB2155583.1; MBT9260311.1; WP_057291367.1; WP_152889815.1; MCU0424922.1;
WP_115619872.1; WP_140733436.1; MBU6434781.1; MCA1571549.1; KKW02676.1; KON27617.1;
WP_243119796.1; WP_094252437.1; TXT66459.1; MCI7474304.1; MBA2633906.1; MCK4380791.1;
WP_077444684.1; NLM52969.1; MCG3211241.1; MXX72885.1; MBS1858462.1; MBN1278801.1;
MBN1583760.1; EPS47825.1; TFG16522.1; WP_231424421.1; WP_277479900.1; MCK4386296.1;
MCG8606126.1; WP_023687504.1; OQX51100.1; OLE12418.1; WP_244562885.1; MBE9478834.1;
WP_023813490.1; WP_224570975.1; WP_173089392.1; MBV9272141.1; WP_222460721.1;
WP_034707168.1; WP_109924457.1; NTE01162.1; MBM3163739.1; MCD6348652.1; NUQ21034.1;
WP_146496826.1; NGR08000.1; RLE78732.1; MCD7948007.1; WP_194139178.1; WP_073107532.1;
MBI5660604.1; WP_121487847.1; MBS3089760.1; MBK7806038.1; MCU1283971.1; MCP5455391.1;
MBK6667317.1; TAJ34997.1; APR81189.1; WP_172200146.1; MCL2265816.1; MCS7229535.1;
GBL39430.1; MBA2669996.1; WP_196099413.1; GIX47441.1; WP_207534076.1; WP_156912168.1;
WP_107651809.1; KUK81625.1; MBI5078809.1; OGO67479.1; WP_184403080.1; WP_236657304.1;
WP_129726591.1; TMB17979.1; MBI2175618.1; MBP2661882.1; MBV8460177.1; NLL29481.1;
MCG7857567.1; MBX3029227.1; MCS7182757.1; WP_114458715.1; MBC7901656.1; HJB81437.1;
WP_149092157.1; HEY71245.1; WP_236026227.1; WP_085660162.1; MBL8861336.1; WP_109667663.1;
UCE02257.1; MCC7489308.1; WP_227389624.1; MBY5957334.1; WP_123853721.1; MBI4372350.1;
MBU5613993.1; VEN38701.1; WP_028308569.1; MXZ36098.1; MBR6779604.1; WP_238663703.1;
MBL8990706.1; WP_003541718.1; MBS0287465.1; MCH7587478.1; WP_072910647.1; WP_073997686.1;
WP_014855562.1; MBX9657955.1; MCT4564876.1; WP_084665579.1; WP_208698576.1; WP_250138036.1;
WP_018978866.1; AXC10966.1; TVP58735.1; MCJ7497287.1; WP_146328520.1; MCL2722569.1;
MCB0722593.1; MBP3928447.1; WP_026850121.1; WP_248253493.1; WP_079686628.1; MBV8498443.1;
NOZ00152.1; WP_131596518.1; PYQ46876.1; KJS29649.1; WP_256635055.1; WP_073487661.1;
MBQ9486846.1; OGU57600.1; MBR2935265.1; MBU6239380.1; WP_053472687.1; WP_197193605.1;
PIQ10094.1; WP_253531503.1

TABLE C
Homo sapiens; Theropithecus gelada; Aotus nancymaae; Pan troglodytes; Bos indicus; Rhinolophus
ferrumequinum; Galeopterus variegatus; Myotis myotis; Equus przewalskii; Balaenoptera acutorostrata
scammoni; Pteronotus parnellii mesoamericanus; Gulo gulo luscus; Phoca vitulina; Phyllostomus discolor;
Prionailurus bengalensis; Ursus maritimus; Castor canadensis; Leopardus geoffroyi; Molossus molossus; Sciurus
carolinensis; Tupaia chinensis; Urocitellus parryii; Vulpes vulpes; Pipistrellus kuhlii; Condylura cristata;
Phodopus roborovskii; Carlito syrichta; Myotis brandtii; Heterocephalus glaber; Onychomys torridus; Microtus
ochrogaster; Balaenoptera physalus; Rattus norvegicus; Myodes glareolus; Phascolarctos cinereus; Sorex
araneus; Mus pahari; Peromyscus leucopus; Suncus etruscus; Strigops habroptila; Accipiter gentilis; Aquila
chrysaetos chrysaetos; Tinamus guttatus; Tachyglossus aculeatus; Dromaius novaehollandiae; Calidris pugnax;
Cricetulus griseus; Apus apus; Cuculus canorus; Glaucidium brasilianum; Apteryx rowi; Rissa tridactyla; Grus
americana; Falco rusticolus; Dryobates pubescens; Gallus gallus; Caretta caretta; Aythya fuligula; Gopherus
flavomarginatus; Platysternon megacephalum; Anser cygnoides; Chelonoidis abingdonii; Motacilla alba alba;
Chelydra serpentina; Tyto alba; Odobenus rosmarus divergens; Latimeria chalumnae; Ficedula albicollis; Passer
montanus; Catharus ustulatus; Coturnix japonica; Camarhynchus parvulus; Acipenser ruthenus; Atractosteus
spatula; Parus major; Sturnus vulgaris; Molothrus ater; Melozone crissalis; Pseudopodoces humilis; Zosterops
borbonicus; Microcaecilia unicolor; Cyanistes caeruleus; Agelaius phoeniceus; Eublepharis macularius; Pitangus
sulphuratus; Gekko japonicus; Protopterus annectens; Cygnus olor; Onychostruthus taczanowskii; Colinus
virginianus; Megalops cyprinoides; Spea bombifrons; Callorhinchus milii; Callipepla squamata; Hemicordylus
capensis; Megalops atlanticus; Rhinatrema bivittatum; Python bivittatus; Lates calcarifer; Channa argus;
Mastacembelus armatus; Archocentrus centrarchus; Solea senegalensis; Thamnophis sirtalis; Pristis pectinata;
Oreochromis niloticus; Engystomops pustulosus; Salarias fasciatus; Sphaeramia orbicularis; Colossoma
macropomum; Myripristis murdjan; Chanos chanos; Crotalus tigris; Pundamilia nyererei; Pseudonaja textilis;
Podarcis raffonei; Notechis scutatus; Micropterus dolomieu; Epinephelus lanceolatus; Simochromis diagramma;
Protobothrops mucrosquamatus; Chelmon rostratus; Podarcis muralis; Egretta garzetta; Mugil cephalus;
Pungitius pungitius; Echeneis naucrates; Seriola lalandi dorsalis; Fundulus heteroclitus; Cyprinodon tularosa;
Gasterosteus aculeatus aculeatus; Acanthochromis polyacanthus; Labeo rohita; Oncorhynchus keta; Nanorana
parkeri; Sphaerodactylus townsendi; Scomber japonicus; Amphiprion ocellaris; Cheilinus undulatus; Seriola
dumerili; Pygocentrus nattereri; Poeciliopsis prolifica; Carassius auratus; Thalassophryne amazonica;
Ophiophagus hannah; Parambassis ranga; Oncorhynchus kisutch; Poecilia formosa; Sebastes umbrosus;
Austrofundulus limnaeus; Nothobranchius furzeri; Neomonachus schauinslandi; Perca fluviatilis; Triplophysa
rosa; Syngnathus scovelli; Epinephelus fuscoguttatus; Sparus aurata; Alosa alosa; Larimichthys crocea;
Syngnathus acus; Puntigrus tetrazona; Electrophorus electricus; Salmo trutta; Albula glossodonta;
Pangasianodon hypophthalmus; Cyprinus carpio; Gambusia affinis; Kryptolebias marmoratus; Periophthalmus
magnuspinnatus; Hymenochirus boettgeri; Astyanax mexicanus; Tetraodon nigroviridis; Salmo salar; Anguilla
anguilla; Etheostoma spectabile; Danio rerio; Melanotaenia boesemani; Toxotes jaculatrix; Esox lucius; Silurus
meridionalis; Hippocampus zosterae; Morone saxatilis; Hippocampus comes; Denticeps clupeoides; Synchiropus
splendidus; Clupea harengus; Myxocyprinus asiaticus; Tachysurus fulvidraco; Anabas testudineus; Misgurnus
anguillicaudatus; Xiphophorus maculatus; Anabarilius grahami; Conger conger; Albula goreensis; Clarias
gariepinus; Oryzias latipes; Poecilia reticulata; Pimephales promelas; Ictalurus punctatus; Gadus morhua;
Eleutherodactylus coqui; Manacus vitellinus; Trachypithecus francoisi; Talpa occidentalis; Mirounga leonina;
Collichthys lucidus; Sceloporus undulatus; Scophthalmus maximus; Aldrovandia affinis; Gymnothorax
javanicus; Pleuronectes platessa; Dicentrarchus labrax; Carassius gibelio; Alligator sinensis; Charadrius
vociferus; Xenopus tropicalis; Scleropages formosus; Dissostichus mawsoni; Cottoperca gobio; Pleurodeles
waltl; Coryphaenoides rupestris; Triplophysa tibetana; Lynx pardinus; Muraenolepis orangiensis; Trematomus
bernacchii, Apostichopus japonicus; Pseudochaenichthys georgianus; Holothuria leucospilota; Branchiostoma
floridae; Myotis davidii; Takifugu flavidus; Chelonia mydas; Megalobrama amblycephala; Pomacea canaliculata;
Sinocyclocheilus anshuiensis; Alosa sapidissima; Ctenopharyngodon idella; Paramormyrops kingsleyae;
Xyrauchen texanus; Tegillarca granosa; Acanthaster planci; Strongylocentrotus purpuratus; Patiria miniata;
Lytechinus pictus; Mytilus coruscus; Crassostrea virginica; Lamellibrachia satsuma; Mytilus californianus;
Nyctereutes procyonoides; Centruroides sculpturatus; Asterias rubens; Crassostrea gigas; Mizuhopecten
yessoensis; Haliotis rubra; Mytilus galloprovincialis; Haliotis rufescens; Ostrea edulis; Capitella teleta; Dreissena
polymorpha; Eurypyga helias; Hemibagrus wyckioides; Ameiurus melas; Patella vulgata; Mya arenaria;
Desmophyllum pertusum; Actinia tenebrosa; Gigantopelta aegis; Plakobranchus ocellatus; Pocillopora
damicornis; Dendronephthya gigantea; Orbicella faveolata; Candidula unifasciata; Mercenaria mercenaria;
Styela clava; Canis lupus dingo; Nematostella vectensis; Stylophora pistillata; Aptenodytes forsteri;
Armadillidium vulgare; Acropora millepora; Sepia pharaonis; Bulinus truncatus; Trichoplax sp. H2; Cherax
quadricarinatus; Chaetura pelagica; Anneissia japonica; Ictalurus furcatus; Dimorphilus gyrociliatus; Bradysia
coprophila; Panonychus citri; Zonotrichia albicollis; Tetranychus urticae; Rotaria sp. Silwood2; Cryptotermes
secundus; Phallusia mammillata; Penaeus vannamei; Lottia gigantea; Camelus ferus; Rotaria magnacalcarata;
Elysia chlorotica; Ladona fulva; Penaeus monodon; Penaeus japonicus; Notodromas monacha; Limulus
polyphemus; Lamprotornis superbus; Octopus bimaculoides; Rotaria sordida; Folsomia candida; Dinothrombium
tinctorium; Oppia nitens; Timema cristinae; Biomphalaria glabrata; Penaeus chinensis; Chionoecetes opilio;
Ischnura elegans; Opisthocomus hoazin; Cimex lectularius; Aplysia californica; Oopsacas minuta; Ciona
intestinalis; Stegastes partitus; Daphnia pulex; Amphimedon queenslandica; Pollicipes pollicipes; Pediculus
humanus corporis; Hyalella azteca; Homarus americanus; Rhipicephalus sanguineus; Megalurothrips usitatus;
Rhipicephalus microplus; Priapulus caudatus; Didymodactylos camosus; Thrips palmi; Pseudolycoriella hygida;
Rotaria sp. Silwood1; Paramacrobiotus metropolitanus; Eurytemora affinis; Homalodisca vitripennis;
Schistocerca americana; Zootermopsis nevadensis; Amphibalanus amphitrite; Frankliniella occidentalis;
Oppiella nova; Ixodes scapularis; Allacma fusca; Elysia marginata; Dermacentor andersoni; Daphnia galeata;
Procambarus clarkii; Bugula neritina; Daphnia magna; Cyprideis torosa; Darwinula stevensoni; Nymphon
striatum; Dermacentor silvarum; Ixodes persulcatus; Halyomorpha halys; Helobdella robusta; Sarcoptes scabiei;
Nezara viridula; Macrosteles quadrilineatus; Paraphysoderma sedebokerense; Hydra vulgaris; Octopus sinensis;
Timema monikensis; Timema bartmani; Leptotrombidium deliense; Timema tahoe; Hypsibius exemplaris;
Laodelphax striatellus; Tyrophagus putrescentiae; Liparis tanakae; Pecten maximus; Oedothorax gibbosus;
Adineta steineri: Araneus ventricosus; Ramazzottius varieornatus; Blomia tropicalis; Caerostris darwini;
Caerostris extrusa; Dermatophagoides farinae; Lepeophtheirus salmonis; Serratia symbiotica; Basidiobolus
meristosporus CBS 931.73; Uloborus diversus; Halotydeus destructor; ; Monosiga brevicollis MX1; Pantoea sp.
MQR6; Serratia microhaemolytica; Bdellovibrionales bacterium; Spirometra erinaceieuropaei; Candidatus
Magasanikbacteria bacterium CG11_big_fil_rev_8_21_14_0_20_39_34; Bowmanella dokdonensis;
Methylocystis sp. ATCC 49242; Ostreobium quekettii; Paragonimus skrjabini miyazakii; Clostridia bacterium;
Flavobacteriaceae bacterium; Oceanisphaera psychrotolerans; Dermatophagoides pteronyssinus; Marinilabiliales
bacterium; Fulvivirga imtechensis; Gammaproteobacteria bacterium; Brenneria salicis; Pseudoalteromonas;
unclassified Pantoea; Macellibacteroides fermentans; Phocaeicola dorei; Brenneria goodwinii; unclassified
Brenneria; Bacteroidales bacterium; Paragonimus westermani; Erwinia phyllosphaerae; Bacteroidetes bacterium
GWA2_40_15; Namao virus; Leptonychotes weddellii; Fulvivirga kasyanovii; Propithecus coquereli; Chania
multitudinisentens; Serratia sp. S1B; Adineta ricciae; Izhakiella australiensis; Trichoplax adhaerens; Erwinia sp.
198; Pantoea; Salinivibrio kushneri; Rahnella sp. AN3-3W3; Fulvivirga marina; Schistocephalus solidus; Pantoea
alhagi; Serratia odorifera; Caballeronia sp. GAWG2-1; Photorhabdus australis; Pseudoalteromonas sp. Tllg23B;
Cytophagales bacterium; Acetobacteroides hydrogenigenes; Stegodyphus dumicola; Salinimonas sediminis;
unclassified Erwinia; Gilliamella apicola; Methylocystis sp. WRRC1; Schistosoma guineensis; Pedobacter
metabolipauper; Schistosoma haematobium; Gallaecimonas xiamenensis; Pantoea sp. RIT388; Syncephalis
fuscata; Flavobacteriales bacterium; unclassified Pseudoalteromonas; Schistosoma mattheei; Sparganum
proliferum; Tieghemiomyces parasiticus; Clostridium sp.; Bacillota bacterium; Erwinia persicina; Saccharobesus
litoralis; Biostraticola tofi; Piptocephalis tieghemiana; Mixta theicola; Bacteroidaceae bacterium; Cytophagaceae
bacterium; Candidatus Pacebacteria bacterium; Alteromonas pelagimontana; Sodalis glossinidius; Paraglaciecola
arctica; Mixta hanseatica; Cloeon dipterum; Schistosoma intercalatum; Marinobacter salicampi; Bacteroidota
bacterium; Candidatus Kaiserbacteria bacterium; Blattella germanica; Cunninghamella echinulata; Mixta
intestinalis; Oceanisphaera arctica; Coemansia sp. RSA 1939; Brenneria sp. EniD312; Lentimierobium
saccharophilum; Bacteroidetes bacterium ADurb.Bin008; Bacteroidia bacterium; Parabacteroides sp.;
Schistosoma spindale; Parcubacteria group bacterium; Dimargaris verticillata; unclassified Gilliamella;
Gonapodya prolifera JEL478; Oceanisphaera litoralis; Alistipes sp. ZOR0009; Trichonephila inaurata
madagascariensis; Rahnella bruchi; Candidatus Peregrinibacteria bacterium
CG10_big_fil_rev_8_21_14_0_10_36_19; Alteromonas sp. CIM14; Spirochaeta cellobiosiphila; Candidatus
Sphingobacterium stercoripullorum; Alteromonas sp. a30; Methylocystis sp.; Pantoea sp. AS-PWVM4;
Aliikangiella marina; Phocaeicola faecicola; Muribaculum intestinale; Clostridium tunisiense; Mixta calida;
Candidatus Kerfeldbacteria bacterium; Paraprevotella sp.; Zobellella juensis; Bacteroidetes bacterium
GWA2_30_7; Candidatus Paraprevotella stercoravium; Tenuifilum thalassicum; Methylosinus sp. Sm6;
Photorhabdus luminescens; Bacteroides sp. OF04-15BH; Linnemannia zychae; Alteromonadaceae bacterium
M269; Cronobacter condimenti; Motilimonas eburnea; Photorhabdus sp. CRCIA-P01; Pantoea ananatis LMG
20103; Affinibrenneria salicis; Paraprevotella clara; Porphyromonadaceae bacterium; Bacteroides sp.; Pantoea
sp. At-9b; Stegodyphus mimosarum; Candidatus Peregrinibacteria bacterium; Syncephalastrum racemosum;
Schistosoma margrebowiei; Oscillospiraceae bacterium; Paraglaciecola sp. L3A3; Marinoscillum sp. 108;
Trichobilharzia szidati; Citrobacter rodentium; Longilinea sp.; Adhaeribacter rhizoryzae; Tigriopus californicus;
Cronobacter sakazakii ATCC BAA-894; Salmonella enterica; Photorhabdus tasmaniensis; Cyclobacteriaceae
bacterium; Ohtaekwangia koreensis; Bacteroidetes bacterium GWF2_38_335; Pseudoalteromonas luteoviolacea;
Phocaeicola paurosaccharolyticus; Fibrobacteria bacterium; Mucilaginibacter paludis; Pectobacterium
punjabense; Lonsdalea quercina; Zobelleila denitrificans; Myxococcales bacterium; Actinobacillus; Cronobacter
sakazakii; Schistosoma japonicum; Pedobacter planticolens; Bowmanella denitrificans; Ningiella ruwaisensis;
Pelistega suis; Paraglaciecola sp. MB-3u-78; Hymenolepis diminuta; Spirochaetia bacterium; Schistosoma
mansoni; Pseudoalteromonas sp. CIP111854; Paraglaciecola psychrophila; Paludibacteraceae bacterium;
Rahnella; Fasciola hepatica; Motilimonas pumila; Odoribacter sp. Z80; Vibrio nigripulchritudo; Erwinia;
Rouxiella aceris; Fulvivirga sedimenti; Guillardia theta CCMP2712; Pedobacter panaciterrae; Chimaeribacter
coloradensis; Pectobacterium aquaticum; Mixta sp. Marseille-Q2659; Paragonimus kellicotti; unclassified
Mannheimia; Pantoea eucrina; Desulfurivibrio alkaliphilus; Escherichia coli; unclassified
Symbiopectobacterium; Hafnia alvei; Nephila pilipes; Hydatigera taeniaeformis; Franconibacter helveticus;
Parabacteroides sp. FAFU027; Pseudoalteromonas ruthenica; Muribaculaceae bacterium; Zophobihabitans
entericus; Buttiauxella izardii; Enterobacillus tribolii; Rikenellaceae bacterium; Agarivorans albus; Agarivorans
albus MKT 106; Erwinia billingiae; Pelistega europaea; Schistosoma rodhaini; Candidatus Sodalis pierantonius;
Candidatus Bacteroides merdipullorum; Enterobacter hormaechei; unclassified Providencia; Gayadomonas
joobiniege; Enterobacter; Paraneptunicella aestuarii; Mesocestoides corti; Bacteroides sp. SM1_62; Glaciecola
sp. XM2; Pectobacterium betavasculorum; Vibrio sonorensis; Candidatus Shapirobacteria bacterium;
Parabacteroides sp. AD58; Bacteroidetes bacterium HGW-Bacteroidetes-9; Opisthorchis felineus; Motilimonas
sp. E26; Cronobacter malonaticus; Serratia sp. M24T3; Mucilaginibacter polytrichastri; Chitinophagaceae
bacterium; Erwiniaceae bacterium PD-1; Candidatus Collierbacteria bacterium RIFOXYB1_FULL_49_13;
Cryomorphaceae bacterium; Schistosoma curassoni; Tolumonas sp.; Eubacteriaceae bacterium;
Amoeboaphelidium occidentale; Marinobacter nauticus; Coprobacter sp. LH1063; Bacteroides graminisolvens;
Vibrio hangzhouensis; Vibrio fluvialis; Wielerella bovis; Kingella kingae; Hafnia; Alteromonas sp. chi3;
Pontibacter litorisediminis; unclassified Sphingobacterium; Xenorhabdus sp. XENO-2; Salmonella enterica
subsp. enterica; Chloroflexota bacterium; Providencia rettgeri DSM 1131; Kangiella geojedonensis; Coemansia
interrupta; Candidatus Peregrinibacteria bacterium CG11_big_fil rev_8_21_14_0_20_46_8; Daejeonella sp.
JGW-45; Salmonella enterica subsp. enterica serovar Typhimurium; Candidatus Avibacteroides
excrementipullorum; Salmonella enterica subsp. enterica serovar Poona; Chryseolinea sp. H1M3-3; Martelella
alba; Rosenbergiella australiborealis; Schistosoma turkestanicum; Sodalis ligni; Trichobilharzia regenti; Pantoea
sp. 1.19; Idotea baltica; Enterobacter hormaechei subsp. steigerwaltii; Xenorhabdus beddingii; Hyphomicrobiales
bacterium; Agaribacter marinus; Xenorhabdus sp. XENO-10; Gilliamella sp. B2772; Dimargaris xerosporica;
Duffyella gerundensis; Cronobacter turicensis z3032; Xenorhabdus szentirmaii; Pasteurella testudinis; Leclercia;
Conchiformibius steedae; Paludibacter sp.; Sphingobacterium alkalisoli; Vitreoscilla massiliensis;
Ruminococcus sp.; Xenorhabdus miraniensis; Thalassotalea euphylliae; Fulvivirga aurantia; Lachnospiraceae
bacterium; Neisseriaceae bacterium; Vibrio metschnikovii; Capsulimonas corticalis; Hordeum vulgare;
Clostridium; Salinivirgaceae bacterium; Salmonella enterica subsp. enterica serovar Braenderup; Alcanivorax
sp.; Taenia asiatica; Psychroflexus planctonicus; Pedobacter sp. ASV12; Echinostoma caproni; Kordiimonas
lipolytica; Photobacterium; Parcubacteria group bacterium GW2011_GWA2_49_9; Pontibacter
beigongshangensis; Kosakonia quasisacchari; Parapedobacter indicus; Parapedobacter soli; Sphingobacterium
litopenaei; Alphaproteobacteria bacterium; Muricauda oceanensis; Linnemannia schmuckeri; Lacimicrobium
alkaliphilum; Muricauda abyssi; Pseudoalteromonas rubra; Buttiauxella agrestis; Alistipes sp.; Rosenbergiella
nectarea; Mucilaginibacter sp. 44-25; Anaerolineae bacterium; Rosenbergiella collisarenosi; Xenorhabdus
lircayensis; Vibrio tetraodonis; Enterobacteriaceae; Oceanisphaera profunda; Oceanobacillus sojae; Alteromonas
confluentis; Candidatus Uhrbacteria bacterium CG22_combo_CG10-13_8_21_14_all_47_17; Parasteatoda
tepidariorum; Arsenophonus endosymbiont of Aleurodicus floccissimus; Phycomyces blakesleeanus NRRL
1555(−); Candidatus Arsenophonus nilaparvatae; Dispira parvispora; Mortierella sp. 14UC; Fulvivirga sp. M361;
Mucilaginibacter achroorhodeus; Vibrio mediterranei; Alteromonas halophila; Zobellella maritima;
Psychrosphaera saromensis; Salmonella enterica subsp. enterica serovar Adjame; Weeksellaceae bacterium;
Sphingobacterium sp. FBM7-1; Clarias magur; Limosa lapponica baueri; Chlorobiota bacterium; Bacteroidetes bacterium
4572_117; Tatumella morbirosei; Sphingobacterium sp. BIGb0165; Enterobacter cloacae complex;
Myroides odoratus; Thermoanaerobacterales bacterium; Muricauda sp.; Candidatus Gracilibacteria bacterium;
Arsenophonus endosymbiont of Bemisia tabaci; Methylocystaceae bacterium; Altibacter sp. HG106; Muricauda;
Citrobacter koseri; Pseudohongiella sp.; Fulvivirga sediminis; Erwinia sp. ErVvl; Paramicrosporidium
saccamoebae; Phlyctochytrium bullatum; Arsenophonus sp. ENCA; Candidatus Dojkabacteria bacterium;
Pedobacter sp. HMF7647; Candidatus Nephrothrix sp. EaCA; Candidatus Azobacteroides sp.; Haplosporangium
sp. Z 11; Edwardsiella tarda ATCC 23685; Alcanivorax sp. VBW004; Pedobacter rhizosphaerae; Saprospiraceae
bacterium; Cuscuta campestris; Enterobacter asburiae; uncultured bacterium; Proteiniphilum propionicum;
SAR86 cluster bacterium; Xenorhabdus nematophila; Dimargaris cristalligena; Enterobacter roggenkampii;
Alteromonas aestuariivivens; Bacteroidetes bacterium GWC2_46_850; Enterovibrio paralichthyis; Pedobacter
sp.; Aliikangiella coralliicola; Vibrio thalassae; Edwardsiella tarda; Neisseria chenwenguii; Dissophora
globulifera; Pseudomonadales bacterium; Spongiibacter tropicus; Xenorhabdus vietnamensis; Ipomoea nil;
Aestuariibacter halophilus; Hyunsoonleella jejuensis; Candidatus Egerieicola faecale; Escherichia albertii;
Coemansia sp. RSA 2607; Pedobacter ghigonis; Mortierella hygrophila; Ruminococcus sp. CAG: 403;
Mortierella sp. GBA35; Candidatus Avibacteroides avistercoris; Oceanisphaera marina; Kingella kingae
PYKK081; Actinobacillus genomo sp. 2; Ipomoea triloba; Pedobacter helvus; Cellulophaga tyrosinoxydans;
Pseudoalteromonas sp. SiAl; Williamwhitmania taraxaci; Pseudoalteromonas sp. MMG005; Vibrio cholerae;
Candidatus Amoebophilus sp. 36-38; Chelonobacter oris; Methylosinus sp. PW1; Penaeicola halotolerans;
Nubsella zeaxanthinifaciens; Coemansia furcata; Alteromonas australica; Enterobacter cloacae; Plesiomonas;
Croceivirga lutea; Vibrio tubiashii; Methylosinus; Pelistega ratti; Arachidicoccus terrestris; Acidimicrobiales
bacterium; Candidatus Chazhemtobacterium aquaticus; Tolumonas osonensis; Sphingobacterium olei;
Marixanthomonas sp. SCSIO 43207; Alteromonas sediminis; Frischella perrara; Carboxylicivirga sp. M1479;
Alcanivorax sp. DG881; Tenacibaculum sp. KUL152; Schleiferiaceae bacterium; Sphingobacterium paludis;
Yersinia enterocolitica; Vibrio hepatarius; Lunatimonas lonarensis; Pseudoalteromonas phenolica; Pedobacter
namyangjuensis; Salvia hispanica; Candidatus Hoaglandella endobia; Tolypothrix sp. LEGE 11397;
Xenorhabdus sp. KK7.4; Fulvivirga sp. W9P-11; Cryptophyta sp. CCMP2293; Mortierella sp. GBA43;
Sphingobacteriales bacterium; Neisseria sp. WF04; Eubacterium sp.; Shewanella litorisediminis; Algicola
sagamiensis; Conchiformibius kuhniae; Gryganskiella cystojenkinii; Coemansia erecta; Jimgerdemannia
flammicorona; Pedobacter sp. JY14-1; Aeromonas jandaei; Glomus cerebriforme; Mortierella sp. GBAus27b;
Vibrio hannami; Xenorhabdus japonica; Eubacterium sp. MSJ-21; Yersinia canariae; Enterovibrio norvegicus;
unclassified Alteromonas; Frischella; Haemophilus paraphrohaemolyticus; Petromyzon marinus; Paraglaciecola
marina; Parapedobacter pyrenivorans; Coprobacter fastidiosus; Mannheimia haemolytica serotype A2 str.
OVINE; Muricauda nanhaiensis; Coemansia aciculifera; Nostoc carneum NIES-2107; unclassified Tolypothrix;
Grimontia sp. AD028; Vibrio metoecus; Solitalea longa; Enterobacterales bacterium CwR94; Alkaliflexus sp. Ai-
910; Pseudoalteromonas sp.; Vibrio salinus; Sphingobacterium sp. SRCM116780; Lacimicrobium sp. SS2-24;
Vitreoscilla sp.; Oceanimonas baumannii; Barnesiella intestinihominis; Coprobacter fastidiosus NSB1;
Sphingobacterium nematocida; Paralysiella testudinis; Edwardsiella hoshinae; Kiloniella laminariae; Dispira
simplex; Abyssalbus ytuae; Sphingobacterium faecium; Pseudoalteromonas tunicata; Aestuariibacter sp. AA17;
Pseudoalteromonas sp. MSK9-3; Myroides fluvii; Nissabacter sp. SGAir0207; Sodalis-like endosymbiont of
Proechinophthirus fluctus; Budvicia aquatica; Tatumella sp. JGM118; Deinococcus radiodurans; Yersinia;
Citrobacter amalonaticus; Candidatus Taylorbacteria bacterium CG11_big_fil_rev_8_21_14_0_20_46_11;
Alteromonas sp. MB-3u-76; Alcanivorax jadensis; Pedobacter sp. SYSU D00823; Corallincola spongiicola;
Zobellella taiwanensis; Citrobacter farmeri; Vibrio galatheae; Spirochaeta isovalerica; Entomohabitans
teleogrylli; Candidatus Vampirococcus lugosii; Ferrimonas marina; Alteromonas stellipolaris; Lentimicrobium
sp.; Brachymonas denitrificans; Oceanimonas sp. GK1; Aerococcus urinaehominis; Thalassobius sp.;
Pseudoalteromonas sp. MMG013; Aestuariibacter sp. A3R04; Kingella negevensis; Betaproteobacteria
bacterium; Sphingobacterium sp. UT-1RO-CII-1; Alcanivorax sediminis

TABLE D
NP_003353.1; XP_025257955.1; XP_012324546.1; NP_001233452.1; XP_019833435.1; KAF6278681.1;
XP_008568006.1; KAF6282970.1; XP_008519145.1; XP_007180156.1; XP_054450320.1; KAI5761723.1;
XP_032253882.1; KAF6083474.1; XP_043413769.1; XP_008705917.2; XP_020025570.1; XP_045313485.1;
XP_036132631.1; MBZ3881121.1; XP_006151552.1; XP_026265002.1; XP_025842431.1; XP_045441593.1;
XP_012586264.1; CAH6778753.1; XP_021561763.1; EPQ09857.1; XP_043413770.1; EHB01549.1;
XP_036028126.1; XP_013204567.1; KAB0394472.1; XP_012317382.1; NP_001013142.1; XP_048294183.1;
XP_020863574.1; XP_004615654.2; XP_021042647.1; XP_037054937.1; XP_049645350.1; XP_030357150.1;
XP_049662483.1; XP_029882455.1; XP_010218957.1; XP_038618614.1; 1AKZ_A; XP_025970451.1;
XP_014795505.1; EGW10622.1; XP_051489843.1; XP_009558788.2; NXL28197.1; XP_025916061.1;
XP_054075986.1; XP_054700828.1; XP_037231584.1; XP_009908009.2; NP_001385002.1; XP_048677469.1;
XP_032054857.1; XP_050779367.1; TFK14052.1; XP_047929815.1; XP_032661323.1; XP_038007989.1;
KAG6932998.1; XP_042661095.1; XP_012418872.1; XP_006004617.1; XP_014349118.1; XP_005055092.1;
XP_039580799.1; XP_032932116.1; XP_015733553.1; XP_030814846.1; XP_033900131.2; MBN3316704.1;
XP_015499054.1; XP_014730339.1; XP_036249012.1; XP_054145031.1; XP_005524365.2; TRZ23161.1;
XP_030075104.1; XP_023793175.1; XP_054501993.1; XP_054852520.1; KAJ7400692.1; XP_015268730.1;
XP_034758288.1; XP_043924453.1; XP_040432692.1; XP_041266048.1; OXB84875.1; XP_036385765.1;
XP_053325038.1; XP_053325046.1; XP_042193637.1; OXB62154.1; XP_053106732.1; KAG7476728.1;
XP_029475192.1; XP_007441965.1; XP_018524161.1; KAF3695811.1; XP_026178605.1; XP_030593745.1;
XP_043882215.1; XP_013918577.1; XP_051888209.1; XP_043882217.1; XP_025752763.1; KAG8596774.1;
XP_029961633.1; XP_029999776.1; XP_036453844.1; XP_029915939.1; XP_030633255.1; XP_029999778.1;
XP_039218712.1; XP_005722242.1; XP_026575165.1; XP_053218300.1; XP_026541263.1; XP_045890231.1;
XP_033476382.1; XP_039875270.1; XP_015671272.1; XP_041792914.1; XP_028559693.1; XP_009634426.1;
XP_053136061.1; XP_047447921.1; XP_037337874.1; XP_029365747.1; XP_023269423.1; XP_035999568.1;
XP_038145998.1; XP_040052629.1; XP_022054340.2; XP_050964876.1; XP_035592121.1; XP_018416220.1;
XP_048370288.1; XP_053181662.1; XP_023154760.2; XP_041643456.1; XP_022609857.1; XP_017571742.1;
XP_054904926.1; XP_026101978.1; XP_034026151.1; ETE71619.1; XP_028270487.1; XP_020345710.1;
XP_007559211.1; XP_020345712.1; XP_037633953.1; XP_013869817.1; KAF7200236.1; XP_044774876.1;
XP_039659493.1; KAI7812448.1; XP_049618483.1; XP_049435071.1; XP_030274272_1; XP_048098547.1;
KAE8292138.1; XP_037115676.1; XP_043094960.1; XP_026865102.2; XP_029618954.1; KAG9335062.1;
XP_053084875.1; KTG29157.1; XP_043968828.1; XP_017269481.1; XP_055080100.1; KAG8455709.1;
XP_037115675.1; XP_022522613.2; XP_049326706.1; CAG08512.1; XP_014016647.2; XP_035245762.1;
KAA8592263.1; XP_035245763.1; XP_035245764.1; XP_017211537.1; XP_041854545.1; XP_040898663.1;
XP_012992167.1; XP_046698403.1; XP_051923749.1; XP_035513527.1; XP_019718171.1; XP_028851732.1;
XP_053726582.1; XP_031426990.1; XP_031426992.1; XP_051528997.1; XP_027001453.1; XP_026198106.1;
XP_055056845.1; XP_014324249.1; KAF6282972.1; ROL44366.1; KAJ8261043.1; KAI1882410.1;
XP_053336421.1; XP_020561603.1; KAF6282971.1; XP_008416899.1; KAG1957564.1; XP_017306682.1;
CAB85707.1; XP_039517667.1; KAG9487077.1; XP_017931661.1; XP_033050470.1; XP_038945446.1;
XP_037369148.1; XP_030214035.1; KAF3826792.1; TKS78741.1; XP_042297313.1; XP_035494966.1;
AWP07077.1; KAJ8393919.1; KAJ8286496.1; CAB1414260.1; XP_051278649.1; XP_052412495.1;
XP_006015208.1; XP_051279128.1; XP_009878677.1; KAE8634233.1; KPP58841.1; AAI67353.1;
KAF3850847.1; XP_028830576.1; XP_029295177.1; KAJ1092919.1; KAG7274710.1; KAA0716254.1;
KAE8634231.1; XP_049435079.1; VFV38557.1; KAJ3603927.1; XP_034004458.1; PIK35447.1;
XP_033946598.1; KAJ8025392.1; XP_035672614.1; TMS03883.1; ELK31458.1; XP_052411871.1;
TWW60398.1; XP_028270375.1; EMP37160.1; 1OKB_A; XP_048043823.1; XP_025094879.1;
XP_016333700.1; XP_041916443.1; KAG5272533.1; XP_051749272.1; XP_030643372.1; XP_051521793.1;
KPP60507.1; XP_023664027.1; XP_051980804.1; KAJ8317720.1; KAJ8263653.1; XP_036418809.1;
XP_051966998.1; XP_022091813.1; XP_791566.1; XP_039514821.1; XP_038070739.1; XP_054765443.1;
XP_026875850.2; CAC5425936.1; XP_022337906.1; XP_017576651.1; KAI0242145.1; XP_047447936.1;
XP_052102150.1; XP_017576650.1; CAD7673851.1; XP_023236003.1; XP_033633272.1; XP_023236002.1;
XP_011415861.2; OWF48535.1; XP_046580924.1; XP_042565360.1; XP_021357381.1; VDI37192.1;
KAA0717115.1; VDI37191.1; XP_046358519.1; XP_033476387.1; XP_048744576.1; ELU04167.1;
XP_048744575.1; KAH3788991.1; XP_052226450.1; XP_010156854.1; KAG7314619.1; XP_030273654.1;
KAF4073137.1; XP_050399737.1; XP_014392330.1; XP_052773941.1; KAJ7392381.1; XP_026778218.3;
XP_031573460.1; XP_041370936.1; XP_017350494.1; KAE8634237.1; GFO16427.1; XP_027043215.1;
XP_028391306.1; XP_020626946.1; CAG5134945.1; XP_027023798.1; XP_045189630.2; XP_039266489.1;
XP_048961937.1; XP_053407280.1; XP_001623190.1; XP_053341975.1; XP_051278652.1; XP_046695045.1;
XP_022791963.1; KA15089390.1; XP_009282221.1; RXG73420.1; XP_029212906.2; CAE1280941.1;
KAH9499176.1; RDD40527.1; XP_030271978.1; XP_053634004.1; XP_009995145.1; XP_033119413.1;
XP_053473551.1; CAD5113127.1; XP_037040401.1; XP_053207671.1; XP_014128266.1; XP_015782855.1;
CAF2644705.1; XP_037040393.1; XP_023714286.1; CAB3267517.1; XP_027231417.1; XP_009051091.1;
EPY87351.1; CAF1292700.1; RUS84099.1; KAG8232402.1; XP_037792370.1; XP_042872292.1;
CAD7275595.1; XP_013779562.1; XP_046695046.1; KAI1233072.1; XP_014786953.2; CAF0817543.1;
XP_021955428.1; RWS17505.1; XP_054168754.1; CAF3819016.1; CAF0866487.1; CAD7404467.1;
KAI8789608.1; XP_047491169.1; KAG0724137.1; XP_046383260.1; XP_046383259.1; XP_009935572.1;
XP_014256502.1; CAF3790558.1; XP_012937409.2; KAI6652833.1; XP_018667776.1; XP_008274838.1;
XP_046450308.1; XP_003383944.1; XP_037090985.1; XP_002422608.1; XP_018013911.1; XP_021357379.1;
XP_042233188.1; XP_051551785.1; XP_037504447.1; KAJ1524847.1; XP_037282647.1; XP_014674769.1;
CAF1012939.1; XP_034254795.1; XP_034254793.1; KAJ6642084.1; XP_037092226.1; XP_034254794.1;
CAF4607548.1; XP_055335137.1; XP_023320045.1; XP_046667956.1; XP_046994588.1; KDR19394.1;
XP_043218828.1; KAE8750889.1; XP_026274135.1; XP_043218827.1; XP_055335136.1; CAD7656650.1;
EEC05554.1; XP_043218825.1; EFX87884.1; XP_055335134.1; CAG7718725.1; CAF1065651.1;
GFS17326.1; XP_050049036.1; CAH0111913.1; RWS01965.1; XP_045615322.1; KAF6019275.1;
KZS12459.1; CAD7223519.1; CAD7246532.1; KAG1681804.1; KAH7979219.1; XP_032791021.2;
KAG0423189.1; CAF3006108.1; XP_014293985.1; XP_009020242.1; KAF7490789.1; CAH1404326.1;
XP_054258788.1; KAI9144391.1; XP_002166406.1; XP_036367980.1; CAF1487692.1; CAD7426261.1;
CAD7442272.1; RWS30851.1; CAD7458856.1; CAF1360805.1; OWA52854.1; RZF36898.1; KAH9398646.1;
TNN69626.1; XP_033757672.1; XP_033757673.1; KAG8194460.1; CAF0875924.1; CAF0956906.1;
CAF0777421.1; GBL91917.1; GAU97779.1; KAJ6222188.1; GIY15625.1; GIY75527.1; XP_046911632.1;
XP_040569398.1; KAH9526884.1; WP_040266633.1; ORX97897.1; XP_054709718.1; KAI1293872.1;
WP_130831072.1; XP_001750874.1; WP_243077377.1; WP_127958881.1; MCB0333308.1; KAG8311591.1;
VZI09842.1; PIR03886.1; MBN7824149.1; WP_036281950.1; CAD7700536.1; KAF7257074.1;
MBQ9354996.1; MAT89555.1; WP_071473194.1; XP_027199873.1; PLW92214.1; KAH9416774.1;
WP_009583033.1; MBR9858980.1; WP_113865980.1; WP_023399579.1; WP_150012655.1;
WP_179399600.1; WP_007832010.1; WP_048637820.1; WP_264091338.1; MBC7124566.1; KAF8561016.1;
WP_246760648.1; OFX39024.1; AXN90995.1; TVR70968.1; XP_030893574.1; WP_155176914.1;
XP_012505674.1; WP_024913202.1; WP_116729449.1; CAF1243154.1; NIG87455.1; WP_078004781.1;
XP_002114621.1; WP_125289464.1; CAF1632393.1; WP_110268112.1; WP_077649195.1; GLX92274.1;
WP_112288176.1; WP_202859020.1; VDM00162.1; WP_085070642.1; WP_004959514.1; WP_072000520.1;
WP_065823907.1; WP_250650628.1; WP_105167576.1; MCG8384196.1; WP_131840474.1;
XP_035211784.1; AXR07529.1; WP_099704407.1; WP_218023582.1; MBP7011416.1; WP_086271185.1;
WP_227965204.1; CAH8563417.1; VZI46287.1; WP_133578303.1; XP_051066893.1; WP_008484185.1;
WP_123800528.1; KAI9598613.1; NUM51057.1; MBN1951233.1; WP_176031951.1; WP_070983832.1;
CAH8543791.1; VZI34105.1; CAH8575158.1; KAJ1930645.1; MCM1109302.1; MBR5925838.1;
WP_137268796.1; WP_194716009.1; AWB67224.1; WP_131867411.1; WP_199915565.1; KA19227478.1;
WP_103060116.1; MBQ2970418.1; MBQ5403003.1; MBX9852622.1; MCK9351643.1; WP_075608613.1;
WP_011411621.1; WP_007620804.1; NLM92084.1; MBN1115668.1; MCQ2299830.1; WP_249891944.1;
MBR0206726.1; CAB3373753.1; CAH8553116.1; WP_166251580.1; CAH8582665.1; NVN94911.1;
MCG8579428.1; MCQ2607600.1; HFC10897.1; PSN50218.1; KAI9302945.1; WP_160622004.1;
WP_104486975.1; HCT71644.1; MCB0319460.1; KAJ2521671.1; HHG84670.1; EHD20518.1; GAP42774.1;
OQC62180.1; HHT03814.1; MBI5010178.1; WP_062042772.1; NLK14938.1; MBP8759857.1;
MBR4793684.1; CAI2729787.1; MCB0513998.1; MBR1991917.1; MBI2121061.1; KAJ1984314.1;
WP_160412647.1; KXS13698.1; MBE6292364.1; WP_239552568.1; WP_047452623.1; GFY76167.1;
WP_120506492.1; PIR55535.1; WP_216030393.1; WP_028972952.1; MBK7712075.1; MBP1666195.1;
HIX55325.1; WP_268409474.1; PPD43686.1; WP_021184395.1; TQV74132.1; WP_178243469.1;
WP_123478339.1; WP_026050912.1; MBI5004288.1; WP_084970425.1; MBI4407882.1; MBO5182505.1;
MBR5706584.1; NLX73325.1; WP_202087260.1; WP_065633188.1; OFX56790.1; MBU3854095.1;
QKG79816.1; HCW07910.1; MCU0363456.1; WP_246075824.1; WP_220429263.1; MBO7651724.1;
NND78178.1; WP_222576485.1; WP_139654964.1; MBR6857693.1; WP_118526140.1; KAF9922663.1;
TDF39389.1; WP_007664251.1; MBR1850355.1; WP_036771354.1; WP_233070777.1; MBR4593195.1;
MCB0838066.1; WP_118985419.1; ADD78071.1; WP_150437547.1; MBW6498825.1; WP_008617080.1;
HCZ20549.1; MBQ7162918.1; MBQ8601462.1; WP_013510135.1; MBR4267829.1; KFM57753.1;
MBI5754440.1; ORZ01472.1; CAH8564062.1; MBS7388824.1; MBQ6046081.1; MBQ2932503.1;
WP_158972051.1; WP_159580226.1; CAH8857441.1; WP_012906702.1; WP_217961472.1; MBQ1973330.1;
MBP7354681.1; WP_150092576.1; WP_215930543.1; TRY68203.1; ABU75959.1; MBR4648331.1;
MCC8172223.1; MBS4014550.1; MBH0368537.1; WP_133815354.1; MBL1179855.1; HDP75905.1;
MBV6460980.1; MCU0367876.1; WP_079686082.1; OFY35198.1; WP_039611678.1; WP_024993669.1;
HBS85072.1; MBR2359783.1; MBF0433309.1; MBO4615209.1; WP_008512622.1; WP_205543179.1;
WP_065619434.1; WP_026741932.1; MBL7904829.1; WP_096778798.1; MCB9527277.1; NVO10887.1;
WP_065791436.1; MCQ2454655.1; WP_005596359.1; WP_142969910.1; AAW26641.1; WP_182921897.1;
MBX2945013.1; WP_102797971.1; WP_136250641.1; WP_257050785.1; WP_101198850.1; VUZ47170.1;
MBQ1677779.1; MBI3020110.1; WP_049582203.1; MBR4136503.1; WP_105546465.1; MBL8995049.1;
XP_018651621.1; WP_261625640.1; WP_007636337.1; MCQ2195342.1; WP_095924877.1; MBE6319069.1;
CAI8520916.1; WP_119908881.1; WP_161875571.1; WP_022607443.1; WP_034916432.1; WP_169401733.1;
WP_198227668.1; WP_225697966.1; XP_005837120.1; WP_104659011.1; MBP5562880.1;
WP_068892343.1; WP_101824545.1; WP_102118563.1; WP_275554681.1; KAF6777280.1;
WP_273748036.1; WP_250341255.1; HET98629.1; HCN6464129.1; WP_264646027.1; WP_072307975.1;
GFU60467.1; MCB9002615.1; VDM23160.1; RPH31447.1; WP_024553700.1; MBE6729077.1;
MCK9339391.1; MCD6023714.1; WP_243347719.1; WP_022943432.1; WP_063360190.1; MBR5550920.1;
WP_208293523.1; MCK5765138.1; MCC6836131.1; WP_120065350.1; QIQ22360.1; WP_115459111.1;
MBE6206693.1; WP_246612340.1; GAD00442.1; MBQ1818990.1; WP_105760851.1; WP_171587856.1;
CAH8581633.1; MCR5115299.1; WP_025246321.1; HIZ00677.1; MBE6192170.1; HAZ0553373.1;
MBR3466266.1; MBN2776065.1; MBQ4358399.1; WP_272686881.1; NWJ50104.1; MBP8992343.1;
MBQ8704542.1; WP_017444697.1; WP_221256348.1; WP_232361432.1; VDD76547.1; MBQ7551231.1;
KPL26064.1; MBX2894361.1; MBT1450433.1; WP_039308775.1; MBP3355555.1; WP_070962620.1;
HAP37873.1; WP_251968398.1; PKP04434.1; MBO7291086.1; TGZ50325.1; MBQ5693790.1;
WP_232801838.1; AHB69352.1; OCG00511.1; HAP02286.1; MCK9449265.1; WP_009636993.1;
MBE7177176.1; NLK57171.1; MBK6481961.1; MBR7036108.1; QKJ88270.1; OGD79472.1; TVR37558.1;
VDP26909.1; MBI9034890.1; TXH62196.1; MBP5496102.1; NLL50234.1; KAI3660180.1; MBY6188071.1;
WP_255028056.1; WP_276964512.1; WP_103879072.1; MCB0329209.1; WP_020330528.1;
WP_249445255.1; WP_042925520.1; MBX2966960.1; HHU01422.1; WP_004847207.1; WP_273642418.1;
WP_276495661.1; WP_132840859.1; WP_273575765.1; EDH3763289.1; HAF9718979.1; NVO20388.1;
RPI87749.1; MCB9425793.1; EAB2941589.1; EFE51614.1; EEC4424362.1; WP_046560614.1;
KAJ2778374.1; PIQ78615.1; WP_276347815.1; HAG15903.1; EFN6439465.1; MBK9390162.1;
MBN1650000.1; ECD6919710.1; HIS11334.1; EDL3262951.1; WP_276374741.1; TKI06787.1;
WP_214215487.1; CAH8512643.1; MBO7646712.1; WP_255306632.1; MBO7114203.1; WP_132926535.1;
CAH8853344.1; WP_075181544.1; MBK7213598.1; MCL4106240.1; HAV1779196.1; MBQ7156796.1;
WP_086113194.1; RTL86488.1; GLR71119.1; WP_273555027.1; WP_267362580.1; KAJ1982966.1;
WP_264290222.1; CBA32947.1; WP_038234641.1; MBQ7960014.1; WP_084256734.1; WP_103180764.1;
RRD91756.1; MTK54187.1; WP_260426471.1; QQS49803.1; WP_136818882.1; MCQ2067030.1;
MBN1183319.1; WP_058355889.1; MCM7068047.1; MBQ8965257.1; PHM49157.1; MCR4659940.1;
WP_116016610.1; WP_155208973.1; MBR1909468.1; MBR0128897.1; WP_186460281.1; MBQ8672484.1;
BDI32833.1; MBR2370066.1; KAI5009760.1; MBT3210224.1; WP_264273433.1; WP_021282931.1;
MBR3566769.1; MBX2901564.1; EIF4093194.1; MCK5884912.1; VDK34832.1; WP_188457976.1;
WP_199141476.1; VDP86594.1; WP_068143708.1; TXI82319.1; WP_116009452.1; MBO7218161.1;
MBP1630061.1; WP_047886455.1; KKW11088.1; NJO68391.1; WP_065605995.1; MBS1580760.1;
WP_187261354.1; WP_131406420.1; MBX2866843.1; WP_090630633.1; MCF6240022.1; WP_262248827.1;
XP_042219577.1; WP_190302238.1; RLD33520.1; MBQ9386169.1; MBQ2845340.1; WP_127136826.1;
KAF9156677.1; WP_062482259.1; MCQ2059633.1; WP_220112060.1; WP_138536285.1; WP_034497756.1;
MBQ3203829.1; WP_092676376.1; OJW15945.1; MAY56093.1; MCC8087798.1; WP_232760911.1;
WP_241580005.1; MCE1255089.1; WP_241649894.1; WP_198690727.1; WP_126490760.1;
WP_114785161.1; PLX01096.1; WP_064327035.1; WP_087034888.1; WP_126542587.1; WP_077603218.1;
WP_070124919.1; PIP60430.1; XP_015918274.2; MBK7028924.1; WP_119711586.1; MCA0427806.1;
XP_018297667.1; WP_032114775.1; KAJ1970278.1; KAF9131340.1; MCI7378199.1; MBO5251260.1;
WP_143396601.1; WP_146270382.1; MCD7710613.1; WP_171388963.1; WP_189408440.1; MBO6052036.1;
WP_198650017.1; MBS1490747.1; HCM60568.1; MCD4769635.1; MCC7432625.1; WP_105052029.1;
QIL32354.1; MBS7333984.1; WP_227694811.1; MBO7124794.1; KAF5909737.1; PKU27378.1;
NOZ33722.1; OQY03052.1; WP_156994587.1; MBD5259243.1; WP_259179584.1; EEV5343293.1;
WP_113656144.1; WP_259188960.1; MBO2504004.1; WP_001587718.1; MBR4996914.1; MBA4743944.1;
MCF7918025.1; WP_181995156.1; MBP5516944.1; NDB68460.1; WP_272851504.1; MBK9275770.1;
WP_108247311.1; WP_130028873.1; GGW97046.1; MAO39119.1; WP_202245375.1; WP_067709548.1;
PJF19399.1; KAJ3113054.1; PAU99985.1; MBR0277069.1; MCQ2068603.1; MBN1331382.1; MBI5412796.1;
WP_160843392.1; MBK9730142.1; PGH39937.1; MCD7971468.1; KAF9186020.1; EFE21742.1;
WP_155135796.1; WP_090883793.1; MBR2909646.1; MCF8296759.1; MCP4553383.1; VFR02280.1;
WP_128334074.1; EKD78244.1; WP_238866705.1; MBC7485728.1; PCI73998.1; WP_010847127.1;
KAJ1985444.1; WP_063436206.1; WP_115591321.1; OFX54396.1; WP_218359953.1; MCQ2297475.1;
RZK79769.1; WP_142892023.1; WP_096991971.1; MBR7067363.1; WP_005295143.1; WP_089035279.1;
KAG0324687.1; SNX45224.1; MBL7112839.1; NKB97478.1; MBL7832686.1; MAD50889.1;
MBO5850239.1; WP_273035260.1; MCK9163787.1; WP_086110363.1; MCR5445586.1; XP_019191463.1;
MCC2618316.1; WP_092578126.1; HIU42629.1; WP_000013025.1; MBT4917289.1; KAJ2369306.1;
MCQ2296240.1; WP_175635165.1; KAF9547725.1; NLJ42969.1; CDE29854.1; KAF9093591.1;
MBL4569887.1; HJD52991.1; HHN48818.1; WP_188630758.1; EIC14122.1; NCC18464.1; MBE6895684.1;
WP_279467767.1; XP_031113714.1; WP_138723959.1; MBQ8761904.1; WP_084061733.1;
WP_215521820.1; SDC89909.1; TVQ12186.1; WP_211056722.1; GHW33873.1; OJW70312.1; VAX76628.1;
WP_034612142.1; WP_170830131.1; WP_036290088.1; MBR0304222.1; WP_226390191.1; RMG25810.1;
MBO4664065.1; WP_113635609.1; KAJ2813928.1; WP_273345950.1; TAT60797.1; WP_047706807.1;
HBN6663560.1; WP_185781396.1; MBP3785266.1; KGY13658.1; MCL4153352.1; WP_182381198.1;
MCR5423556.1; KAA3674195.1; WP_142862486.1; WP_204319023.1; MBO4879456.1; MCK9254486.1;
MCB0380068.1; WP_224070408.1; EJE3067579.1; MBQ23949.1; MCH1434645.1; NYV47805.1;
WP_161931486.1; WP_188027068.1; WP_136901765.1; WP_136678827.1; WP_223107938.1;
MBR3481865.1; WP_124025906.1; WP_276920876.1; MCR4769273.1; WP_143299822.1; MBL4572480.1;
WP_007148651.1; GFD89982.1; MBM3261264.1; MCT4622982.1; WP_133639680.1; WP_011815748.1;
EGQ7689116.1; WP_215986863.1; WP_010853320.1; WP_058030875.1; PHR63635.1; WP_113653357.1;
XP_047958596.1; WP_067568225.1; WP_228045390.1; PHM55109.1; MBT3249917.1; WP_099121899.1;
WP_233769960.1; KAJ1488299.1; MCB9361723.1; NKB31903.1; KAG0227641.1; NLJ08503.1;
MBL7849091.1; WP_256359879.1; MBP3762600.1; MBO5609773.1; MBE6591352.1; WP_203326542.1;
WP_018692098.1; WP_027008854.1; KAG0043183.1; MCR5312370.1; MBL0911018.1; KAJ2828623.1;
RUS32940.1; MBO4489242.1; WP_276088662.1; WP_279011647.1; RIA90421.1; KAI8359770.1;
WP_063706231.1; MSU73703.1; WP_277829033.1; MCQ2052124.1; WP_092519196.1; WP_216507992.1;
NOR88129.1; WP_145555029.1; WP_017009141.1; WP_202262291.1; MBO4397784.1; WP_187756255.1;
WP_005707994.1; MBP6429015.1; XP_032836495.1; WP_133468977.1; WP_188504627.1;
WP_270214247.1; MBP3641290.1; MBR4229243.1; EEY10547.1; MBK7627708.1; WP_116183379.1;
GIV23872.1; KAJ2889648.1; BAY35093.1; WP_045867628.1; MBR6292195.1; MBD5422363.1;
WP_046303907.1; MBO5133665.1; WP_154171419.1; WP_103790025.1; MCB9335128.1; PKH21878.1;
WP_250723494.1; MCK4465198.1; MBI4932163.1; PHQ94467.1; MBO7338387.1; NBT22103.1;
MBN2813482.1; WP_232766276.1; WP_234510546.1; MBK8846866.1; WP_088329923.1; MBP6552766.1;
WP_094277933.1; WP_239825180.1; WP_008862791.1; ERM88588.1; WP_079643827.1; MAC69138.1;
WP_268866648.1; QRQ81581.1; MZP54827.1; WP_024523801.1; WP_269421438.1; KAJ1650250.1;
WP_255842908.1; WP_045753405.1; RMF46977.1; ATC95626.1; WP_263710709.1; MCG8394556.1;
WP_119861689.1; WP_158962987.1; WP_137762360.1; WP_066597315.1; XP_033730346.1;
WP_029096626.1; WP_212713231.1; RUO96743.1; MBR6371513.1; HBC0991878.1; MBQ2023477.1;
MBK9356324.1; MCQ2269884.1; 4UQM_A; WP_038635570.1; WP_040643665.1; BCU49725.1;
PIQ68867.1; WP_100972814.1; WP_272975390.1; MBL4582214.1; WP_207532173.1; WP_130566453.1;
WP_106452514.1; GJL45960.1; MBS1557639.1; MBQ2284493.1; WP_045956387.1; WP_184746510.1;
MBR2428468.1; RZK75151.1; WP_058913188.1; MBS8121423.1; WP_067664232.1; WP_057789864.1;
NTW25238.1; MBR7170563.1; MCF8378520.1; WP_200785760.1; MCQ2272347.1; WP_014293006.1;
WP_067980281.1; MBT29516.1; WP_210995654.1; WP_216025025.1; WP_032137494.1; MCE9550767.1;
WP_268231227.1; WP_153500961.1

1. A fusion protein comprising:
(1) a nucleic acid programmable DNA binding domain (napDNAbd) capable of binding a target dsDNA comprising:
(a) a first deoxyribonucleotide (e.g., dG (deoxyguanosine), dT (thymidine), dC (deoxycytidine)) in a protospacer sequence on a nontarget strand (edited strand) of the target dsDNA, and
(b) a second deoxyribonucleotide (e.g., dC (deoxycytidine), dA (deoxyadenosine), dG (deoxyguanosine)) base pairing with the first deoxyribonucleotide (e.g., dG, dT, dC) and in a target sequence on the target strand (non-edited strand) of the target dsDNA, wherein the protospacer sequence is fully reverse complementary to the target sequence; and
(2) a base excising domain capable of excising the base (e.g., guanine, thymine, cytosine) of the first deoxyribonucleotide;
wherein the fusion protein does not comprise a deaminase domain, e.g., an adenine or cytosine deaminase domain, e.g., TadA and variants thereof; and
wherein the first deoxyribonucleotide is deoxyguanosine (dG), thymidine (dT), or deoxycytidine (dC).

2. The fusion protein of any preceding claim, wherein the conversion of the first deoxyribonucleotide to the fourth deoxyribonucleotide is dG-to-dA, dG-to-dT, dG-to-dC, dT-to-dA, dT-to-dC, dT-to-dG, dC-to-dA, dC-to-dT, or dC-to-dG.

3. The fusion protein of any preceding claim, wherein the base excising domain comprises a glycosylase.

4. The fusion protein of any preceding claim, wherein the glycosylase is selected from the group consisting of N-methylpurine DNA glycosylase (MPG), 8-oxoguanine DNA glycosylase (OGG1), methyl-CpG binding domain 4, DNA glycosylase (MBD4), thymine DNA glycosylase (TDG), uracil DNA glycosylase (UNG), single-strand-selective monofunctional uracil-DNA glycosylase 1 (SMUG1), mutY DNA glycosylase (MUTYH), nth like DNA glycosylase 1 (NTHL1), nei like DNA glycosylase 1 (NEIL1), nei like DNA glycosylase 2 (NEIL2), nei like DNA glycosylase 3 (NEIL3), and mutants thereof capable of recognizing and excising a base from a nucleotide of a nucleic acid.

5. The fusion protein of any preceding claim, wherein the base excising domain comprises an N-methylpurine DNA glycosylase (MPG).

6. The fusion protein of any preceding claim, wherein the MPG comprises an amino acid substitution relative to a reference MPG of SEQ ID NO: 7 at a position that is corresponding to a position or that is a position selected from the group consisting of N169, D175, C178, and/or Q294 of the reference MPG, wherein the position is numbered according to SEQ ID NO: 1.

7. The fusion protein of any preceding claim, wherein the amino acid substitution is a substitution with R, A, N, or G.

8. The fusion protein of any preceding claim, wherein the MPG comprises an amino acid substitution relative to a reference MPG of SEQ ID NO: 7 that is corresponding to a substitution or that is a substitution selected from the group consisting of N169G, D175R, C178N, Q294R, and a combination of any two or more substitutions thereof, wherein the position is numbered according to SEQ ID NO: 1.

9. The fusion protein of any preceding claim, wherein the MPG comprises a combination substitution relative to a reference MPG of SEQ ID NO: 7 that is corresponding to a combination substitution of N169G, D175R, C178N, and Q294R, wherein the position is numbered according to SEQ ID NO: 1.

10. The fusion protein of any preceding claim, wherein the MPG comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 8, 16, 18, 20, 22, 24, 26, 28, 30, 32, 34, 36, or 38.

11. The fusion protein of any preceding claim, wherein the MPG is (substantially) capable of excising guanine of dG.

12. The fusion protein of any preceding claim, wherein the base excising domain comprises an uracil-DNA glycosylase (UNG).

13. The fusion protein of any preceding claim, wherein the UNG comprises an amino acid substitution relative to a reference UNG of SEQ ID NO: 135 or 137 at a position that is corresponding to a position or that is a position selected from the group consisting of K184, A214, Q259, and/or Y284 of the reference UNG, wherein the position is numbered according to SEQ ID NO: 133.

14. The fusion protein of any preceding claim, wherein the amino acid substitution is a substitution with A, D, V, or T.

15. The fusion protein of any preceding claim, wherein the UNG comprises an amino acid substitution relative to a reference UNG of SEQ ID NO: 135 or 137 that is corresponding to a substitution or that is a substitution selected from the group consisting of K184A, A214V, A214T, Q259A, Y284D, and a combination of any two or more substitutions thereof, wherein the position is numbered according to SEQ ID NO: 133.

16. The fusion protein of any preceding claim, wherein the UNG comprises a deletion of amino acids at positions that are corresponding to positions or that are positions 1-65, 1-66, 1-67, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of the reference UNG of SEQ ID NO: 135 or 137, wherein the position is numbered according to SEQ ID NO: 133.

17. The fusion protein of any preceding claim, wherein the UNG comprises an amino acid substitution relative to a reference UNG of SEQ ID NO: 135 that is corresponding to a substitution or that is a substitution selected from the group consisting of A214T, Q259A, Y284D, and a combination of any two or more substitutions thereof, and comprises a deletion of amino acids at positions that are corresponding to positions or that are positions 1-88 of the reference UNG, wherein the position is numbered according to SEQ ID NO: 133.

18. The fusion protein of any preceding claim, wherein the UNG comprises an amino acid substitution relative to a reference UNG of SEQ ID NO: 137 that is corresponding to a substitution or that is a substitution selected from the group consisting of K184A, A214V, and a combination of the two substitutions, and comprises a deletion of amino acids at positions that are corresponding to positions or that are positions 1-88 of the reference UNG, wherein the position is numbered according to SEQ ID NO: 133.

19. The fusion protein of any preceding claim, wherein the UNG comprising said amino acid mutation comprises, consists essentially of, or consists of an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 56, 58, 60, 62, 135, 137, 139, 141, 144, 146, 148, 150, 152, 155, 157, and 159 or an N-terminal truncation thereof lacking the most N-terminal Methionine (M) (coded by start codon ATG).

20. The fusion protein of any preceding claim, wherein the UNG is (substantially) capable of excising thymine of dT.

21. The fusion protein of any preceding claim, wherein the UNG is (substantially) capable of excising cytosine of dC.

22. The fusion protein of any preceding claim, wherein the napDNAbd is RNA programmable DNA binding protein.

23. The fusion protein of any preceding claim, wherein the napDNAbd is selected from the group consisting of CRISPR-associated (Cas) protein, IscB, IsrB, Argonaute, and TnpB.

24. The fusion protein of any preceding claim, wherein the napDNAbd is a nickase, e.g., a Cas9 nickase, an IscB nickase.

25. The fusion protein of any preceding claim, wherein the napDNAbd is nuclease-inactive, e.g., dead Cas9, dead Cas12i.

26. The fusion protein of any preceding claim, wherein the napDNAbd comprise an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 2, 48, 50, 52, or 163.

27. The fusion protein of any preceding claim, wherein the fusion protein comprises, from N-terminal to C-terminal, (1) the napDNAbp and the base excising domain; or (2) the base excising domain and the napDNAbp.

28. The fusion protein of any preceding claim, wherein the napDNAbd (e.g., Cas9) is a two-part napDNAbd, for example, a two-part split Cas9, comprising a N-terminal portion and a C-terminal portion, and wherein the fusion protein comprises, from N-terminal to C-terminal, (1) the N-terminal portion of the napDNAbd, the base excising domain, and the C-terminal portion of the napDNAbd; (2) the C-terminal portion of the napDNAbd, the base excising domain, and the N-terminal portion of the napDNAbd; or (3) the base excising domain, the C-terminal portion of the napDNAbd (e.g., amino acids at positions 1249-1368), and the N-terminal portion (e.g., amino acids at positions 1-1248) of the napDNAbd.

29. The fusion protein of any preceding claim, wherein the napDNAbd is SpCas9 (e.g., a SpCas9 nickase) or a mutant thereof (e.g., a SpG Cas9 nickase).

30. The fusion protein of any preceding claim, wherein the N-terminal portion of the napDNAbd is the amino acids of the napDNAbp at positions 1 or 2 to 1012, 1028, 1041, 1046, 1047, 1248, 1249, or 1300.

31. The fusion protein of any preceding claim, wherein the C-terminal portion of the napDNAbd is the amino acids of the napDNAbp at positions 1013, 1029, 1042, 1047, 1048, 1249, 1063, 1064, 1230, 1249, or 1301 to 1368.

32. The fusion protein of any preceding claim, wherein the fusion protein comprises the base excising domain embedded between positions 2-1248 of nCas9 (SEQ ID NO: 2) and positions 1249-1368 of nCas9 (SEQ ID NO: 2), wherein the first amino acid residue D of nCas9 (SEQ ID NO: 2) was designated as position 2; or embedded between positions 2-1047 of nCas9 (SEQ ID NO: 2) and positions 1064-1368 of nCas9 (SEQ ID NO: 2), wherein the first amino acid residue D of nCas9 (SEQ ID NO: 2) was designated as position 2.

33. The fusion protein of any preceding claim, wherein the fusion protein comprises an amino acid sequence having a sequence identity of at least about 60% (e.g., at least about 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 12, 14, 15, 17, 19, 21, 23, 25, 27, 29, 31, 33, 35, 37, 39, 55, 57, 59, 61, 63, 136, 138, 140, 142, 143, 145, 147, 149, 151, 153, 154, 156, 158, 160, 161, 162, and 164.

34. A system comprising:
(i) the fusion protein of any preceding claim or a polynucleotide encoding the fusion protein; and
(ii) a guide nucleic acid or a polynucleotide encoding the guide nucleic acid, the guide nucleic acid comprising:
(1) a scaffold sequence capable of forming a complex with the napDNAbd; and
(2) a guide sequence capable of hybridizing to the target sequence on the target strand of the target dsDNA, thereby guiding the complex to the target dsDNA.

35. The fusion protein of any preceding claim, wherein the guide nucleic acid is a guide RNA (gRNA).

36. The fusion protein of any preceding claim, wherein the scaffold sequence has substantially the same secondary structure as the secondary structure of the sequence of SEQ ID NO: 40, 73, or 74, or wherein the scaffold sequence comprises (1) a sequence of SEQ ID NO: 40, 73, or 74 or a 5′ or 3′ end truncation thereof with 1, 2, 3, 4, 5, or 6, nucleotides truncated at the 5′ or 3′ end; or (2) a sequence having a sequence identity of at least about 70%, 75%, 80%, 85%, 90%, 95%, or 100% to SEQ ID NO: 40, 73, or 74 or a 5′ or 3′ end truncation thereof with 1, 2, 3, 4, 5, or 6 nucleotides truncated at the 5′ or 3′ end; or (3) a sequence having at most 1, 2, 3, 4, 5, or 6 nucleotide differences, whether consecutive or not, compared to SEQ ID NO: 40, 73, or 74.

37. The fusion protein or system of any preceding claim, wherein the fusion protein or system further comprises a translesion synthesis (TLS) polymerase or a recruiting domain or component capable of recruiting a TLS polymerase.

38. The fusion protein or system of any preceding claim, wherein TLS polymerase is selected from the group consisting of Polα (alpha), Polβ (beta), Polδ (delta) (PCNA), Polγ (gamma), Polη (eta), Polι (iota), Polκ (kappa), Polλ (lamda), Polμ (mu), Polν (nu), Polθ (theta), and REV1.

39. A method of modifying a target dsDNA, comprising contacting the target dsDNA with the system of any preceding claim, the target dsDNA comprising:
(a) a first deoxyribonucleotide (e.g., dG (deoxyguanosine), dT (thymidine), dC (deoxycytidine)) in a protospacer sequence on a nontarget strand (edited strand) of the target dsDNA, and
(b) a second deoxyribonucleotide (e.g., dC (deoxycytidine), dA (deoxyadenosine), dG (deoxyguanosine)) base pairing with the first deoxyribonucleotide (e.g., dG, dT, dC) and in a target sequence on the target strand (non-edited strand) of the target dsDNA, wherein the protospacer sequence is fully reverse complementary to the target sequence;
wherein the method does not include deamination of the base of the first deoxyribonucleotide before the excision of the base of the first deoxyribonucleotide.

40. The method of any preceding claim, wherein the method does not include deamination of the base of the first deoxyribonucleotide.

41. An MPG as defined in any preceding claim.

42. An UNG as defined in any preceding claim.
